(19) United States
(12) Patent Application Publication
(10) Pub. No.: US 2026/0308282 A1
(43) Pub. Date: Oct. 8, 2026

(54) ANTI-NME ANTIBODY AND METHOD OF TREATING CANCER OR CANCER METASTASIS

(71) Applicant: MINERVA BIOTECHNOLOGIES CORPORATION, Waltham, MA (US)

(72) Inventors: Cynthia BAMDAD, Boston, MA (US); Benoit SMAGGHE, Honolulu, HI (US)

(21) Appl. No.: 19/287,506

(22) Filed: Jul. 31, 2025

Related U.S. Application Data
(62) Division of application No. 18/892,093, filed on Sep. 20, 2024, now Pat. No. 12,415,868.
Application No. 18/892,093 is a division of application No. 18/002,832, filed on Jun. 27, 2021, now abandoned.
Application No. 18/002,832 is a continuation of application No. PCT/US2021/036500, filed on Jun. 8, 2021.
(60) Provisional application No. 63/044,670, filed on Jun. 26, 2020.
(60) Provisional application No. 63/046,852, filed on Jul. 1, 2020.

(51) Int. Cl. C07K 16/46; A61P 35/00; C07K 16/40

(52) U.S. Cl. CPC C07K 16/468; A61P 35/00; C07K 16/40; C07K 2317/20; C07K 2317/31; C07K 2317/33; C07K 2317/565; C07K 2317/622; C07K 2317/73; C07K 2317/76

(57) ABSTRACT

The present application discloses anti-NMR antibodies and their use in treating or preventing diseases.

### CROSS REFERENCE

This application is a divisional of U.S. patent application Ser. No. 18/892,093, filed Sep. 20, 2024, which is a divisional of U.S. patent application Ser. No. 18/002,832, filed Dec. 21, 2022, which is a National Stage Entry of International Patent Application No. PCT/US2021/039291, filed Jun. 27, 2021, which claims priority to U.S. Patent Application No. 63/046,852, filed Jul. 1, 2020; claims priority to U.S. Patent Application No. 63/044,670, filed Jun. 26, 2020; and is a continuation of International Patent Application No. PCT/US2021/036500, filed Jun. 8, 2021, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jul. 31, 2025, is named 56699-744402.xml and is 998,499 bytes in size.

### BACKGROUND OF THE INVENTION

### 1. Field of the Invention

The present application relates to NME proteins, peptides derived from NME proteins, and antibodies generated from the peptides thereof or antibody or antibody fragments selected by virtue of their ability to bind to said peptides. The present application also relates to treating or preventing diseases associated with the expression of NME in a patient.

### 2. General Background and State of the Art

NDPK (nucleoside diphosphate protein kinase) proteins are a family of proteins grouped together because they all contain an NDPK domain. The first NME proteins discovered, previously called NM23 proteins, were NM23-H1 and NM23-H2. For decades it was unclear whether they induced differentiation or prevented differentiation of hematopoietic cells. The inventors previously discovered that NM23-H1 prevents differentiation when it is a dimer, which binds to the MUC1* growth factor receptor, but at higher concentrations NM23-H1 becomes a hexamer, which does not bind to MUC1*, and it induces differentiation. NM23 used to be called a metastasis suppressor when it was found that it was under-expressed in some very aggressive cancers. The present inventors previously disclosed that NM23-H1 dimers bind to and dimerize the extracellular domain of the MUC1* growth factor receptor that is over expressed on the vast majority of cancers and such binding promotes the growth of cancer cells. Conversely, at higher concentrations, NM23 forms tetramers and hexamers that do not bind to MUC1* and do not promote tumorigenesis. Very recently more NME family proteins (NME 1-10) have been discovered although until now, their functions have not been elucidated. NME7 is a newly discovered NME family protein, but its NDPK domain has no enzymatic activity, unlike other NME family members. NME7 is either not expressed at all in adult tissues or is expressed at extremely low levels.

### SUMMARY OF THE INVENTION

The present application is directed to a method of treating or preventing cancer in a subject, comprising administering to the subject an antibody made against a member of the NME family. The NME family may be NME7 family. The antibody may bind to NME7. The antibody may bind to NME7_{AB} or NME7_{AB}-like protein. The antibody may bind to NME7-X1. The antibody may inhibit binding between NME7 and its cognate binding partner. The cognate binding partner may be MUC1*. The cognate binding partner may be PSMGFR portion of the MUC1* extracellular domain. In one aspect, the antibody may be generated or selected for its ability to bind to a peptide selected from those listed in FIGS. 6-9 (SEQ ID NOS: 88 to 145). Preferably, the peptide may be selected from those listed in FIG. 9 (SEQ ID NOS: 141 to 145).

The peptide may be highly homologous to, or to which is added or subtracted up to 7, up to 6, up to 5, up to 4, up to 3, up to 2, or up to 1 amino acid residues at the N-terminus or C-terminus, of the peptides listed in FIG. 6-9 (SEQ ID NOS: 88 to 145). In one aspect, the antibody may be selected for its ability to bind to NME7_{AB} or NME7-X1 but not to NME1. The antibody may be polyclonal, monoclonal, bivalent, monovalent, bispecific, an antibody fragment containing the variable region, or an antibody mimic. The antibody may be human or humanized. The antibody may be a single chain scFv.

In another aspect, the invention is directed to a method of treating or preventing cancer in a subject, comprising administering to the subject a peptide that is highly homologous or identical to regions of NME7_{AB}. The peptide may be at least 80% homologous to one or more of the peptides listed in FIG. 6. The peptide may be at least 80% homologous to one or more of the peptides listed in FIG. 7. The peptide may be at least 80% homologous to one or more of the peptides listed in FIG. 8. The peptide may be at least 80% homologous to one or more of the peptides listed in FIG. 9. The peptide may be selected from peptides listed in FIG. 6-9 (SEQ ID NOS: 88 to 145). The peptide may be selected from those listed in FIG. 9 (SEQ ID NOS: 141 to 145). Or, the peptide may be highly homologous to, or to which is added or subtracted up to 7, up to 6, up to 5, up to 4, up to 3, up to 2, or up to 1 amino acid residues at the N-terminus or C-terminus, of the peptides listed in FIG. 6-9 (SEQ ID NOS: 88 to 145). The peptide may be connected to another peptide via a spacer or linker.

In another aspect, the invention is directed to a chimeric antigen receptor (CAR), for the treatment or prevention of cancer wherein the targeting extracellular portion of the CAR comprises at least a peptide fragment of a member of the NME family. NME family may be NME7 family. The member of the NME7 family may be NME7. Or, the member of the NME7 family may be NME7_{AB} or NME7_{AB}-like protein. The member of the NME7 family may be also NME7-X1. The targeting extracellular portion of the CAR may include a peptide of the peptides listed in FIG. 6-9 (SEQ ID NOS: 88 to 145). The peptide may be selected from those listed in FIG. 9 (SEQ ID NOS: 141 to 145). The peptide may include a peptide, which is highly homologous to, or to which is added or subtracted up to 7, up to 6, up to 5, up to 4, up to 3, up to 2, or up to 1 amino acid residues at the N-terminus or C-terminus, of the peptides listed in FIG. 6-9 (SEQ ID NOS: 88 to 145). The peptide may be connected to another peptide via a spacer or linker.

In yet another aspect, the invention is directed to a method of treating or preventing cancer or cancer metastasis, comprising engineering the chimeric antigen receptor according to claim 3, into an immune system cell and administering the cell to a subject in need thereof.

In another aspect, the invention is directed to a chimeric antigen receptor (CAR), for the treatment or prevention of cancer, wherein the targeting extracellular portion of the chimeric antigen receptor comprises a portion of an antibody that binds to NME7_{AB}, NME7_{AB}-like protein or NME7-X1. The portion of the antibody may be a single chain scFv or may be human or humanized.

In yet another aspect, the invention is directed to a method of vaccinating a person against cancer or metastatic cancer comprising immunizing the person with a peptide fragment of a member of the NME family. The NME family may be NME7 family. The member of the NME7 family may be NME7 or NME7b. The member of the NME7 family may be NME7_{AB} or NME7_{AB}-like protein. The NME7 family may be NME7-X1. The immunizing peptide may be a peptide from the peptides listed in FIG. 6-9 (SEQ ID NOS: 88 to 145). Preferably, the peptide may be selected from those listed in FIG. 9 (SEQ ID NOS: 141 to 145). The immunizing peptide may include a peptide, which is highly homologous to, or to which is added or subtracted up to 7, up to 6, up to 5, up to 4, up to 3, up to 2, or up to 1 amino acid residues at the N-terminus or C-terminus, of the peptides listed in FIG. 6-9 (SEQ ID NOS: 88 to 145). The immunizing peptide may be connected to another peptide via a spacer or linker.

In yet another aspect, the invention is directed to a method of treating or preventing cancer in a subject, comprising administering to the subject a nucleic acid that inhibits the expression of NME7, NME7b, NME7_{AB}-like protein or NME7-X1. The nucleic acid may be an anti-sense nucleic acid that suppresses expression of NME7, NME7_{AB}-like protein or NME7-X1. The nucleic acid may be an inhibitory RNA, siRNA, RNAi, or shRNA that inhibits expression of NME7, NME7_{AB}-like protein or NME7-X1.

In another aspect, the invention is directed to a method of treating or preventing cancer in a subject, comprising administering to the subject genetically edited nucleic acids that inhibit expression of NME7, NME7b, NME7_{AB}-like protein or NME7-X1. The genetically edited nucleic acids that inhibit expression of NME7, NME7b, NME7_{AB}-like protein or NME7-X1 may be inserted into cells that may be then administered to the patient. The genetically edited nucleic acids that inhibit expression of NME7, NME7b, NME7_{AB}-like protein or NME7-X1 may be inserted into cells using a viral vector. The viral vector may be a lentiviral system.

In another aspect, the invention is directed to a method of growing cancer cells comprising contacting the cells with NME7_{AB}, NME7b, NME7_{AB}-like protein or NME7-X1, 2i or 5i. The method may include culturing the cells in a medium that contains NME7_{AB}, NME7b, NME7_{AB}-like protein or NME7-X1, 2i or 5i, or growing cells in an animal that expresses human NME7_{AB}, NME7b, NME7_{AB}-like protein or NME7-X1, or to which NME7_{AB}, NME7b, NME7_{AB}-like protein or NME7-X1 is administered. The cancer cells may be breast, prostate, ovarian, colorectal, pancreatic, liver, melanoma or brain cancer cells. Drug candidates may be tested on the cells. The efficacy of the drugs may be assessed by comparing cancer growth to a no drug control or comparing expression levels of metastatic markers or stem cell markers to a no drug control or comparing the ability of the resultant cells to form tumors in animals from low cell copy number compared to a no drug control and determining the efficacy of a candidate drug for the treatment of cancer or metastasis. The cells may be obtained from a patient being assessed for treatment for cancer and drugs that would be effective for that patient are selected based on results using methods described above. The cells may not be obtained from a patient being assessed for treatment for cancer but drugs that would be effective for that patient are selected based on results using the methods described above.

In another aspect, the invention is directed to a method of generating antibodies or antibody-like molecules from peptides or peptide mimics having a sequence derived from the sequence of NME. The NME may be NME7. The peptide may be used as an immunogen to generate antibodies or antibody-like molecules. The peptide may be administered to an animal to generate anti-NME7 antibodies. The peptide may be administered to a human to generate anti-NME7 antibodies. The peptide may have a sequence listed in FIG. 6-9 (SEQ ID NOS: 88 to 145). Preferably, the peptide may be selected from those listed in FIG. 9 (SEQ ID NOS: 141 to 145). The peptide may include a peptide, which is highly homologous to, or to which is added or subtracted up to 7, up to 6, up to 5, up to 4, up to 3, up to 2, or up to 1 amino acid residues at the N-terminus or C-terminus, of the peptides listed in FIG. 6-9 (SEQ ID NOS: 88 to 145).

In another aspect, the invention is directed to a method of detecting presence of cancer or the progression of cancer, comprising the steps of:
1) obtaining a sample from a patient having cancer or at risk of developing a cancer;
2) subjecting that sample to an assay capable of detecting or measuring levels of a member of the NME7 family, or levels of nucleic acids encoding a member of the NME7 family;
3) comparing levels of the measured member of the NME7 family or the member of the NME7 family-encoding nucleic acids in the test sample to levels in control patients or control cells;
4) determining that the levels of the member of the NME7 family or nucleic acids encoding the member of the NME7 family are elevated compared to the controls; and
5) concluding that the donor of the test sample has cancer or has had a progression of cancer if the control to which the test was compared came from a donor previously diagnosed with a cancer. In this method, the detection of the member of the NME7 family in circulation or in a tissue may be an indicator of cancer in a patient. The member of the NME7 family may be NME7, NME7b, NME7-X1, or NME7_{AB}-like protein.

In yet another aspect, the invention is directed to a method comprising:
detecting presence of a member of the NME7 family or MUC1* in a patient; and
administering anti-NME7 or anti-MUC1* antibody or antibodies to the patient exhibiting the member of the NME7 family or MUC1* expression. The member of the NME7 family may be NME7, NME7b, NME7-X1, or NME7_{AB}-like protein.

In yet another aspect, the invention is directed to a method for treating or preventing cancer comprising:
1) obtaining a sample from a patient suspected of having a cancer or at risk of developing a cancer or at risk of developing a metastatic cancer;
2) measuring an amount of the member of an NME7 family or a member of the NME7 family encoding nucleic acid, wherein the measured levels are significantly above those measured in a control sample;
3) determining that the patient has a cancer or has developed a more aggressive or a metastatic cancer;
4) administering to the patient an effective amount of a therapeutic agent that suppresses expression of the member of the NME7 family, inhibits cleavage of NME7 or inhibits NME7 binding to its targets. The target of the member of the NME7 family may be MUC1*. The target of the member of the NME7 family may be PSMGFR portion of the MUC1* extracellular domain. The member of the NME7 family may be NME7, NME7b, NME7-X1, or NME7_{AB}-like protein.

In any of the methods above regarding cancer, cancer may include breast, prostate, ovarian, colorectal, pancreatic, liver, melanoma or brain cancer.

In one aspect, the invention is directed to an NME7 specific antibody or fragment thereof that binds to the NME7 B3 peptide of SEQ ID NO:145 or SEQ ID NO:169. The antibody may be monoclonal antibody or bivalent, monovalent, an Fab, or a single chain variable fragment antibody (scFv). The antibody may be linked to an antibody drug conjugate. The drug may be linked to a toxin or pro-toxin.

The invention is also directed to an isolated nucleic acid encoding the antibody.

The invention is also directed to an isolated hybridoma expressing the monoclonal antibody discussed above. The antibody may specifically bind to NME7_{AB} or NME7-X1, but not to NME1. The antibody may disrupt interaction between NME7_{AB} and MUC1* extra cellular domain or between NME7-X1 and MUC1* extra cellular domain. Or, the antibody may disrupt binding between NME7_{AB} and PSMGFR or between NME7-X1 and PSMGFR. Further, the antibody may disrupt binding between NME7_{AB} and N-10 or between NME7-X1 and N-10.

In another aspect, the antibody may not disrupt interaction between NME7_{AB} and MUC1* extra cellular domain or between NME7-X1 and MUC1* extra cellular domain. NME7_{AB} or NME7-X1 may bind to the N-10 peptide (SEQ ID NO:170) but not to a C-10 peptide (SEQ ID NO:171). In particular, the antibody may be 5A1, 4A3, 5D4, or 4P3.

The antibody may comprise an amino acid sequence in the heavy chain variable region comprising the following:
in the CDR1 region YTFTNYGMN (SEQ ID NO:439);
in the CDR2 region WINTYTGEPTYVDDFKG (SEQ ID NO:440); and
in the CDR3 region LRGIRPGPLAY (SEQ ID NO:441); and
an amino acid sequence in the light chain variable region comprising the following:
in the CDR1 region SASSSVSYMN (SEQ ID NO:444);
in the CDR2 region GISNLAS (SEQ ID NO:445); and
in the CDR3 region QQRSSYPPT (SEQ ID NO:446).

An example of such an antibody above is 5A1.

In another aspect, the antibody may comprise an amino acid sequence in the heavy chain variable region comprising the following:
in the CDR1 region NTFTEYTMH (SEQ ID NO:429);
in the CDR2 region GFNPNNGVTNYNQKFKG (SEQ ID NO:430); and
in the CDR3 region RYYHSTYVFYFDS (SEQ ID NO:431); and
an amino acid sequence in the light chain variable region comprising the
following:
in the CDR1 region SASQGISNYLN (SEQ ID NO:434);
in the CDR2 region YTSSLHS (SEQ ID NO:435); and
in the CDR3 region QQYSKLPYT (SEQ ID NO:436).

An example of such an antibody above is 5D4.

In another aspect, the antibody may comprise an amino acid sequence in the heavy chain variable region comprising the following:
in the CDR1 region NTFTEYTMH (SEQ ID NO:388);
in the CDR2 region GFNPNNGVTNYNQKFKG (SEQ ID NO:389); and
in the CDR3 region RYYHSLYVFYFDY (SEQ ID NO:390); and
an amino acid sequence in the light chain variable region comprising the
following:
in the CDR1 region SASQGISNYLN (SEQ ID NO:434);
in the CDR2 region YTSSLHS (SEQ ID NO:435); and
in the CDR3 region QQYSKLPYT (SEQ ID NO:436).

An example of such an antibody above is 4A3.

In another aspect, the antibody may comprise an amino acid sequence in the heavy chain variable region comprising the following:
in the CDR1 region NTFTEYTMH (SEQ ID NO:388);
in the CDR2 region GFNPNNGVTNYNQKFKG (SEQ ID NO:389); and
in the CDR3 region RYYHSLYVFYFDY (SEQ ID NO:390); and
an amino acid sequence in the light chain variable region comprising the following:
in the CDR1 region ITSTDIDDDMN (SEQ ID NO: 1144);
in the CDR2 region EGNTLRP (SEQ ID NO: 1145); and
in the CDR3 region LQSDNLPLT (SEQ ID NO: 1146).

An example of such an antibody above is 4P3.

The antibody may be human, humanized or an engineered antibody mimic.

The antibody may be non-human, such as murine or camelid.

The invention is also directed to a method of administering to a patient for prevention or treatment of cancer comprising administering to the patient a composition comprising the antibody described above.

The invention is also directed to a method for preventing or treating cancer metastasis in a patient, comprising administering to the patient a composition comprising the antibody described above.

The invention is also directed to a method for diagnosing cancer or cancer metastasis comprising contacting a patient specimen and normal specimen with the antibody above, and comparing the results from both specimen, wherein presence of positive binding to the antibody in the patient specimen indicates the presence of cancer or cancer metastasis in the patient. The antibody may be linked to an imaging agent. The patient specimen may be blood, bodily fluid, tissue, circulating cells, in vitro, in vivo, including intra-operative.

The invention is also directed to a cell that is engineered to express an anti-NME7_{AB} antibody or fragment thereof. The cell may be an immune cell, such as T cell or NK cell, or a stem or progenitor cell, preferably stem or progenitor cell that is then differentiated to become a T cell.

The cell may comprise a chimeric antigen receptor (CAR) that recognizes tumor associated antigen. Expression of the anti-NME7 antibody may be inducible. Nucleic acid encoding an anti-NME7_{AB} antibody may be inserted into the Foxp3 enhancer or promoter. The anti-NME7_{AB} antibody may be in an NFAT-inducible system. NFATc1 response element may be inserted upstream of the antibody sequence that is inserted into Foxp3 enhancer or promoter region.

The anti-NME7_{AB} antibody or fragment thereof may bind to the NME7 B3 peptide, or disrupt binding of NME7_{AB} or NME7-X1 to the PSMGFR peptide of the MUC1* extra cellular domain.

The CAR may recognize a tumor associated antigen and an anti-NME7 antibody. The tumor associated antigen may be MUC1*.

The invention is also directed to an anti-cancer vaccine comprising a composition comprising one or more peptides derived from NME7_{AB} listed in FIG. 6-FIG. 9 or a peptide having at least 80%, 85%, 90%, 95%, 97% sequence identity thereof as the immunogenicity eliciting portion. The peptide may be a peptide of SEQ ID NOS: 141-145 or a peptide having at least 80%, 85%, 90%, 95%, 97% sequence identity thereof. The peptide may be a peptide of SEQ ID NO: 145 or a peptide having at least 80%, 85%, 90%, 95%, 97% sequence identity thereof.

In another aspect, the invention is directed to a BiTE comprising the above-described antibody.

In yet another aspect, the invention is directed to a method of generating anti-NME7_{AB} antibodies wherein Cysteine residue in the NME7 B3 peptide is mutated to avoid disulfide bonding.

In yet another aspect, the invention is directed to a method of generating cells with enhanced metastatic potential comprising culturing the cells with NME7_{AB} or NME7-X1.

The invention is also directed to a cell that is engineered to express NME7_{AB} or NME7-X1, a transgenic animal that expresses NME7_{AB} or NME7-X1, wherein the NME7_{AB} or NME7-X1 may be human, and also wherein expression of NME7_{AB} or NME7-X1 may be inducible.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIGS. 1A-1B show a graph of HRP signal from ELISA sandwich assay showing NME7_{AB} dimerizes MUC1* extra cellular domain peptide.

FIG. 2 is a graph of RT-PCR measurements of gene expression for stem cell markers and cancer stem cell markers for T47D cancer cells after being cultured in traditional media or a media containing NME7, wherein cells that became non-adherent (floaters) were analyzed separate from those that remained adherent.

FIG. 3 is a graph of RT-PCR measurements of gene expression for a variety of stem and putative cancer stem cell markers for DU145 prostate cancer cells. Cells were cultured either in traditional media or a media containing NME1 dimers (“NM23”) or NME7 (NME7_{AB}). Rho kinase inhibitor was not used because by passage 2, cells remained adherent.

FIG. 4 is a graph of RT-PCR measurement of the metastatic markers and pluripotent stem cell markers showing that the 2i inhibitors (GSK3-beta and MEK inhibitors), which were previously shown to revert stem cells to a more naïve state, also induce cancer cells to a more metastatic state although not as well as NME7_{AB}.

FIG. 5 is a sequence alignment between human NME1 and human NME7-A or -B domain. Figure discloses SEQ ID NOS 1137, 1140, 1137 and 1143, respectively, in order of appearance.

FIG. 6 lists immunogenic peptides from human NME7 with low sequence identity to NME1 and selected for their ability to generate therapeutic anti-NME7 antibodies for the treatment or prevention of cancers. Figure discloses SEQ ID NOS 88-118, respectively, in order of appearance.

FIG. 7 lists immunogenic peptides from human NME7 that may be important for structural integrity or for binding to MUC1* selected for their ability to generate therapeutic anti-NME7 antibodies for the treatment or prevention of cancers. Figure discloses SEQ ID NOS 122-133, respectively, in order of appearance.

FIG. 8 lists immunogenic peptides from human NME1 that may be important for structural integrity or for binding to MUC1* and selected for their ability to generate therapeutic anti-NME7 antibodies for the treatment or prevention of cancers. Figure discloses SEQ ID NOS 134-140, respectively, in order of appearance.

FIG. 9 lists immunogenic peptides from human NME7 selected for their low sequence identity to NME1 and for their homology to bacterial NME1 proteins that have been implicated in cancers. These peptides are preferred for their ability to generate therapeutic anti-NME7 antibodies for the treatment or prevention of cancers. The peptides shown in this Figure include and added Cysteine covalently bound at the C-terminal end. Figure discloses SEQ ID NOS 141-145, and 169, respectively, in order of appearance.

FIGS. 10A-10B show graphs of ELISA assays in which either NME7_{AB} (FIG. 10A) or NME1 (FIG. 10B) is adsorbed to the plate and anti-NME7 antibodies generated by NME7 peptides A1, A2, B1, B2 and B3 are tested for their ability to bind to NME7 but not to NME1. C20 is an anti-NME1 antibody.

FIG. 11 shows graphs of ELISA assays in which anti-NME7 antibodies generated are tested for their ability to inhibit binding of NME7_{AB} to a surface immobilized MUC1* peptide but not inhibit binding of NME1.

FIG. 12 shows a graph of a cancer cell growth experiment in which breast cancer cells were grown in the presence or absence of NME7 antibodies or short peptides derived from NME7, which were used to generate or select the antibodies. In addition, an antibody generated by immunization with nearly the entire NME7_{AB} peptide, amino acids 100-376, was shown to inhibit cancer cell growth.

FIG. 13 shows a graph of a cancer cell growth experiment in which breast cancer cells were grown in the presence or absence of combinations of NME7 antibodies or combinations of the short peptides derived from NME7, which were used to generate or select the antibodies. Both antibodies as well as their immunizing NME7_{AB} peptides inhibited growth of cancer cells.

FIGS. 14A-14B show tables of scientist observations when cancer cells were grown in either NME7_{AB} or 2i inhibitors, which both are able to transform cancer cells to a more metastatic state, and in the presence or absence of NME7 derived peptides A1, A2, B1, B2 and B3. The NME7_{AB} peptides inhibited the transition of adherent cancer cells to the floater cells, which RT-PCR measurements show have increased expression of metastatic markers, especially CXCR4.

FIGS. 15A-15C show graphs of RT-PCR measurements of expression of CXCR4 and other metastatic markers in T47D breast cancer cells that were grown in either NME7_{AB} or 2i inhibitors, each of which transform cancer cells to a more metastatic state, and the inhibitory effect of anti-NME7 antibodies on the metastatic transformation. FIG. 15A shows PCR graph of CXCR4 expression of T47D cancer cells grown in NME7_{AB} or 2i in the presence or absence of anti-NME7 antibodies. FIG. 15B shows a graph of RT-PCR measurements of CXCR4, CHD1 and SOX2 expression in T47D breast cancer cells that were grown in 2i inhibitors for 72 hours or 144 hours, in the presence of NME7_{AB} immunizing peptides and shows the peptides are themselves inhibitory to the metastatic transformation. Peptides A1, A2 and B1 which were used in the inhibitory Combo 2 and 3 in FIG. 15A are also inhibitory as peptides. Peptide B3 is the most inhibitory and is the immunizing peptide for antibody 61 which was the most inhibitory antibody tested in FIG. 15A. FIG. 15C shows the graph of FIG. 15B with the scale of the Y-axis reduced.

FIG. 16 shows a table of recorded RNA levels in samples that were used for RT-PCR measurement of CXCR4 in FIG. 31 as well as the threshold cycle number for CXCR4 expression as well as for the control housekeeping gene.

FIG. 17 shows a graph of RT-PCR measurement of the expression of NME7-X1 in a panel of human stem cells and cancer cells.

FIG. 18 shows a graph of RT-PCR measurement of the expression of NME7, NME7a, NME7b and NME7-X1 in a panel of human stem cells and cancer cells. NME7a is full-length NME7, NME7b is missing a small portion of the DM10 domain, NME7-X1 is missing all of the DM10 domain and a small portion of the N-terminus of the first NDPK A domain. The bar labeled NME7 means that primers were used that detected both NME7a and NME7b.

FIGS. 19A-19F show photographs of Western blots in which various cancer cell lines are probed for expression of NME7 species using antibodies generated by immunization with NME7 derived peptides. FIG. 19A shows Western blot wherein antibody 52 that binds to the A1 peptide is used to probe a panel of cells for the presence of full-length NME7, NME7_{AB} or NME7-X1. FIG. 19B shows Western blot wherein antibody 56 that binds to the B1 peptide is used to probe a panel of cells for the presence of full-length NME7, NME7_{AB} or NME7-X1. FIG. 19C shows Western blot wherein antibody 61 that binds to the B3 peptide is used to probe a panel of cells for the presence of full-length NME7, NME7_{AB} or NME7-X1. FIG. 19D shows Western blot wherein commercially available polyclonal antibody H278, raised against both the NME7 A and B domain, is used to probe a panel of cells for the presence of NME7. As the figure shows, antibody H278 also recognizes NME1. FIG. 19E shows a gel published on website for commercially available anti-NME7 antibody B9, showing it binds to a species with an apparent molecular weight of full-length NME7. FIG. 19F shows a Western blot in which we used anti-NME7 antibody B9 to probe a gel that was loaded only with NME1. As can be seen in the figure, antibody B9 recognizes NME1 as well as full-length NME7. This is not surprising because like antibody H278, B9 was raised against both A and B domains of NME7 where the A domain of NME1 is highly homologous to the A domain of NME7_{AB}.

FIGS. 20A-20C show graphs of RT-PCR measurements of metastatic markers in cancer cells after being cultured in a serum-free media containing NME7_{AB} compared to the standard media. FIG. 20A shows SK-OV3, a MUC1-positive ovarian cancer cell line increased expression of metastatic markers CXCR4, CDH1 aka E-cadherin, SOX2 and NME7-X1; FIG. 20B shows OV-90 a MUC1-negative ovarian cancer cell line increased expression of metastatic markers CXCR4 and NME7-X1; FIG. 20C shows MDA-MB a breast cancer cell line that expresses minimal levels of MUC1 increased expression of metastatic markers CDH1 aka E-cadherin and SOX2.

FIGS. 21A-21F show photographs of Western blots and description of cancer cell lines analyzed. For Western blots in FIGS. 21A and 21B, all cancer samples were normalized such that they were loaded onto gel at a concentration of 40 ug/mL. In FIG. 21A, various cancer cell lines are probed for the expression of full-length MUC1 using an anti-tandem repeat monoclonal antibody VU4H5. In FIG. 21B, various cancer cell lines are probed for the expression of cleaved form MUC1* using a polyclonal anti-PSMGFR antibody. FIG. 21C is a description of the cancer cell lines analyzed. FIG. 21D shows that HER2 positive BT474 breast cancer cells, marked “BT474 (parent cells)” express little to no MUC1 or MUC1* until they acquire resistance to Herceptin and other chemotherapy drugs, marked “BTRes1” in figure. Parent cells were made resistant to Herceptin, Taxol, Doxorubicin and cyclophosphamide by culturing the cells in sub-lethal levels of Herceptin. FIG. 21D shows that the expression level of HER2 has not changed but expression of MUC1* has dramatically increased as the cells acquired resistance to Herceptin. FIG. 21E shows a graph of the growth of the parent BT474 cells compared to the drug resistant metastatic cells in response to treatment with Herceptin in the presence or absence of an anti-MUC1* Fab. As can be seen in the figure, the BT474 parent cells show a Herceptin concentration dependent decrease in cell growth, whereas the two Herceptin resistant cell lines, BTRes 1 and BTRes2, show no decrease in cancer cell growth in response to treatment with Herceptin. However, when treated with an anti-MUC1* Fab, the resistant cell lines show a Herceptin concentration dependent decrease in cancer cell growth. FIG. 21F shows a graph of the percent cell death of the parent BT474 cells compared to the drug resistant BTRes1 cells, in response to treatment with Taxol in the presence or absence of an anti-MUC1* Fab.

FIGS. 22A-22E show photographs of Western blots of a co-immunoprecipitation experiment. T47D breast cancer cell extracts were incubated with an antibody against the MUC1 cytoplasmic tail, Ab-5, or a control antibody, IgG, and co-immunoprecipitated. The gels were blotted with two different commercially available anti-NME7 antibodies B9 (FIG. 22A) and CF7 (FIG. 22B). Both gels show unique NME7 bands at ~33 kDa and ~30 kDa. The gels were stripped and re-probed with an antibody against the extracellular domain of MUC1*, anti-PSMGFR (FIG. 22C) and (FIG. 22D), which shows that the NME7 species and MUC1* interact. A recombinant NME7_{AB} and a recombinant NME7-X1 were mixed together and run on a gel, then probed with an anti-NME7 antibody, showing that the two unique NME7 species that are naturally occurring in breast cancer cells and that interact with MUC1* are an NME7_{AB}-like species and NME7-X1 (FIG. 22E).

FIGS. 23A-23C show photographs of Western blots of a co-immunoprecipitation experiment. Human induced pluripotent stem, iPS7, or embryonic stem, HES3, cell extracts were incubated with an antibody against the MUC1 cytoplasmic tail, Ab-5, or a control antibody, IgG, and co-immunoprecipitated. The gel was blotted with a commercially available anti-NME7 antibody B9 (FIG. 23A). Both cell types show unique NME7 bands at ~33 kDa and ~30 kDa. The gel was stripped and re-probed with an antibody against the extracellular domain of MUC1*, anti-PSMGFR (FIG. 23B), which shows that the NME7 species and MUC1* interact. A recombinant NME7_{AB} and a recombinant NME7-X1 were mixed together and run on a gel, then probed with an anti-NME7 antibody, showing that the two unique NME7 species that are naturally occurring in breast cancer cells and that interact with MUC1* are an NME7_{AB}-like species and NME7-X1 (FIG. 23C).

FIG. 24 shows a graph of an ELISA experiment assaying new anti-NME7 antibodies for their ability to bind to NME7_{AB}. NME7_{AB} is known to bind to the extra cellular domain of MUC1*. The surface of the multi-well plate was coated with a recombinant NME7_{AB}. Anti-NME7_{AB} antibodies were separately added to wells. Standard washes were performed and visualized by adding an HRP-conjugated secondary antibody. As can be seen, 7 of the 10 new anti-NME7 antibodies bound strongly to NME7_{AB}.

FIG. 25 shows a graph of an ELISA experiment assaying new anti-NME7 antibodies for their ability, or preferably inability, to bind to NME1. The surface of the multi-well plate was coated with a recombinant NME1-S120G dimers, which are also known to bind to the MUC1* extra cellular domain. Anti-NME7_{AB} antibodies were separately added to wells. Standard washes were performed and visualized by adding an HRP-conjugated secondary antibody. As can be seen only one antibody showed just minimal binding to NME1.

FIG. 26 shows a graph of an ELISA competitive inhibition assay. NME7_{AB}/anti-NME7 antibody complexes were made before adding to a multi-well plate coated with MUC1* extra cellular domain peptide, PSMGFR. Recall that NME7_{AB} has two pseudo-identical domains A and B that are each able to bind to MUC1* extra cellular domain. Antibodies that bind to the NME7 B3 peptide, which is in the B domain, do not bind to the NME7 A domain. Therefore, only partial inhibition of the NME7_{AB}/MUC1* interaction is expected.

FIG. 27 shows a graph of an ELISA displacement assay. NME7_{AB} was first bound to surface-immobilized MUC1* extra cellular domain peptide on the plate, then disrupted by the addition of anti-NME7 antibodies.

FIG. 28 shows a graph of an ELISA displacement assay. In this case, the multi-well plate was coated with a truncated MUC1* peptide, N-10, which has the 10 N-terminal amino acids missing of the PSMGFR sequence. NME7_{AB} is known to bind to the N-10 peptide. NME7_{AB} was bound to surface-immobilized N-10 peptide on the plate, then disrupted by the addition of anti-NME7 antibodies.

FIG. 29 shows a graph of the amount of RNA present in samples of T47D breast cancer cells were cultured in either their normal recommended media, RMPI, serum-free media containing only NME7_{AB} as the growth factor at 4 nM, which is optimal, or 8 nM, or serum-free media containing only NME1 S120G dimers as the growth factor at 8 nM; because NME1 is a homodimer and NME7_{AB} is a monomer comprised of two pseudo-identical domains, 8 nM NME1 is the molar equivalent of 4 nM NME7_{AB}. The cancer cells were cultured in the presence or absence of anti-NME7 B3 antibodies. In this experiment, floating cells were separated from the adherent cells and analyzed separately. Significant data argues that the floater cells are the cancer stem cells. An increase or decrease in the amount of RNA in a sample argues that an agent increased or decreased, respectively, the number of cells in a given population that were generated.

FIG. 30 shows a graph of a PCR measurement of metastatic marker CXCR4 in T47D breast cancer cells that were cultured in either their normal recommended media, RPMI, serum-free media containing only NME7_{AB} as the growth factor at 4 nM, which is optimal, or 8 nM, or serum-free media containing only NME1 S120G dimers as the growth factor at 8 nM; because NME1 is a homodimer and NME7_{AB} is a monomer comprised of two pseudo-identical domains, 8 nM NME1 is the molar equivalent of 4 nM NME7_{AB}. The cancer cells were cultured in the presence or absence of anti-NME7 B3 antibodies. In this experiment, floating cells were separated from the adherent cells and analyzed separately. Significant data argues that the floater cells are the cancer stem cells. As can be seen in the figure, growth in NME7_{AB} media increases CXCR4 in the floater population of cells and anti-NME7 B3 antibodies decreased its expression, arguing that anti-NME7 antibodies decreased generation of cancer stem cells.

FIG. 31 shows a graph of a PCR measurement of stem cell marker and metastatic marker SOX2 in T47D breast cancer cells that were cultured in either their normal recommended media, RMPI, serum-free media containing only NME7_{AB} as the growth factor at 4 nM, which is optimal, or 8 nM, or serum-free media containing only NME1 S120G dimers as the growth factor at 8 nM; because NME1 is a homodimer and NME7_{AB} is a monomer comprised of two pseudo-identical domains, 8 nM NME1 is the molar equivalent of 4 nM NME7_{AB}. The cancer cells were cultured in the presence or absence of anti-NME7 B3 antibodies. In this experiment, floating cells were separated from the adherent cells and analyzed separately. Significant data argues that the floater cells are the cancer stem cells. As can be seen in the figure, growth in NME7_{AB} media increases SOX2 expression in the floater population of cells and anti-NME7 B3 antibodies decreased its expression, arguing that anti-NME7 antibodies decreased generation of cancer stem cells.

FIG. 32 shows a graph of a PCR measurement of stem cell marker and metastatic growth factor receptor MUC1 in T47D breast cancer cells that were cultured in either their normal recommended media, RMPI, serum-free media containing only NME7_{AB} as the growth factor at 4 nM, which is optimal, or 8 nM, or serum-free media containing only NME1 S120G dimers as the growth factor at 8 nM; because NME1 is a homodimer and NME7_{AB} is a monomer comprised of two pseudo-identical domains, 8 nM NME1 is the molar equivalent of 4 nM NME7_{AB}. The cancer cells were cultured in the presence or absence of anti-NME7 B3 antibodies. In this experiment, floating cells were separated from the adherent cells and analyzed separately. Significant data argues that the floater cells are the cancer stem cells. As can be seen in the figure, growth in NME7_{AB} media increases MUC1 expression in the floater population of cells and anti-NME7 B3 antibodies decreased its expression, arguing that anti-NME7 antibodies decreased generation of cancer stem cells.

FIGS. 33A-33B show IVIS photographs of immune compromised nu/nu mice Day 6 post tail vein injection of cancer cells. FIG. 33A shows IVIS photographs of mice injected with 500,000 T47D-wt breast cancer cells. FIG. 33B shows IVIS photographs of mice injected with 10,000 T47D breast cancer cells that were grown for 10 days in NME7_{AB} in a minimal media. The floating cells were collected. These floating cells are referred to herein as cancer stem cells, CSCs. As can be seen in the figure, the mice injected with wild type cancer cells show no signs of metastasis. However, the mice injected with 50-times less cells, but cancer stem cells, show that the injected cancer cells are clearly metastasizing.

FIGS. 34A-34D show IVIS photographs of immune compromised nu/nu mice Day 10 post tail vein injection of cancer cells. FIG. 34A shows IVIS photographs of mouse injected with 500,000 T47D-wt breast cancer cells. FIG. 34B shows IVIS photographs of mouse injected with 10,000 T47D-CSC (cancer stem cells). FIG. 34C shows IVIS photographs of mouse injected with 10,000 T47D-CSC (cancer stem cells) and injected on Day 7 with anti-NME7 antibody. FIG. 34D shows the hand recording of the IVIS measure of emitted photons. As can be seen in the figure, the mouse chosen for treatment is more metastatic than the comparable T47D-CSC mouse. The efficacy of the first antibody injection may have been blocked by the Day 6 injection of free NME7_{AB}. Control mouse injected with 500,000 T47D-wt cells shows some weak emission of photons that may be background or surviving cancer cells.

FIG. 35A-35C shows IVIS photographs of immune compromised nu/nu mice Day 12 post tail vein injection of cancer cells. FIG. 35A shows IVIS photographs of mouse injected with 500,000 T47D-wt breast cancer cells. FIG. 35B shows that mouse injected with 10,000 T47D-CSC (cancer stem cells) that was not treated with anti-NME7 antibody died from excess tumor burden before IVIS photograph could be taken. FIG. 35C shows IVIS photographs of mouse injected with 10,000 T47D-CSC (cancer stem cells) and injected on Day 7 and Day 10 with anti-NME7 antibody. As can be seen in the figure, the mouse treated with anti-NME7 antibody is clearing away the cancer metastases. Control mouse injected with 500,000 T47D-wt cells shows less photon emissions indicating fewer surviving cancer cells or may be background.

FIGS. 36A-36B shows IVIS photographs of immune compromised nu/nu mice Day 14 post tail vein injection of cancer cells. FIG. 36A shows IVIS photographs of mouse injected with 500,000 T47D-wt breast cancer cells. FIG. 36B shows IVIS photographs of mouse injected with 10,000 T47D-CSC (cancer stem cells) and injected on Day 7, Day 10, and Day 12 with anti-NME7 antibody. As can be seen in the figure, the mouse treated with anti-NME7 antibody nearly completely free of cancer cell metastases. Control mouse injected with 500,000 T47D-wt cells shows no photon emissions.

FIGS. 37A-37V shows time course of IVIS photographs of immune compromised nu/nu mice from Day 6 to Day 26 post cancer cell tail vein injection. FIGS. 37A, 37C, 37E, 37G, 37I, 37K, 37M and 37O show IVIS photographs of mouse that had been injected Day 0 into the tail vein with 500,000 T47D-wt cells. FIGS. 37B, 37D, 37F, 37H, 37J, 37L, 37N and 37P show IVIS photographs of mouse that had been injected Day 0 into the tail vein with 10,000 T47D cancer stem cells, to which anti-NME7 antibody was administered from Day 7 to Day 17, whereupon treatment was suspended, then resumed on Day 21. FIGS. 37Q, 37R, 37S, 37T, and 37U show enlarged IVIS photographs of the treated mouse between Day 17, when anti-NME7 antibody treatment was suspended, through Day 21, when antibody treatment was resumed to Day 26. FIG. 37V shows the scale bar of the IVIS measurements. As can be seen in this time course, cancer cells that had been grown in NME7 readily metastasize and such metastasis can be effectively treated, prevented or reversed by treatment with an antibody that binds to NME7.

FIG. 38A-38C shows time course of IVIS photographs of immune compromised nu/nu mice from Day 6 to Day 19 post injection with either 500,000 T47D wild type breast cancer cells or 10,000 T47D cancer stem cells. FIG. 38A shows mice that were injected into the tail vein (i.v.). FIG. 38B shows mice that were injected intra-peritonealy (i.p.). FIG. 38C shows mice that were injected sub-cutaneously (s.c.).

FIGS. 39A-39C shows human lung tissue specimens stained with an anti-NME7 antibody that binds to the B3 peptide. The figure shows lack of NME7 expression on normal tissues, increasing expression of NME7 as tumor grade and metastasis increase.

FIGS. 40A-40C shows human small intestine tissue specimens stained with an anti-NME7 antibody that binds to the B3 peptide. The figure shows lack of NME7 expression on normal tissues, increasing expression of NME7 as tumor grade and metastasis increase.

FIGS. 41A-41D show human colon tissue specimens stained with an anti-NME7 antibody that binds to the B3 peptide. The figure shows lack of NME7 expression on normal tissues, increasing expression of NME7 as tumor grade and metastasis increase.

FIGS. 42A-42F shows photographs of female nu/nu mice weighing approximately 20 g each, which were injected into the tail vein with 10,000 Luciferase positive T47D metastatic breast cancer stem cells and treated with the anti-NME7_{AB} antibody 4A3 also known as 8F9A4A3. To image cancer cells, the Luciferase substrate, Luciferin, is intraperitoneally injected 10 minutes before being photographed in IVIS instrument. FIGS. 42A-42C show IVIS photographs with animals face down. FIG. 42D-42F show IVIS photographs with animals face up. FIGS. 42A and 42D show control animals injected with phosphate buffered saline solution. FIGS. 42B and 42E show a prevention model in which animals were injected with anti-NME7_{AB} antibody 4A3 24 hrs before injection of the metastatic cancer cells, then approximately every other day for a total of 12 antibody injections over 22 days. FIGS. 42C and 42F show a reversal model in which animals were injected with anti-NME7_{AB} antibody 4A3 24 hrs after injection of the metastatic cancer cells, then approximately every other day for a total of 11 antibody injections over 20 days.

FIGS. 43A-43F shows photographs of female nu/nu mice weighing approximately 20 g each, which were injected into the tail vein with 10,000 Luciferase positive T47D metastatic breast cancer stem cells and treated with the anti-NME7_{AB} antibodies 5A1, also known as 8F9A5A1, or 5D4, also known as 5F3A5D4. To image cancer cells, the Luciferase substrate, Luciferin, is intraperitoneally injected 10 minutes before being photographed in IVIS instrument. FIGS. 43A-43C show IVIS photographs with animals face down. FIGS. 43D-43F show IVIS photographs with animals face up. FIGS. 43A and 43D show control animals injected with phosphate buffered saline solution. FIGS. 43B, 43E, 43C and 43F show a prevention model in which animals were injected with anti-NME7_{AB} antibodies 24 hours before injection of the metastatic cancer cells, then approximately every other day for a total of 12 antibody injections over 22 days. Images were taken on Day 27.

FIGS. 44A-44D shows photographs of female nu/nu mice that on Day 0 were injected into the tail vein with 10,000 Luciferase positive T47D metastatic breast cancer stem cells mixed with NME7_{AB} to a final concentration of 32 nM. On Day 1 and Day 2 animals were injected into the tail vein with more 32 nM NME7_{AB}, which we have shown increases metastases. This is a system to demonstrate reversion of established metastases. On Day 7 animals were treated with individual anti-NME7_{AB} antibodies 8F9A5A1, 8F9A4A3, or 5F3A5D4. FIG. 44A shows control animals injected with phosphate buffered saline solution. FIG. 44B shows animals treated with anti-NME7_{AB} monoclonal antibody 8F9A5A1, also known as 5A1. FIG. 44C shows animals treated with anti-NME7_{AB} monoclonal antibody 8F9A4A3, also known as 4A3. FIG. 44D shows animals treated with anti-NME7_{AB} monoclonal antibody 5F3A5D4, also known as 5D4. Green arrows indicate low antibody dosage (5-7 mg/kg) over the indicated period and Red arrows indicate high dosage (15 mg/kg). As can be seen in the figure, animals treated with anti-NME7_{AB} antibodies have less metastases than the control animals even though many of the animals in the groups to be treated with antibody have more metastasis before any treatment. Higher concentrations of anti-NME7_{AB} antibody are more effective than low concentrations. For example between Day 11 and Day 17, animals were treated with high dose and most of the treated animals have cleared metastases by about Day 17. However, 1 low dose of antibody resulted in metastasis recurrence. Animals again respond to high dose treatment by Day 32.

FIGS. 45A-45B shows photographs of female nu/nu mice that on Day 0 were injected sub-cutaneously into the right flank with 10,000 Luciferase positive T47D metastatic breast cancer stem cells, mixed with NME7_{AB} to a final concentration of 32 nM, then mixed in a 1:1 vol:vol with Matrigel. Tumor engraftment was allowed to progress Day 0-Day 6. Animals were then treated i.v. by tail vein injection with anti-NME7_{AB} antibodies. Control animals were injected with PBS. FIG. 45A shows IVIS photographs of control animals. FIG. 45B shows IVIS photographs of animals injected into tail vein with a cocktail of anti-NME7_{AB} antibodies 5A1, 4A3 and 5D4 to a total concentration of 15 mg/kg. Antibodies or PBS were administered 4 times between Day 7 and Day 18. As can be seen in the figure, the anti-NME7_{AB} antibody treated animals show less metastases than the control group. In the treated group, 2 of the 5 animals have primary tumors that are larger than those in the control group. This could be because the anti-NME7_{AB} antibodies prevented the spread of the cancer cells, so they remained concentrated in the primary tumor. In this experiment, PCR analysis showed that after 11 days in culture with NME7_{AB}, the T47D breast cancer cells had upregulated CXCR4 by 109-fold, OCT4 by 2-fold, NANOG by 3.5-fold and MUC1 by 2.7-fold.

FIGS. 46A-46Q shows photographs of female nu/nu mice that on Day 0 were injected sub-cutaneously into the right flank with 10,000 Luciferase positive T47D metastatic breast cancer stem cells, mixed with NME7_{AB} to a final concentration of 32 nM, then mixed in a 1:1 vol:vol with Matrigel. Tumor engraftment was allowed to progress Day 0-Day 6. Animals were then treated i.v., by tail vein injection, with anti-NME7_{AB} antibodies. Control animals were injected with PBS. On Day 38 animals were sacrificed and livers harvested then analyzed by IVIS to detect cancer cells that had metastasized to the liver. FIGS. 46A-46B show whole body IVIS photographs of control animals that were injected with only PBS. FIGS. 46C-46D show whole body IVIS photographs of control animals that were injected with the anti-NME7_{AB} antibody 5A1. FIGS. 46E-46F show whole body IVIS photographs of control animals that were injected with the anti-NME7_{AB} antibody 4A3. FIGS. 46G-46H show whole body IVIS photographs of control animals that were injected with the anti-NME7_{AB} antibody 5D4. FIGS. 46A, 46C, 46E, and 46G are IVIS photographs taken at Day 7 before any treatment. FIGS. 46B, 46D, 46F, and 46H are IVIS photographs taken at Day 31 after anti-NME7_{AB} antibody treatment or mock treatment. As can be seen in the figure, animals in the PBS control group show metastasis (blue dots) in the whole body IVIS photographs, while animals treated with anti-NME7_{AB} antibodies do not. FIGS. 46I-46P show photographs and IVIS photographs of livers and lung harvested from animals after sacrifice. FIGS. 46I, 46K, 46M, and 46O are regular photographs. FIGS. 46J, 46L, 46N, and 46P are IVIS photographs, illuminating the cancer cells that have metastasized there. As can be seen in the figure, the anti-NME7_{AB} antibodies greatly inhibited metastasis to the liver, which is a primary site for breast cancer metastasis. FIG. 46Q is a bar graph of the measured photons emitted and enumerated by IVIS instrument for livers harvested from control animals versus the treated animals.

FIGS. 47A-47F shows photographs of immunofluorescent experiments in which various cancer cell lines are stained for the presence of NME7_{AB}. FIG. 47A shows T47D breast cancer cells stained with varying concentrations of anti-NME7_{AB} antibody 5D4. FIG. 47B shows ZR-75-1 breast cancer cells, also known as 1500s, stained with varying concentrations of anti-NME7_{AB} antibody 5D4. FIG. 47C shows H1975 non-small cell lung cancer cells stained with varying concentrations of anti-NME7_{AB} antibody 5D4. FIG. 47D shows H292 non-small cell lung cancer cells stained with varying concentrations of anti-NME7_{AB} antibody 5D4. FIG. 47E shows HPAFII pancreatic cancer cells stained with varying concentrations of anti-NME7_{AB} antibody 5D4. FIG. 47F shows DU145 prostate cancer cells stained with varying concentrations of anti-NME7_{AB} antibody 5D4. As can be seen in the figure, all the cancer cell lines we tested show strong and membranous staining for NME7_{AB}. The monoclonal antibody used in these experiments was 5D4. In parallel, NME7_{AB} antibodies 5A1 and 4A3 were used to stain the same cell lines and produced the same results.

FIGS. 48A-48I shows photographs of immunofluorescent experiments in which various lung cancer cell lines are stained for the presence of NME7_{AB}. FIGS. 48A-48C shows H1975 non-small cell lung cancer cells, which are an adenocarcinoma, stained with varying concentrations of anti-NME7_{AB} antibody 5D4. FIG. 48A is an overlay of DAPI and anti-NME7_{AB} staining. FIG. 48B shows anti-NME7_{AB} staining alone. FIG. 48C is a magnified view of the overlay of DAPI and anti-NME7_{AB} staining. FIGS. 48D-48F shows H292 non-small cell lung cancer cells, which are a mucoepidermoid pulmonary carcinoma, stained with varying concentrations of anti-NME7_{AB} antibody 5D4. FIG. 48D is an overlay of DAPI and anti-NME7_{AB} staining. FIG. 48E shows anti-NME7_{AB} staining alone. FIG. 48F is a magnified view of the overlay of DAPI and anti-NME7_{AB} staining. FIGS. 48G-48I shows H358 non-small cell lung cancer cells, which are a metastatic bronchioalveolar carcinoma, stained with varying concentrations of anti-NME7_{AB} antibody 5D4. FIG. 48G is an overlay of DAPI and anti-NME7_{AB} staining. FIG. 48H shows anti-NME7_{AB} staining alone. FIG. 48I is a magnified view of the overlay of DAPI and anti-NME7_{AB} staining.

FIG. 49A-49I shows PCR graphs of cancer cell lines, breast T47D, Lung H1975, lung H358 and pancreatic HPAFII before and after culture in NME7_{AB}. FIG. 49A measured breast metastatic marker CXCR4. FIG. 49B measured stem cell marker OCT4. FIG. 49C measured metastatic marker ALDH1. FIG. 49D measured stem cell marker SOX2. FIG. 49E measured stem cell marker NANOG. FIG. 49F measured marker CDH1, also known as E-cadherin. FIG. 49G measured metastatic marker CD133. FIG. 49H measured stem cell marker ZEB2. FIG. 49I measured stem, cancer and metastatic marker MUC1. The floater cells, also known as tumor spheres become able to grow anchorage independently and show markers of metastasis that are more elevated than the adherent cells. Animals injected with cancer stem cells are those injected with the NME7_{AB} grown floater cells. As can be seen in the figure markers of metastasis, stem cell markers, or markers of epithelial to mesenchymal transition (EMT) are elevated after culture in NME7_{AB}, indicating a transition to a more metastatic state.

FIG. 50A-50D shows IVIS photographs of NSG mice injected into the tail vein with 10,000 cancer cells that were either NCI-H358 parent cells or NCI-H358 cells after 10 days in culture with NME7_{AB}. FIGS. 50A and 50C show IVIS photographs of the mouse that was injected with the NCI-H358 lung cancer cells that had been grown in NME7_{AB} for 10 days. FIGS. 50B and 50D show IVIS photographs of the mouse that was injected with the parental NCI-H358 cells. FIGS. 50A and 50B show the IVIS photographs where mice are imaged face down. FIGS. 50C and 50D show the IVIS photographs where mice are imaged face up. As can be seen in the figure, the NME7_{AB} grown cells have greatly increased metastatic potential.

FIG. 51 shows PCR graph of a MUC1 negative prostate cancer line PC3 before and after 2 or 3 passages in culture in either dimeric NM23-H1, also known as NME1, or NME7_{AB}. The graph shows the fold difference in markers of stem cells, cancer cells as well as metastatic markers. As can be seen in the figure, repeated culture in NME1 or NME7_{AB} induces upregulation of stem, cancer and metastatic markers but also upregulates expression of MUC1 by 5-8 times.

FIGS. 52A-52E shows photographs of female nu/nu mice that on Day 0 were injected into the tail vein with 10,000 Luciferase positive T47D metastatic breast cancer stem cells mixed with NME7_{AB} to a final concentration of 32 nM. On Day 1 and Day 2 animals were injected into the tail vein with more 32 nM NME7_{AB}, which we have shown increases metastases. This is a system to demonstrate reversion of established metastases. On Day 5 animals were injected intravenously with 15 mg/kg of 8F9A4A3, aka 4A3, wherein the antibody was either the murine sequence or humanized 4A3 variant 4.8, or humanized 4A3 variant 12.3. FIG. 52A shows control animals injected with phosphate buffered saline solution. FIG. 52B shows animals treated with anti-NME7_{AB} humanized 4A3 4.8. FIG. 52C shows animals treated with anti-NME7_{AB} humanized 4A3 12.3. FIG. 52D shows animals treated with anti-NME7_{AB} monoclonal antibody murine 4A3. FIG. 52E shows a graph of the quantification of bioluminescence measurement of tumor volume, taken in an IVIS instrument. Animals were injected with either PBS or an anti-NME7 antibody at 15 mg/kg once every 3 days. Antibody hu 4A3 4.8 denotes that the sequences derived from human heavy chain antibody sequence 1.46 (SEQ ID NO:1102) and light chain sequence 1.6 (SEQ ID NO:1104) were used. Antibody hu 4A3 12.3 denotes that the sequences derived from human heavy chain antibody sequence 4.4 (SEQ ID NO:1106) and light chain sequence 4.1 (SEQ ID NO:1108) were used. Note that half the animals treated with hu 4A3 4.8 were given a partial half dose.

### DETAILED DESCRIPTION OF THE PREFERRED EMBODIMENTS

### Definitions

In the present application, “a” and “an” are used to refer to both single and a plurality of objects.

As used herein, “about” or “substantially” generally provides a leeway from being limited to an exact number. For example, as used in the context of the length of a polypeptide sequence, “about” or “substantially” indicates that the polypeptide is not to be limited to the recited number of amino acids. A few amino acids add to or subtracted from the N-terminus or C-terminus may be included so long as the functional activity such as its binding activity is present.

As used herein, administration “in combination with” one or more further therapeutic agents includes simultaneous (concurrent) and consecutive administration in any order.

As used herein, “amino acid” and “amino acids” refer to all naturally occurring L-α-amino acids. This definition is meant to include norleucine, ornithine, and homocysteine.

As used herein, in general, the term “amino acid sequence variant” refers to molecules with some differences in their amino acid sequences as compared to a reference (e.g. native sequence) polypeptide. The amino acid alterations may be substitutions, insertions, deletions or any desired combinations of such changes in a native amino acid sequence.

Substitutional variants are those that have at least one amino acid residue in a native sequence removed and a different amino acid inserted in its place at the same position. The substitutions may be single, where only one amino acid in the molecule has been substituted, or they may be multiple, where two or more amino acids have been substituted in the same molecule.

Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Also included within the scope of the invention are proteins or fragments or derivatives thereof which exhibit the same or similar biological activity and derivatives which are differentially modified during or after translation, e.g., by glycosylation, proteolytic cleavage, linkage to an antibody molecule or other cellular ligand, and so on.

Insertional variants are those with one or more amino acids inserted immediately adjacent to an amino acid at a particular position in a native amino acid sequence. Immediately adjacent to an amino acid means connected to either the ox-carboxy or «-amino functional group of the amino acid.

Deletional variants are those with one or more amino acids in the native amino acid sequence removed. Ordinarily, deletional variants will have one or two amino acids deleted in a particular region of the molecule.

As used herein, “fragments” or “functional derivatives” refers to biologically active amino acid sequence variants and fragments of the polypeptide of the present invention, as well as covalent modifications, including derivatives obtained by reaction with organic derivatizing agents, post-translational modifications, derivatives with nonproteinaceous polymers, and immunoadhesins.

As used herein, “carriers” include pharmaceutically acceptable carriers, excipients, or stabilizers which are nontoxic to the cell or mammal being exposed thereto at the dosages and concentrations employed. Often the pharmaceutically acceptable carrier is an aqueous pH buffered solution. Examples of pharmaceutically acceptable carriers include without limitation buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

As used herein “pharmaceutically acceptable carrier and/or diluent” includes any and all solvents, dispersion media, coatings antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active compound is generally present in from about 0.5 μg/ml of carrier. In the case of compositions containing supplementary active ingredients, the dosages are determined by reference to the usual dose and manner of administration of the said ingredients.

As used herein, “vector”, “polynucleotide vector”, “construct” and “polynucleotide construct” are used interchangeably herein. A polynucleotide vector of this invention may be in any of several forms, including, but not limited to, RNA, DNA, RNA encapsulated in a retroviral coat, DNA encapsulated in an adenovirus coat, DNA packaged in another viral or viral-like form (such as herpes simplex, and adeno-structures, such as polyamides.

As used herein, “host cell” includes an individual cell or cell culture which can be or has been a recipient of a vector of this invention. Host cells include progeny of a single host cell, and the progeny may not necessarily be completely identical (in morphology or in total DNA complement) to the original parent cell due to natural, accidental, or deliberate mutation and/or change.

As used herein, “subject” is a vertebrate, preferably a mammal, more preferably a human.

As used herein, “mammal” for purposes of treatment refers to any animal classified as a mammal, including humans, domestic and farm animals, and zoo, sports, or pet animals, such as dogs, cats, cattle, horses, sheep, pigs, and so on. Preferably, the mammal is human.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” refers to both therapeutic treatment and prophylactic or preventative measures. Those in need of treatment include those already with the disorder as well as those in which the disorder is to be prevented. “Palliating” a disease means that the extent and/or undesirable clinical manifestations of a disease state are lessened and/or the time course of the progression is slowed or lengthened, as compared to a situation without treatment.

As used herein, “A1” peptide, “A2” peptide, “B1” peptide, “B2” peptide and “B3” peptide refer to peptides derived from NME7 that are used to generate or select antibodies that bind to human NME7_{AB}, but not (or significantly less) to human NME1. The peptides used to generate these antibodies are common to both NME7_{AB} and NME7-X1, and are set forth as below.
A1 is NME7A peptide 1 (A domain): MLSRKEALDFHVDHQS (SEQ ID NO:141)
A2 is NME7A peptide 2 (A domain): SGVARTDASES (SEQ ID NO:142)
B1 is NME7B peptide 1 (B domain): DAGFEISAMQMFNMDRVNVE (SEQ ID NO: 143)
B2 is NME7B peptide 2 (B domain): EVYKGVVTEYHDMVTE (SEQ ID NO: 144)
B3 is NME7B peptide 3 (B domain): AIFGKTKIQNAVHCTDLPEDGLLEVQYFF (SEQ ID NO:145)

Further, for the sake of clarity, NME7A (with capital letter “A”) refers to the subunit A portion of NME7. NME7a (with small letter “a”) refers to the full-length NME7 that is described elsewhere in this application. And, NME7B (with capital letter “B”) refers to the subunit B portion of NME7. NME7b (with small letter “b”) refers to a species of NME7 that is partially devoid of the DM10 region, which is described elsewhere in this application.

As used herein, the term “antibody-like” means a molecule that may be engineered such that it contains portions of antibodies but is not an antibody that would naturally occur in nature. Examples include but are not limited to CAR (chimeric antigen receptor) T cell technology and the Ylanthia® technology. The CAR technology uses an antibody epitope fused to a portion of a T cell so that the body's immune system is directed to attack a specific target protein or cell. The Ylanthia® technology consists of an “antibody-like” library that is a collection of synthetic human fabs that are then screened for binding to peptide epitopes from target proteins. The selected Fab regions can then be engineered into a scaffold or framework so that they resemble antibodies.

As used herein, an “effective amount of an agent to inhibit an NME family member protein” refers to the effective amount of the agent in hindering the activating interaction between the NME family member protein and its cognate receptor such as

As used herein, “NME derived fragment” refers to a peptide sequence that is either a fragment of the NME or is highly homologous to the peptide sequence that is a fragment of the NME.

As used herein, the “MUC1*” extra cellular domain is defined primarily by the PSMGFR sequence (GTINVHDVETQFNQYKTEAASRYNLTISDVSVSDVPFPFSAQSGA (SEQ ID NO:6)). Because the exact site of MUC1 cleavage depends on the enzyme that clips it, and that the cleavage enzyme varies depending on cell type, tissue type or the time in the evolution of the cell, the exact sequence of the MUC1* extra cellular domain may vary at the N-terminus.

As used herein, the term “PSMGFR” is an acronym for Primary Sequence of MUC1 Growth Factor Receptor as set forth as GTINVHDVETQFNQYKTEAASRYNLTISDVSVSDVPFPFSAQSGA (SEQ ID NO:6). In this regard, the “N-number” as in “N-10 PSMGFR” or simply “N-10”, “N-15 PSMGFR” or simply “N-15”, or “N-20 PSMGFR” or simply “N-20” refers to the number of amino acid residues that have been deleted at the N-terminal end of PSMGFR. Likewise “C-number” as in “C-10 PSMGFR” or simply “C-10”, “C-15 PSMGFR” or simply “C-15”, or “C-20 PSMGFR” or simply “C-20” refers to the number of amino acid residues that have been deleted at the C-terminal end of PSMGFR. A mixture of deletions and additions is also possible. For instance, N+20/C-27 refers to a peptide fragment of wild-type MUC1 in which 20 amino acids are added to the PSMGFR at the N-terminus and 27 amino acids are deleted from the C-terminus.

As used herein, the “extracellular domain of MUC1*” refers to the extracellular portion of a MUC1 protein that is devoid of the tandem repeat domain. In most cases, MUC1* is a cleavage product wherein the MUC1* portion consists of a short extracellular domain devoid of tandem repeats, a transmembrane domain and a cytoplasmic tail. The precise location of cleavage of MUC1 is not known perhaps because it appears that it can be cleaved by more than one enzyme. The extracellular domain of MUC1* will include most of the PSMGFR sequence but may have an additional 10-20 N-terminal amino acids.

As used herein, “high homology” is considered to be at least 30%, 35%, 40%, 45%, 50%, 55%, 60%, 65%, 70%, 75%, 80%, 85%, 90%, 95%, or 97% identity in a designated overlapping region between any two polypeptides.

As used herein, “NME family proteins” or “NME family member proteins”, numbered 1-10, are proteins grouped together because they all have at least one NDPK (nucleotide diphosphate kinase) domain. In some cases, the NDPK domain is not functional in terms of being able to catalyze the conversion of ATP to ADP. NME proteins were formerly known as NM23 proteins, numbered H1 and H2. Recently, as many as ten (10) NME family members have been identified. Herein, the terms NM23 and NME are interchangeable. Herein, terms NME1, NME2, NME5, NME6, NME7, NME8 and NME9 are used to refer to the native protein as well as NME variants. In some cases these variants are more soluble, express better in E. coli or are more soluble than the native sequence protein. For example, NME7 as used in the specification can mean the native protein or a variant, such as NME7_{AB} that has superior commercial applicability because variations allow high yield expression of the soluble, properly folded protein in E. coli. NME7_{AB} consists primarily of the NME7 A and B domains but is devoid of most of the DM10 domain (SEQ ID NO:39), which is at the N-terminus of the native protein. “NME1” as referred to herein is interchangeable with “NM23-H1”. It is also intended that the invention not be limited by the exact sequence of the NME proteins. The mutant NME1-S120G, also called NM23-S120G, are used interchangeably throughout the application. The S120G mutants and the P96S mutant are preferred because of their preference for dimer formation, but may be referred to herein as NM23 dimers, NME1 dimers, or dimeric NME1, or dimeric NM23.

NME7 as referred to herein is intended to mean native NME7 having a molecular weight of about 42 kDa.

A “family of NME7” refers to full length NME7 as well as naturally occurring or artificially created cleaved form having a molecular weight about 30 kDa, 33 kDa, or a cleaved form having a molecular weight of about 25 kDa, a variant devoid or partially devoid of the DM10 leader sequence (SEQ ID NO:162), which is NME7 amino acids 1-91 of NME7 represented by SEQ ID NO:82 or 147, such as NME7b, NME7-X1, NME7_{AB} or a recombinant NME7 protein, or variants thereof whose sequence may be altered to allow for efficient expression or that increase yield, solubility or other characteristics that make the NME7 more effective or commercially more viable. The “family of NME7” may also include “NME7_{AB}-like” protein, which is a protein in the range of 30 to 33 kDa that is expressed in cancer cells.

As used herein, an “an agent that maintains stem cells in the naïve state or reverts primed stem cells to the naïve state” refers to a protein, small molecule or nucleic acid that alone or in combination maintains stem cells in the naïve state, resembling cells of the inner cell mass of an embryo. Examples include but are not limited to human NME1 dimers, bacterial, fungal, yeast, viral or parasitic NME proteins that have high sequence identity to human NME proteins, especially NME1, NME7, NME7-X1, NME7_{AB}, NME6, 2i (Silva J et al, 2008; Hanna et al, 2010), 5i (Theunissen T W et al, 2014), nucleic acids such as siRNA that suppress expression of MBD3, CHD4 (Rais Y1 et al, 2013), BRD4, or JMJD6 (Liu W et al 2013).

As used herein, the terms “NME7_{AB}”, “NME7_{AB}” and “NME-AB” are used interchangeably.

As used herein, an “an agent that promotes pluripotency” or “reverts somatic cells to a stem-like or cancer-like state” refers to a protein, small molecule or nucleic acid that alone or in combination induces expression of or suppresses expression of certain genes such that the genetic signature shifts to one that more closely resembles stem cells or cancer cells. Examples include but are not limited to NME1 dimers, NME7, NME7-X1, NME7_{AB}, 21, 5i, nucleic acids such as siRNA that suppress expression of MBD3, CHD4, BRD4, or JMJD6, microbial NME proteins that have high sequence homology to human NME1, NME2, NME5, NME6, NME7, NME8, or NME9, preferably with the regions that house NDPK domains.

As used herein, in reference to an agent being referred to as a “small molecule”, it may be a synthetic chemical or chemically based molecule having a molecular weight between 50 Da and 2000 Da, more preferably between 150 Da and 1000 Da, still more preferably between 200 Da and 750 Da.

As used herein, in reference to an agent being referred to as a “natural product”, it may be chemical molecule or a biological molecule, so long as the molecule exists in nature.

As used herein, FGF, FGF-2 or bFGF refer to fibroblast growth factor (Xu R H et al, 2005; Xu C et al, 2005).

As used herein, “Rho associated kinase inhibitors” may be small molecules, peptides or proteins (Rath N, et al, 2012). Rho kinase inhibitors are abbreviated here and elsewhere as ROCi or ROCKi, or Ri. The use of specific rho kinase inhibitors are meant to be exemplary and can be substituted for any other rho kinase inhibitor.

As used herein, the term “cancer stem cells” or “tumor initiating cells” refers to cancer cells that express levels of genes that have been linked to a more metastatic state or more aggressive cancers. The terms “cancer stem cells” or “tumor initiating cells” can also refer to cancer cells for which far fewer cells are required to give rise to a tumor when transplanted into an animal. Cancer stem cells and tumor initiating cells are often resistant to chemotherapy drugs.

As used herein, the terms “stem/cancer”, “cancer-like”, “stem-like” refers to a state in which cells acquire characteristics of stem cells or cancer cells, share important elements of the gene expression profile of stem cells, cancer cells or cancer stem cells. Stem-like cells may be somatic cells undergoing induction to a less mature state, such as increasing expression of pluripotency genes. Stem-like cells also refers to cells that have undergone some dedifferentiation or are in a meta-stable state from which they can alter their terminal differentiation. Cancer like cells may be cancer cells that have not yet been fully characterized but display morphology and characteristics of cancer cells, such as being able to grow anchorage-independently or being able to give rise to a tumor in an animal.

As used herein, “spacers” or “linkers” of different lengths can be incorporated anywhere in the peptide. Spacer attachment is usually through an amide linkage but other functionalities are possible.

### NME, NME7 and Protein Family of NME7

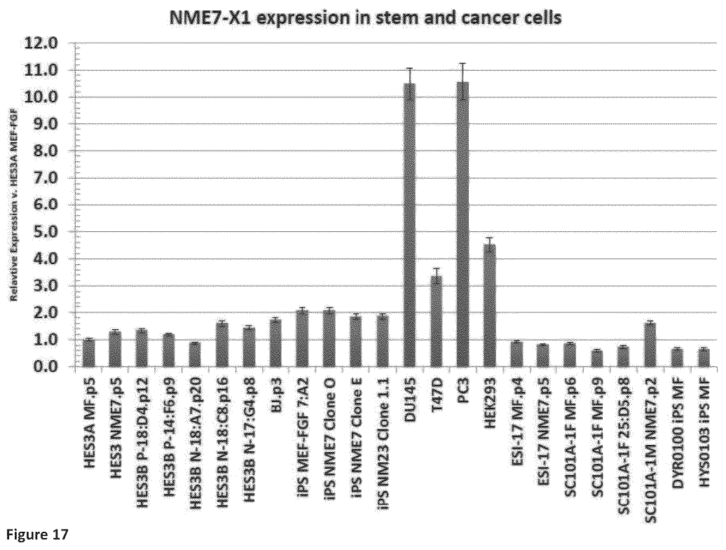

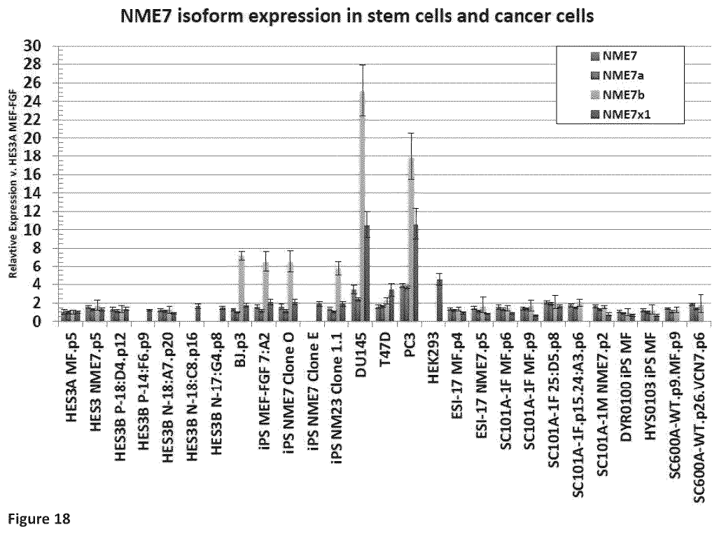

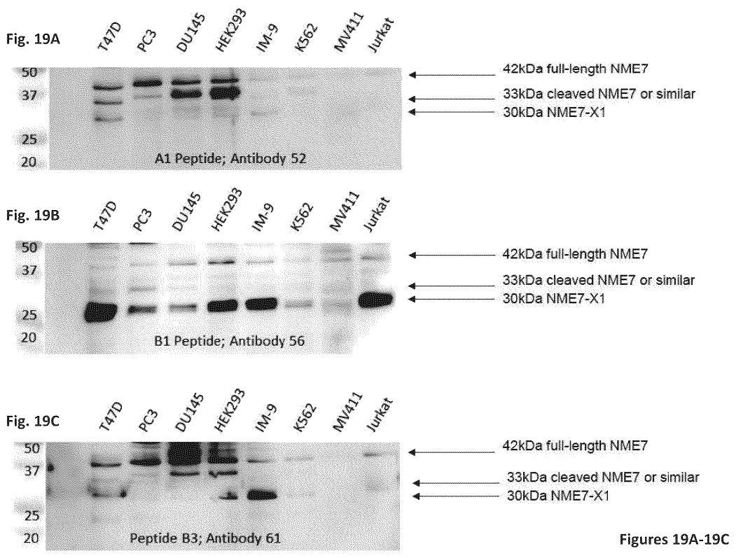

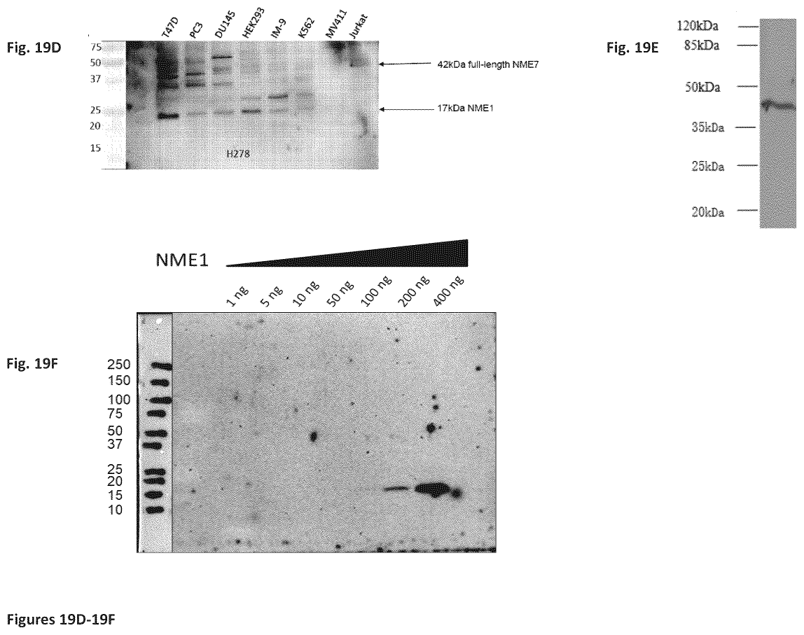

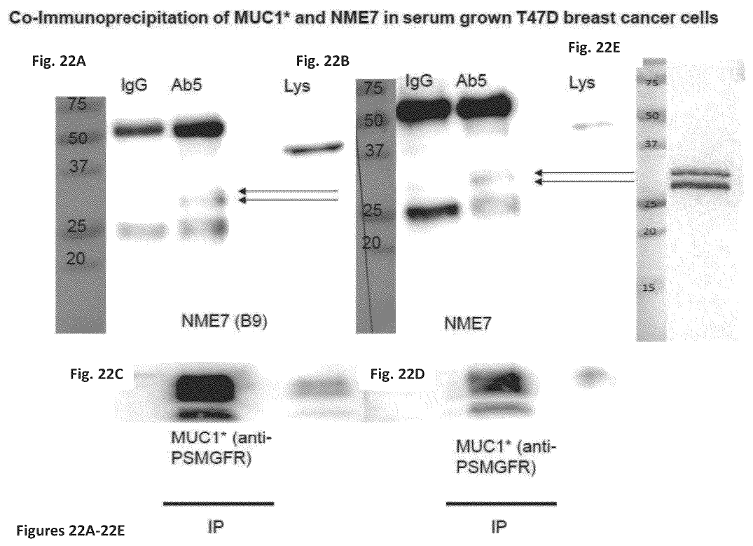

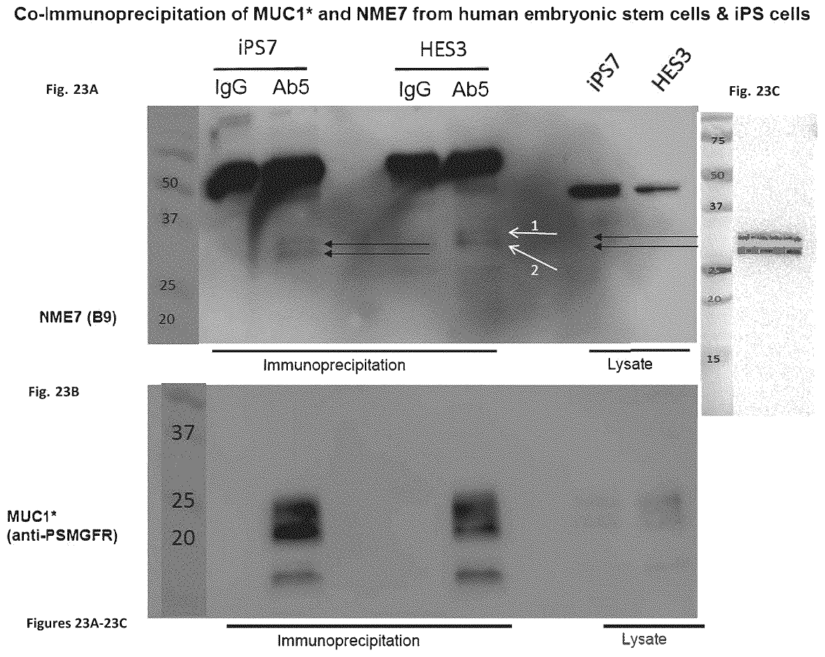

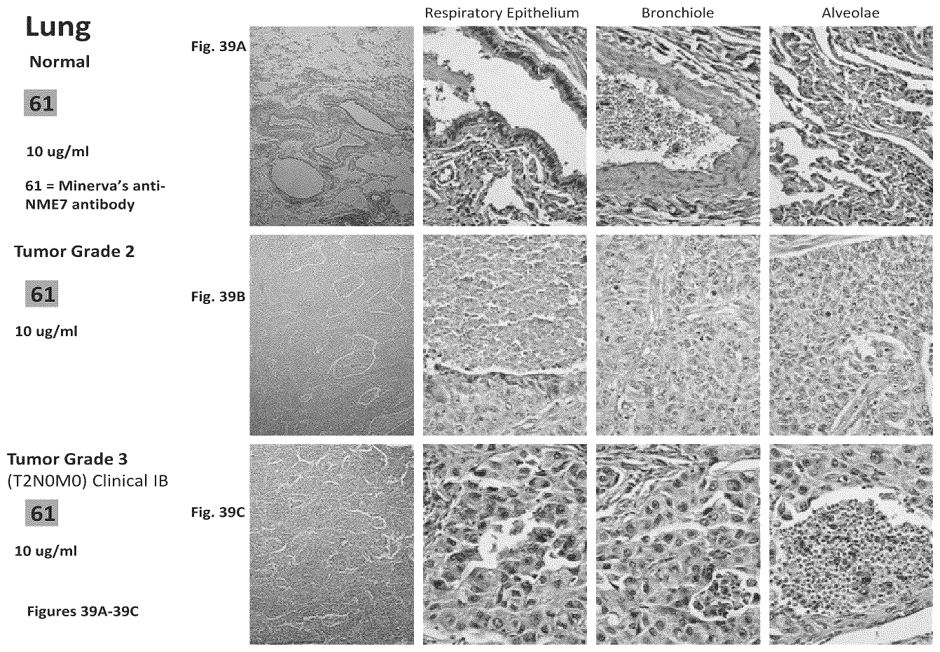

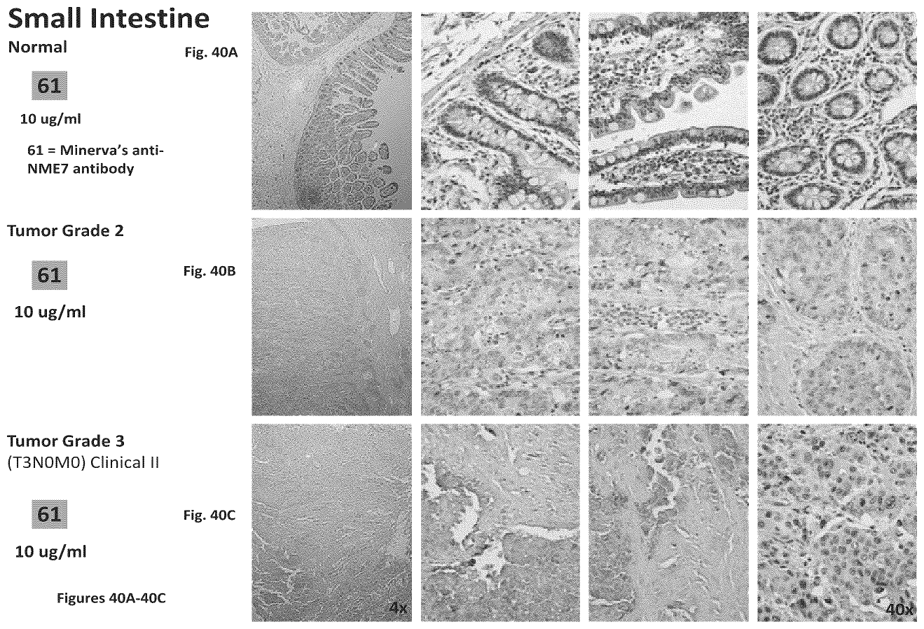

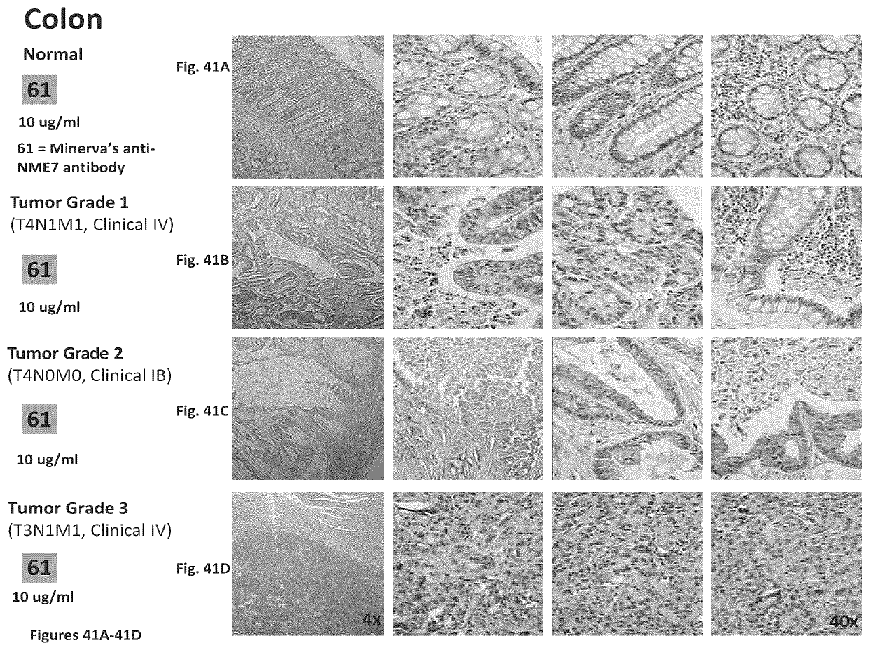

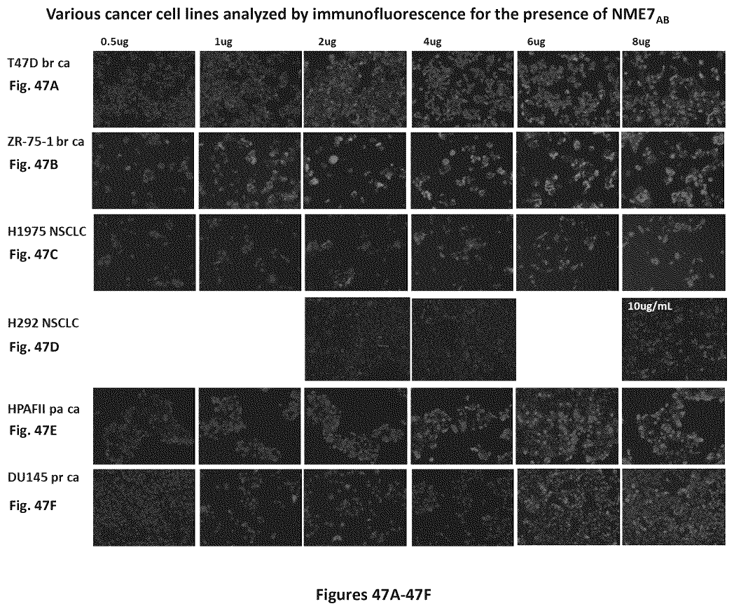

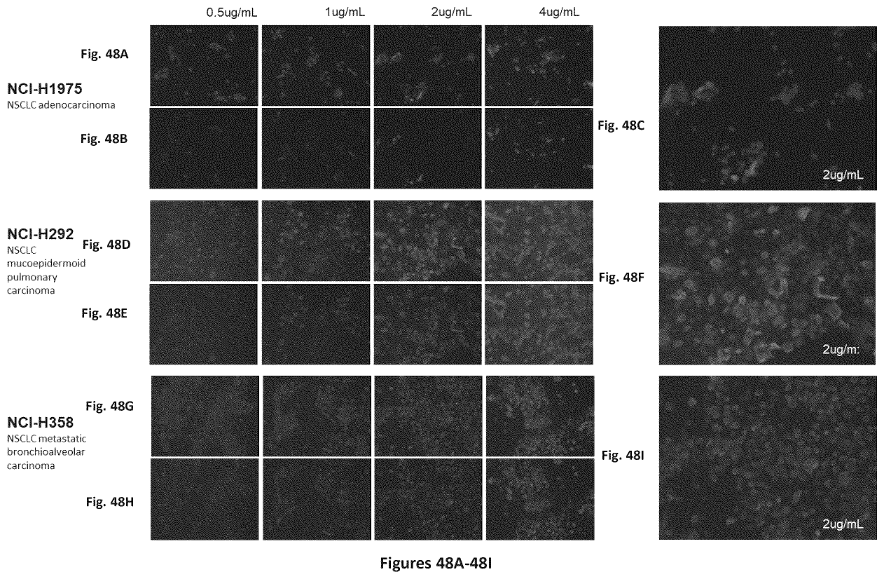

The present inventors discovered that NME7 and NME7-X1 are highly expressed in early human stem cells and also in most cancer cells (FIG. 17, FIG. 18, FIG. 19A-FIG. 19F, FIG. 22, FIG. 23, FIG. 39, FIG. 40, FIG. 41, FIG. 47, FIG. 48. FIG. 17 shows a graph of RT-PCR measurement of the expression of NME7-X1 in a panel of human stem cells and cancer cells. FIG. 18 shows a graph of RT-PCR measurement of the expression of NME7, NME7a, NME7b and NME7-X1 in a panel of human stem cells and cancer cells. NME7a is full-length NME7, NME7b is missing a small portion of the DM10 domain, NME7-X1 is missing all of the DM10 domain and a small portion of the N-terminus of the first NDPK A domain. The bar labeled NME7 means that primers were used that detected both NME7a and NME7b. FIGS. 19A-19F show photographs of Western blots in which various cancer cell lines are probed for expression of NME7 species using antibodies generated by immunization with NME7 derived short peptides. FIG. 19A shows Western probed with antibody of the invention #52 which binds to NME7 derived peptide A1. FIG. 19B shows Western probed with antibody of the invention #56 which binds to NME7 derived peptide B1. FIG. 19C shows Western probed with antibody of the invention #61 which binds to NME7 derived peptide B3. FIGS. 22A-22E show photographs of Western blots of a co-immunoprecipitation experiment. T47D breast cancer cell extracts were incubated with an antibody against the MUC1 cytoplasmic tail, Ab-5, or a control antibody, IgG, and co-immunoprecipitated. The gels were blotted with two different commercially available anti-NME7 antibodies B9 (FIG. 22A) and CF7 (FIG. 22B). Both gels show unique NME7 bands at ~33 kDa and ~30 kDa. The gels were stripped and re-probed with an antibody against the extracellular domain of MUC1*, anti-PSMGFR (FIG. 22C) and (FIG. 22D), which shows that the NME7 species and MUC1* interact. A recombinant NME7_{AB} and a recombinant NME7-X1 were mixed together and run on a gel, then probed with an anti-NME7 antibody, showing that the two unique NME7 species that are naturally occurring in breast cancer cells and that interact with MUC1* are an NME7_{AB}-like species and NME7-X1 (FIG. 22E). FIGS. 23A-23C show photographs of Western blots of a co-immunoprecipitation experiment. Human induced pluripotent stem, iPS7, or embryonic stem, HES3, cell extracts were incubated with an antibody against the MUC1 cytoplasmic tail, Ab-5, or a control antibody, IgG, and co-immunoprecipitated. The gel was blotted with a commercially available anti-NME7 antibody B9 (FIG. 23A). Both cell types show unique NME7 bands at ~33 kDa and ~30 kDa. The gel was stripped and re-probed with an antibody against the extracellular domain of MUC1*, anti-PSMGFR (FIG. 23B), which shows that the NME7 species and MUC1* interact. A recombinant NME7_{AB} and a recombinant NME7-X1 were mixed together and run on a gel, then probed with an anti-NME7 antibody, showing that the two unique NME7 species that are naturally occurring in breast cancer cells and that interact with MUC1* are an NME7_{AB}-like species and NME7-X1 (FIG. 23C). FIGS. 39A-39C shows human lung tissue specimens stained with an anti-NME7 antibody that binds to the B3 peptide. The figure shows lack of NME7 expression on normal tissues, increasing expression of NME7 as tumor grade and metastasis increase. FIGS. 40A-40C shows human small intestine tissue specimens stained with an anti-NME7 antibody that binds to the B3 peptide. The figure shows lack of NME7 expression on normal tissues, increasing expression of NME7 as tumor grade and metastasis increase. FIGS. 41A-41D show human colon tissue specimens stained with an anti-NME7 antibody that binds to the B3 peptide. The figure shows lack of NME7 expression on normal tissues, increasing expression of NME7 as tumor grade and metastasis increase. FIG. 47 and FIG. 48 show immunofluorescent photographs showing that NME7 is secreted by and binds to an extra cellular receptor of a wide variety of cancer cell lines.

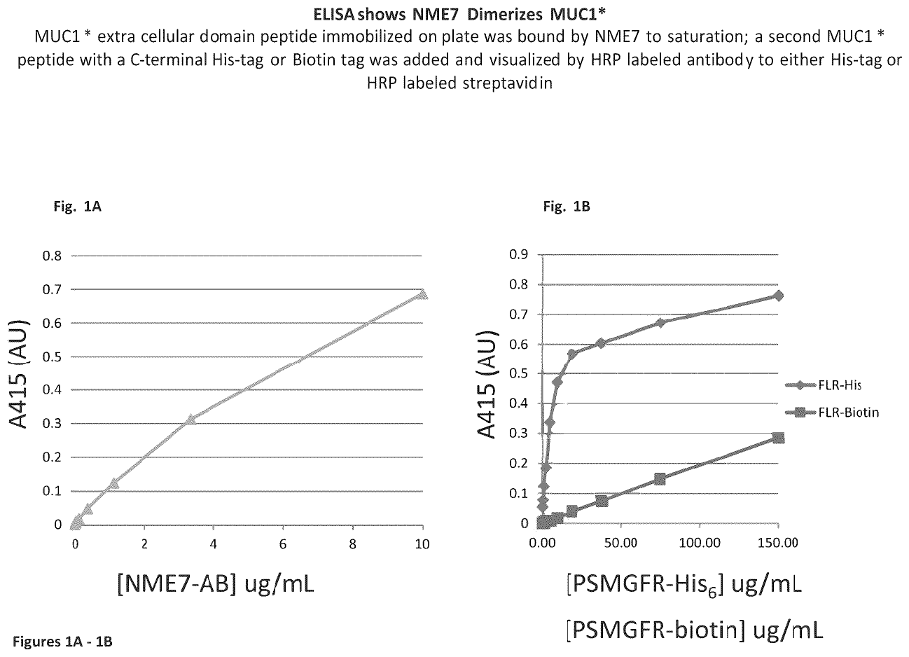

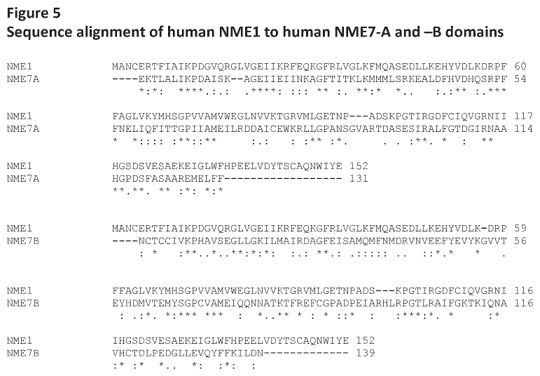

Further, we demonstrated that like NM23-H1, NME7 binds to and dimerizes the MUC1* growth factor receptor on both stem cells and cancer cells (FIG. 1). FIG. 5 shows a sequence alignment of NME1 and NME7 A and B domains.

The inventors recently discovered that NME7 is a primitive form of NME1 (NM23-H1) that is expressed in very early embryonic stem cells. NME7 is either not expressed at all, or is expressed at extremely low levels, in adult tissues. However, the inventors discovered that NME7 is expressed at high levels in cancerous cells and tissues and at even higher levels in metastatic cancer cells and tissues. A cleaved form of NME7 may be a secreted form allowing it to bind to and activate extracellular receptors. We detect full-length NME7, MW 42 kDa, as well as NME7 species that are approximately 33 kDa and 30 kDa. The 33 kDa and 30 kDa species are secreted from cancer cells. Western blots detect full-length NME7 in cell lysates, but smaller 30-33 kDa NME7 species in their conditioned media. Western blots probed with either an antibody that recognizes NME7 or an antibody that only recognizes the DM10 domain show that the lower molecular weight NME7 species that are secreted into the conditioned media are devoid of the DM10 domain. These data are consistent with the idea that naturally occurring NME7 species are comparable to the recombinant NME7_{AB} we generated as they have nearly the same molecular weight, both are secreted and are both devoid of the 91 amino acids of the DM10 domain which may keep the protein retained within the cell.

We discovered a new NME7 isoform, NME7-X1, and also discovered that it is over-expressed in stem cells and cancer cells and is particularly over-expressed in prostate cancers (FIG. 17, FIG. 18, FIG. 19, and FIG. 22). NME7-X1, molecular weight ~30 kDa, comprises NME7 amino acids 125-376, whereas the recombinant NME7_{AB}, molecular weight ~33 kDa, that we generated spans amino acids 92-376, so includes 33 more N-terminal amino acids. NME7b spans amino acids 37-376 and is devoid of only 37 amino acids of the DM10 domain is also overexpressed in prostate cancers (FIG. 18). We generated a human recombinant NME7-X1 and show that it is the secreted 30 kDa NME7 species in cancer cells that runs just lower than a naturally occurring ~33 kDa NME7 species that appears to be a naturally occurring “NME7_{AB}-like” protein that is a cleavage product or alternative isoform.

We tested a panel of cancer cell lines and found that they express high levels of NME7 and lower molecular weight species that may be truncations similar to NME7_{AB}, such as NME7_{AB}-like protein, or alternate isoforms such as NME7-X1.

Whereas NM23-H1 (aka NME1) has to be a dimer, NME7 is a monomer with two binding sites for MUC1* extracellular domain. We generated a recombinant human NME7 that is devoid of the DM10 domain, which we call NME7_{AB}. A sandwich ELISA binding assay that shows that a recombinant NME7_{AB} simultaneously binds to two PSMGFR peptides wherein the extracellular domain of MUC1* is comprised of most or all of the PSMGFR sequence (FIG. 1). In a nanoparticle binding assay, NME7 was also shown to be able to bind to and dimerize the PSMGFR portion of the MUC1* extracellular domain.

Agents that disable NME7, block its interaction with its binding partners or suppress its expression are potent anti-cancer therapeutics. Such agents may be antibodies, small molecules or nucleic acids. They may act on NME7 directly, on molecules that regulate NME7 expression, or on enzymes that cleave NME7 to cancer-promoting forms.

We discovered that like NM23-H1 dimers, a recombinant NME7_{AB} monomer was fully able to support pluripotent human stem cell growth in the absence of any other growth factor, cytokine or serum. Competitively inhibiting the interaction between NME7 and MUC1* extracellular domain, comprised essentially of the PSMGFR sequence, induced differentiation of stem cells, showing that it is the interaction of NME7 and MUC1* that promotes stem cell growth and inhibits differentiation.

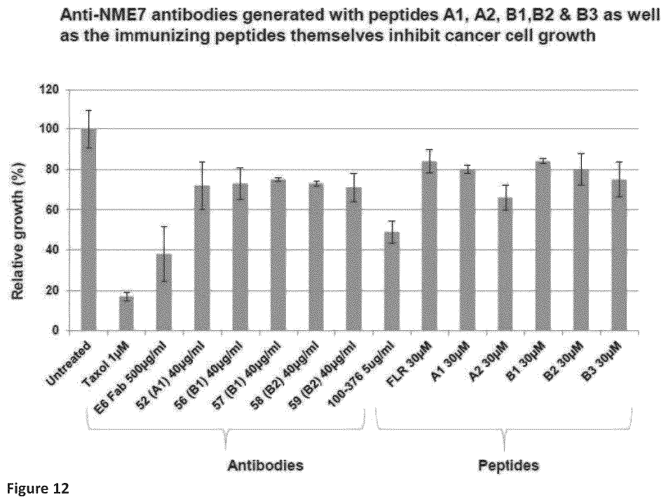

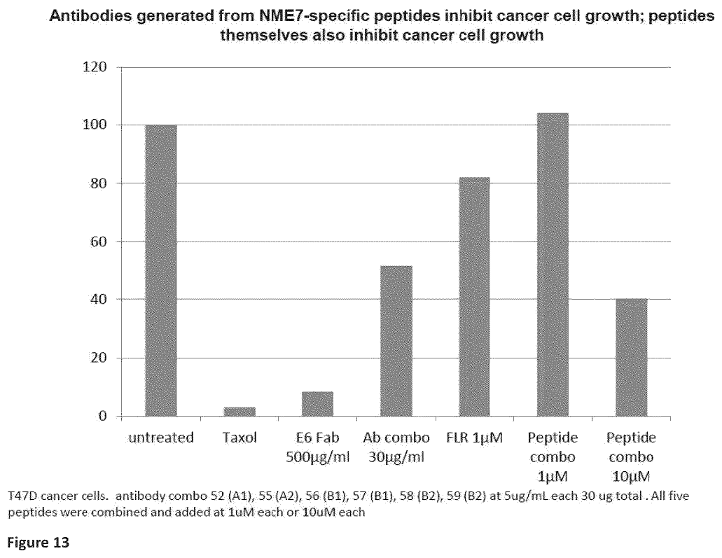

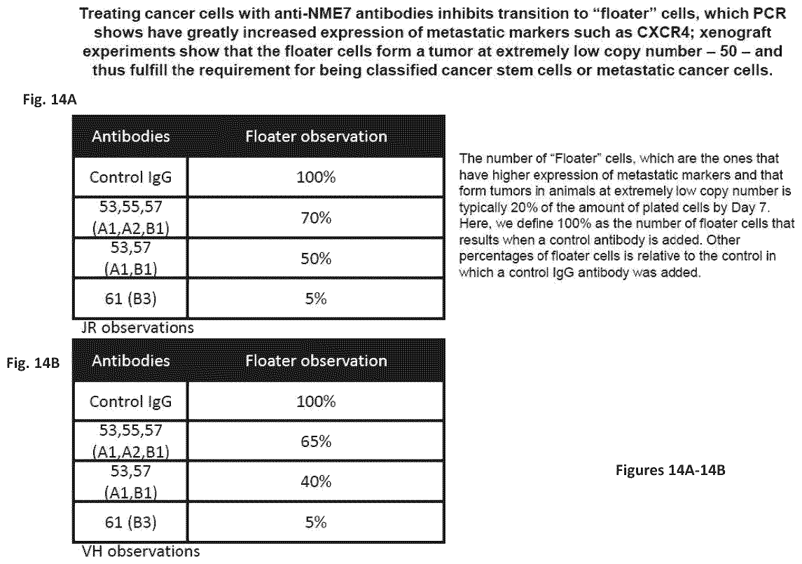

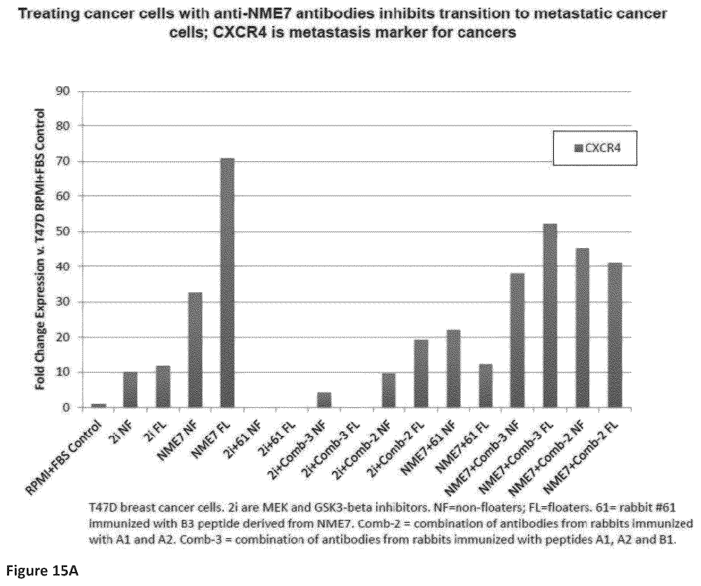

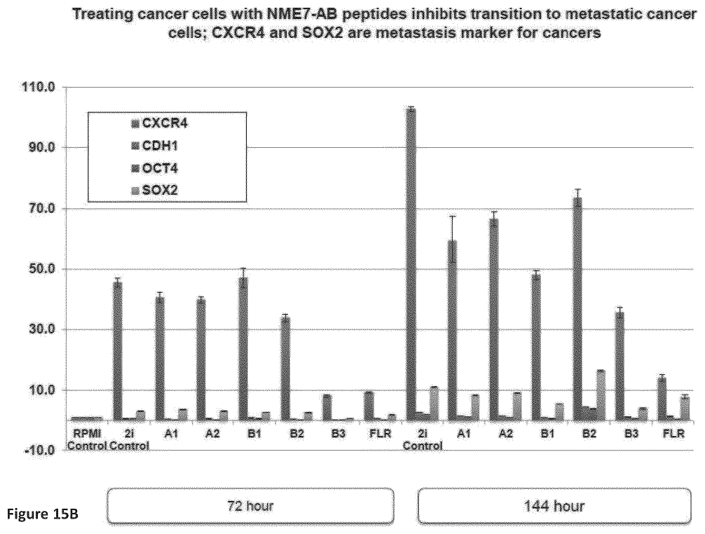

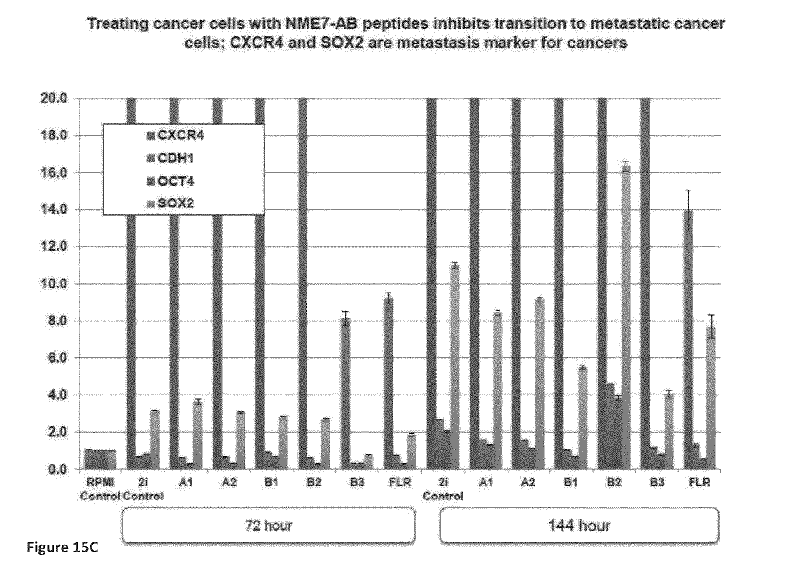

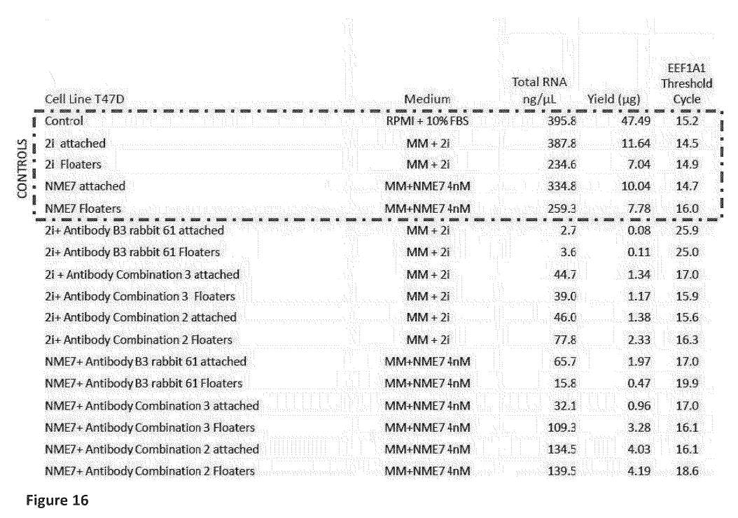

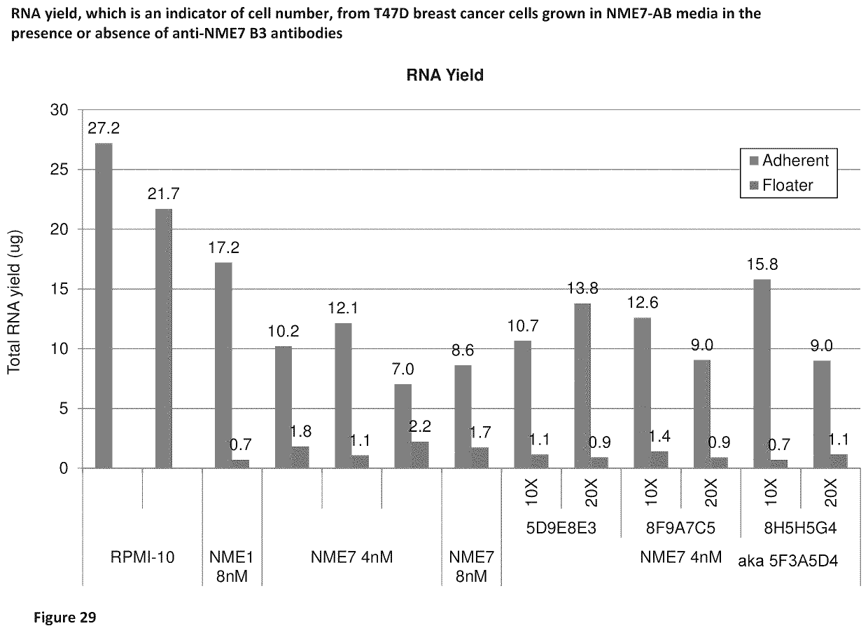

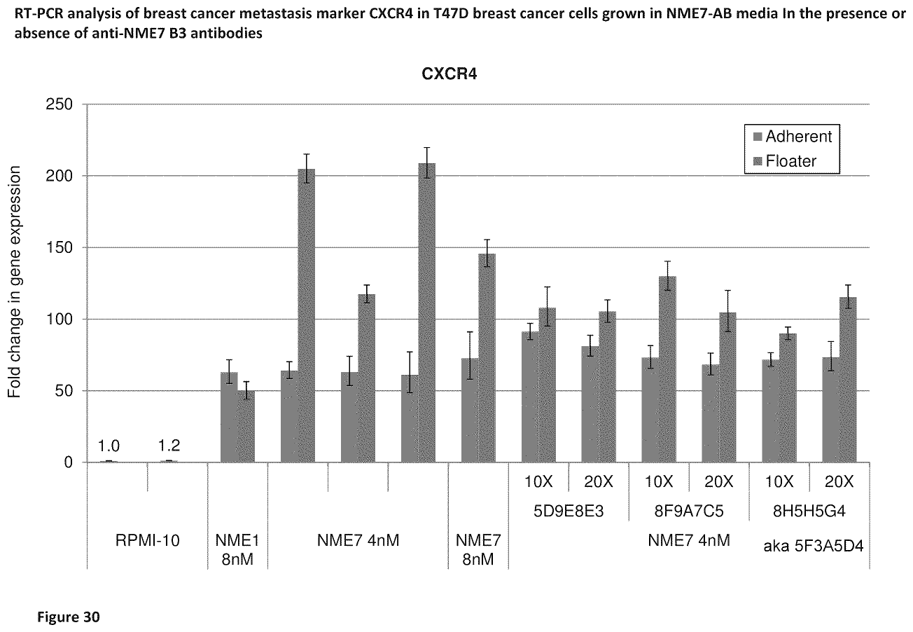

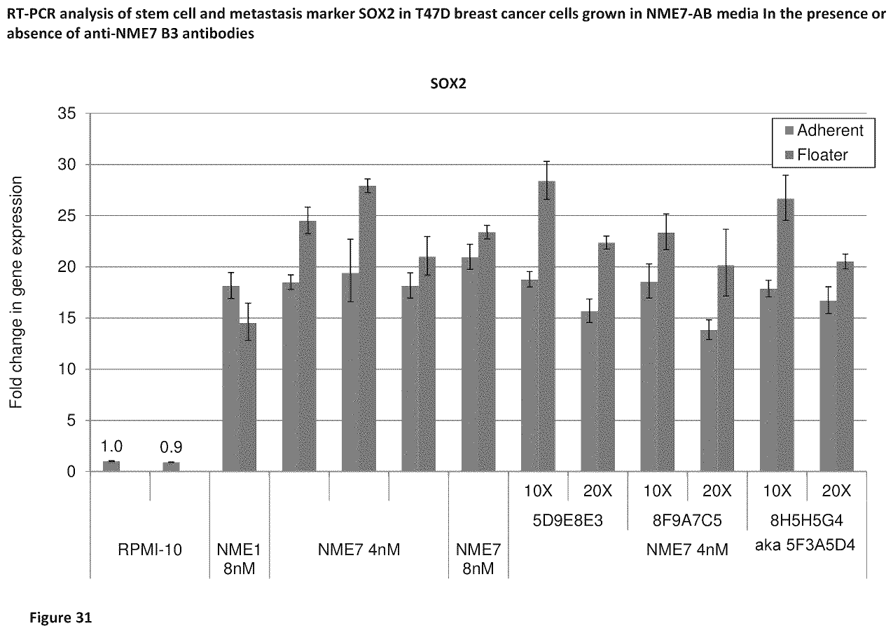

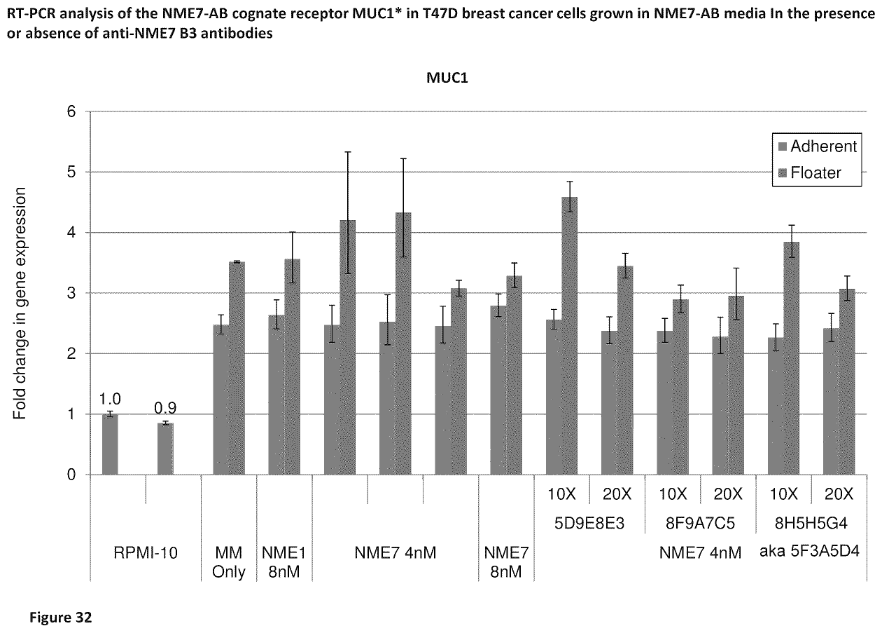

Next, we showed that NME7_{AB} alone is also able to fully support human cancer cell growth. NME7_{AB}, when added to regular cancer cell growth media, stimulated cancer cell growth and in particular the growth of MUC1-positive and MUC1*-positive cancer cells. Inhibiting the interaction of NME7 with MUC1* inhibited cancer cell growth. Blocking the MUC1* growth factor receptor with an anti-MUC1* Fab potently inhibited cancer cell growth. Similarly, antibodies that bind to NME7 inhibit cancer cell growth. In one example of inhibition of cancer growth by anti-NME7 antibody, the polyclonal antibody was generated from immunizing an animal with the portion of NME7 that spans amino acids 100-376 (FIG. 12 and FIG. 13). However, we found that antibodies generated from immunizing with shorter peptides from NME7_{AB} or from NME7-X1 also inhibit cancer growth. In particular, they inhibit the growth of MUC1 and MUC1*-positive cancers. Anti-NME7 antibodies of the invention inhibited the formation of the non-adherent “floater” cells that are able to form tumor spheres and which can travel from primary tumor and metastasize (FIG. 14, FIG. 16, FIG. 29). Anti-NME7 antibodies of the invention inhibited the upregulation of metastatic and stem cell markers, now believed to also be characteristic of metastasis (FIG. 15, FIG. 30, FIG. 31, FIG. 32).

### NME7 Causes Cancer Metastasis

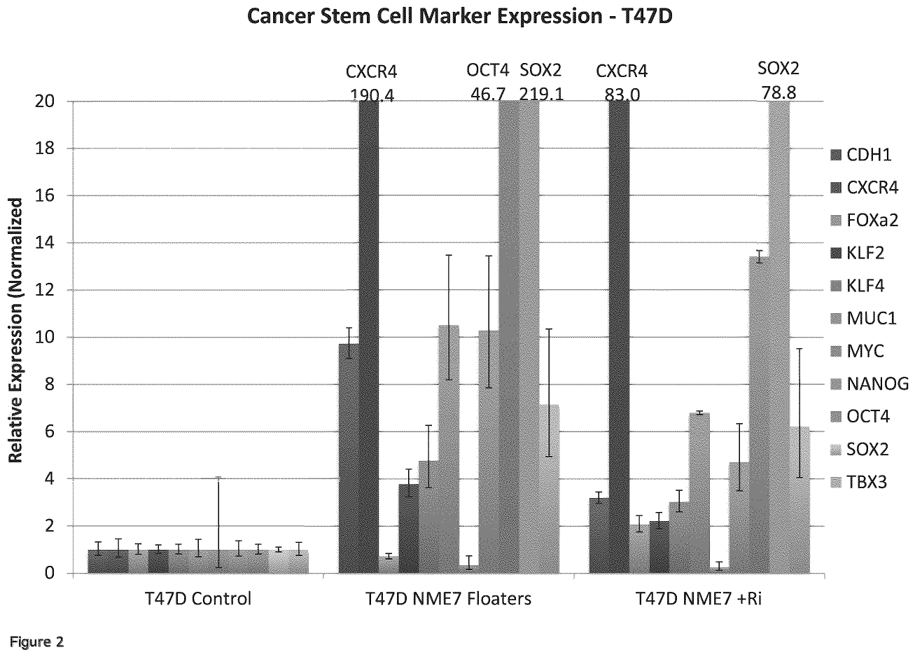

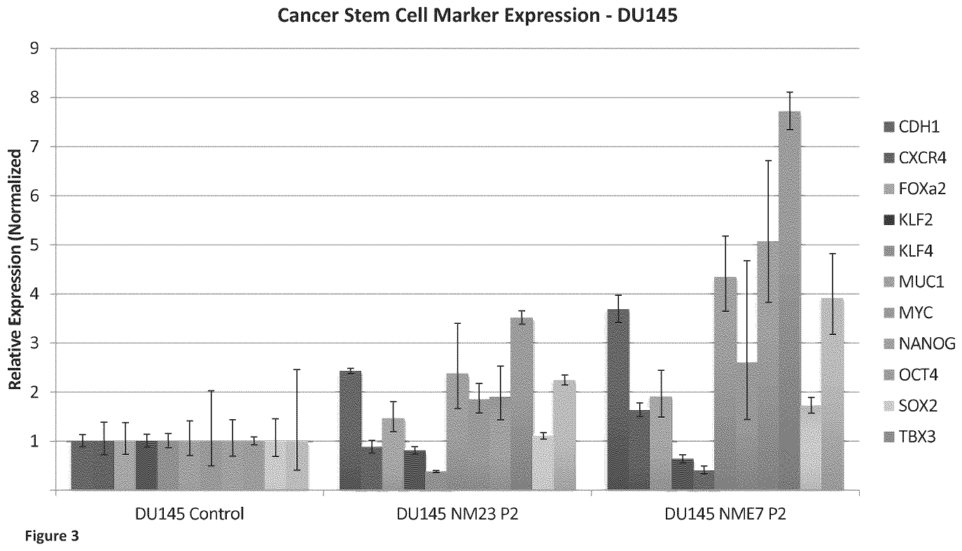

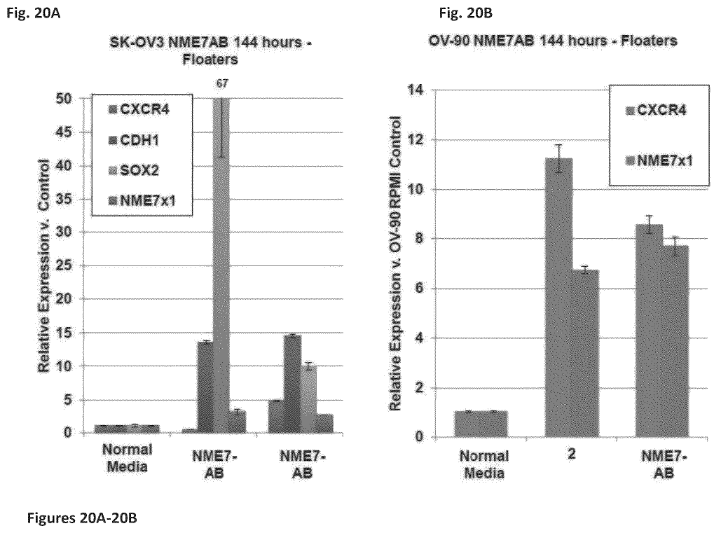

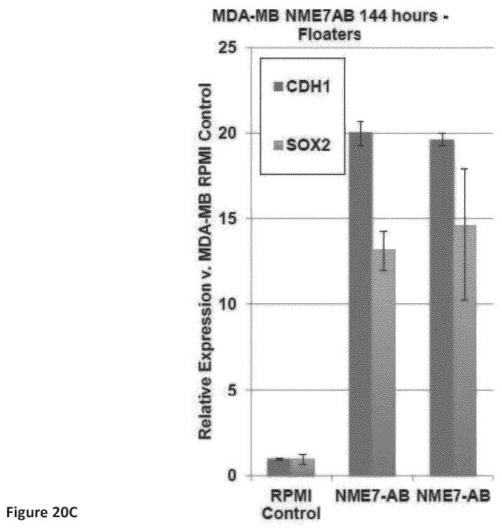

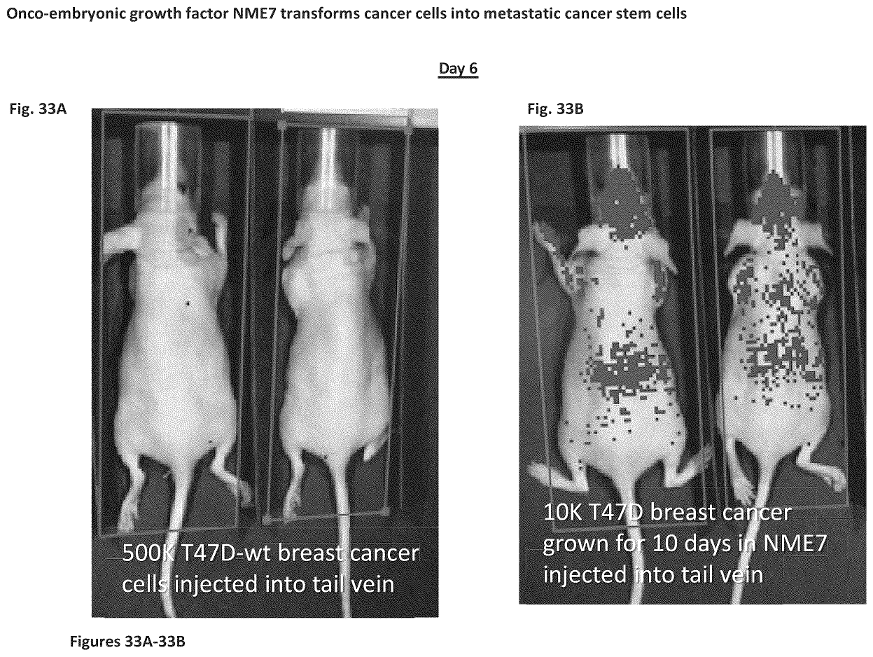

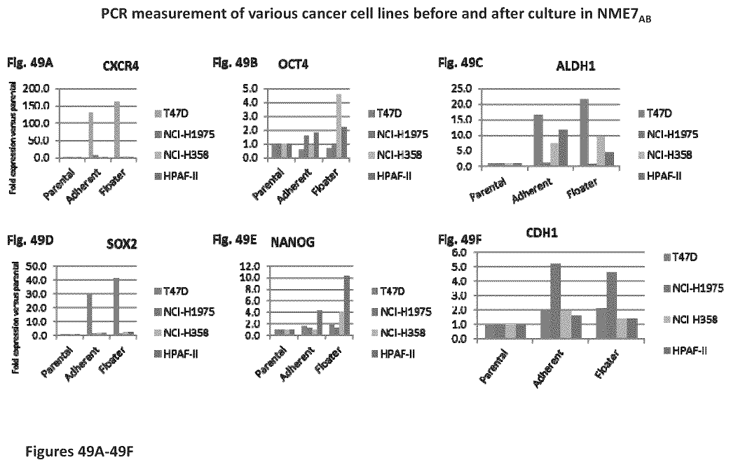

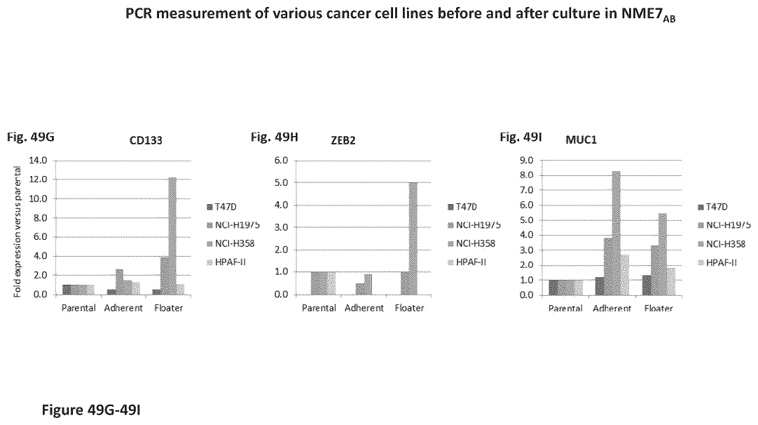

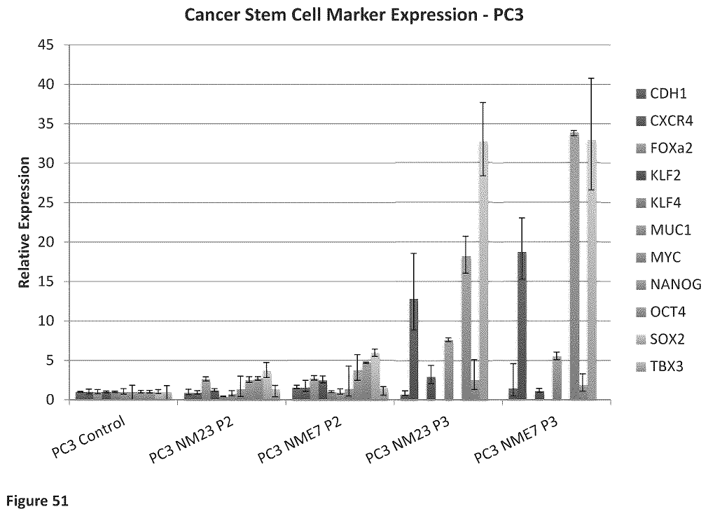

The inventors further discovered that culturing cancer cells in a minimal media containing NME7_{AB} induced a wide variety of cancer cells to become transformed to a more metastatic state. Evidence of this induced metastatic state include a change from adherent cell growth to non-adherent cell growth, aka, “floater” cells and accompanying up-regulation of specific metastatic markers that were especially upregulated in the floating cells. These metastatic markers that are upregulated after culture in NME7_{AB} include but are not limited to CXCR4, CHD1 aka E-cadherin, MUC1, ALDH1, CD44, and pluripotent stem cell markers such as OCT4, SOX2, NANOG, KLF2/4, FOXa2, TBX3, ZEB2 and c-Myc (FIG. 2, FIG. 3, FIG. 20, FIG. 49, FIG. 51). Cancer cells cultured in NME7_{AB} had dramatically higher engraftment rates when xenografted into test animals, which were over 90%. In addition, very low numbers of implanted cancer cells formed tumors in the test animals, which is evidence that NME7_{AB} had transformed them into cancer stem cells also known as metastatic cancer cells. Cancer cells cultured in NME7_{AB} and injected into the tail vein of NOD/SCID/GAMMA mice bearing estrogen release pellets metastasized in animals from low numbers of cells compared to the parent cells, grown in regular media (FIG. 33-FIG. 38). Because cancer cells make either an NME7 cleavage product or alternative isoform that is essentially equivalent to NME7_{AB}, the methods described here are not limited to using NME7_{AB}; other NME7 species could work as well. For example, we discovered another NME7 isoform, NME7-X1, is expressed by cancer cells. It is identical to our recombinant NME7_{AB} with the exception that the X1 isoform is missing 33 amino acids from the N-terminus. NME7-X1 is expected to function like NME7_{AB}. “NME7_{AB}-like” protein has also been detected in cancer cells as being about 33 Da species.

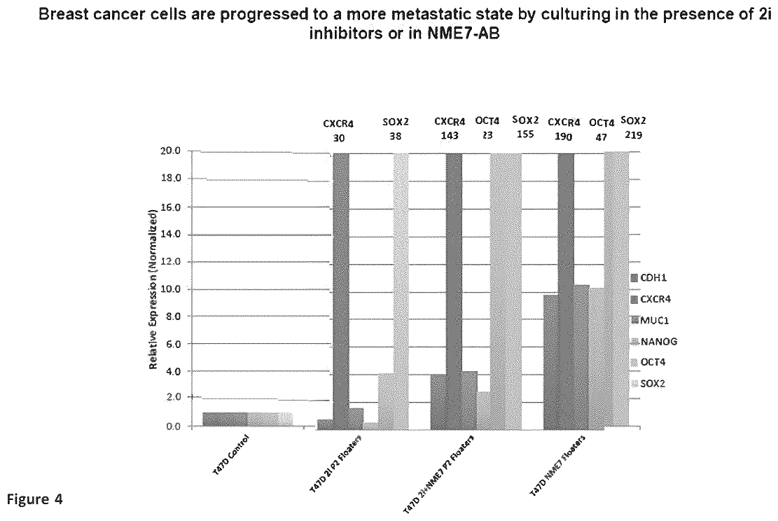

We note that the inventors' previous work showed that NME7_{AB} alone is able to revert human stem cells to an earlier naïve state. We discovered that culturing cancer cells in the presence of other reagents that make stem cells revert to a more naïve state, makes the cancer cells transform to a more metastatic state. We demonstrated that culturing cancer cells NME7_{AB} (FIG. 2), or in dimeric NME1 (FIG. 3), or “2i” inhibitors (FIG. 4), are each able to transform regular cancer cells into metastatic cancer cells, which are also called cancer stem cells “CSCs” or tumor initiating cells “TICs”. However, NME7_{AB} induced cancer cells to enter a more metastatic state better than NME1, also known as NM23-H1, which was better than 2i.

2i is the name given to two biochemical inhibitors that researchers found made human stem cells revert to a more naïve state. 2i are MEK and GSK3-beta inhibitors PD0325901 and CHIR99021, which are added to culture medium to final concentrations of about 1 mM and 3 mM, respectively. NME7_{AB} and NME7-X1 are at a final concentration of about 4 nM when added to separate batches of minimal medium to make cancer cells transform to metastatic cells, although lower and higher concentrations also work well in the range of about 1 nM to 16 nM. Human or bacterial NME1 dimers are used at a final concentration of 4 nM to 32 nM, with 16 nM typically used in these experiments, wherein the human NME bears the S120G mutation. Lower concentrations may be required if using wild type. It is not intended that these exact concentrations are important. It is important that the NME1 proteins are dimers and the range of concentrations over which this happens is in the low nanomolar range although certain mutations allow higher concentrations to remain as dimers. Similarly, the concentrations of NME7 proteins can vary. NME7_{AB} and NME7-X1 are monomers and concentrations used to transform cancer cells to metastatic cells should allow the proteins to remain as monomers.

In addition to NME7, NME7_{AB}, NME7-X1, and the 2i inhibitors MEKi and GSK3i, other reagents and inhibitors have been shown by others to cause stem cells to revert to a more naïve state. These inhibitors, “i's” include JNKi, p38i, PKCi, ROCKi, BMPi, BRAFi, SRCi as well as growth factors activing and LIF (Gafni et al 2013, Chan et al 2013, Valamehr et al 2014, Ware et al 2014, Theunissen et al 2014). These reagents can also be used to make cancer cells progress to a more metastatic state. Cells that have been induced to transform to a more metastatic state using any single factor or combination of the inhibitors or growth factors, that make stem cells revert to a more naïve state, can then be used as discovery tools to identify or test drugs to treat or prevent cancer metastasis.

Various molecular markers have been proposed as being indicators of metastatic cancer cells. Different cancer types may have different molecules that are up-regulated. For example, the receptor CXCR4 is up-regulated in metastatic breast cancers while E-cadherin, also known as CHD1, is up-regulated more in metastatic prostate cancers. In addition to these specific metastasis markers, typical markers of pluripotency such as OCT4, SOX2, NANOG, and KLF4 are up-regulated as cancers become metastatic. The starting cancer cells and the later metastatic cancer cells are assayed by PCR to measure expression levels of these genes. We demonstrated that these cancer cells, cultured in agents such as NME7_{AB} that cause them to be transformed to a more metastatic state, as evidenced by increased expression of metastatic markers and pluripotent stem cell markers, function as metastatic cancer cells.

A functional test of whether or not a population of cancer cells is metastatic is to implant very low numbers, e.g. 200, of the cells in immuno-compromised mice and see if they develop into a tumor. Typically 5-6 million cancer cells are required to form a tumor in an immuno-compromised mouse. We showed that as few as 50 of the NME-induced metastatic cancer cells formed tumors in mice. In addition, mice that were injected throughout the test period with human NME7_{AB}, NME1, or NME7-X1 developed remote metastases.

In one particular experiment, T47D human breast cancer cells were cultured in standard RPMI media for 14 days with media changes every 48 hours and passed by trypsinization when approximately 75% confluent. The cells were then plated into 6-well plates and cultured in minimal stem cell media (see Example 1) that was supplemented with 4 nM NME7_{AB} B. Media was changed every 48 hours. By about Day 4, some cells become detached from the surface and float. Media is carefully changed so as to retain the “floaters” as these are the cells that have the highest metastatic potential as evidence by RT-PCR measurement of metastatic markers. On Day 7 or 8, the floaters are harvested and counted. Samples are retained for RT-PCR measurement. The key marker measured is CXCR4, which is up-regulated by 40-200-times after being briefly cultured in NME7_{AB}.

The freshly harvested floater metastatic cells were xenografted into the flank of female nu/nu athymic mice that have been implanted with 90-day slow release estrogen pellets. Floater cells were xenografted with 10,000, 1,000, 100 or 50 cells each. Half of the mice in each group of 6 were also injected daily with 32 nM NME7_{AB} near the original implantation site. The parent T47D cells that were cultured in RPMI media without NME7_{AB} were also implanted into mice at 6 million, 10,000 or 100 as controls. Mice implanted with the NME7-induced floater cells developed tumors even when as few as 50 cells were implanted. Mice that were implanted with the floater cells and that received daily injections of NME7_{AB} also developed remote tumors or remote metastases in various organs. 11 out of the 12 mice, or 92%, that were injected with human NME7_{AB} after implantation of the NME7_{AB} cultured cancer cells developed tumors at the injection site. Only 7 out of the 12 mice, or 58%, that were not injected with human NME7_{AB} after implantation developed tumors. 9 out of the 11 mice, or 82%, that exhibited tumors and were injected with human NME7_{AB} developed multiple tumors remote from the injection site. None of the mice that were not injected with NME7_{AB} developed multiple, visible tumors.

After sacrifice, RT-PCR and Western blots showed that the remote bumps on the mice injected with NME7_{AB} were indeed human breast tumors. Similar analysis of their organs showed that in addition to remote bumps, mice had randomly metastasized to the liver and lung with human breast cancer characteristic of the human breast cancer cells that were implanted. As expected, only the mice implanted with 6 million cells grew tumors.

We have demonstrated that human recombinant NME7_{AB} is comparable in size and sequence to NME7-X1 and to a 30-33 kDa NME7 cleavage product. We have shown that NME7_{AB} promotes cancerous growth and causes cancer cells to accelerate to the highly metastatic cancer stem cell (CSC) state also called tumor initiating cells (TIC). Therefore, we conclude that NME7-X1 and an NME7 cleavage product that removes the DM10 domain also promote cancerous growth and causes cancer cells to accelerate to the highly metastatic cancer stem cell (CSC) state also called tumor initiating cells (TIC). In one example, NME7_{AB} Was added to cancer cells in a serum-free media and in the absence of any other growth factors or cytokines. Within 7-10 days, the cancer cells had reverted to the highly metastatic CSCs/TICs as evidenced by more than 100-fold increase in the expression of molecular markers such as CXCR4, which are indicators of metastatic cancer cells. In one example, T47D breast cancer cells were cultured in either standard RPMI media or in a Minimal Stem Cell Media (Example 1) to which was added recombinant NME7_{AB} to a final concentration of 16 nM. After 10 days cells were collected and analyzed by RT-PCR for expression of molecular markers of CSCs which were elevated by 10-200-times (FIG. 2). This is a specific, detailed example of how we transformed one cancer cell type to a more metastatic state. It is not intended that the invention be limited by these details as there are a range of cancer cells that are transformed in this way, a range of reagents that revert stem cells to a more naïve state that also progress cancer cells to a more metastatic state and a range of concentrations over which the added reagents transform the cancer cells. Other types of cancer cells have required longer periods of culture in NME7_{AB} for dramatic upregulation of metastatic markers and ability to form tumors from very low numbers of cancer cells implanted. For example, prostate cancer cells cultured in NME7_{AB}, 2i, human NME1 or bacterial NME1 that has high homology to human NME1 or human NME7 showed dramatic increase in metastatic markers after 2-3 passages.

Metastasis marker CXCR4 is particularly elevated in metastatic breast cancer cells, while CHD1 is particularly elevated in metastatic prostate cancer. Here we show that pluripotent stem cell markers such as OCT4, SOX2, NANOG, KLF2/4 and TBX3 are also up-regulated when cancer cells transform to more metastatic cells.

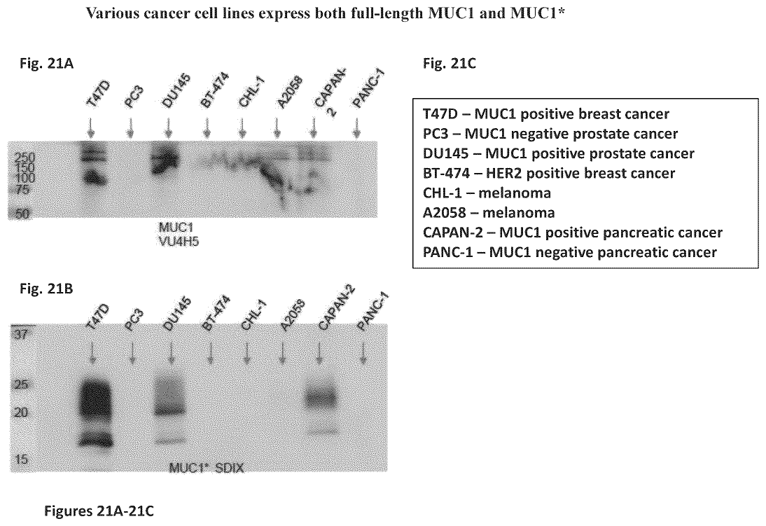

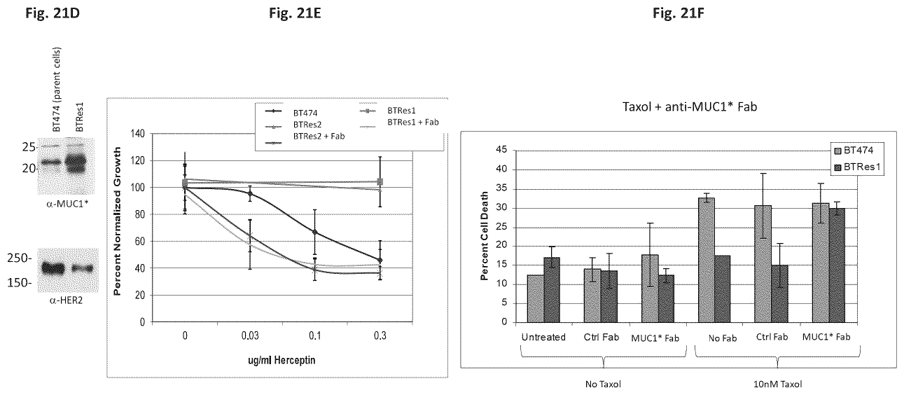

DU145 prostate cancer cells were cultured similarly and those cells cultured in NME7_{AB} also showed dramatic increases in expression of CSC markers (FIG. 3). In prostate cancer cells, CHD1 (aka E-cadherin) and CXCR4 were up-regulated compared to the control cancer cells, which were not grown in NME7_{AB}, along with other pluripotent stem cell markers. FIG. 20A-20C shows that ovarian cancer cell lines SK-OV3, OV-90 and breast cancer cell line MDA-MB all transitioned from adherent to non-adherent floater cells and increased expression of metastatic markers after 72 or 144 hours in culture with NME7_{AB}. Ovarian, prostate, pancreatic cancer cells and melanoma cells were also cultured in NME7_{AB} and were transformed to a more metastatic state after as few as 3 days in culture. FIG. 21 shows that breast, ovarian, prostate, pancreatic cancer cells and melanoma cells express MUC1 and MUC1*.

Here we have shown that NME7_{AB} transforms a wide range of cancer cells to a more metastatic state. We have also shown that cancer cells express a naturally occurring species that is approximately the same molecular weight as recombinant NME7_{AB} 33 kDa (FIG. 17, FIG. 18. FIG. 19, and FIG. 22 and is also devoid of the DM10 domain like NME7_{AB} and also express an alternative isoform NME7-X1 30 kDa which is the same sequence as NME7_{AB} except is missing 33 amino acids from the N-terminus. A co-immunoprecipitation experiment was performed on T47D breast cancer cells, wherein the cell extracts were incubated with an antibody against the MUC1 cytoplasmic tail, Ab-5, or a control antibody, IgG, and co-immunoprecipitated. The immunoprecipitated species were separated by gel electrophoresis. The gels were blotted with two different commercially available anti-NME7 antibodies. Both gels show unique NME7 bands at ~33 kDa and ~30 kDa (FIG. 22A-22B). The gels were stripped and re-probed with an antibody against the extracellular domain of MUC1*, anti-PSMGFR (FIG. 22C-22D), which shows that the NME7 species and MUC1* interact. A recombinant NME7_{AB} and a recombinant NME7-X1 that we made were mixed together and run on a gel, then probed with an anti-NME7 antibody, showing that the two unique NME7 species that are naturally occurring in breast cancer cells and that interact with MUC1* are an NME7_{AB}-like species and NME7-X1 (FIG. 22E). A similar experiment was carried out in human stem cells. FIG. 23A-23C show photographs of Western blots of a co-immunoprecipitation experiment. Human induced pluripotent stem, iPS7, or embryonic stem, HES3, cell extracts were incubated with an antibody against the MUC1 cytoplasmic tail, Ab-5, or a control antibody, IgG, and co-immunoprecipitated. The gel was blotted with a commercially available anti-NME7 antibody B9 (FIG. 23A). Both cell types show unique NME7 bands at ~33 kDa and ~30 kDa. The gel was stripped and re-probed with an antibody against the extracellular domain of MUC1*, anti-PSMGFR (FIG. 23B), which shows that the NME7 species and MUC1* interact. A recombinant NME7_{AB} and a recombinant NME7-X1 that we made were mixed together and run on a gel, then probed with an anti-NME7 antibody, showing that the two unique NME7 species that are naturally occurring in breast cancer cells and that interact with MUC1* are an NME7_{AB}-like species and NME7-X1 (FIG. 23C). Because NME7_{AB} is a recombinant protein, we do not know if the naturally occurring species may contain an extra 1-15 additional amino acids or devoid of 1-15 additional amino acids than the recombinant NME7_{AB}, yet run with the same apparent molecular weight. By “NME7_{AB}-like”, we mean an NME7 species that runs with an apparent molecular weight of approximately 33 kDa that is able to function the way the recombinant NME7_{AB} does, in that it is able to stimulate cancer cell growth, induce transition of cancer cells to a more metastatic state and is able to fully support pluripotent growth of human stem cells.

We conclude that cancer cell lines and cancer cell populations that express NME7 and lower molecular weight NME7 species contain some cancer cells that are CSCs or metastatic cancer cells. These cancers can be made more metastatic or increase the population of cells that are metastatic by culturing the cells in NME7_{AB}, NME7-X1 or lower molecular weight NME7 species. FIG. 19 shows a Western blot of a panel of cancer cells all expressing NME7 as well as lower molecular weight species NME7_{AB}-like at 33 kDa and NME7-X1 at 30 kDa. FIG. 21 shows that cancer cell lines T47D breast cancer, PC3 and DU145 prostate cancer, BT-474 breast cancer, CHL-1 and A2058 both melanoma cell lines and CAPAN-2 and PANC-1 both pancreatic cell lines all express MUC1 and MUC1*. In FIG. 21A, BT474 cells appear not to express MUC1 or MUC1* however, we previously showed (Fessler et al 2009) that when these HER2 positive breast cancer cells become resistant to chemotherapy drugs, i.e. metastatic, they do so by increasing expression of MUC1* (FIG. 21D). Blocking the MUC1* receptor with an anti-MUC1* Fab reversed their resistance to Herceptin (FIG. 21E), Taxol (FIG. 21F) as well as other chemo agents. These cancer types and other cancer types that express NME7 and lower molecular weight NME7 species such as 33 kDa, 30 kDa can be made more metastatic or increase the population of cells that are metastatic by culturing the cells in NME7_{AB}, NME7-X1 or lower molecular weight NME7 species.

Conversely, the metastatic potential of these and other cancer types that express NME7 and lower molecular weight NME7 species such as 33 kDa or 30 kDa can be reversed by treating the cells with anti-NME7 antibodies. Anti-NME7 antibodies or antibodies that bind to NME7_{AB} or NME7-X1 are administered to a patient for the treatment or prevention of cancers including breast, prostate, ovarian, pancreatic and liver cancers. Because we have shown that NME7_{AB} exerts its tumorigenic effects by binding to and activating the MUC1* growth factor receptor, anti-NME7 antibodies will be effective against any MUC1*-positive cancers, which include but are not limited to breast, lung, liver, pancreatic, gastric colorectal, prostate, brain, melanoma, kidney and others. Anti-NME7, anti-NME7_{AB} or anti-NME7-X1 antibodies are administered to patients for the treatment or prevention of cancers that are NME7_{AB}, NME7_{AB}-like, or NME7-X1 positive or a MUC1* positive.

### Testing Patient Cancer Cells for Effective Therapies

NME7_{AB}, NME7-X1 as well as 2i and other reagents that revert stem cells to a more naïve state also induce cancer cells to transform to a more metastatic state. After treatment with any one or combination of these reagents, cancer cells have a higher engraftment rate and require up to 100,000-times less cells to cause a tumor to form in a test animal. Therefore, methods described in this disclosure can be used to enable xenografting of a patient's primary tumor cells into a test animal.

Candidate therapeutic agents can then be tested on the recipient animal. Effective therapeutic agents identified in this way can be used to treat the donor patient or other patients with similar cancers. In one embodiment, a method of identifying effective therapeutics for a particular patient or a particular type of cancer comprises the steps of: 1) cancer cells are obtained from a cell line, a patient or a patient to whom the therapeutic being tested will be administered; 2) cancer cells are cultured in NME7_{AB}, NME7-X1, human NME1, bacterial NME1 that has high homology to human NME1 or NME7, 2i, or other reagents shown to revert stem cells to a more naïve state; 3) resultant cancer cells are implanted into a test animal to which human NME7_{AB}, NME7-X1, human NME1, bacterial NME1 that has high homology to human NME1 or NME7, 2i, or other reagents shown to revert stem cells to a more naïve state may also be administered or animal is transgenic for human NME7_{AB} or NME7-X1; 4) candidate anti-cancer therapeutic agents are administered to the animal; 5) efficacy of the therapeutic agents are assessed; and 6) effective therapeutic agent is administered to the donor patient or to another patient with similar cancer.

### Anti-NME7 Antibodies

Anti-NME7 antibodies are potent anti-cancer agents. NME7 is a growth factor that promotes the growth of cancer cells and also promotes their progression to a more metastatic state or a more aggressive state. NME7 and a truncated form of NME7 that is ~ 33 kDa or 30 kDa have been shown to fully support cancer growth even in serum-free media devoid of any other growth factors or cytokines. In pull-down assays, ELISAs and nanoparticle binding experiments, we have shown that the growth factor receptor MUC1* is a binding partner of NME7 and NME7_{AB}. Promotion of this interaction by eliminating all other growth factors or cytokines increased expression of cancer stem cell markers. Blocking this interaction even in the presence of serum, using a polyclonal antibody that specifically binds to NME7 actively killed the cancer cells. Thus, anti-NME7 or anti-NME7_{AB} antibodies are potent anti-cancer agents that can be administered to a patient for the treatment or prevention of cancers. More than 75% of all cancers are MUC1* positive. MUC1* is the transmembrane cleavage product of MUC1 wherein most of the extracellular domain has been shed, leaving a portion of the extracellular domain that contains most of the PSMGFR sequence and may contain 9-20 additional amino acids N-terminal to the boundary of the of the PSMGFR sequence.

One aspect of the invention is a method of treating or preventing cancer in a subject, comprising administering to the subject an effective amount of an anti-NME7 antibody. In one instance, the anti-NME7 antibody is able to bind to NME7_{AB}. In another instance, the anti-NME7 antibody is able to bind to NME7-X1. In yet another instance, the anti-NME7 antibody that is administered to a patient inhibits or prevents its binding to its target in the promotion of cancers. In one case, the target is the extracellular domain of a cleaved MUC1. More specifically, the NME7 target that promotes cancer is the PSMGFR region of the MUC1* extracellular domain. In one aspect, an effective therapeutic agent is one that disrupts or prevents the interaction between an NME7 species and MUC1* extracellular domain, consisting primarily of the PSMGFR portion of MUC1* or the PSMGFR peptide. Agents for the treatment or prevention of cancers are those agents that directly or indirectly inhibit the expression or function of NME7, an NME7_{AB}-like cleavage product or alternative isoform, including NME7-X1. In one case an effective anti-cancer therapeutic agent is one that binds to the NME7 species or disables its tumorigenic activity. An effective therapeutic agent for the treatment or prevention of cancers is an agent that binds to or disables NME7, an NME7_{AB}-like cleavage product or alternative isoform, or NME7-X1. In one aspect, the therapeutic agents that binds to the NME7 species is an antibody. The antibody may be polyclonal, monoclonal, bispecific, bivalent, monovalent, single chain, scFv, or an antibody mimic that may be animal in origin, human-animal chimera, humanized or human. The antibody can be generated by inoculation or immunization with an NME7 species or fragment thereof or selected, for example from a library or a pool of antibodies, for their ability to bind to an NME7 species, including NME7, an NME7_{AB}-like cleavage product or alternative isoform, including NME7-X1.

### Generation of Anti-NME7 Antibodies

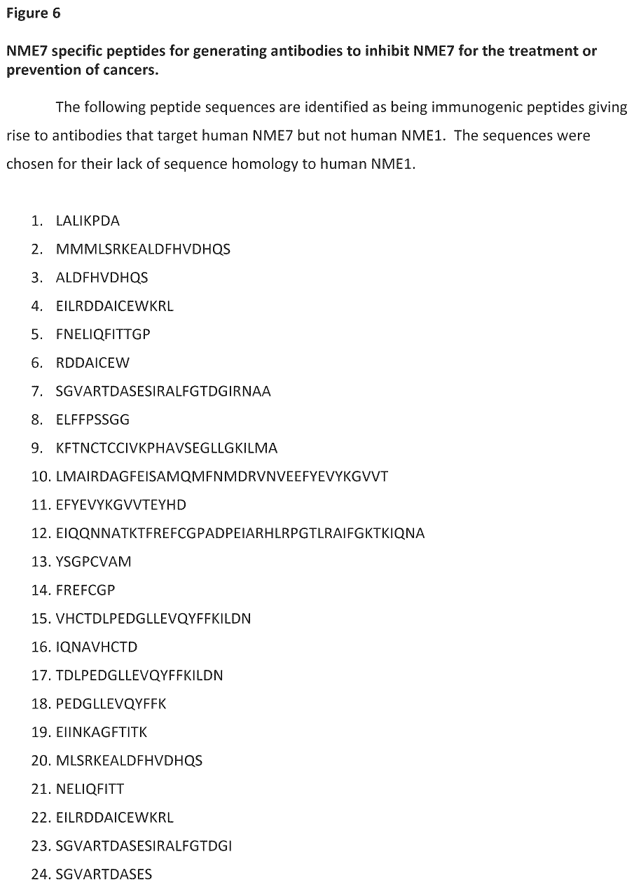

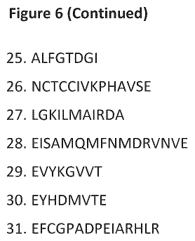

Anti-NME7 antibodies can be generated outside of the patient such as in a host animal or in a patient. Antibodies can be generated by immunization of NME7 or NME7 fragments or selected from a library or pool of antibodies that may be natural, synthetic, whole or antibody fragments based on their ability to bind to desired NME7 species such as NME7_{AB} or NME7-X1. In one aspect, the antibody is generated from immunization with, or selected for its ability to bind to, a peptide selected from those listed in FIG. 6-9. In another aspect, the antibody is generated from peptides whose sequences are not identical to those of human NME1 or the antibodies are selected for their ability to bind to NME7 species and their inability to bind to human NME1.

One method used to identify NME7 or NME7-X1 derived peptides that give rise to antibodies that inhibit cancer growth and inhibit transition to metastasis or peptides that are themselves inhibitory is as follows: 1) protein sequences of human NME1, human NME7, human NME7-X1 and several bacterial or fungal NME proteins that have high sequence homology to either human NME1 or human NME7 are aligned; 2) regions of high sequence homology among all the NMEs are identified; 3) peptide sequences that are unique to NME7 or NME7-X1 but are flanking the regions of high sequence homology are identified. The peptides are then synthesized and used to generate antibodies in a human or host animal. The resultant antibodies are selected for therapeutic use if: 1) they bind to NME7_{AB} or NME7-X1, but not to NME1; 2) have the ability to inhibit cancer growth; 3) have the ability to inhibit the transition of cancer cells to a more metastatic state; or 4) inhibit metastasis in vivo. In some cases, antibodies for therapeutic use are selected for their ability to disrupt binding of NME7_{AB} or NME7-X1 to the MUC1* extra cellular domain, to the PSMGFR peptide or to the N-10 peptide.

### Use of Anti-NME7 Antibody for Treatment of Cancer

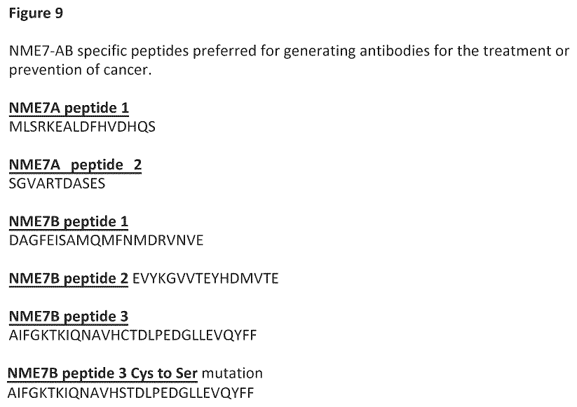

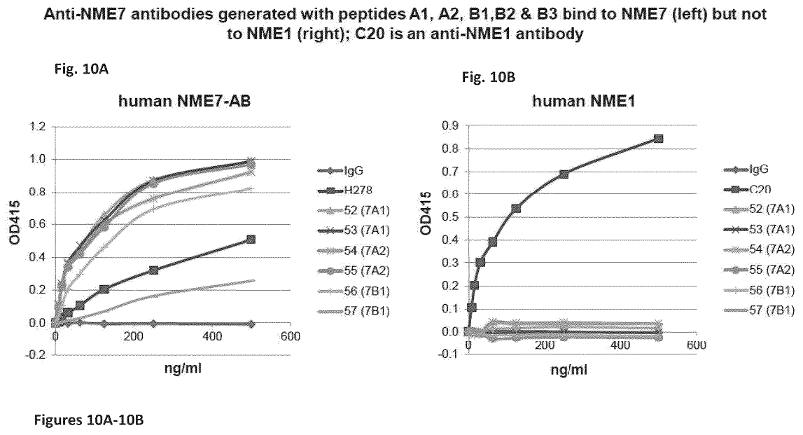

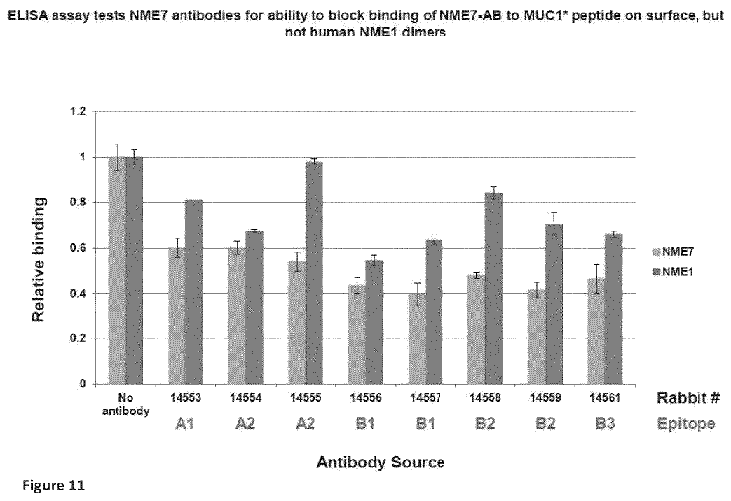

Those antibodies that inhibit cancer growth or transition to a more metastatic state are selected for use as anti-cancer therapeutics and may be administered to a patient for the treatment or prevention of cancers. Selected antibodies may be further optimized for example by engineering or making human chimera antibodies or fully human antibodies. To demonstrate the efficacy of this approach, we selected NME7 peptides from regions of NME7 suspected to be critical to its cancerous function. We then generated antibodies using these peptides and then tested both the resultant antibodies as well as the immunizing peptides for their ability to: a) inhibit cancerous growth; and b) inhibit the induced transition from cancer cells to metastatic cancer cells. NME7 peptides were selected as immunizing agents for antibody production and as inhibitory agents themselves (FIG. 9, Example 7). Peptides A1 (SEQ ID NO:141), A2 (SEQ ID NO:142), B1 (SEQ ID NO:143), B2 (SEQ ID NO:144) and B3 (SEQ ID NO:145), wherein A refers to the domain from which the peptide is derived, i.e. the NDPK A domain and the B denotes that the peptide is derived from the NDPK B domain (FIG. 5). Each peptide was used as an immunogen and injected into 2 rabbits each for production of polyclonal antibodies. The antibodies that were harvested from the blood of the immunized rabbits were purified over a column derivatized with the immunizing peptide. The purified antibodies were then tested for their ability to bind to human NME7. All of the resultant antibodies bound to human NME7 but not human NME1 as desired (FIG. 10A-10B, Example 8). These results show that by choosing peptides whose sequence is found in NME7 but not exactly identical in NME1, antibodies are generated that specifically bind to NME7 but not NME1. Because NME1 has healthy function, it is in most cases desirable to generate antibodies that do not interfere with NME1. The antibodies were also tested for their ability to inhibit the binding of NME7 to a MUC1* extracellular domain peptide. The ELISA experiment shown in FIG. 11 shows that the antibodies inhibited the binding of NME7_{AB} to a MUC1* extracellular domain peptide much more than they inhibited binding of NME1. Recall that each of the NME7 A domain and B domain can bind to a PSMGFR peptide. Therefore, complete inhibition of NME7_{AB} binding to a PSMGFR peptide cannot be accomplished with a single antibody or peptide that is derived from just one domain. These antibodies and their respective immunizing peptides also inhibited cancer cell growth (FIG. 12-13). These antibodies also inhibited the formation of non-adherent “floater” cells that result from growing cancer cells in NME7_{AB} (FIG. 14). As can be seen in the figure, the polyclonal antibody generated by immunization with the B3 peptide reduced the number of metastatic floater cells by 95%, indicating that anti-NME7 antibodies that bind to the B3 peptide are most effective at inhibiting cancer metastasis. Similarly, the antibodies inhibited the expression of metastatic marker CXCR4 (FIG. 15A). Again, the B3 antibodies were most efficient at inhibiting expression of CXCR4; bar labeled NME7 FL (NME7 floater cells) shows 70-fold increase in CXCR4 that B3 antibody 61 decreased to 20-fold (bar labeled NME7+61 FL). In addition, the immunizing peptides themselves inhibited the upregulation of CXCR4 and other metastatic markers when T47D cancer cells were grown in NME7_{AB} or 2i.

This is but one example of selecting peptides that generate antibodies that inhibit the cancerous function of NME7 and NME7 species. Sequence alignment among human NME1, human NME7, human NME7-X1 and bacterial NME proteins that had high sequence homology to human NME1 or NME7 identified five regions of homology. The fact that peptides A1, A2, B1, B2 and B3 all generated antibodies that inhibited cancer growth or their transition to a metastatic state means that the five regions from which these peptides were derived are regions of NME7 that are important for its function in the promotion of cancer. Other peptides from these regions will also give rise to anti-NME7 antibodies that will inhibit cancer growth and metastasis and are therefore potent anti-cancer therapeutics. Antibodies generated from peptides A1, A2, B1, B2 and B3 were shown to inhibit cancer growth and inhibited the transition to a more metastatic state. Monoclonal antibodies generated by immunization with the same or similar peptides and subsequent testing of the monoclonals will identify antibodies that, after humanizing or other engineering known to those skilled in the art, would be administered to a patient for the treatment or prevention of cancers.

In a particular experiment, the antibodies generated by immunization with peptides A1, A2, B1, B2 and B3, as well as the immunizing peptides themselves, were added to cancer cells in culture to see if the addition of the antibodies or the immunizing peptides would inhibit cancer cell growth. At low concentrations and added separately, the antibodies as well as the immunizing peptides inhibited cancer cells growth (FIG. 12 for one example). However, when added at higher concentrations or combined, the antibodies as well as the immunizing peptides robustly inhibited cancer cell growth (FIG. 13). The corresponding human NME7 amino acid numbers of immunizing peptides A1, A2, B1, B2 and B3 are 127-142, 181-191, 263-282, 287-301, 343-371, respectively, from human full-length NME7 having SEQ ID NO:82 or 147.

To clarify, when residue numbers of NME7 are discussed, they refer to the residue numbers of NME7 as set forth in SEQ ID NO:82 or 147.

The antibody used in cancer growth inhibition experiments and one of the antibodies shown in FIG. 12 was generated by immunizing with NME7 peptide corresponding to amino acids 100-376 of NME7 (SEQ ID NO:82 or 147). To generate higher affinity and specific anti-NME7 antibodies, the following steps are followed: immunize animal with a peptide containing human NME7 amino acids 100-376, then: 1) de-select those antibodies that bind to human NME1; 2) select those antibodies that inhibit NME7_{AB}, 2i, or other NME induced transition of cancer cells to a more metastatic state; 3) select those antibodies that inhibit the growth of cancer cells; 4) select those antibodies that inhibit the growth of MUC1* positive cancer cells; 5) select those antibodies that inhibit binding of NME7_{AB} or NME7-X1 to MUC1* extracellular domain, essentially inhibit binding to the PSMGFR peptide; and/or 6) select those antibodies that bind to one or more of the peptides listed in FIG. 9—A1, A2, B1, B2 or B3 peptides.

Higher affinity monoclonal antibodies or monoclonal antibodies generated from longer peptides may be more effective antibody therapeutics. Alternatively, combinations of ani-NME7, anti-NME7_{AB} or anti-NME7-X1 antibodies are administered to a patient to increase efficacy.

### Anti-NME7 Antibodies Inhibit the Transition of Cancer Cells to Metastatic Cancer Cells.

Anti-NME7 antibodies inhibit transition of cancer cells to metastatic cancer cells also called cancer stem cells (CSCs) or tumor initiating cells (TICs). Recall that we have demonstrated that culturing a wide variety of cancer cells in the presence of NME7_{AB} causes them to transition from regular cancer cells to the metastatic CSCs or TICs. Thus, antibodies that bind to NME7, NME7_{AB} or NME7-X1 will inhibit the progression of cancer cells to a more metastatic state.

Cancer cells transform to a more metastatic state when cultured in the presence of agents that revert stem cells to a more naïve state. We have demonstrated that culturing cancer cells in NME7_{AB}, human NME1 dimers, bacterial NME1 dimers or MEK and GSK3-beta inhibitors, called “2i”, causes the cells to become more metastatic. As the cells transition to a more metastatic state, they become non-adherent or less adherent and float off of the culture plate. These floating cells, “floaters” were collected separately from those that were adherent and were shown to: a) express much higher levels of metastatic genes; and b) generated tumors when xenografted into mice at very low copy number. RT-PCR measurement of specific metastatic markers such as CXCR4 for breast cancers, CHD1 for prostate cancer, and other pluripotent stem cell markers such as OCT4, SOX2, NANOG, KLF4 and others were dramatically over-expressed in cancer cells that were cultured in NME7_{AB} and most over-expressed in the cells that became non-adherent, called “floaters” here and in figures.

In one example, NME7_{AB} specific antibodies, generated by immunization with NME7-derived peptides A1, A2, B1, B2 and B3, as well as the immunizing peptides themselves, were added into the media along with either NME7_{AB} or 2i to determine if they inhibited the transformation of regular cancer cells to metastatic cancer stem cells. Antibodies and peptides were separately added along with the agent that causes metastatic transformation; in this case NME7_{AB} or the 2i inhibitors PD0325901 and CHIR99021. NME7_{AB} and 2i were separately used to induce the cancer cells to be transformed to a more aggressive metastatic state. 2i was used so that it could not be argued that the antibodies that were added to the media simply sopped up all of the NME7_{AB} so that the causative agent effectively was not there (Example 10).

Visual observation was independently recorded by two scientists as the experiment progressed (FIG. 14). The most striking observation was that the antibodies and the peptides dramatically reduced the number of floater cells, which was the first indication that the antibodies and peptides inhibit the transformation to metastatic cancer cells. In particular, cells to which the antibody generated from immunization with the B3 peptide barely generated any floater cells. mRNA was extracted from both the floater cells, the adherent cells and the control cancer cells. The amount of mRNA, which indicates cell viability and growth, was measured. Cells that were treated with antibody had much less mRNA, indicating less live dividing cells (FIG. 16), which confirms that anti-NME7_{AB} antibodies inhibit cancer cell growth as well as their transition to a more metastatic state. RT-PCR was used to measure expression levels of metastatic markers, including CXCR4. Treatment with the anti-NME7 antibodies greatly reduced the amount of metastatic markers, such as CXCR4, indicating that the anti-NME7 antibodies or peptides inhibit the transition to metastatic cancer (FIG. 15A-15C). These results show that antibodies that bind to NME7_{AB} can be administered to a patient for the treatment or prevention of metastatic cancers.

Peptides Derived from NME7_{AB} or NME7-X1 Competitively Inhibit the Binding of Intact NME7_{AB} and NME7-X1 and are Anti-Cancer Agents.

In another aspect of the invention, therapeutic agents for the treatment or prevention of cancers are peptides derived from the NME7 sequence, which are administered to a patient for the treatment or prevention of cancers. In one aspect, the NME7-derived peptides are administered to a patient so that the peptides, which should be shorter than the entire NME7 and unable to confer the oncogenic activity of NME7, bind to the targets of NME7 and competitively inhibit the interaction of intact NME7 with its targets, wherein such interactions promote cancer. Since NME7_{AB} is fully able to confer oncogenic activity, the sequence of NME7_{AB} is preferred as the source for the shorter peptide(s), wherein it must be confirmed that the peptides themselves are not able to promote cancerous growth or other tumorigenic or oncogenic activity. In a preferred embodiment, one or more peptides having the sequence of a portion of NME7_{AB} and being preferably about 12-56 amino acids in length are administered to a patient. To increase half-life, the peptides may be peptide mimics, such as peptides with unnatural backbone or D-form amino acids for L. In yet another case, the anti-cancer therapeutic agent is a peptide or peptide mimic wherein the peptide has a sequence highly homologous to at least a portion of NME7, NME7_{AB}, or NME7-X1 or its target the MUC1* extracellular domain, comprising the PSMGFR peptide, also called “FLR” in some cases herein.

FIG. 6-FIG. 9 provide a listing of preferred amino acid sequences that are predicted to inhibit NME7 binding to its cognate target. In a still more preferred embodiment, the peptides that are chosen for administration to a patient suffering from cancer or at risk of developing cancer are chosen because they bind to an NME7 binding partner and they do not themselves confer tumorigenic activity. In a yet more preferred embodiment, the NME7 binding partner is the extracellular domain of MUC1*. In a still more preferred embodiment, the NME7 binding partner is the PSMGFR peptide.

By the term “conferring tumorigenic activity or oncogenic activity”, it is meant that the peptides themselves cannot support or promote the growth of cancers. Another way of testing whether or not a peptide or peptides derived from NME7 can promote tumorigenesis is to test whether or not the peptides can support pluripotent growth of human stem cells. NME proteins and peptides that support pluripotent human stem cell growth also support cancer growth. In yet another method, peptides are de-selected if they can cause somatic cells to revert to a less mature state.

Fragments of NME7_{AB} inhibit cancer cell growth and the transition of cancer cells to a more metastatic state. As a demonstration, NME7 peptides A1, A2, B1, B2 and B3 added separately (FIG. 12) or in combinations (FIG. 13) inhibit the growth of cancer cells. In addition, NME7 peptides A1, A2, B1, B2 and B3 inhibited the transition of cancer cell to a more metastatic state (FIG. 15).

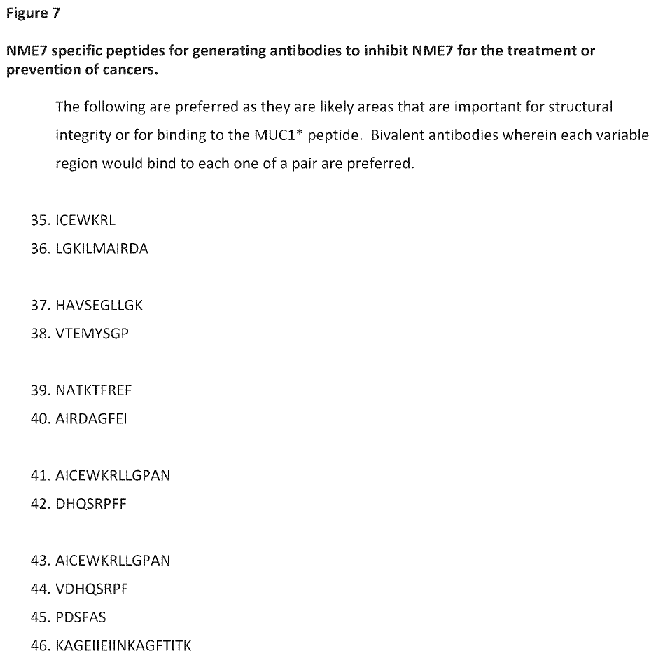

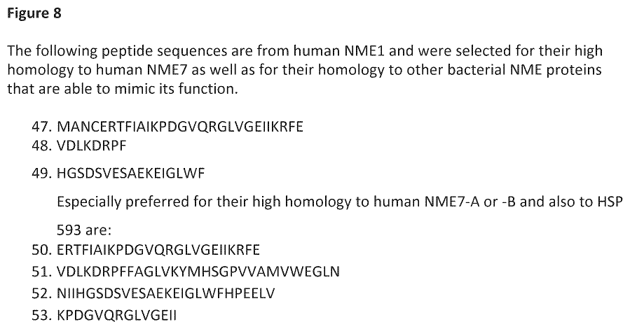

Thus, antibodies generated by immunizing with peptides specific to NME7, and specific to NME7_{AB} or NME7-X1 will block the cancerous action of NME7 species and will be potent anti-cancer agents. Similarly, these results show that the peptides specific to NME7, and specific to NME7_{AB} or NME7-X1 will block the cancerous action of NME7 species. In one aspect of the invention, the peptides are chosen from the list shown in FIG. 6. In one aspect of the invention the peptides are chosen from the list shown in FIG. 7. In one aspect of the invention the peptides are chosen from the list shown in FIG. 8. In yet another aspect of the invention the peptides are chosen from the list shown in FIG. 9. These antibodies may be generated by immunizing or may be generated or selected by other means, then selected for their ability to bind to NME7, NME7_{AB}, NME7-X1 or NME7 derived peptides, including but not limited to NME7 derived peptides A1 (SEQ ID NO: 141), A2 (SEQ ID NO: 142), B1 (SEQ ID NO: 143), B2 (SEQ ID NO: 144) or B3 (SEQ ID NO: 145). Such antibodies may be polyclonal, monoclonal, bispecific, bivalent, monovalent, single chain, scFv, human or humanized or may be an antibody mimic such as protein scaffolds that present recognition regions that bind to a specific target.

Anti-NME7 antibodies for use in the treatment or prevention of cancers can be generated by standard methods known to those skilled in the art wherein those methods are used to generate antibodies or antibody-like molecules that recognize NME7, NME7_{AB} or a shorter form of NME7_{AB} wherein an additional 10-25 amino acids form the N-terminus are not present. Such antibodies may be human or humanized. Such antibodies may be polyclonal, monoclonal, bispecific, bivalent, monovalent, single chain, scFv, human or humanized or may be an antibody mimic such as protein scaffolds that present recognition regions that bind to a specific target.

Anti-NME7 antibodies that are generated by immunization with the NME7 derived peptides A1 (SEQ ID NO: 141), A2 (SEQ ID NO: 142), B1 (SEQ ID NO: 143), B2 (SEQ ID NO: 144) or B3 (SEQ ID NO: 145) or antibodies that bind to the A1, A2, B1, B2 or B3 peptides are antibodies that bind to NME7_{AB} and NME7-X1, but resist binding to NME1 which may be required for the function of some healthy cells. Such antibodies inhibit the binding of NME7_{AB} or NME7-X1 to their target receptor, MUC1*. Antibodies that bind to A1, A2, B1, B2 or B3 peptides are antibodies can be administered to a patient diagnosed with or at risk of developing a cancer or metastasis. Such antibodies may be human or humanized. Such antibodies may be polyclonal, monoclonal, bispecific, bivalent, monovalent, single chain, scFv, or may be an antibody mimic such as protein scaffolds that present recognition regions that bind to a specific target.

Anti-NME7 antibodies that are generated by immunization with the B3 peptide or antibodies that bind to the B3 peptide are especially specific for the recognition of NME7_{AB} and NME7-X1. Such antibodies are also very efficient at inhibiting the binding of NME7_{AB} or NME7-X1 to their target receptor, MUC1*. Antibodies that bind to the B3 peptide are also exceptionally efficient at preventing, inhibiting and reversing cancer or cancer metastases. Such antibodies may be human or humanized. Such antibodies may be polyclonal, monoclonal, bispecific, bivalent, monovalent, single chain, scFv, or may be an antibody mimic such as protein scaffolds that present recognition regions that bind to a specific target.

Note that the polyclonal antibody #61, which was generated in rabbits immunized with the B3 peptide, inhibited the transformation of cancer cells to cancer stem cells as evidenced by antibody #61 blocking upregulation of metastatic marker CXCR4 (FIG. 15).

The B3 peptide (SEQ ID NO: 145) derived from NME7 has a Cysteine at position 14, which complicates the generation of anti-NME7 antibodies. We mutated Cysteine 14 to Serine to make AIFGKTKIQNAVHSTDLPEDGLLEVQYFF (SEQ ID NO:169) and immunized animals to generate anti-NME7 monoclonal antibodies. The resultant antibodies bind to the native B3 sequence as well as the B3Cys14Ser peptide. Seven (7) high affinity and specific monoclonal antibodies were generated: 8F9A5A1, 8F9A4A3, 5F3A5D4, 5D9E2B11, 5D9E10E4, 5D9G2C4, and 8H5H5G4. However, various sequence alignments showed that there are only three (3) unique sequence antibodies: 8F9A5A1, 8F9A4A3, 8F9A4P3 and 5F3A5D4 as seen below. Bolded and underlined regions indicate CDR sequences.

| HEAVY CHAIN ALIGNMENT | | |
|---|---|---|
| 8F9A5A1H | IQLVQSGPELKKPGETVKISCKASGYTFTNYGMNWVKQAPGKGLKWMGWINTYTGEPTYV | 60 |
| 8F9A4A3H | VQLQQSGPELVKPGASVKISCKTSGNTFTEYTMHWVKQSHGKSLEWIGGFNPNNGVTNYN | 60 |
| 5D9E2B11H | VQLQQSGPDLVKPGTSVKISCKTSGNTFTEYTMHWVKQSHGKSLEWIGGFNPNNGVTNYN | 60 |
| 5D9E10E4H | VQLQQSGPDLVKPGTSVKISCKTSGNTFTEYTMHWVKQSHGKSLEWIGGFNPNNGVTNYN | 60 |
| 5D9G2C4H | VQLQQSGPDLVKPGTSVKISCKTSGNTFTEYTMHWVKQSHGKSLEWIGGFNPNNGVTNYN | 60 |
| 5F3A5D4H | VQLQQSGPDLVKPGTSVKISCKTSGNTFTEYTMHWVKQSHGKSLEWIGGFNPNNGVTNYN | 60 |
| 8H5H5G4H | VQLQQSGPDLVKPGTSVKISCKTSGNTFTEYTMHWVKQSHGKSLEWIGGFNPNNGVTNYN | 60 |
| | :** ****:* *** :******:** ***:* *:****: **.*:*:* :* .* .* | |
| 8F9A5A1H | DDFKGRFAFSLETSATCAYLQINNLKNEDTSTYFCARLR--GIRPGPLAYWGQGTLVTVS | 118 |
| 8F9A4A3H | QKFKGKATLTVDKSSSIAYMELRSLTSEDSAVYYCARRYYHSLYVFYFDYWGQGTTLTVS | 120 |
| 5D9E2B11H | QKFKGKATLTVDKSSSTAYMELRSLTSEDSAVYYCARRYYHSTYVFYFDSWGQGTT---- | 116 |
| 5D9E10E4H | QKFKGKATLTVDKSSSTAYMELRSLTSEDSAVYYCARRYYHSTYVFYFDSWGQGTTLTVS | 120 |
| 5D9G2C4H | QKFKGKATLTVDKSSSTAYMELRSLTSEDSAVYYCARRYYHSTYVFYFDSWGQGTTLTVS | 120 |
| 5F3A5D4H | QKFKGKATLTVDKSSSTAYMELRSLTSEDSAVYYCARRYYHSTYVFYFDSWGQGTTLTVS | 120 |
| 8H5H5G4H | QKFKGKATLTVDKSSSTAYMELRSLTSEDSAVYYCARRYYHSTYVFYFDSWGQGTTLTVS | 120 |
| | :.***: :::::.*::***:::..*..**::.*:*** . : ***** | |
| 8F9A5A1H | A | 119 (SEQ ID NO: 172) |
| 8F9A4A3H | S | 121 (SEQ ID NO: 1205) |
| 5D9E2B11H | - | 116 (SEQ ID NO: 174) |
| 5D9E10E4H | S | 121 (SEQ ID NO: 175) |
| 5D9G2C4H | S | 121 (SEQ ID NO: 176) |
| 5F3A5D4H | S | 121 (SEQ ID NO: 177) |
| 8H5H5G4H | S | 121 (SEQ ID NO: 178) |
| 8F9A4P3H | VQLQQSGPELVKPGASVKISCKTSGNTFTEYTMHWVKQSHGKSLEWIGGFNPNNGVTNYN | 60 |
| 8F9A4A3H | VQLQQSGPELVKPGASVKISCKTSGNTFTEYTMHWVKQSHGKSLEWIGGFNPNNGVTNYN | 60 |
| 5D9E2B11H | VQLQQSGPDLVKPGTSVKISCKTSGNTFTEYTMHWVKQSHGKSLEWIGGFNPNNGVTNYN | 60 |
| 5D9E10E4H | VQLQQSGPDLVKPGTSVKISCKTSGNTFTEYTMHWVKQSHGKSLEWIGGFNPNNGVTNYN | 60 |
| 5D9G2C4H | VQLQQSGPDLVKPGTSVKISCKTSGNTFTEYTMHWVKQSHGKSLEWIGGFNPNNGVTNYN | 60 |
| 5F3A5D4H | VQLQQSGPDLVKPGTSVKISCKTSGNTFTEYTMHWVKQSHGKSLEWIGGFNPNNGVTNYN | 60 |
| 8H5H5G4H | VQLQQSGPDLVKPGTSVKISCKTSGNTFTEYTMHWVKQSHGKSLEWIGGFNPNNGVTNYN | 60 |
| | ********:*****:********************************************* | |
| 8F9A4P3H | QKFKGKATLTVDKSSSTAYMELRSLTSEDSAVYYCARRYYHSLYVFYFDYWGQGTTLTVS | 120 |
| 8F9A4A3H | QKFKGKATLTVDKSSSTAYMELRSLTSEDSAVYYCARRYYHSLYVFYFDYWGQGTTLTVS | 120 |
| 5D9E2B11H | QKFKGKATLTVDKSSSTAYMELRSLTSEDSAVYYCARRYYHSTYVFYFDSWGQGTT---- | 116 |
| 5D9E10E4H | QKFKGKATLTVDKSSSTAYMELRSLTSEDSAVYYCARRYYHSTYVFYFDSWGQGTTLTVS | 120 |
| 5D9G2C4H | QKFKGKATLTVDKSSSTAYMELRSLTSEDSAVYYCARRYYHSTYVFYFDSWGQGTTLTVS | 120 |
| 5F3A5D4H | QKFKGKATLTVDKSSSTAYMELRSLTSEDSAVYYCARRYYHSTYVFYFDSWGQGTTLTVS | 120 |
| 8H5H5G4H | QKFKGKATLTVDKSSSTAYMELRSLTSEDSAVYYCARRYYHSTYVFYFDSWGQGTTLTVS | 120 |
| | ****************************************** ****** ****** | |
| 8F9A4P3H | S | 121 (SEQ ID NO: 173) |
| 8F9A4A3H | S | 121 (SEQ ID NO: 1205) |
| 5D9E2B11H | - | 116 (SEQ ID NO: 174) |
| 5D9E10E4H | S | 121 (SEQ ID NO: 175) |
| 5D9G2C4H | S | 121 (SEQ ID NO: 176) |
| 5F3A5D4H | S | 121 (SEQ ID NO: 177) |
| 8H5H5G4H | S | 121 (SEQ ID NO: 178) |
| LIGHT CHAIN ALIGNMENT | | |
| 8F9A4P3L | ETTVTQSPASLSMAIGEKVTIRCITSTDIDDDMNWYQQKPGEPPKLLISEGNTLRPGVPS | 60 |
| 8F9A4A3L | DIQMTQTTSSLSASLGDRVTLSCSASQGISNYLNWYQQKPDGTVELLIFYTSSLHSGVPS | 60 |
| 5D9E2B11L | DIQMTQTTSSLSASLGDRVTISCSASQGISNYLNWFQQKPDGTIKLLIYYTSSLHSGVPS | 60 |
| 5D9E10E4L | DIQMTQTTSSLSASLGDRVTISCSASQGISNYLNWFQQKPDGTIKLLIYYTSSLHSGVPS | 60 |
| 5D9G2C4L | DIQMTQTTSSLSASLGDRVTISCSASQGISNYLNWFQQKPDGTIKLLIYYTSSLHSGVPS | 60 |
| 5F3A5D4L | DIQMTQTTSSLSASLGDRVTISCSASQGISNYLNWFQQKPDGTIKLLIYYTSSLHSGVPS | 60 |
| 8H5H5G4L | DIQMTQTTSSLSASLGDRVTISCSASQGISNYLNWFQQKPDGTIKLLIYYTSSLHSGVPS | 60 |
| 8F9A5A1L | EILLTQSPAIIAASPGEKVTITCSASSSV-SYMNWYQQKPGSSPKIWEYGISNLASGVPA | 60 |
| | : :**: : :: : *::*** * :* .: . :**:****. *: * ..* ***: | |
| 8F9A4P3L | RFSSSGYGTDFVFTIENMLSEDVADYYCLQSDNLPLTFGSGTKLEIKR | 108 (SEQ ID NO: 185) |
| 8F9A4A3L | RFSGSGSGTDYSLTISNLEPEDIATYYCQQYSKLPYTFGGGTKLEIK | 108 (SEQ ID NO: 1109) |
| 5D9E2B11L | RFSGSGSGTDYSLTISNVEPEDIATYYCQQYSKLPYTFGGGTKLEIKR | 108 (SEQ ID NO: 186) |
| 5D9E10E4L | RFSGSGSGTDYSLTISNVEPEDIATYYCQQYSKLPYTFGGGTKLEIKR | 108 (SEQ ID NO: 187) |
| 5D9G2C4L | RFSGSGSGTDYSLTISNVEPEDIATYYCQQYSKLPYTFGGGTKLEIKR | 108 (SEQ ID NO: 188) |
| 5F3A5D4L | RFSGSGSGTDYSLTISNVEPEDIATYYCQQYSKLPYTFGGGTKLEIKR | 108 (SEQ ID NO: 189) |
| 8H5H5G4L | RFSGSGSGTDYSLTISNVEPEDIATYYCQQYSKLPYTFGGGTKLEIKR | 108 (SEQ ID NO: 190) |
| 8F9A5A1L | RFSGSGSGTSFSFTINSMEAEDVATYYCQQRSSYPPTFGGGTKLEIKR | 108 (SEQ ID NO: 191) |
| | ***.** **.: :**..: **:* *** * .. * ***.******** | |
| 5D9E2B11L | DIQMTQTTSSLSASLGDRVTISCSASQGISNYLNWFQQKPDGTIKLLIYYTSSLHSGVPS | 60 |
| 5D9E10E4L | DIQMTQTTSSLSASLGDRVTISCSASQGISNYLNWFQQKPDGTIKLLIYYTSSLHSGVPS | 60 |
| 5D9G2C4L | DIQMTQTTSSLSASLGDRVTISCSASQGISNYLNWFQQKPDGTIKLLIYYTSSLHSGVPS | 60 |
| 5F3A5D4L | DIQMTQTTSSLSASLGDRVTISCSASQGISNYLNWFQQKPDGTIKLLIYYTSSLHSGVPS | 60 |
| 8H5H5G4L | DIQMTQTTSSLSASLGDRVTISCSASQGISNYLNWFQQKPDGTIKLLIYYTSSLHSGVPS | 60 |
| | ************************************************************ | |
| 5D9E2B11L | RFSGSGSGTDYSLTISNVEPEDIATYYCQQYSKLPYTFGGGTKLEIKR | 108 (SEQ ID NO: 192) |
| 5D9E10E4L | RFSGSGSGTDYSLTISNVEPEDIATYYCQQYSKLPYTFGGGTKLEIKR | 108 (SEQ ID NO: 193) |
| 5D9G2C4L | RFSGSGSGTDYSLTISNVEPEDIATYYCQQYSKLPYTFGGGTKLEIKR | 108 (SEQ ID NO: 194) |
| 5F3A5D4L | RFSGSGSGTDYSLTISNVEPEDIATYYCQQYSKLPYTFGGGTKLEIKR | 108 (SEQ ID NO: 195) |
| 8H5H5G4L | RFSGSGSGTDYSLTISNVEPEDIATYYCQQYSKLPYTFGGGTKLEIKR | 108 (SEQ ID NO: 196) |
| | ************************************************ | |
| 8F9A4P3L | ETTVTQSPASLSMAIGEKVTIRCITSTDIDDDMNWYQQKPGEPPKLLISEGNTLRPGVPS | 60 |
| 8F9A5A1L | EILLTQSPAIIAASPGEKVTITCSASSSV-SYMNWYQQKPGSSPKIWIYGISNLASGVPA | 60 |
| | * :***** :: : ****** * :*:.: . *********. **: * ..*: ***: | |
| 8F9A4P3L | RFSSSGYGTDFVFTIENMLSEDVADYYCLQSDNLPLTFGSGTKLEIKR | 108 (SEQ ID NO: 197) |
| 8F9A5A1L | RFSGSGSGTSFSFTINSMEAEDVATYYCQQRSSYPPTFGGGTKLEIKR | 108 (SEQ ID NO: 198) |
| | ***.** **.* ***:.* :**** *** * .. * ***.******** | |

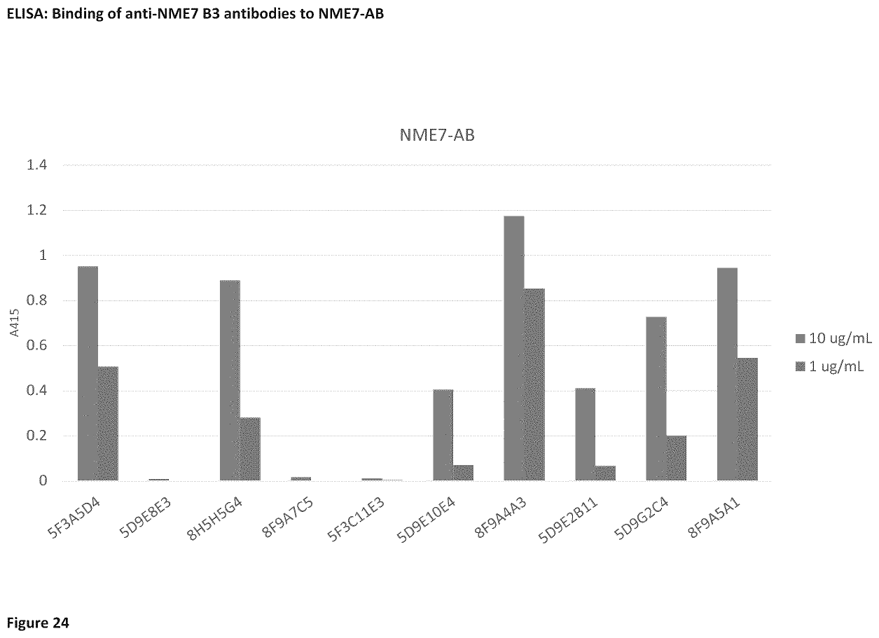

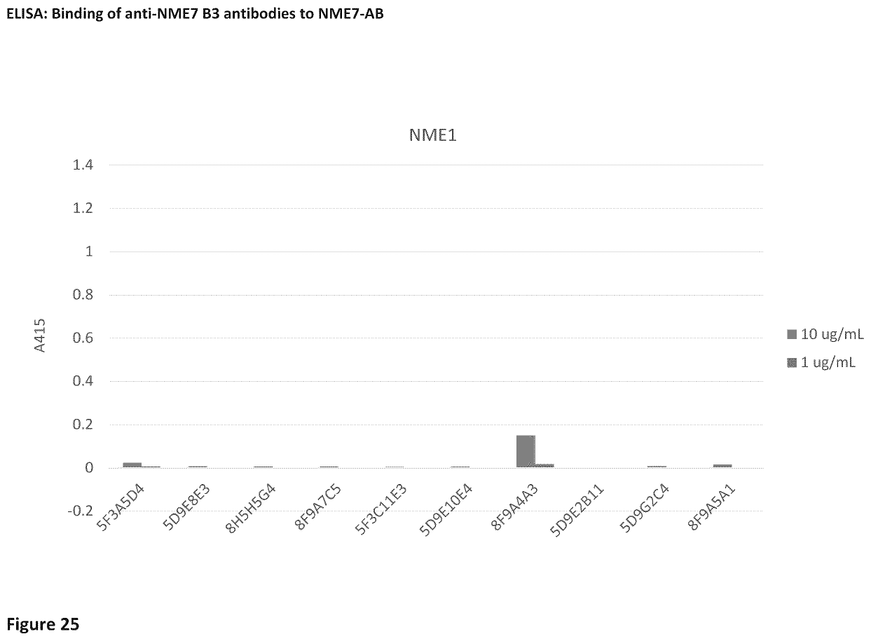

Monoclonal antibodies 5D9E2B11, 5D9E10E4, 5D9G2C4, and 8H5H5G4 all have the same sequence as 5F3A5D4, also known as 5D4. Herein, when we refer to antibody 5F3A5D4, aka 5D4, it is understood that it also applies to 5D9E2B11, 5D9E10E4, 5D9G2C4, and 8H5H5G4. As can be seen in FIG. 24 and FIG. 25 anti-NME7 antibodies 8F9A5A1, 8F9A4A3, and 5F3A5D4 all bind to NME7_{AB} but not to NME1. This is important because the A domain of NME7 has high homology to NME1, which is required for normal cell function. For an anti-cancer therapeutic or an anti-metastasis therapeutic it will be imperative to inhibit NME7_{AB} but not NME1.

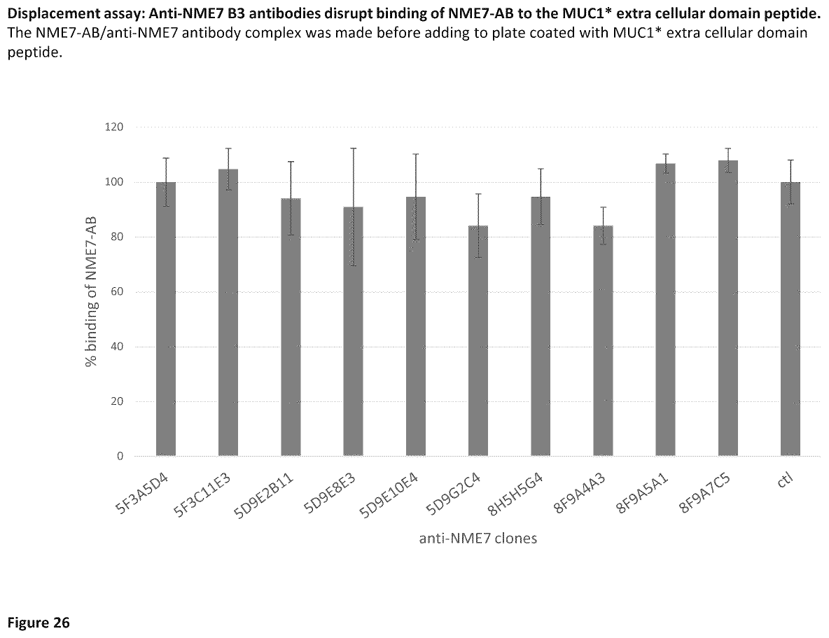

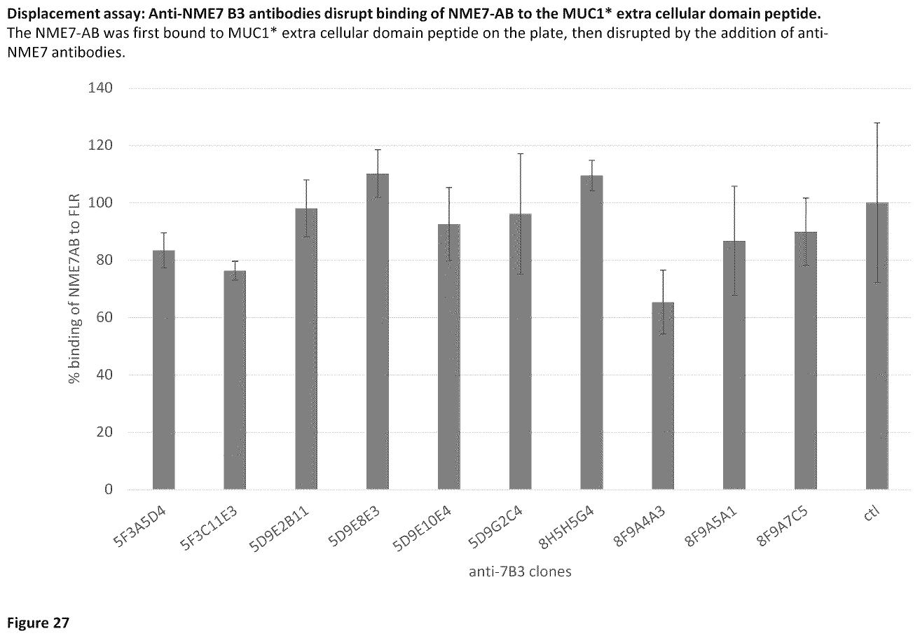

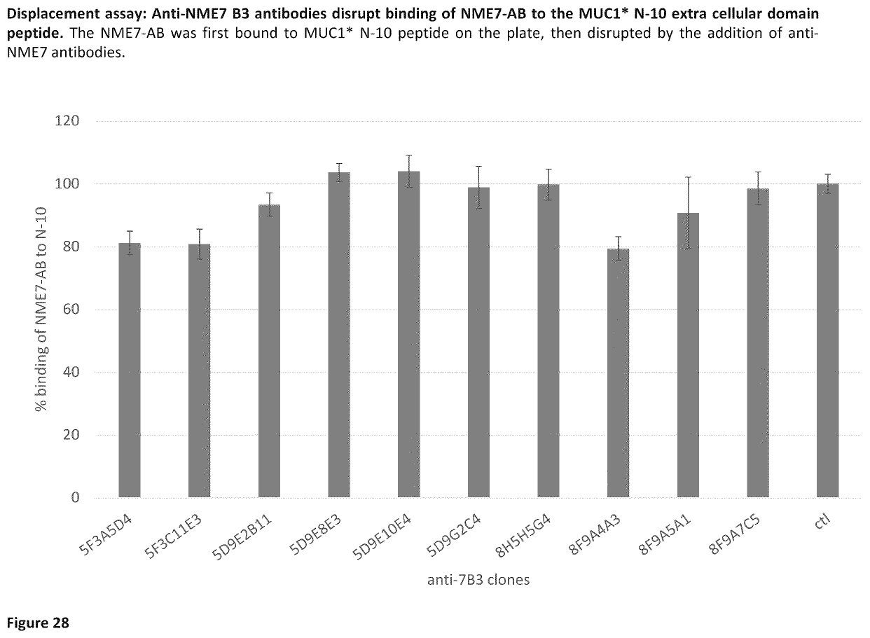

FIG. 26, FIG. 27 and FIG. 28 show that these anti-NME7 antibodies are also able to disrupt the binding of NME7_{AB} to the MUC1* PSMGFR peptide and the N-10 PSMGFR peptide. As can be seen, there is not a total displacement of NME7_{AB} from the MUC1* peptides. However, recall that NME7_{AB} is comprised of an A domain and a B domain, each of which are capable of binding to MUC1*. These antibodies were designed to disrupt binding of the B domain to MUC1*; the A domain of NME7_{AB} would still be able to bind to the MUC1* peptide on the plate surface. For a useful therapeutic, the antibody would only need to disrupt the binding of one domain to MUC1* and in so doing ligand-induced dimerization and activation of MUC1* growth factor receptor would be blocked. Antibodies or antibody mimics that bind to the NME7 B3 peptide or the B3 Cys14Ser peptide (SEQ ID NO:169) are antibodies can be administered to a patient diagnosed with or at risk of developing a cancer or metastasis.

It is well known in the field that it is difficult to make cancer cells metastasize in an animal model. It is estimated that in a human tumor only about 1 in 100,000 or even 1 in 1,000,000 cancer cells is able to break away from the tumor and implant elsewhere to initiate a metastasis [Al-Hajj et al., 2003]. Some researchers report that T47D breast cancer cells injected into an immune compromised mouse will metastasize after about 12 weeks [Harrell et al 2006]. Other researchers report that AsPC-1 pancreatic cancer cells will metastasize after about 4 weeks [Suzuki et al, 2013].

Here, we show that T47D breast cancer cells grown for 10 days in a serum-free media containing recombinant NME7_{AB} as the only growth factor. It was observed that when grown in NME7_{AB}, about 25% of the cancer cells began floating, stopped dividing but were still viable. PCR measurement showed that these “floating” cells greatly upregulated expression of the breast cancer metastatic factor CXCR4.

In some of the figures presented herein, these floater cells are referred to as cancer stem cells (CSCs). Immune compromised female nu/nu mice were implanted with 90-day release estrogen pellets. Either 500,000 T47D-wt cells or 10,000 T47D-CSCs (cancer stem cells) were injected into the tail vein (i.v.), sub-cutaneously (s.c.), or into the intra-peritoneal space (i.p.) of the nu/nu mice. These cancer cells were engineered to express Luciferase. To visualize the tumors or cancer cells, animals are injected with Luciferin, then visualized on an IVIS instrument 10 minutes later. As can be seen in the IVIS measurements of FIG. 33A-FIG. 33B, by Day 6 the 500,000 T47D-wt cells injected into the tail vein show no signs of live cancer cells or cancer cell engraftment.

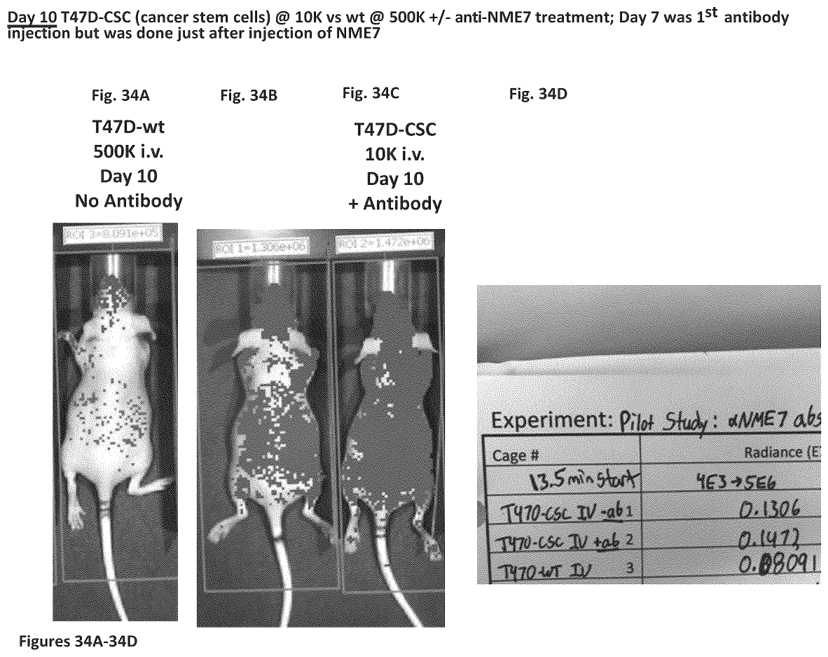

In stark contrast, the 10,000 T47D-CSC injected into the tail vein have metastasized. Before the Day 6 IVIS measurement, the T47D-CSC mice were injected with 32 nM recombinant NME7_{AB}. The next day, one of the two CSC mice was injected with a cocktail of anti-NME7 monoclonal antibodies 8F9A5A1, 8F9A4A3, and 5F3A5D4 in a volume of 200 μL at a concentration that corresponds to 15 mgs/kg. The nearly coincident injection of NME7_{AB} and anti-NME7 antibody likely nullified the effect of the antibody. FIG. 34 shows that by Day 10, the treated mouse is almost entirely metastatic. As can be seen in the figure, the mouse chosen for treatment is more metastatic than the comparable T47D-CSC mouse.

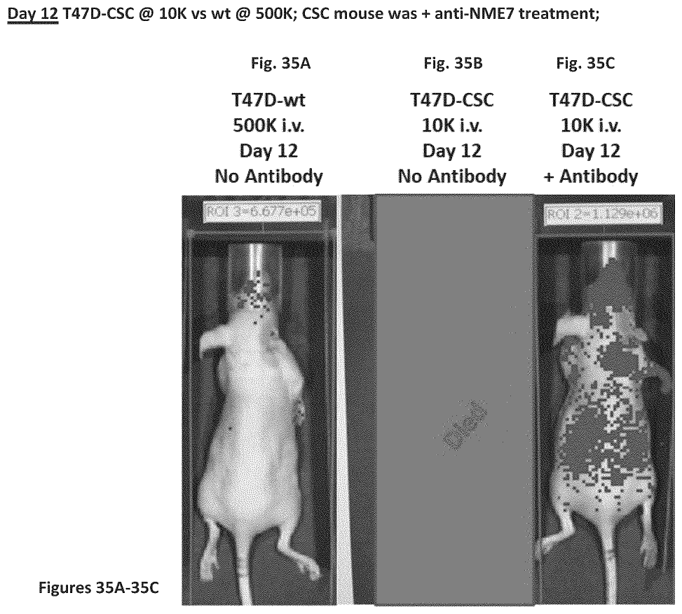

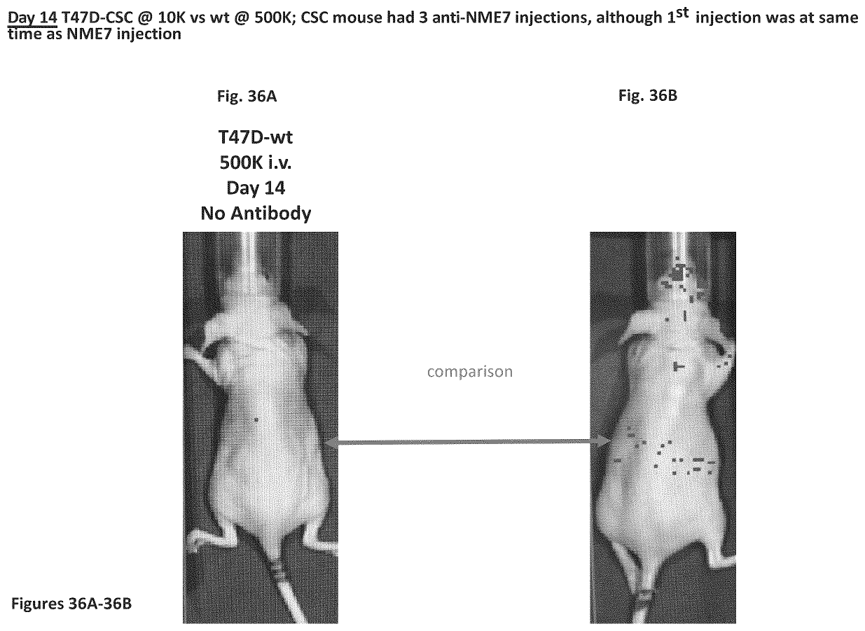

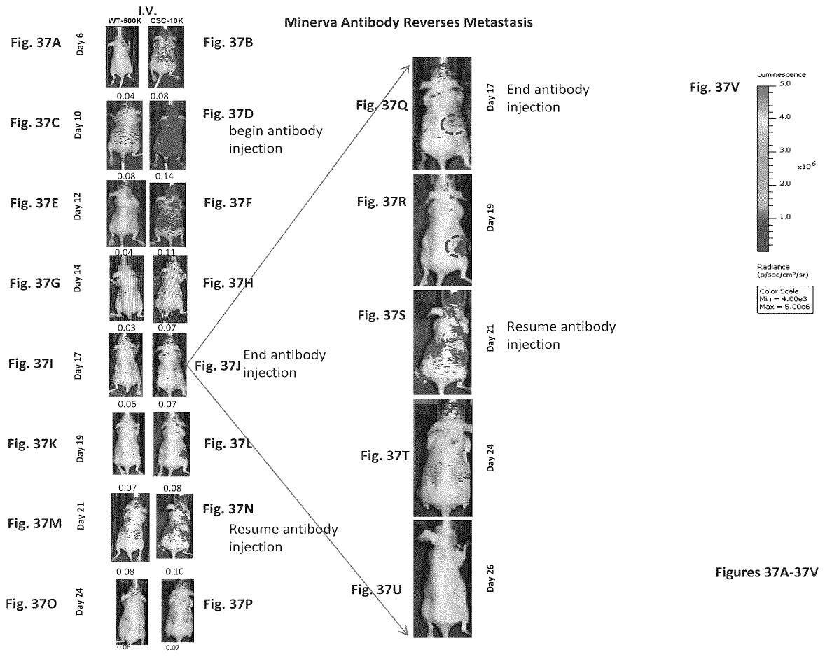

That animal was again injected with the anti-NME7 antibodies on Day 10. The IVIS measurement of Day 12 (FIG. 35) shows that the antibody treated mouse is beginning to clear the metastases. By Day 14 (FIG. 36) the untreated mouse has died from rampant metastases and the treated mouse has cleared the metastases. FIG. 37 shows the time course of IVIS measurements for the mouse injected with 500,000 T47D-wt cells and the mouse injected with T47D-CSCs that received anti-NME7 treatment until Day 17 when antibody treatment was suspended. As can be seen, on Day 17 there remained a small cluster of cancer cells, which by Day 19 had grown larger. By Day 21 the metastases had spread and antibody treatment was resumed. As is shown in the figure, after resumption of anti-NME7 antibody treatment, the animal was cleared of all metastases and shows no signs of ill health.

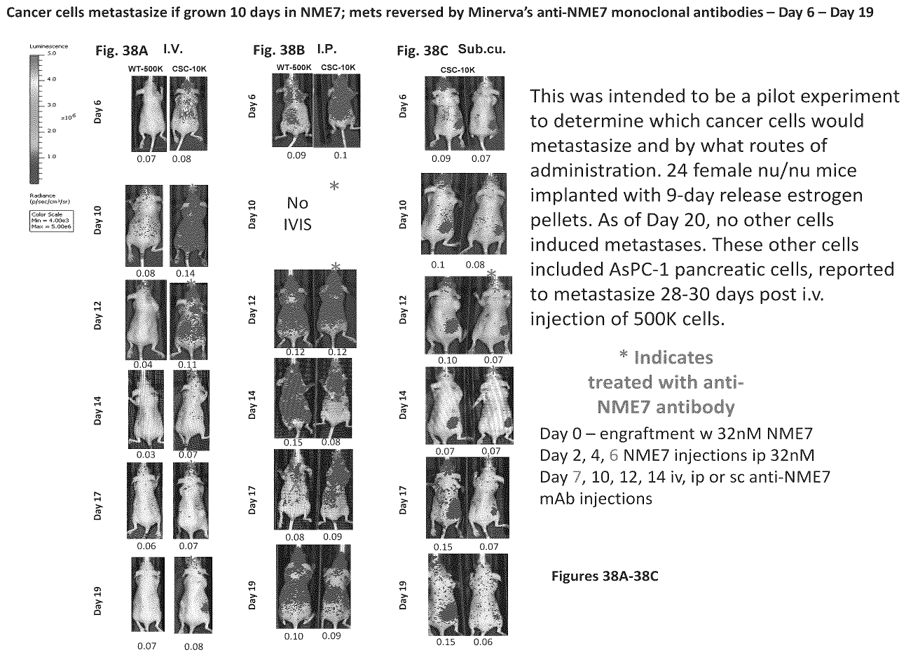

FIG. 38 shows the IVIS time course for animals that were injected sub-cutaneously or intra-peritoneally. Antibody injections for animals injected with CSCs sub-cutaneously or intra-peritoneally were also injected with anti-NME7 antibodies s.c. or i.p. In these animals, antibody injections stopped at Day 17 and did not resume. FIG. 39-FIG. 40 show that a polyclonal anti-NME7 antibody generated by immunization with the B3 peptide stains advanced cancers and metastatic cancers but not normal tissues or low-grade cancers, where only 1 in 100,000 or 1 in 1,000,000 cancer cells would be a metastatic cancer cells. Taken together, these data show that anti-NME7 antibodies 8F9A5A1, 8F9A4A3, and 5F3A5D4 or 8F9A5A1, or 8F9A4A3, or 5F3A5D4 administered to a patient diagnosed with or at risk of developing a cancer would prevent, inhibit the formation of, or reverse cancer metastases.

In addition to treating metastatic animals with a cocktail of anti-NME7AB antibodies, we also administered monoclonal anti-NME7AB antibodies individually and showed they were capable of preventing as well as reversing cancer metastases. In one demonstration, female nu/nu mice weighing approximately 20 g each, were implanted with 90-day estrogen release pellets between 8-10 weeks of age. Cancer cells were made metastatic by culturing for 10-15 days in a serum-free media supplemented with growth factor NME7_{AB}. Both adherent and floating cells show upregulation of metastatic markers and in animals are able to metastasize within 4-7 days. In this case, the floating cells were harvested on Day 11 of in vitro culture and injected into the tail vein of the test animals. To test a prevention model, one group of animals was injected into the tail vein, 24 hours before injection of the metastatic cancer cells, with anti-NME7_{AB} antibody 8F9A4A3 at 15 mg/kg and injected thereafter with the same dosage approximately every 48 hours. FIG. 42A-FIG. 42F shows photographs of female nu/nu mice, which were injected into the tail vein with 10,000 Luciferase positive T47D metastatic breast cancer stem cells and treated with the anti-NME7_{AB} antibody 4A3 also known as 8F9A4A3. To image cancer cells, the Luciferase substrate, Luciferin, is intraperitoneally injected 10 minutes before being photographed in IVIS instrument. FIG. 42A-42C show IVIS photographs with animals face down. FIG. 42D-42F show IVIS photographs with animals face up. FIGS. 42A and 42D show control animals injected with phosphate buffered saline solution. FIGS. 42B and 42E show a prevention model in which animals were injected with anti-NME7_{AB} antibody 4A3 24 hrs before injection of the metastatic cancer cells, then approximately every other day for a total of 12 antibody injections over 22 days. FIGS. 42C and 42F show a reversal model in which animals were injected with anti-NME7_{AB} antibody 4A3 24 hrs after injection of the metastatic cancer cells, then approximately every other day for a total of 11 antibody injections over 20 days. As can be seen in the figure, anti-NME7_{AB} antibody 8F9A4A3 can prevent, as well as reverse an established metastasis.

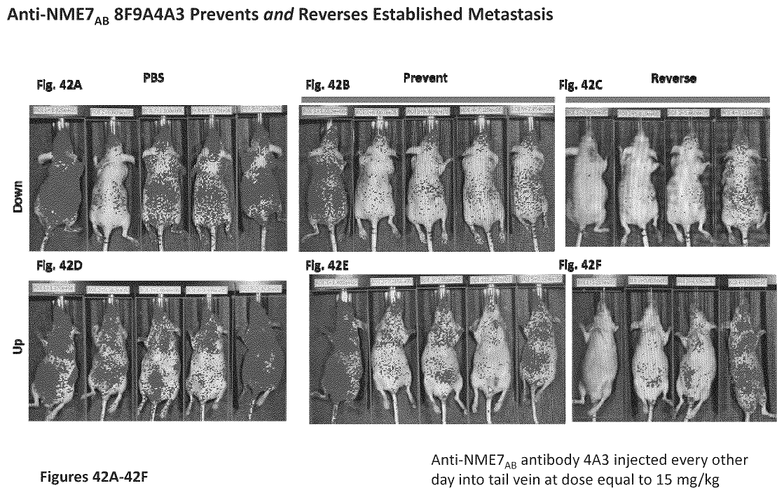

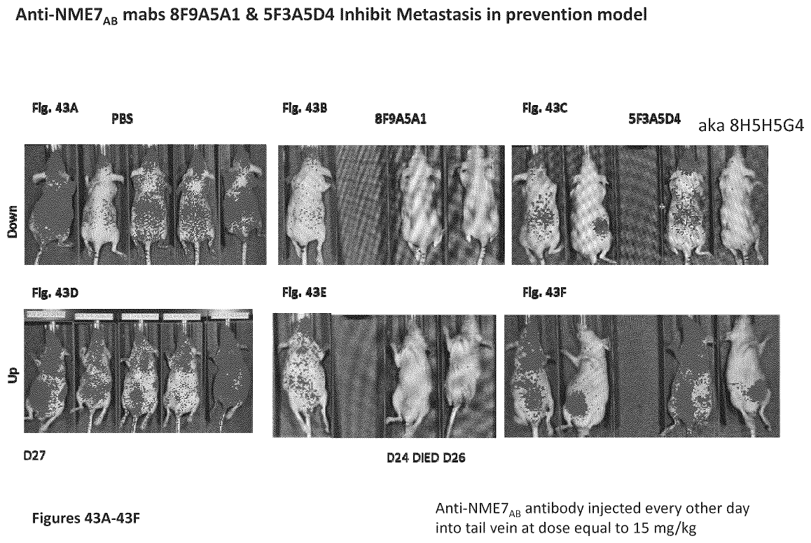

Anti-NME7_{AB} antibodies 5A1 and 5D4 were also tested in a metastasis prevention model and shown to greatly inhibit cancer metastasis. FIG. 43A-43F shows photographs of female nu/nu mice weighing approximately 20 g each, which were injected into the tail vein with 10,000 Luciferase positive T47D metastatic breast cancer stem cells and treated with the anti-NME7_{AB} antibodies 5A1, also known as 8F9A5A1, and 5D4, also known as 5F3A5D4. To image cancer cells, the Luciferase substrate, Luciferin, is intraperitoneally injected 10 minutes before being photographed in IVIS instrument. FIG. 43A-43C show IVIS photographs with animals face down. FIG. 43D-43F show IVIS photographs with animals face up. FIGS. 43A and 43D show control animals injected with phosphate buffered saline solution. FIGS. 43B, 43E, 43C and 43F show a prevention model in which animals were injected with anti-NME7_{AB} antibodies, at 15 mg/kg 24 hours before injection of the metastatic cancer cells, then approximately every other day for a total of 12 antibody injections over 22 days. Photographs were taken either at Day 24 or at Day 27. Specifically, mouse #1 in the group treated with antibody 5A1 was photographed at Day 27 while mouse #2 and #3 were photographed on Day 24 because animals died on Day 26.

Anti-NME7_{AB} antibodies 5A1 and 5D4 were also tested in a metastasis reversal model and shown to greatly inhibit established cancer metastases. In this experiment, animals were injected on Day 0 into the tail vein with 10,000 T47D metastatic cancer cells mixed with NME7_{AB} at a final concentration of 32 nM. Further, animals were injected twice, Day 3 and Day 4, with more NME7_{AB} which our experiments have shown make the metastasis more difficult to reverse. The first antibody injection was on Day 7. Because the degree of metastasis in each test animal is somewhat variable, we wanted to make certain that the apparent clearance of metastatic cancer cells was due to the anti-NME7_{AB} treatment. We therefore treated the animals with alternating high dose and low doses. As can clearly be seen in FIG. 44, high dose anti-NME7_{AB} results in clearance of the metastasis, which if not completely eradicated comes back and even increases with lower dose. This experiment shows that all three anti-NME7_{AB} antibodies tested, 5A1, 4A3 and 5D4, which are able to bind to the NME7-B3 peptide, inhibit cancer metastasis in a concentration dependent manner. FIG. 44A-44D show photographs of female nu/nu mice that were injected into the tail vein with 10,000 Luciferase positive T47D metastatic breast cancer stem cells mixed with NME7_{AB} at a final concentration of 32 nM. Animals were then injected into the tail vein with 32 nM NME7_{AB} before being treated with individual anti-NME7_{AB} antibodies. FIG. 44A shows control animals injected with phosphate buffered saline solution. FIG. 44B shows animals treated with anti-NME7_{AB} monoclonal antibody 8F9A5A1. FIG. 44C shows animals treated with anti-NME7_{AB} monoclonal antibody 8F9A4A3. FIG. 44D shows animals treated with anti-NME7_{AB} monoclonal antibody 5F3A5D4. Green arrows indicate low antibody dosage (5-7 mg/kg) over the indicated period and Red arrows indicate high dosage (15 mg/kg). As can be seen in the figure, the metastasis clears considerably when antibody is administered at 15 mg/kg.

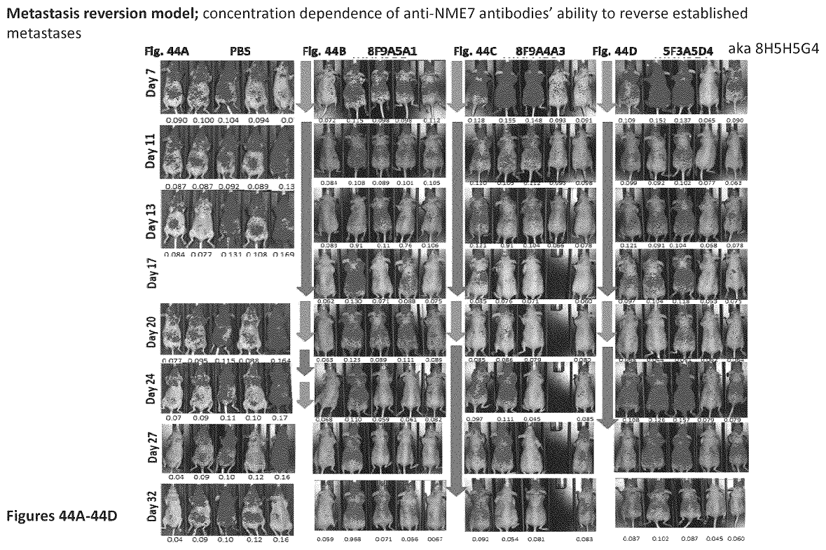

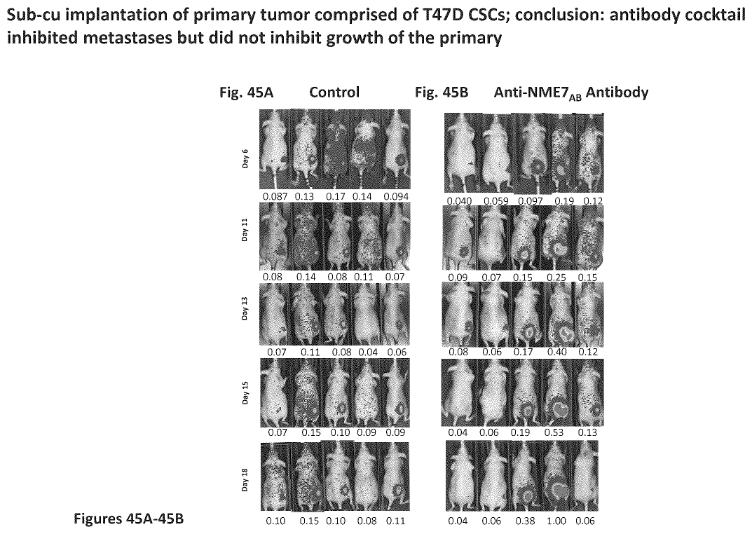

In addition to demonstrating that the anti-NME7_{AB} antibodies of the invention can inhibit metastasis, we tested their effect on metastasis from a primary tumor, which would more closely mimic the physiology of cancer metastasis. We generated T47D metastatic breast cancer cells, also known as cancer stem cells (CSCs) by culturing the cancer cells in a minimal serum-free media containing NME7_{AB} for 10-15 days. These T47D CSCs were then implanted sub-cutaneously into the right flank of NSG mice into which had been implanted a 90-day estrogen release pellet. The implanted cancer cells were Luciferase positive so that after injection of the Luciferase substrate, Luciferin, the cancer cells emit photons and can be photographed in an IVIS instrument to measure and locate the implanted cancer cells. FIG. 45A-45B shows photographs of female nu/nu mice that on Day 0 were injected sub-cutaneously into the right flank with 10,000 Luciferase positive T47D metastatic breast cancer stem cells, mixed with NME7_{AB} to a final concentration of 32 nM, then mixed in a 1:1 vol:vol with Matrigel. Tumor engraftment was allowed to progress Day 0-Day 6. Animals were then treated i.v. by tail vein injection with anti-NME7_{AB} antibodies. Control animals were injected with PBS. FIG. 45A shows IVIS photographs of control animals. FIG. 45B shows IVIS photographs of animals injected into tail vein with a cocktail of anti-NME7_{AB} antibodies 5A1, 4A3 and 5D4 to a total concentration of 15 mg/kg. Antibodies or PBS were administered 4 times between Day 7 and Day 18. As can be seen in the figure, the anti-NME7_{AB} antibody treated animals show less metastases (blue dots in whole body) than the control group. In the treated group, 2 of the 5 animals have primary tumors that are larger than those in the control group. This could be because the anti-NME7_{AB} antibodies prevented the spread of the cancer cells, so they remained concentrated in the primary tumor. In this experiment, PCR analysis, performed prior to injection of the cancer cells, showed that after 11 days in culture with NME7_{AB}, the T47D breast cancer cells had upregulated CXCR4 by 109-fold, OCT4 by 2-fold, NANOG by 3.5-fold and MUC1 by 2.7-fold.

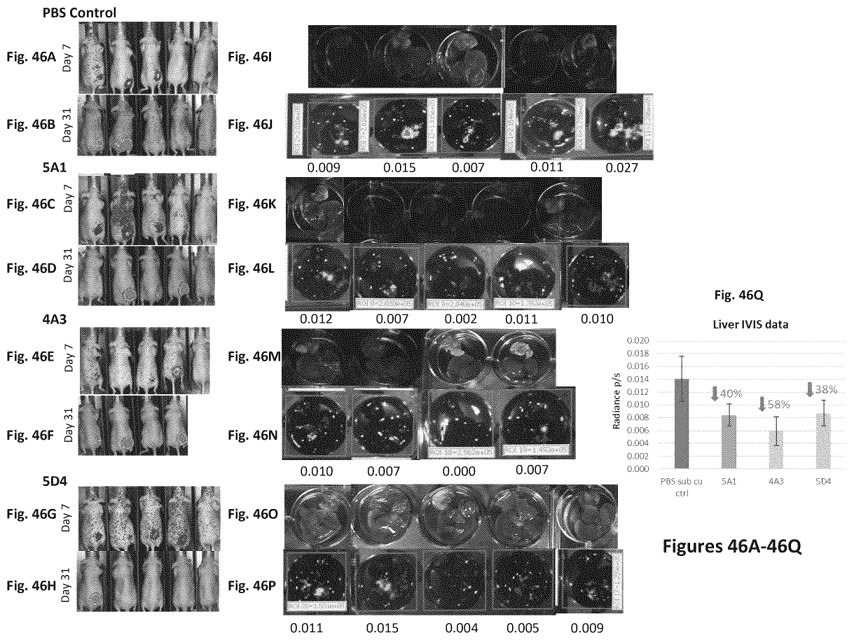

In another experiment, we tested the effect of anti-NME7_{AB} antibodies of the invention on metastasis from a primary tumor to organs that breast cancers typically metastasize to. Breast cancers commonly metastasize to liver, lung, bone and brain, in that order. We generated T47D metastatic breast cancer cells by culturing in a minimal serum-free media containing NME7_{AB} for 11 days. These T47D CSCs were then implanted sub-cutaneously into the right flank of NSG mice into which had been implanted a 90-day estrogen release pellet. FIG. 46A-46P shows photographs of female nu/nu mice that on Day 0 were injected sub-cutaneously into the right flank with 10,000 Luciferase positive T47D metastatic breast cancer stem cells, mixed with NME7_{AB} to a final concentration of 32 nM, then mixed in a 1:1 vol:vol with Matrigel. Tumor engraftment was allowed to progress Day 0-Day 6. Animals were then treated i.v., by tail vein injection, with anti-NME7_{AB} antibodies. Control animals were injected with PBS. On Day 38 animals were sacrificed and livers harvested then analyzed by IVIS to detect cancer cells that had metastasized to the liver. FIG. 46A-46B show whole body IVIS photographs of control animals that were injected with only PBS. FIG. 46C-46D show whole body IVIS photographs of control animals that were injected with the anti-NME7_{AB} antibody 5A1. FIG. 46E-46F show whole body IVIS photographs of control animals that were injected with the anti-NME7_{AB} antibody 4A3. FIG. 46G-46H show whole body IVIS photographs of control animals that were injected with the anti-NME7_{AB} antibody 5D4. FIGS. 46A, 46C, 46E, and 46G are IVIS photographs taken at Day 7 before any treatment. FIGS. 46B, 46D, 46F, and 46H are IVIS photographs taken at Day 31 after anti-NME7_{AB} antibody treatment or mock treatment. As can be seen in the figure, animals in the PBS control group show metastasis (blue dots) in the whole body IVIS photographs, while animals treated with anti-NME7_{AB} antibodies do not. FIG. 46I-46P show photographs and IVIS photographs of livers and lung harvested from animals after sacrifice. FIGS. 46I, 46K, 46M, and 46O are regular photographs. FIGS. 46J, 46L, 46N, and 46P are IVIS photographs, illuminating the cancer cells that have metastasized there. As can be seen in the figure, the anti-NME7_{AB} antibodies greatly inhibited metastasis to the liver, which is a primary site for breast cancer metastasis. FIG. 46Q is a bar graph of the measured photons emitted and enumerated by IVIS instrument for livers harvested from control animals versus the treated animals. As can be seen in the inserted graph of IVIS measurements, the inhibition of metastasis to the liver follows the rank order of inhibition of metastasis when cells were injected into the tail vein, which also matches the rank order of potency in being able to disrupt the NME7_{AB}-MUC1* interaction.

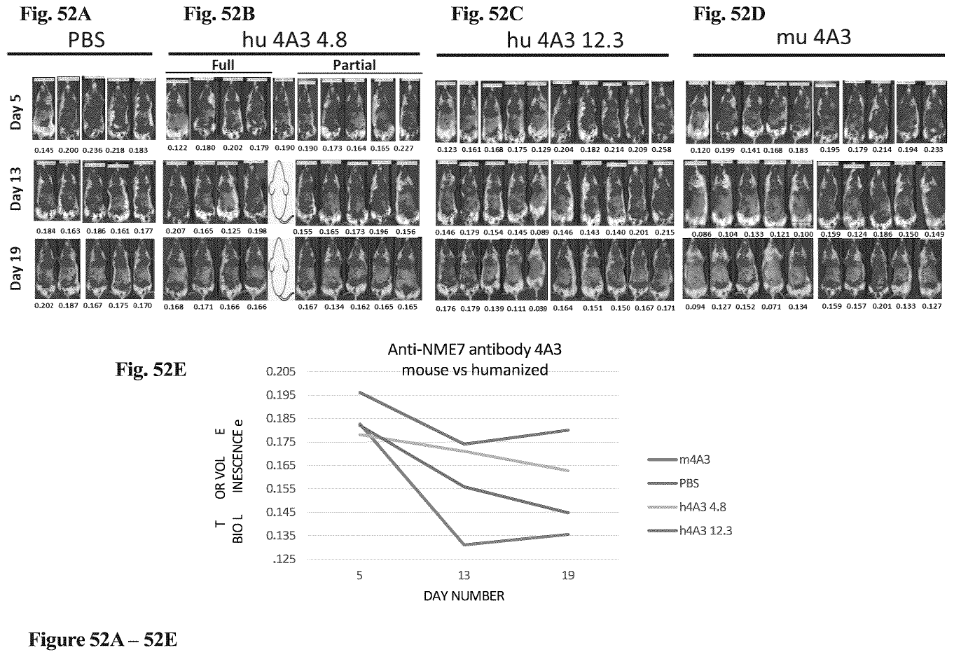

FIGS. 52A-52E shows photographs of female nu/nu mice that on Day 0 were injected into the tail vein with 10,000 Luciferase positive T47D metastatic breast cancer stem cells mixed with NME7_{AB} to a final concentration of 32 nM. On Day 1 and Day 2 animals were injected into the tail vein with more 32 nM NME7_{AB}, which we have shown increases metastases. This is a system to demonstrate reversion of established metastases. On Day 5 animals were injected intravenously with 15 mg/kg of 8F9A4A3, aka 4A3, wherein the antibody was either the murine sequence or humanized 4A3 variant 4.8, or humanized 4A3 variant 12.3. FIG. 52A shows control animals injected with phosphate buffered saline solution. FIG. 52B shows animals treated with anti-NME7_{AB} humanized 4A3 4.8. FIG. 52C shows animals treated with anti-NME7_{AB} humanized 4A3 12.3. FIG. 52D shows animals treated with anti-NME7_{AB} monoclonal antibody murine 4A3. FIG. 52E shows a graph of the quantification of bioluminescence measurement of tumor volume, taken in an IVIS instrument. Animals were injected with either PBS or an anti-NME7 antibody at 15 mg/kg once every 3 days. Antibody hu 4A3 4.8 denotes that the sequences derived from human heavy chain antibody sequence 1.46 (SEQ ID NO:1102) and light chain sequence 1.6 (SEQ ID NO:1104) were used. Antibody hu 4A3 12.3 denotes that the sequences derived from human heavy chain antibody sequence 4.4 (SEQ ID NO:1106) and light chain sequence 4.1 (SEQ ID NO:1108) were used. Note that half the animals treated with hu 4A3 4.8 were given a partial half dose.

Humanized 4A3 variant 4.8 was generated from human heavy chain IGHV1-46*01 (IMGT accession number X92343) and from human light chain IGKV1-6*01 (IMGT accession number M64858). Humanized 4A3 variant 4.8 was also caller 4A3 146-16.

Humanized 4A3 variant 12.3 was generated from human heavy chain IGHV4-4*01 (IMGT accession number X05713) and from human light chain IGKV4-1*01 (IMGT accession number Z00023). Humanized 4A3 variant 12.3 was also caller 4A3 44-41.

The accession number are from the IMGT data base (the international ImMunoGeneTics information system).

IGHVx-x or IGKVx-x correspond to the IMGT gene name. * x corresponds to the IMGT allele name

We performed immunofluorescent imaging of many cancer cell lines to determine if cultured cancer cell lines express NME7_{AB}. As FIG. 47A-47F and FIG. 48A-48I clearly show, each MUC1 positive cancer cell line we tested is positive for NME7_{AB} and its binding is membranous, consistent with NME7_{AB} being secreted from cancer cells whereupon it binds to the extra cellular domain of MUC1*. FIG. 47A-47F shows photographs of immunofluorescent experiments in which various cancer cell lines are stained for the presence of NME7_{AB}. FIG. 47A shows T47D breast cancer cells stained with varying concentrations of anti-NME7_{AB} antibody 5D4. FIG. 47B shows ZR-75-1 breast cancer cells, also known as 1500s, stained with varying concentrations of anti-NME7_{AB} antibody 5D4. FIG. 47C shows H1975 non-small cell lung cancer cells stained with varying concentrations of anti-NME7_{AB} antibody 5D4. FIG. 47D shows H292 non-small cell lung cancer cells stained with varying concentrations of anti-NME7_{AB} antibody 5D4. FIG. 47E shows HP AFII pancreatic cancer cells stained with varying concentrations of anti-NME7_{AB} antibody 5D4. FIG. 47F shows DU145 prostate cancer cells stained with varying concentrations of anti-NME7_{AB} antibody 5D4. As can be seen in the figure, all the cancer cell lines we tested show strong and membranous staining for NME7_{AB}. The monoclonal antibody used in these experiments was 5D4. In parallel, NME7_{AB} antibodies 5A1 and 4A3 were used to stain the same cell lines and produced the same results.

FIG. 48A-48I shows photographs of immunofluorescent experiments in which various lung cancer cell lines are stained for the presence of NME7_{AB}. FIG. 48A-48C shows H1975 non-small cell lung cancer cells, which are an adenocarcinoma, stained with varying concentrations of anti-NME7_{AB} antibody 5D4. FIG. 48A is an overlay of DAPI and anti-NME7_{AB} staining. FIG. 48B shows anti-NME7_{AB} staining alone. FIG. 48C is a magnified view of the overlay of DAPI and anti-NME7_{AB} staining. FIG. 48D-48F shows H292 non-small cell lung cancer cells, which are a mucoepidermoid pulmonary carcinoma, stained with varying concentrations of anti-NME7_{AB} antibody 5D4. FIG. 48D is an overlay of DAPI and anti-NME7_{AB} staining. FIG. 48E shows anti-NME7_{AB} staining alone. FIG. 48F is a magnified view of the overlay of DAPI and anti-NME7_{AB} staining. FIG. 48G-48I shows H358 non-small cell lung cancer cells, which are a metastatic bronchioalveolar carcinoma, stained with varying concentrations of anti-NME7_{AB} antibody 5D4. FIG. 48G is an overlay of DAPI and anti-NME7_{AB} staining. FIG. 48H shows anti-NME7_{AB} staining alone. FIG. 48I is a magnified view of the overlay of DAPI and anti-NME7_{AB} staining.

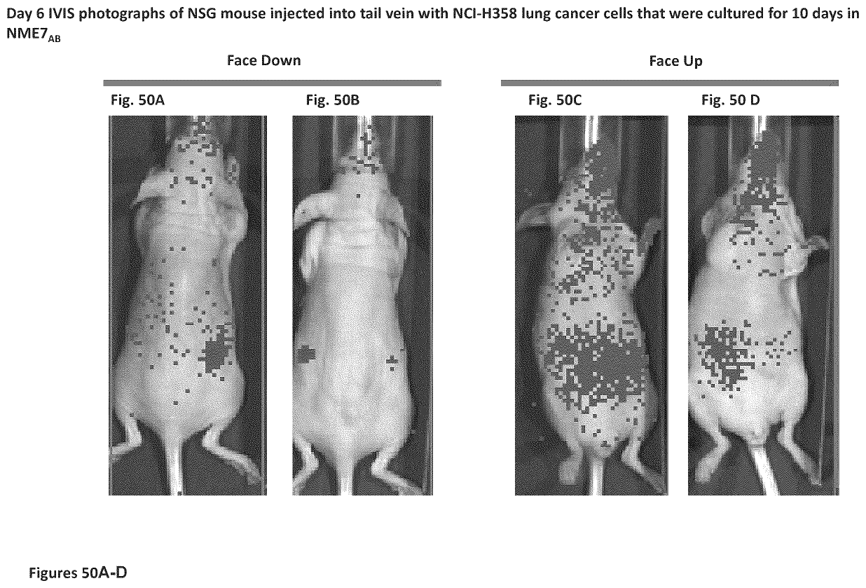

In addition, culturing these cell lines in a serum-free media containing NME7_{AB} even further increased their expression of stem cell and metastatic markers. In particular, the cells that became non-adherent, referred to here as floaters, have even higher expression of stem cell and metastatic markers than their adherent counterparts. FIG. 49A-49I shows PCR graphs of cancer cell lines, breast T47D, Lung H1975, lung H358 and pancreatic HPAFII before and after culture in NME7_{AB}. FIG. 49A measured breast metastatic marker CXCR4. FIG. 49B measured stem cell marker OCT4. FIG. 49C measured metastatic marker ALDH1. FIG. 49D measured stem cell marker SOX2. FIG. 49E measured stem cell marker NANOG. FIG. 49F measured marker CDH1, also known as E-cadherin. FIG. 49G measured metastatic marker CD133. FIG. 49H measured stem cell marker ZEB2. FIG. 49I measured stem, cancer and metastatic marker MUC1. The floater cells, also known as tumor spheres become able to grow anchorage independently and show markers of metastasis that are more elevated than the adherent cells. Animals injected with cancer stem cells are those injected with the NME7_{AB} grown floater cells. As can be seen in the figure markers of metastasis, stem cell markers, or markers of epithelial to mesenchymal transition (EMT) are elevated after culture in NME7_{AB}, indicating a transition to a more metastatic state. FIG. 50 shows Day 6 IVIS photographs of NSG mice injected into the tail vein with either 10,000 H358 lung cancer parent cells or H358 cells after 10-12 days in culture with NME7_{AB}. As can be seen in the figure, the NCI-H358 lung cancer cells grown in NME7_{AB} have greatly increased metastatic potential compared to the parent cells, which are themselves reportedly metastatic cells. The functional increase in metastasis in 6 days from the NCI-H358 NME7_{AB} metastatic cancer stem cells from just 10,000 cells is consistent with FIG. 49, showing that H358 cells greatly increased expression of metastatic markers after culture in NME7_{AB}.

FIG. 51 shows PCR graph of a MUC1 negative prostate cancer line PC3 before and after 2 or 3 passages in culture in either dimeric NM23-H1, also known as NME1, or NME7_{AB}. The graph shows the fold difference in markers of stem cells, cancer cells as well as metastatic markers. As can be seen in the figure, repeated culture in NME1 or NME7_{AB} induces upregulation of stem, cancer and metastatic markers but also upregulates expression of MUC1 by 5-8 times.

Collectively, these data have demonstrated that an NME7 that is devoid of the DM10 domain is secreted by cancer cells and binds to the extra cellular domain of a MUC1 that is devoid of tandem repeat domain, whereupon the NME7 dimerizes the MUC1* extra cellular domain which results in increased cancer cell growth and an increase in the cancer cells' metastatic potential. It stands to reason that antibodies that disrupt the interaction between NME7_{AB} and MUC1* extra cellular domain would inhibit cancer cell growth and would inhibit cancer metastasis. Here, we have shown that anti-NME7_{AB} antibodies that inhibit interaction between NME7_{AB} and MUC1* extra cellular domain do in fact inhibit cancer cell growth and cancer metastasis. Therefore, it follows that anti-NME7_{AB} antibodies can be administered to a patient, diagnosed with or at risk of developing a cancer or metastasis, for the treatment or prevention of cancers.

Because NME1 is expressed in the cytoplasm of all cells and can be lethal if knocked out, and importantly the NME1 A domain has high sequence homology to the NME7 A domain, it is critical that anti-NME7_{AB} antibodies for therapeutic use bind to NME7_{AB} or NME7-X1, but not to NME1. In one aspect of the invention antibodies that would be optimal for therapeutic use were selected for their ability to bind to peptides that were unique to NME7_{AB} or NME7-X1 and were not present in the NME1 sequence. FIG. 6-FIG. 9 lists NME7_{AB} unique peptides.

In a preferred embodiment, antibodies suitable for administration to a patient for the treatment or prevention of cancer or cancer metastasis are selected from the group of antibodies that bind to the NME7 B3 peptide. In yet a more preferred embodiment, antibodies suitable for administration to a patient for the treatment or prevention of cancer or cancer metastasis are selected from the group of antibodies that bind to the NME7 B3 peptide, bind to NME7_{AB} but do not bind to NME1. Examples of antibodies suitable for therapeutic use for the treatment or prevention of cancers or cancer metastasis, which have demonstrated such anti-cancer activity and anti-metastatic activity in vitro and in vivo here, include anti-NME7 antibodies 5A1, 4A3 and 5D4. These are but examples and other antibodies generated as described here and selected as described here will have the same anti-cancer and anti-metastatic activity. Such antibodies may be full antibodies or fragment thereof, including scFvs or antibody mimics wherein the variable domains of the antibody are incorporated into a protein scaffold that mimic an antibody. The antibodies may be of human or non-human species, including murine, camelid, llama, human or humanized and may be monoclonal, polyclonal, scFvs or fragments thereof.

Anti-NME7 antibodies for treatment or prevention of cancers or metastases can be used in many different therapeutic formats. For example, any of the antibodies described herein, or a fragment thereof, can be administered to a patient as a stand-alone antibody or antibody fragment, or attached to a toxin such as an antibody drug conjugate (ADC), or incorporated into a bi-specific antibody or incorporated into a BiTE (bispecific T cell engager), or incorporated into a chimeric antigen receptor (CAR) or engineered to be expressed by a cell that also expresses a CAR. The cell may be an immune cell, a T cell, an NK cell or a stem or progenitor cell, which may then be differentiated into a T cell or an NK cell.

Any of the antibodies described herein, or a fragment thereof, can be used as a diagnostic reagent to probe a bodily fluid, cell, tissue or bodily specimen for the presence of NME7_{AB} or NME7-X1, which would be an indicator of cancer or susceptibility to cancers. Antibodies for diagnostic uses may be connected to an imaging agent, a nucleic acid tag, may be of any species including camelid, and can be used in whole body applications or on a bodily fluid, such as blood, cell, or tissue, in vitro, in vivo or intra-operatively.

The selection criteria, for therapeutically useful or diagnostically useful anti-NME7 antibodies, depends on the format or modality of the therapeutic or diagnostic into which the antibody will be incorporated. If the antibody or antibody fragment is to be administered to a patient as a stand-alone agent for the treatment or prevention of cancers or cancer metastases, then the antibody is selected for its ability to: i) bind to NME7_{AB} or NME7-X1, but not to NME1; ii) bind to the PSMGFR peptide; iii) bind to the N-10 peptide and iv) disrupt the interaction between NME7_{AB} or NME7-X1 and the MUC1* extra cellular domain or the interaction between NME7_{AB} or NME7-X1 and the N-10 peptide. The antibody may also be selected for its ability to bind to the NME7 B3 peptide. This therapeutic format also encompasses a cell that has been engineered to express a CAR and a secreted anti-NME7 antibody.

Other modalities require other selection criteria for anti-NME7 antibodies. If the anti-NME7 antibody is to be incorporated into an ADC, the ADC must be internalized by the target cell to trigger killing of the target cell. Recall that NME7_{AB} or NME7-X1 will be bound to the extra cellular domain of MUC1*. If the antibody disrupts binding of the NME to MUC1* extra cellular domain, then the toxin-conjugated antibody will not be internalized and the cell will not be killed. Similarly, if the anti-NME7 antibody is to be incorporated into a CAR or a BiTE, the interaction between NME7_{AB} or NME7-X1 cannot be disrupted or the immune cell will no longer be able to direct its killing agents to the cancer cell. If the anti-NME7 antibody is to be used as a diagnostic reagent, the interaction between NME7_{AB} or NME7-X1 cannot be disrupted or antibody and associated label will be washed away. Therefore, for ADC, CAR T, or CAR-NK, BiTEs or diagnostic applications, the anti-NME7 antibody is selected for its ability to: i) bind to NME7_{AB} or NME7-X1, but not to NME1; ii) bind to the PSMGFR peptide; iii) bind to the N-10 peptide and iv) bind to NME7_{AB} or NME7-X1 without disrupting the interaction with the MUC1* extra cellular domain or the interaction between NME7_{AB} or NME7-X1 and the N-10 peptide. The antibody may also be selected for its ability to bind to the NME7 B3 peptide.

In one aspect of the invention, a cell is engineered to express an anti-NME7_{AB} antibody of the invention or fragment thereof. The cell may be an immune cell, such as a T cell or NK cell or it may be a stem or progenitor cell, which may be differentiated into a more mature immune cell such as a T cell or NK cell. In a preferred embodiment, the cell that is engineered to express an anti-NME7_{AB} antibody is also engineered to express a chimeric antigen receptor (CAR). In a preferred embodiment, the CAR recognizes a tumor associated antigen. In a preferred embodiment, the CAR targets MUC1*. In a more preferred embodiment, the CAR is directed to the tumor by anti-MUC1* antibody MNC2. In another aspect of the invention, cell that is engineered to express a CAR is also engineered to inducibly express an anti-NME7 antibody. In one example, the nucleic acid encoding an anti-NME7_{AB} antibody is inserted into the Foxp3 enhancer or promoter. In another example, the anti-NME7_{AB} antibody is in an NFAT-inducible system. In one aspect, the NFAT-inducible system incorporates NFATc1 response elements inserted upstream of an anti-NME7_{AB} antibody sequence. They may be inserted into an IL-2 promoter, a Foxp3 enhancer or promoter or other suitable promoter or enhancer.

In another aspect of the invention, peptides that are unique to NME7_{AB} or NME7-X1 are incorporated into an entity used to immunize or vaccinate people against cancers or cancer metastases. In a preferred embodiment, the peptide comprises all or part of the NME7 B3 peptide, which may be the NME7 B3 peptide with Cys-14-Ser mutation.

Another aspect of the invention involves a method of generating anti-NME7_{AB} antibodies in a host animal, where the animal is immunized with the NME7 B3 peptide. In a preferred embodiment, the NME7 B3 peptide has Cysteine 14 mutated to Serine (SEQ ID NO: 169) to avoid disulfide bond formation which inhibits NME7 specific antibody generation.

Another aspect of the invention involves a method of generating cells with enhanced metastatic potential involving culturing the cells with NME7_{AB} or NME7-X1. These cells can then be used in many aspects of drug discovery.

Another aspect of the invention involves a cell that is engineered to express NME7_{AB} or NME7-X1. The NME7_{AB} or NME7-X1 may be of human sequence. Their expression may be inducible. In one aspect the cell is an egg which is then developed into an animal that may be a transgenic animal able to express human NME7_{AB} or NME7-X1.

NME7 binds to and dimerizes the extra cellular domain of the MUC1* growth factor receptor. Tissue studies show that MUC1* increases as tumor grade and metastasis increase. Here we show that NME7 expression increases as tumor grade and metastasis increases (FIG. 39-FIG. 41). Here, we have shown that antibodies that inhibit the interaction of NME7 and MUC1* inhibit tumor growth and metastases.

Other NME family members may bind to and dimerize the extra cellular domain of the MUC1* growth factor receptor. For example, we have shown that NME1, NME2 and NME6 can exist as dimers and that they bind to and dimerize the MUC1* extra cellular domain. NME7_{AB} and NME7-X1 have two domains that can bind to the MUC1* extra cellular domain so as monomers they dimerize and activate the MUC1* growth factor receptor. We have now shown that anti-NME7 antibodies inhibit cancer and cancer metastases. Similarly, antibodies or antibody mimics that bind to these other NME proteins may be anti-cancer or anti-metastasis therapeutics that can be administered to a patient diagnosed with or at risk of developing a cancer or a metastasis. In one aspect of the invention, antibodies that can be used therapeutically for the treatment of cancers or metastases are antibodies that bind to NME1, NME2, NME3, NME4, NME5, NME6, NME7, NME8, NME9 or NME10. In one aspect of the invention, the therapeutic antibody or antibody mimic inhibits the binding of the NME protein and its cognate growth factor receptor. In one aspect of the invention, the therapeutic antibody or antibody mimic inhibits the interaction of the NME protein with the extra cellular domain of MUC1*. In another aspect of the invention, the therapeutic antibody or antibody mimic binds to a peptide, derived from NME1, NME2, NME3, NME4, NME5, NME6, NME7, NME8, NME9 or NME10, wherein the peptide is homologous to the NME7 A1, A2, B1, B2 or B3 peptide.

Below is a sequence alignment that shows a homology and identity alignment between NME7 and other NME family members. The underlined or underlined and bolded sequences correspond to NME7 peptides A1 (SEQ ID NO: 141), A2 (SEQ ID NO: 142), B1 (SEQ ID NO: 143), B2 (SEQ ID NO: 144) and B3 (SEQ ID NO: 145).

| nucleoside diphosphate kinase 7 isoform a [Homo sapiens] (Hu_7) | |
|---|---|
| MNHSERFVFIAEWYDPNASLLRRYELLFYPGDGSVEMHDVKNHRTFLKRTKYDNLHLEDLFIGNKVNVFSRQLVL | |
| IDYGDQYTARQLCSRKEKTLALIKPDAISKAGEIIEIINKAGFTITKLKMMMLSRKEALDFHVDHQSRPFFNELI | |
| QFITTGPIIAMEILRDDAICEWKRLLGPANSGVARTDASESIRALFGTDGIRNAAHGPDSFASAAREMELFFPSS | |
| GGCGPANTAKFTNCTCCEVKPHAVSEGLLGKILMAIRDAGFEISAMQMFNMDRVNVEEFYEVYKGVVTEYHDMVT | |
| EMYSGPCVAMEIQQNNAEKTFREFCGPADPEIARHLRPGTLRAIFGKTKIQNAVHCTDLPEDGLLEVQYFFKILD | |
| N (SEQ ID NO: 1141) | |
| NME2 Theoretical pI/Mw: 8.52/17298.04 | |
| MANLERTFIAIKPDGVQRGLVGEIIKRFEQKGFRLVAMKFLRASEEHLKQHYIDLKDRPFFPGLVKYMNSGPVVA | |
| MVWEGLNVVKTGRVMLGETNPADSKPGTIRGDFCIQVGRNIIHGSDSVKSAEKEISLWFKPEELVDYKSCAHDWV | |
| YE (SEQ ID NO: 200) | |
| global/global (N-W) score: 171; 26.5% identity (56.8% similar) in 155 aa | |
| overlap (1-131:1-152) | |
| | 10 20 30 40 50 |
| 7A | ----EKTLALIKPDAISKA--GEIIEIINKAGFTITKLKMMMLSRKEALDFHVDHQSRPF |
| | :.:. ::::..... ::::. ... :: .. .:.. :... . ..: ..::: |
| 2 | MANLERTFIAEKPDGVQRGLVGEIIKRFEQKGFRLVAMKFLRASEEHLKQHYIDLKDRPF |
| | 10 20 30 40 50 60 |
| | 60 70 80 90 100 110 |
| 7A | FNELIQFITTGPIIAMEILRDDAICEWKRLLGPANSGVARTDASESIRALFGTDGIRNAA |
| | : :......::..:: ... . .:: .: . .. .::. : . :: |
| 2 | FPGLVKYMNSGPVVAMVWEGLNVVKTGRVMLGETNPADSKPG---TIRGDFCIQVGRNII |
| | 70 80 90 100 110 |
| | 120 130 |
| 7A | HGPDSFASAAREMELFF---------------------------- (SEQ ID NO: 199) |
| | :: :: :: .:. :.: |
| 2 | HGSDSVKSAEKEISLWFKPEELVDYKSCAHDWVYE---------- (SEQ ID NO: 200) |
| | 120 130 140 150 |
| global/global (N-W) score: 104; 24.4% identity (51.3% similar) in 156 aa | |
| overlap (1-134:1-152) | |
| | 10 20 30 40 50 |
| 7B | NC----TCCIVKPHAVSEGLLGKILMAIRDAGFEISAMQMFNMDRVNVEEFYEVYKGVVT |
| | : .:: .:..::.:.:. ... ::.. ::... .. .... : : |
| 2 | MANLERTFIAEKPDGVQRGLVGEIIKRFEQKGFRLVAMKFLRASEEHLKQHYIDLKDR-P |
| | 10 20 30 40 50 |
| | 60 70 80 90 100 110 |
| 7B | EYHDMVTEMYSGPCVAMEIQQNNATKTFREFCGPADPEIARHLRPGTLRAIFGKTKIQNA |
| | . .: : ::: ::: . :..:: : . : ..: .. :::.:. : .: |
| 2 | FFPGLVKYMNSGPVVAMVWEGLNVVKTGRVMLGETNPADSK---PGTIRGDFCIQVGRNI |
| | 60 70 80 90 100 110 |
| | 120 130 |
| 7B | VHCTDLPEDGLLEVQYFF------------------ (SEQ ID NO: 201) |
| | .: .: ... :.. .: |
| 2 | IHGSDSVKSAEKEISLWFKPEELVDYKSCAHDWVYE (SEQ ID NO: 202) |
| | 120 130 140 150 |
| >NME3 Theoretical pI/Mw: 5.96/19088.97 | |
| MICLVLTIFANLFPSAYSGVNERTFLAVKPDGVQRRLVGEIVRRFERKGFKLVALKLVQASEELLREHYVELRER | |
| PFYSRLVKYMGSGPVVAMVWQGLDVVRASRALIGATDPGDATPGTIRGDFCVEVGKNVIHGSDSVESAQREIALW | |
| FREDELLCWEDSAGHWLYE (SEQ ID NO: 206) | |
| | 10 20 30 40 50 |
| 7A | EKTLALIKPDAISK--AGEIIEIINKAGFTITKLKMMMLSRKEALDFHVDHQSRPFFNEL |
| | :.:. .:::.... .:::.. ... :: .. ::... :.. . .:. . :::...: |
| 3 | ERTFLAVKPDGVQRRLVGEIVRRFERKGFKLVALKLVQASEELLREHYVELRERPFYSRL |
| | 30 40 50 60 70 80 |
| | 60 70 80 90 100 110 |
| 7A | IQFITTGPIIAMEILRDDAICEWKRLLGPANSGVARTDASE-SIRALFGTDGIRNAAHGP |
| | .... .::..:: :.. . :.: .. : ::. .::. : .. .:. :: |
| 3 | VKYMGSGPVVAMVWQGLDVVRASRALIGATDPG---DATPGTIRGDFCVEVGKNVIHGS |
| | 90 100 110 120 130 |
| | 120 130 |
| 7A | DSFASAAREMELFF (SEQ ID NO: 203) |
| | :: :: ::. :.: |
| 3 | DSVESAQREIALWF (SEQ ID NO: 204) |
| | 140 150 |
| | 10 20 30 |
| 7B | N-C--------------------TCCIVKPHAVSEGLLGKILMAIRDAGFEISAMQMFNM |
| | : : ::: .:.. :.:.:. .. ::.. :... . |
| 3 | MICLVLTIFANLFPSAYSGVNERTFLAVKPDGVQRRLVGEIVRRFERKGFKLVALKLVQA |
| | 10 20 30 40 50 60 |
| | 40 50 60 70 80 90 |
| 7B | DRVNVFEFYEVYKGVVTEYHDMVTEMYSGPCVAMEIQQNNATKTFREFCGPADPEIARHL |
| | .. ..: : : : .: : ::: ::: : ..... : . : .:: : |
| 3 | SEELLREHY-VELRERPFYSRLVKYMGSGPVVAMVWQGLDVVRASRALIGATDPGDAT-- |
| | 70 80 90 100 110 |
| | 100 110 120 130 |
| 7B | RPGTLRAIFGKTKIQNAVHCTDLPEDGLLEVQYFF------------------ (SEQ ID NO: 205) |
| | :::.:. : .:..: .: :.. :. .: |
| 3 | -PGTIRGDFCVEVGKNVIHGSDSVESAQREIALWFREDELLCWEDSAGHWLYE (SEQ ID NO: 206) |
| | 120 130 140 150 160 |
| NME4 Theoretical pI/Mw: 10.30/20658.59 | |
| MGGLFWRSALRGLRCGPRAPGPSLLVRHGSGGPSWTRERTLVAVKPDGVQRRLVGDVIQRFERRGFTLVGMKMLQ | |
| APESVLAEHYQDLRRKPFYPALIRYMSSGPVVAMVWEGYNVVRASRAMIGHTDSAEAAPGTIRGDFSVHISRNVI | |
| HASDSVEGAQREIQLWFQSSELVSWADGGQHSSIHPA (SEQ ID NO: 1110) | |
| 29.3% identity (68.4% similar) in 133 aa overlap (1-131:56-185) | |
| | 10 20 30 40 50 |
| 7A | EKTLALIKPDAISK--AGEIIEIINKAGFTITKLKMMMLSRKEALDFHVDHQSRPFFNEL |
| | :.::. .:::.... .:..:. ... :::.. .::.. .. . . : . .::. : |
| 4 | ERTLVAVKPDGVQRRLVGDVIQRFERRGFTLVGMKMLQAPESVLAEHYQDLRRKPFYPAL |
| | 60 70 80 90 100 110 |
| | 60 70 80 90 100 110 |
| 7A | IQFITTGPIIAMEILRDDAICEWKRLLGPANSGVARTDASESIRALFGTDGIRNAAHGPD |
| | :.....::..:: ... . ..: ..:. : : .::. :.. ::. :. : |
| 4 | IRYMSSGPVVAMVWEGYNVVRASRAMIGHTDSAEA---APGTIRGDFSVHISRNVIHASD |
| | 120 130 140 150 160 170 |
| | 120 130 |
| 7A | SFASAAREMELFF (SEQ ID NO: 207) |
| | : .: ::..:.: |
| 4 | SVEGAQREIQLWF (SEQ ID NO: 208) |
| | 180 |
| 28.8% identity (56.8% similar) in 132 aa overlap (3-134:40-167) | |
| | 10 20 30 40 50 60 |
| 7B | TCCIVKPHAVSEGLLGKILMAIRDAGFEISAMQMFNMDRVNVEEFYEVYKGVVTEYHDMV |
| | : ::: .:.. :.: ... .. :: . .:.:.. . . : :. . : .. |
| 4 | TLVAVKPDGVQRRLVGDVIQRFERRGFTLVGMKMLQAPESVLAEHYQDLRRK-PFYPALI |
| | 40 50 60 70 80 90 |
| | 70 80 90 100 110 120 |
| 7B | TEMYSCPCVAMEIQQNNATKTFREFCGPADPEIARHLRPGTLRAIFGKTKIQNAVHCTDL |
| | : ::: ::: . :.... : . : .: : :::.:. :. .:..: .: |
| 4 | RYMSSGPVVAMVWEGYNVVRASRAMIGHTDSAEAA---PGTIRGDFSVHISRNVIHASDS |
| | 100 110 120 130 140 150 |
| | 130 |
| 7B | PEDGLLEVQYFF (SEQ ID NO: 209) |
| | : . :.: .: |
| 4 | VEGAQREIQLWF (SEQ ID NO: 210) |
| | 160 |
| NME5 Theoretical pI/Mw: 6.08/29296.23 | |
| MEISMPPPQIYVEKTLAIIKPDIVDKEEEIQDIILRSGFTIVQRRKLRLSPEQCSNFYVEKYGKMFFPNLTAYMS | |
| SGPLVAMILARHKAISYWLELLGPNNSLVAKETHPDSLRAIYGTDDLRNALHGSNDFAAAEREIRFMFPEVIVEP | |
| IPIGQAAKDYLNLHIMPTLLEGLTELCKQKPADPLFWYMCCRREHWTLRSILLVCMSGIRMSLPHCADYCSFVEG | |
| FEIWLADWLLKNNPNKPKLCHHPIVEEPY (SEQ ID NO: 1111) | |
| 44.3% identity (74.8% similar) in 131 aa overlap (1-131:13-143) | |
| | 10 20 30 40 50 60 |
| 7A | EKTLALIKPDAISKAGEIIEIINKAGFTITKLKMMMLSRKEALDFHVDHQSRPFFNELIQ |
| | :::::.:::: ..: :: .:: ..::::.. . . :: .:.:.. .. .. .: |
| 5 | EKTLAIIKPDIVDKEEEIQDIILRSGFTIVQRRKLRLSPEQCSNFYVEKYGKMFFPNLTA |
| | 20 30 40 50 60 70 |
| | 70 80 90 100 110 120 |
| 7A | FITTGPIIAMEILRDDAICEWKRLLGPANSGVARTDASESIRALFGTDGIRNAAHGPDSF |
| | ....::..:: . : :: : .:::: :: ::. .:.::..::: .::: :: ..: |
| 5 | YMSSGPLVAMILARHKAISYWLELLGPNNSLVAKETHPDSLRAIYGTDDLRNALHGSNDF |
| | 80 90 100 110 120 130 |
| | 130 |
| 7A | ASAAREMELFF (SEQ ID NO: 211) |
| | :.: ::....: |
| 5 | AAAEREIRFMF (SEQ ID NO: 212) |
| | 140 |
| 28.0% identity (58.3% similar) in 132 aa overlap (3-134:15-143) | |
| | 10 20 30 40 50 60 |
| 7B | TCCIVKPHAVSEGLLGKILMAIRDAGFEISAMQMFNMDRVNVEEFYEVYKGVVTEYHDMV |
| | : :.:: :.. .: : .:: : . . .. . .:: : : . ... |
| 5 | TLAIIKPDIVDKEE--EIQDIILRSGFTIVQRRKLRLSPEQCSNFY-VEKYGKMFFPNLT |
| | 20 30 40 50 60 70 |
| | 70 80 90 100 110 120 |
| 7B | TEMYSCPCVAMEIQQNNATKTFREFCGPADPEIARHLRPGTLRAIFGKTKIQNAVHCTDL |
| | . : ::: ::: . ...: . . :. :: . .:.. .: .::::.: ..::.: .. |
| 5 | AYMSSGPLVAMILARHKAISYWLELLGPNNSLVAKETHPDSLRAIYGTDDLRNALHGSND |
| | 80 90 100 110 120 130 |
| | 130 |
| 7B | PEDGLLEVQYFF (SEQ ID NO: 213) |
| | . :....: |
| 5 | FAAAEREIRFMF (SEQ ID NO: 214) |
| | 140 |
| NME6 Theoretical pI/Mw: 7.81/22003.16 | |
| MTQNLGSEMASILRSPQALQLTLALIKPDAVAHPLILEAVHQQILSNKFLIVRMRELLWRKEDCQRFYREHEGRF | |
| FYQRLVEFMASGPIRAYILAHKDAIQLWRTLMGPTRVFRARHVAPDSIRGSFGLTDTRNTTHGSDSVVSASREIA | |
| AFFPDFSEQRWYEEEEPQLRCGPVCYSPEGGVHYVAGTGGLGPA (SEQ ID NO: 65) | |
| 37.6% identity (68.4% similar) in 133 aa overlap (3-131:22-153) | |
| | 10 20 30 40 50 |
| 7A | TLALIKPDAISKAGEIIEIINKA----GFTITKLKMMMLSRKEALDFHVDHQSRPFFNEL |
| | :::::::::... :.: ... : :.... .. ... :. .:..: :...: |
| 6 | TLALIKPDAVAHP-LILEAVHQQILSNKFLIVRMRELLWRKEDCQRFYREHEGRFFYQRL |
| | 30 40 50 60 70 80 |
| | 60 70 80 90 100 110 |
| 7A | IQFITTGPIIAMEILRDDAICEWKRLLGPANSGVARTDASESIRALFGTDGIRNAAHGPD |
| | ..:...::: :. . . ::: :. :.::. :: : .:::. :: ::..:: : |
| 6 | VEFMASGPIRAYILAHKDAIQLWRTLMGPTRVFRARHVAPDSIRGSFGLTDTRNTTHGSD |
| | 90 100 110 120 130 140 |
| | 120 130 |
| 7A | SFASAAREMELFF (SEQ ID NO: 215) |
| | : .::.::. :: |
| 6 | SVVSASREIAAFF (SEQ ID NO: 216) |
| | 150 |
| 29.3% identity (57.9% similar) in 133 aa overlap (3-134:22-153) | |
| | 10 20 30 40 50 60 |
| 7B | TCCIVKPHAVSEGL-LGKILMAIRDAGFEISAMQMFNMDRVNVEEFYEVYKGVVTEYHDM |
| | : ..:: ::.. : : . . : . : : :. . . . ..::. ..: :. . |
| 6 | TLALIKPDAVAHPLILEAVHQQILSNKFLIVRMRELLWRKEDCQRFYREHEGRFF-YQRL |
| | 30 40 50 60 70 80 |
| | 70 80 90 100 110 120 |
| 7B | VTEMYSGPCVAMEIQQNNATKTFREFCGPADPEIARHLRPGELRAIFGKTKIQNAVHCTD |
| | : : ::: :. . ...: . .: . ::. :::. : ..:. :: : .:..: .: |
| 6 | VEFMASGPIRAYILAHKDAIQLWRTLMGPTRVFRARHVAPDSIRGSFGLTDTRNTTHGSD |
| | 90 100 110 120 130 140 |
| | 130 |
| 7B | LPEDGLLEVQYFF (SEQ ID NO: 217) |
| | .. :. :: |
| 6 | SVVSASREIAAFF (SEQ ID NO: 218) |
| | 150 |
| NME8 Theoretical pI/Mw: 4.90/67269.94 | |
| MASKKREVQLQTVINNQSLWDEMLQNKGLTVIDVYQAWCGPCRAMQPLFRKLKNELNEDEILHFAVAEADNIVTL | |
| QPFRDKCEPVFLFSVNGKIIEKIQGANAPLVNKKVINLIDEERKIAAGEMARPQYPEIPLVDSDSEVSEESPCES | |
| VQELYSIAIIKPDAVISKKVLEIKRKITKAGFIIEAEHKTVLTEEQVVNFYSRIADQCDFEEFVSFMTSGLSYIL | |
| VVSQGSKHNPPSEETEPQTDTEPNERSEDQPEVEAQVTPGMMKNKQDSLQEYLERQHLAQLCDIEEDAANVAKFM | |
| DAFFPDFKKMKSMKLEKTLALLRPNLFHERKDDVLRIIKDEDFKILEQRQVVLSEKEAQALCKEYENEDYFNKLI | |
| ENMTSGPSLALVLLRDNGLQYWKQLLGPRTVEEAIEYFPESLCAQFAMDSLPVNQLYGSDSLETAEREIQHFFPL | |
| QSTLGLIKPHATSEQREQILKIVKEAGFDLTQVKKMFLTPEQIEKIYPKVTGKDFYKDLLEMLSVGPSMVMILTK | |
| WNAVAEWRRLMGPTDPEEAKLLSPDSIRAQFGISKLKNIVHGASNAYEAKEVVNRLFEDPEEN (SEQ ID NO: 1112) | |
| 36.1% identity (69.2% similar) in 133 aa overlap (1-131:316-448) | |
| | 10 20 30 40 50 |
| 7A | EKTLALIKPDAIS-KAGEIIEIINKAGFTITKLKMMMLSRKEALDFHVDHQSRPFFNELI |
| | :::::::::. . . ....::. : : . ....::.::: . ..... .::.:: |
| 8 | EKTLALLRPNLFHERKDDVLRIIKDEDFKILEQRQVVLSEKEAQALCKEYENEDYFNKLI |
| | 320 330 340 350 360 370 |
| | 60 70 80 90 100 110 |
| 7A | QFITTGPIIAMEILRDDAICEWKRLLGPANSGVARTDASESIRALFGTDGIR-NAAHGPD |
| | . .:.:: .:. .:::... ::.:::: . : ::. : :. :.. : .: : |
| 8 | ENMTSGPSLALVLLRDNGLQYWKQLLGPRTVEEAIEYFPESLCAQFAMDSLPVNQLYGSD |
| | 380 390 400 410 420 430 |
| | 120 130 |
| 7A | SFASAAREMELFF (SEQ ID NO: 219) |
| | :. .: ::.. :: |
| 8 | SLETAEREIQHFF (SEQ ID NO: 220) |
| | 440 |
| Waterman-Eggert score: 269; 85.9 bits; E(1) < 1.1e−21 | |
| 33.6% identity (72.7% similar) in 128 aa overlap (1-127:451-577) | |
| 7A | 10 20 30 40 50 |
| | EKTLALIKPDAISKAGE-IIEIINKAGFTITKLKMMMLSRKEALDFHVDHQSRPFFNELI |
| | ..::.:::: : :. : :..:...::: .:..: :.:. .. .. .. :...:. |
| 8 | QSTLGLIKPHATSEQREQILKIVKEAGFDLTQVKKMFLTPEQIEKIYPKVTGKDFYKDLL |
| | 460 470 480 490 500 510 |
| | 60 70 80 90 100 110 |
| 7A | QFITTGPIIAMEILRDDAICEWKRLLGPANSGVARTDASESFRALFGTDGIRNAAHGPDS |
| | .....:: ..: . . .:. ::.::.::.. :. . .:::: :: . ..: .:: .. |
| 8 | EMLSVGPSMVMILTKWNAVAEWRRLMGPTDPEEAKLLSPDSIRAQFGISKLKNIVHGASN |
| | 520 530 540 550 560 570 |
| | 120 |
| 7A | FASAAREM (SEQ ID NO: 221) |
| | : :.:. |
| 8 | -AYEAKEV (SEQ ID NO: 222) |
| Waterman-Eggert score: 119; 40.4 bits; E(1) < 5.3e−08 | |
| 33.8% identity (73.8% similar) in 65 aa overlap (3-65:156-220) | |
| | 10 20 30 40 50 60 |
| 7A | TLALIKPDAI--SKAGEIIEIINKAGFTITKLKMMMLSRKEALDFHVDHQSRPFFNELIQ |
| | ..:.:::::. .:. :: . :.:::: : . .:.......:. .. :.:... |
| 8 | SIAIIKPDAVISKKVLEIKRKITKAGFIIEAEHKTVLTEEQVVNFYSRIADQCDFEEFVS |
| | 160 170 180 190 200 210 |
| 7A | FITTG (SEQ ID NO: 223) |
| | :.:.: |
| 8 | FMTSG (SEQ ID NO: 224) |
| | 220 |
| 33.6% identity (65.5% similar) in 116 aa overlap (3-118:453-566) | |
| | 10 20 30 40 50 60 |
| 7B | TCCIVKPHAVSEGLLGKILMAIRDAGFEISAMQMFNMDRVNVEEFYEVYKGVVTEYHDMV |
| | : ..::::.:: .:: ...:::... .. . . ..:..: : :.:.. |
| 8 | TLGLIKPHATSEQRE-QILKIVKEAGFDLTQVKKMFLTPEQEEKIYPKVTGK-DFYKDLL |
| | 460 470 480 490 500 510 |
| | 70 80 90 100 110 |
| 7B | TEMYSGPCVAMEIQQNNATKTFREFCGPADPEIARHLRPGTLRAIFGKTKIQNAVH (SEQ ID NO: 225) |
| | . :: ..: . . ::. .:.. ::.::: :. : : ..:: :: .:..: :: |
| 8 | EMLSVGPSMVMILTKWNAVAEWRRLMGPTDPEEAKLLSPDSIRAQFGISKLKNIVH (SEQ ID NO: 226) |
| | 520 530 540 550 560 |
| Waterman-Eggert score: 128; 41.3 bits; E(1) < 2.9e−08 | |
| 23.3% identity (60.3% similar) in 116 aa overlap (20-134:334-448) | |
| | 20 30 40 50 60 70 |
| 7B | ILMAIRDAGFEISAMQMFNMDRVNVEEFYEVYKGVVTEYHDMVTEMYSGPCVAMEIQQNN |
| | .: :.: :.: ... ... ... . . :.. .. .. .: ::: .:. . ..: |
| 8 | VLRIIKDEDFKILEQRQVVLSEKEAQALCKEYENE-DYFNKLIENMTSGPSLALVLLRDN |
| | 340 350 360 370 380 390 |
| | 80 90 100 110 120 130 |
| 7B | ATKTFREFCGPADPEIARHLRPGTLRAIFGKTKIQ-NAVHCTDLPEDGLLEVQYFF (SEQ ID NO: 227) |
| | . . .... :: : : . : .: : :. .. : .. .: : . :.:.:: |
| 8 | GLQYWKQLLGPRTVEEAIEYFPESLCAQFAMDSLPVNQLYGSDSLETAEREIQHFF (SEQ ID NO: 228) |
| | 400 410 420 430 440 |
| Waterman-Eggert score: 76; 26.4 bits; E(1) < 0.00088 | |
| 23.4% identity (46.8% similar) in ill aa overlap (6-105:159-268) | |
| | 10 20 30 40 50 60 |
| 7B | IVKPHAVSEGLLGKILMAIRDAGFEISAMQMFNMDRVNVEEFYEVYKGVVTEYHDMVTEM |
| | :.:; :: . .: : ::: : : . . . .: .:: .....:. : |
| 8 | IIKPDAVISKKVLEIKRKITKAGFIIEAEHKTVLTEEQVVNFYSRIADQC-DFEEFVSFM |
| | 160 170 180 190 200 210 |
| | 70 80 90 100 |
| 7B | YSGPCVAMEIQQNNATKTFREFCGPA-----------DPEIARHLRPGTLR (SEQ ID NO: 229) |
| | :: . ..:.. . : : .::. .. :: .. |
| 8 | TSGLSYILVVSQGSKHNPPSEETEPQTDTEPNERSEDQPEVEAQVTPGMMK (SEQ ID NO: 230) |
| | 220 230 240 250 260 |
| NME9 | |
| MLSSKGLTVVDVYQGWCGPCKPVVSLFQKMRIEVGLDLLHFALAEADRLDVLEKYRGKCE | |
| PTFLFYAIKDEALSDEDECVSHGKNNGEDEDMVSSERTCTLAIIKPDAVAHGKTDEIIMK | |
| IQEAGFEILTNEERTMTEAEVRLFYQHKAGESPSSVRHRNALQCRPWKPGQRRC (SEQ ID NO: 231) | |
| 41.3% identity (67.4% similar) in 46 aa overlap (3-46:100-145) | |
| | 10 20 30 40 |
| 7A | TLALIKPDAIS--KAGEIIEIINKAGFTITKLKMMMLSRKEALDFH |
| | :::.:::::.. :. ::: :..::: : . ... :. :. |
| 9 | TLAIIKPDAVAHGKTDEIIMKIQEAGFEILTNEERTMTEAEVRLFY |
| | 100 110 120 130 140 |
| >-- | |
| Waterman-Eggert score: 30; 13.5 bits; E(1) < 0.85 | |
| 28.6% identity (71.4% similar) in 14 aa overlap (69-82:100-113) | |
| | 70 80 |
| 7A | AMEILRDDAICEWK (SEQ ID NO: 232) |
| | .. :.. ::. . : |
| 9 | TLAIIKPDAVAHGK (SEQ ID NO: 233) |
| | 100 110 |
| Waterman-Eggert score: 29; 13.2 bits; E(1) < 0.91 | |
| 25.8% identity (74.2% similar) in 31 aa overlap (12-42:121-149) | |
| | 20 30 40 |
| 7A | ISKAGEIIEIINKAGFTITKLKMMMLSRKEA (SEQ ID NO: 234) |
| | :..:: .::... :.:. .. .. ...: |
| 9 | IQEAG--FEILTNEERTMTEAEVRLFYQHKA (SEQ ID NO: 235) |
| | 130 140 |
| 39.6% identity (69.8% similar) in 53 aa overlap (1-53:98-150) | |
| | 10 20 30 40 50 |
| 7B | NCTCCIVKPHAVSEGLLGKILMAIRDAGFEISAMCMFNMDRVNVEEFYEVYKG (SEQ ID NO: 236) |
| | .:: :.:: ::..: .:.: :..::::: . . .: ...:. ::. : |
| 9 | TCTLAIIKPDAVAHGKTDEIIMKIQEAGFEILTNEERTMTEAEVRLFYQHKAG (SEQ ID NO: 237) |
| | 100 110 120 130 140 150 |
| NME10 NP_008846.2 protein XRP2 [Homo sapiens] | |
| MGCFFSKRRKADKESRPENEEERPKQYSWDQREKVDPKDYMFSGLKDEEVGRLPGTVAGQQFLIQDCENC | |
| NIYIFDHSATVTIDDCTNCIIFLGPVKGSVFFRNCRDCKCTLACQQFRVRDCRKLEVFLCCATQPIIESS | |
| SNIKFGCFQWYYPELAFQFKDAGLSIFNNTWSNIHDFTPVSGELNWSLLPEDAVVQDYVPIPTTEELKAV | |
| RVSTEANRSIVPISRGQRQKSSDESCLVVLFAGDYTIANARKLIDEMVGKGFFLVQTKEVSMKAEDAQRV | |
| FREKAPDFLPLLNKGPVEALEFNGDGAVEVCQLIVNEIFNGTKMFVSESKETASGDVDSFYNFADIQMGI (SEQ ID NO: 238) | |
| 23.5% identity (66.2% similar) in 68 aa overlap (11-78:246-308) | |
| | 20 30 40 50 60 70 |
| 7A | AISKACEIIEEINKAGFTITKLKMMMLSRKEALDFHVDHQSRPFFNELIQFITTGPIIAM |
| | .:..: ..:. . :: ... : . .. ..: .: ... : ... ... ::.::. |
| 10 | TIANARKLIDEMVGKGFFLVQTKEVSMKAEDAQ--RVFREKAP---DFLPLLNKGPVIAL |
| | 250 260 270 280 290 300 |
| 7A | EILRDDAI (SEQ ID NO: 239) |
| | :. : :. |
| 10 | EFNGDGAV (SEQ ID NO: 240) |
| Waterman-Eggert score: 30; 15.1 bits; E(1) < 0.73 | |
| 28.9% identity (57.8% similar) in 45 aa overlap (66-108:200-244) | |
| | 70 80 90 100 |
| 7A | PIIAMEILRDDAIC-EWKRLLGPANSGVARTDASES-IRALFGTD (SEQ ID NO: 241) |
| | :: . : : . : .: . : . : . ...:: . .::. : |
| 10 | PIPTTEELKAVRVSTEANRSIVPISRGQRQKSSDESCLVVLFAGD (SEQ ID NO: 242) |
| | 200 210 220 230 240 |
| Waterman-Eggert score: 33; 14.4 bits; E(1) < 0.87 | |
| 14.7% identity (52.0% similar) in 75 aa overlap (7-80:35-109) | |
| | 10 20 30 40 50 60 |
| 7A | IKPDAISKAGEIIEIINKAGFTITKLKMMMLSRKEALDFHVDHQSRPFFNELIQ-FITTG |
| | . : .: : ... :.. .... . .. . ::.. ... .: : |
| 10 | VDPKDYMFSGLKDETVGRLPGTVAGQQFLIQDCENCNIYIFDHSATVTIDDCTNCIIFLG |
| | 40 50 60 70 80 90 |
| | 70 80 |
| 7A | PIIAMEILRDDAICE (SEQ ID NO: 243) |
| | :. . ..:. :. |
| 10 | PVKGSVFFRNCRDCK (SEQ ID NO: 244) |
| | 100 |
| Waterman-Eggert score: 45; 17.5 bits; E(1) < 0.22 | |
| 21.6% identity (58.8% similar) in 51 aa overlap (4-50:130-180) | |
| | 10 20 30 40 50 |
| 7B | CCIVKP--HAVSEGLLGKILMAIRDAGFEI--SAMQMFNMDRVNVEEFYEV (SEQ ID NO: 245) |
| | :: ..: .. :. .: . . .:.. .....:: :...: : |
| 10 | CCATQPIIESSSNIKFGCFQWYYPELAFQFKDAGLSIFNNTWSNIHDFTPV (SEQ ID NO: 246) |
| | 130 140 150 160 170 180 |

As an example, antibodies or antibody mimics that bind to the NME7 homologous peptides (“homologous peptides”) in particular homologous to A1, A2, B1, B2 or B3 peptides, may be administered to a patient diagnosed with or at risk of developing a cancer or cancer metastasis.

### Homologous Peptides to A1, A2, B1, B2 or B3 Peptides

Homologous peptides to A1, A2, B1, B2 or B3 peptides may include without limitation the following:

NME2A1
(amino acids)
(SEQ ID NO: 247)
RASEEHLKQHYIDLKD
NME2A2
(amino acids)
(SEQ ID NO: 248)
PADSKPGT
NME2B1
(amino acids)
(SEQ ID NO: 249)
QKGFRLVAMKFLRASEEHLK
NME2B2
(amino acids)
(SEQ ID NO: 250)
IDLKDRPFPGLVKY
NME2B3
(amino acids)
(SEQ ID NO: 251)
GDFCIQVGRNIIHGSDSVKSAEKEISLWF
NME3A1
(amino acids)
(SEQ ID NO: 252)
QASEELLREHYVELRE
NME3A1
(amino acids)
(SEQ ID NO: 253)
PGDATPGT
NME3B1
(amino acids)
(SEQ ID NO: 254)
RKGFKLVALKLVQASEELLR
NME3B2
(amino acids)
(SEQ ID NO: 255)
VELRERPFYSRLVKY
NME3B3
(amino acids)
(SEQ ID NO: 256)
GDFCVEVGKNVIHGSDSVESAQREIALWF
NME4A1
(amino acids)
(SEQ ID NO: 257)
QAPESVLAEHYQDLRR
NME4A2
(amino acids)
(SEQ ID NO: 258)
SAEAAPGT
NME4B1
(amino acids)
(SEQ ID NO: 259)
RRGFTLVGMKMLQAPESVLA
NME4B2
(amino acids)
(SEQ ID NO: 260)
QDLRRKPFYPALIRY
NME4B3
(amino acids)
(SEQ ID NO: 261)
GDFSVHISRNVIHASDSVEGAQREIQLWF
NME5A1
(amino acids)
(SEQ ID NO: 262)
RLSPEQCSNFYVEKYG
NME5A2
(amino acids)
(SEQ ID NO: 263)
SLVAKETHPDS
NME5B1
(amino acids)
(SEQ ID NO: 264)
RSGFTIVQRRKLRLSPEQCS
NME5B2
(amino acids)
(SEQ ID NO: 265)
VEKYGKMFFPNLTAY
NME5B3
(amino acids)
(SEQ ID NO: 266)
AIYGTDDLRNALHGSNDFAAAEREIRFMF
NME6A1
(amino acids)
(SEQ ID NO: 267)
LWRKEDCQRFYREHEG
NME6A2
(amino acids)
(SEQ ID NO: 268)
VFRARHVAPDS
NME6B1
(amino acids)
(SEQ ID NO: 269)
SNKFLIVRMRELLWRKEDCQ
NME6B2
(amino acids)
(SEQ ID NO: 270)
REHEGRFFYQRLVEF
NME6B3
(amino acids)
(SEQ ID NO: 271)
GSFGLTDTRNTTHGSDSVVSASREIAAFF
NME8A1
(amino acids)
(SEQ ID NO: 272)
VLSEKEAQALCKEYEN
NME8A2
(amino acids)
(SEQ ID NO: 273)
VEEAIEYFPES
NME8A3
(amino acids)
(SEQ ID NO: 274)
FLTPEQIEKIYPKVTG
NME8A4
(amino acids)
(SEQ ID NO: 275)
PEEAKLLSPDS
NME8A5
(amino acids)
(SEQ ID NO: 276)
VLTEEQVVNFYSRIAD
NME8B1
(amino acids)
(SEQ ID NO: 277)
EAGFDLTQVKKMFLTPEQIE
NME8B2
(amino acids)
(SEQ ID NO: 278)
PKVTGKDFYKDLLEM
NME8B3
(amino acids)
(SEQ ID NO: 279)
AQFGISKLKNIVH
NME8B4
(amino acids)
(SEQ ID NO: 280)
DEDFKILEQRQVVLSEKEAQ
NME8B5
(amino acids)
(SEQ ID NO: 281)
KEYENEDYFNKLIEN
NME8B6
(amino acids)
(SEQ ID NO: 282)
AQFAMDSLPVNQLYGSDSLETAEREIQHFF
NME8B7
(amino acids)
(SEQ ID NO: 283)
KAGFIIEAEHKTVLTEEQVV
NME8B8
(amino acids)
(SEQ ID NO: 284)
SRIADQCDFEEFVSF
NME9A1
(amino acids)
(SEQ ID NO: 285)
TMTEAEVRLFY
NME9B1
(amino acids)
(SEQ ID NO: 286)
EAGFEILTNEERTMTEAEVR
NME10A1
(amino acids)
(SEQ ID NO: 287)
SMKAEDAQRVFREK
NME10A2
(amino acids)
(SEQ ID NO: 288)
GQRQKSSDES
NME10A3
(amino acids)
(SEQ ID NO: 289)
IQDCENCNIYIFDHSA
NME10B1
(SEQ ID NO: 290)
ELAFQFKDAGLSIFNNTWSNIH

In some cases, peptides derived from other NME proteins can be made more homologous to NME7 A1, A2, B1, B2 or B3 peptides by shifting the frame or extending the NME7 peptides such that the extended peptides are more homologous to the NME7 peptides that gave rise to antibodies that inhibit cancer or cancer metastases. As another example, antibodies or antibody mimics that bind to the NME7 homologous extended peptides (“extended peptides”) may be administered to a patient diagnosed with or at risk of developing a cancer or cancer metastasis.

### Homologous Extended Peptides to A1, A2, B1, B2 or B3 Peptides

Homologous peptides to A1, A2, B1, B2 or B3 peptides that are extended peptides may include without limitation the following:

NME2A1
(amino acids)
(SEQ ID NO: 291)
RASEEHLKQHYIDLKDRPFFPGL
NME2A2
(amino acids)
(SEQ ID NO: 292)
LGETNPADSKPGTIRGDF
NME2B1
(amino acids)
(SEQ ID NO: 293)
GLVGEIIKRFEQKGFRLVAMKFLRASEEHLKQHY
NME2B2
(amino acids)
(SEQ ID NO: 294)
YIDLKDRPFFPGLVKYMNSGPVVAM
NME2B3
(amino acids)
(SEQ ID NO: 295)
PGTIRGDFCIQVGRNIIHGSDSVKSAEKEISLWF
NME3A1
(amino acids)
(SEQ ID NO: 296)
LKLVQASEELLREHYVELRERPFYSRL
NME3A1
(amino acids)
(SEQ ID NO: 297)
LIGATDPGDATPGTIRGDF
NME3B1
(amino acids)
(SEQ ID NO: 298)
LVGEIVRRFERKGFKLVALKLVQASEELLRE
NME3B2
(amino acids)
(SEQ ID NO: 299)
EHY-VELRERPFYSRLVKYMGSGPVVAM
NME3B3
(amino acids)
(SEQ ID NO: 300)
PGTIRGDFCVEVGKNVIHGSDSVESAQREIALWF
NME4A1
(amino acids)
(SEQ ID NO: 301)
GFTLVGMKMLQAPESVLAEHYQDLRRKPF
NME4A2
(amino acids)
(SEQ ID NO: 302)
GHTDSAEAAPGTIRGDF
NME4B1
(amino acids)
(SEQ ID NO: 303)
LVGDVIQRFERRGFTLVGMKMLQAPESVLAEHY
NME4B2
(amino acids)
(SEQ ID NO: 304)
EHYQDLRRKPFYPALIRYMSSGPVVAM
NME4B3
(amino acids)
(SEQ ID NO: 305)
PGTIRGDFSVHISRNVIHASDS VEGAQREIQLWF
NME5A1
(amino acids)
(SEQ ID NO: 306)
GFTIVQRRKLRLSPEQCSNFYVEKYGKMFF
NME5A2
(amino acids)
(SEQ ID NO: 307)
LLGPNNSLVAKETHPDSLRAIYGTD
NME5B1
(amino acids)
(SEQ ID NO: 308)
IQDIILRSGFTIVQRRKLRLSPEQCSNFY
NME5B2
(amino acids)
(SEQ ID NO: 309)
FYVEKYGKMFFPNLTAYMSSGPLVAM
NME5B3
(amino acids)
(SEQ ID NO: 310)
PDSLRAIYGTDDLRNALHGSNDFAAAEREIRFMF
NME6A1
(amino acids)
(SEQ ID NO: 311)
FLIVRMRELLWRKEDCQRFYREHEGRFFYQRL
NME6A2
(amino acids)
(SEQ ID NO: 312)
LMGPTRVFRARHVAPDSIRGSFG
NME6B1
(amino acids)
(SEQ ID NO: 313)
ILSNKFLIVRMRELLWRKEDCQRFY
NME6B2
(amino acids)
(SEQ ID NO: 314)
FYREHEGRFFYQRLVEFMASGPIRA
NME6B3
(amino acids)
(SEQ ID NO: 315)
ARHVAPDSIRGSFGLTDTRNTTHGSDSVVSASREIAAFF
NME8A1
(amino acids)
(SEQ ID NO: 316)
FKILEQRQVVLSEKEAQALCKEYENEDYFNKLI
NME8A2
(amino acids)
(SEQ ID NO: 317)
WKQLLGPRTVEEAIEYFPESLCAQFAMD
NME8A3
(amino acids)
(SEQ ID NO: 318)
AGFDLTQVKKMFLTPEQIEKIYPKVTGKDFYKDL
NME8A4
(amino acids)
(SEQ ID NO: 319)
EWRRLMGPTDPEEAKLLSPDSIRAQFG
NME8A5
(amino acids)
(SEQ ID NO: 320)
KAGFIIEAEHKTVLTEEQVVNFYSRIADQCDFEE
NME8B1
(amino acids)
(SEQ ID NO: 321)
ILKIVKEAGFDLTQVKKMFLTPEQIEKIY
NME8B2
(amino acids)
(SEQ ID NO: 322)
YPKVTGKDFYKDLLEMLSVGP
NME8B3
(amino acids)
(SEQ ID NO: 323)
DPEEAKLLSPDSIRAQFGISKLKNIVH
NME8B4
(amino acids)
(SEQ ID NO: 324)
LRIIKDEDFKILEQRQVVLSEKEAQ
NME8B5
(amino acids)
(SEQ ID NO: 325)
KEYENE-DYFNKLIENMTSGPSLA
NME8B6
(amino acids)
(SEQ ID NO: 326)
PESLCAQFAMDSLPVNQLYGSDSLETAEREIQHFF
NME8B7
(amino acids)
(SEQ ID NO: 327)
IKRKITKAGFIIEAEHKTVLTEEQVVNFY
NME8B8
(amino acids)
(SEQ ID NO: 328)
FYSRIADQCDFEEFVSFMTSG
NME9A1
(amino acids)
(SEQ ID NO: 329)
AGFEILTNEERTMTEAEVRLFY
NME9B1
(amino acids)
(SEQ ID NO: 330)
IIMKIQEAGFEILTNEERTMTEAEVRLFY
NME10A1
(amino acids)
(SEQ ID NO: 331)
GFFLVQTKEVSMKAEDAQRVFREKAP
NME10A2
(amino acids)
(SEQ ID NO: 332)
EANRSIVPISRGQRQKSSDESCLVVLFAGD
NME10A3
(amino acids)
(SEQ ID NO: 333)
IQDCENCNIYIFDHSA
NME10B1
(SEQ ID NO: 334)
ELAFQFKDAGLSIFNNTWSNIHDFTPVDCT

Some NME proteins exert a function that is necessary for normal cell growth or development. For example, NME1 is thought to be required for normal cell function. Other NME proteins have catalytic domains whose function is required in normal cells or tissues. In these cases, therapeutic antibodies can be selected based on their ability to bind to the targeted, cancer associated NME, but not to a non-targeted NME. For example, the anti-NME7 antibodies presented here, 8F9A5A1, 8F9A4A3, and 5F3A5D4, were selected for their ability to bind to NME7_{AB} but not to NME1; they were further selected based on their ability to inhibit cancer and cancer metastases.

In another aspect of the invention, anti-NME7 antibodies, antibody fragments, for example scFvs, or fragments of antibody mimics are incorporated into chimeric antigen receptors (CARs) which are engineered to be expressed in immune cells. The immune cell can be engineered to express an anti-NME7 CAR, an anti-MUC1* CAR, or both. One of the CARs may be expressed off of an inducible promoter. Alternatively, an immune cell may be engineered to express a CAR such as an anti-MUC1* CAR and an inducible anti-NME7 antibody or antibody fragment. In some instances the inducible promoter may contain NFAT response elements. In one aspect, these engineered species are expressed in T cells, NK cells or dendritic cells. The immune cells may be obtained from the patient or from a donor. In some cases, immune molecules such as MHCs, checkpoint inhibitors or receptors for checkpoint inhibitors are mutated or cut out, for example using CrisPR or CrisPR-like technology. In another aspect, ITAM molecules, Fos, or Jun are mutated or genetically excised via Talens, Sleeping Beauty, CrisPR or CrisPR-like technologies in patient or donor derived immune cells.

In one aspect of the invention, the anti-NME7 antibodies or antibody mimics for use in CAR T format are chosen from among the group of antibodies or antibody mimics that are specific for NME7 but do not disrupt the binding of NME7 to the extra cellular domain of MUC1*. In this way, the anti-NME7 antibody or antibody mimic that targets the CAR T to the tumor will not simply pluck the ligand from the receptor, whereupon the T cell would be unable to inject the target cancer cell with Granzyme B. Such antibodies or antibody mimics are generated by immunizing an animal with an NME7 peptide, such as NME7 peptides A1, A2, B1, B2 or B3 or selected by virtue of their ability to bind to NME7 peptides A1, A2, B1, B2 or B3. Antibodies or antibody mimics can be screened for their ability to specifically bind to NME7, but not to NME1 or NME2, and also for their inability to disrupt binding between NME7 and MUC1* extra cellular domain. For example, in an ELISA setup, the PSMGFR peptide is immobilized to the surface. A labeled NME7_{AB} is allowed to bind to the surface immobilized MUC1* extra cellular domain, and detection of the NME7_{AB} label is measured in the presence or absence of the test antibody or antibody mimic. In one aspect of the invention, an antibody that does not diminish binding between NME7_{AB} and surface-immobilized MUC1* extra cellular domain peptide is selected as an antibody that is incorporated into a CAR and engineered to be expressed in an immune cell and then administered to a patient for the treatment or prevention of cancer or cancer metastases.

In one aspect of the invention, an anti-NME7 antibody or fragment thereof is administered to a patient diagnosed with or at risk of developing a cancer or cancer metastasis. In one aspect, the anti-NME7 antibody or antibody fragment binds to an NME peptide discussed above in particular under sections “Homologous peptides to A1, A2, B1, B2 or B3 peptides” and the “Homologous extended peptides to A1, A2, B1, B2 or B3 peptides”.

In another aspect, the antibody, antibody fragment or antibody mimic binds to an NME7 derived peptide chosen from among A1, A2, B1, B2 or B3 (SEQ ID NOS: 141-145). In yet another aspect, the antibody, antibody fragment or antibody mimic binds to an NME7 peptide comprising most or all of the B3 peptide. In one aspect of the invention, the anti-NME7 antibody, antibody fragment or antibody mimic comprises sequences derived from the variable domains of anti-NME7 antibodies 8F9A4A3 (“4A3”) (SEQ ID NOS: 1001-1015), 8F9A5A1 (“5A1”) (SEQ ID NOS: 1016-1030), or 8H5H5G4 (“5G4”) (SEQ ID NOS: 1031-1045) shown below.

Anti-NME7 B3 peptide monoclonal antibodies
Monoclonal antibody 8F9A4A3 “4A3”
Heavy chain variable region sequence
(SEQ ID NO: 1138)
Gaggtccagctgcaacagtctggacctgaactggtgaagcctggggcttcagtgaagatatcctgcaagacttctgg
aaacacattcactgaatacaccatgcactgggtgaagcagagccatggaaagagccttgagtggattggaggttttaatcctaacaatg
gtgttactaactacaaccagaagttcaagggcaaggccacattgactgtagacaagtcctccagcacagcctacatggagctccgcag
cctgacatctgaggattctgcagtctattactgtgcaagacggtactaccatagtctctacgtgttttactttgactactggggccaaggca
ccactctcacagtctcctca
Translated protein, wherein the underlined sequence is the complementarity
determining region (CDR):
(SEQ ID NO: 1139)
EVQLQQSGPELVKPGASVKISCKTSGNTFTEYTMHWVKQSHGKSLEWIG
GFNPNNGVTNYNQKFKGKATLTVDKSSSTAYMELRSLTSEDSAVYYCARRYYHSLY
VFYFDYWGQGTTLTVSS
Mouse 8F9A4A3 heavy chain variable domain framework 1 (FR1) sequence
(SEQ ID NO: 1113)
gaggtccagctgcaacagtctggacctgaactggtgaagcctggggcttcagtgaagatatcctgcaagacttctgga
(SEQ ID NO: 1114)
EVQLQQSGPELVKPGASVKISCKTSG
Heavy chain variable region CDR1:
(SEQ ID NO: 1115)
Aacacattcactgaatacaccatgcac
(SEQ ID NO: 388)
NTFTEYTMH
Mouse 8F9A4A3 heavy chain variable domain framework 2 (FR2) sequence
(SEQ ID NO: 1116)
Tgggtgaagcagagccatggaaagagccttgagtggattgga
(SEQ ID NO: 1117)
WVKQSHGKSLEWIG
Mouse 8F9A4A3 Heavy chain variable region CDR2:
(SEQ ID NO: 1118)
Ggttttaatcctaacaatggtgttactaactacaaccagaagttcaagggc
(SEQ ID NO: 389)
GFNPNNGVTNYNQKFKG
Mouse 4A3 heavy chain variable domain framework 3 (FR3) sequence
(SEQ ID NO: 1119)
Aaggccacattgactgtagacaagtcctccagcacagcctacatggagctccgcagcctgacatctgaggattctgc
agtctattactgtgcaaga
(SEQ ID NO: 1120)
KATLTVDKSSSTAYMELRSLTSEDSAVYYCAR
Mouse 8F9A4A3 Heavy chain variable region CDR3:
(SEQ ID NO: 1121)
cggtactaccatagtctctacgtgttttactttgactac
(SEQ ID NO: 390)
RYYHSLYVFYFDY
Mouse 8F9A4A3 Light chain variable region sequence
(SEQ ID NO: 1136)
gatatccagatgacacagactacatcctccctgtctgcctctctgggagacagagtcaccctcagttgcagtgcaagtc
agggcattagcaattatttaaactggtatcagcagaaaccagatggaactgttgaactcctgatcttttacacatcaagtttacactcagga
gtcccatcaaggttcagtggcagtgggtctgggacagattattctctcaccatcagcaacctggaacctgaagatattgccacttactatt
gtcagcagtatagtaagcttccttacacgttcggaggggggaccaagctggaaataaaa
Translated protein, wherein the underlined sequence is the complementarity
determining region (CDR):
(SEQ ID NO: 1109)
DIQMTQTTSSLSASLGDRVTLSCSASQGISNYLNWYQQKPDGTVELLIFYT
SSLHSGVPSRFSGSGSGTDYSLTISNLEPEDIATYYCQQYSKLPYTFGGGTKLEIK
Mouse 8F9A4A3 light chain variable domain framework 1 (FR1) sequence
(SEQ ID NO: 1122)
gatatccagatgacacagactacatcctccctgtctgcctctctgggagacagagtcaccctcagttgc
(SEQ ID NO: 1123)
DIQMTQTTSSLSASLGDRVTLSC
Mouse 8F9A4A3 Light chain variable region CDR 1:
(SEQ ID NO: 1124)
Agtgcaagtcagggcattagcaattatttaaac
(SEQ ID NO: 393)
SASQGISNYLN
Mouse 4A3 light chain variable domain framework 2 (FR2) sequence
(SEQ ID NO: 1125)
Tggtatcagcagaaaccagatggaactgttgaactcctgatcttt
(SEQ ID NO: 1126)
WYQQKPDGTVELLIF
Mouse 8F9A4A3 Light chain variable region CDR2:
(SEQ ID NO: 1127)
Tacacatcaagtttacactca
(SEQ ID NO: 394)
YTSSLHS
Mouse 8F9A4A3 light chain variable domain framework 3 (FR3) sequence
(SEQ ID NO: 1128)
Ggagtcccatcaaggttcagtggcagtgggtctgggacagattattctctcaccatcagcaacctggaacctgaagat
attgccacttactattgt
(SEQ ID NO: 1129)
GVPSRFSGSGSGTDYSLTISNLEPEDIATYYC
Mouse 8F9A4A3 Light chain variable region CDR3:
(SEQ ID NO: 1130)
Cagcagtatagtaagcttccttacacg
(SEQ ID NO: 395)
QQYSKLPYT
Humanized 8F9A4A3 H-ori heavy chain variable domain sequence
(SEQ ID NO: 1131)
Caggttcagctggttcagtctggtgcagaagtgaagaaacctggcgcctctgtgaaggtgtcctgcaaggtgtccgg
aaataccttcaccgagtacaccatgcactgggtccgacaggcccctggcaaaggacttgaatggatgggcggcttcaaccccaacaa
cggcgtgaccaactacaaccagaaattcaagggccgcgtgaccatgaccgaggacacaagcacagacaccgcctacatggaactg
agcagcctgagaagcgaggacaccgccgtgtactactgcgccagaaggtactaccacagcctgtacgtgttctacttcgactactggg
gccagggcaccctggtcacagtttcttct
(SEQ ID NO: 1206)
QVQLVQSGAEVKKPGASVKVSCKVSGNTFTEYTMHWVRQAPGKGLEW
MGGFNPNNGVTNYNQKFKGRVTMTEDTSTDTAYMELSSLRSEDTAVYYCARRYYH
SLYVFYFDYWGQGTLVTVSS
Humanized 8F9A4A3 H-1.46 heavy chain variable domain sequence
(SEQ ID NO: 1101)
caagtgcagctggtgcagagcggcgccgaggtgaagaaacctggcgccagcgtgaaagtgtcctgcaaggccag
cggcaatacattcaccgagtacacaatgcactgggtcagacaggcccccggccagggcctggaatggatcggcggatttaacccca
acaacggcgtgacaaactacaaccagaagttcaagggcaaggtgaccatcacaagagacaccagcagcagcaccgtgtacatgga
actgtcttctctgcggagcgaggataccgccgtgtactattgtgccagacggtactaccacagcctgtacgtgttctacttcgactactgg
ggacagggcaccctggttaccgtgtcctct
(SEQ ID NO: 1102)
QVQLVQSGAEVKKPGASVKVSCKASGNTFTEYTMHWVRQAPGQGLEWI
GGFNPNNGVTNYNQKFKGKVTITRDTSSSTVYMELSSLRSEDTAVYYCARRYYHSL
YVFYFDYWGQGTLVTVSS
Humanized 8F9A4A3 H-3.15 heavy chain variable domain sequence
(SEQ ID NO: 1132)
Gaggtgcagctggtggaaagcggcggcggcctggttaagcctggcggatctctgagactgagctgtgccgcttctg
gcaataccttcaccgagtacaccatgcactgggtgcggcaggcccctggaaaaggcctggaatggatcggcggatttaaccccaac
aacggcgtgacaaattacaaccagaaattcaagggcaagttcaccatcacaagagataagagcaagaacaccctgtacctgcaaatg
aacagcctgaagtccgaggacaccgccgtgtactactgcgccagacggtactaccacagcctctatgtgttctacttcgactactgggg
ccagggcacactggtcaccgtgtccagc
(SEQ ID NO: 1133)
EVQLVESGGGLVKPGGSLRLSCAASGNTFTEYTMHWVRQAPGKGLEWIG
GFNPNNGVTNYNQKFKGKFTITRDKSKNTLYLQMNSLKSEDTAVYYCARRYYHSLY
VFYFDYWGQGTLVTVSS
Humanized 8F9A4A3 H-4.4 heavy chain variable domain sequence
(SEQ ID NO: 1105)
caagtgcagctgcaggagagcggacctggcctggttaagcctggaggcaccctgtctctgacatgtgctgtgtctgg
caatacctttaccgagtacaccatgcactgggtgcggcagcctccaggcaagggcctggaatggatcggcggcttcaaccccaacaa
cggcgtgacaaattacaaccagaaattcaagggaaaagtgaccatcaccgtggataagtccaagaacaccttcagcctcaagctgag
cagcgtgacagccgccgacaccgccgtgtactactgcgccagaagatactatcacagcctgtacgtgttctacttcgactactggggc
cagggcacactggtcaccgtgtccagc
(SEQ ID NO: 1106)
QVQLQESGPGLVKPGGTLSLTCAVSGNTFTEYTMHWVRQPPGKGLEWIG
GFNPNNGVTNYNQKFKGKVTITVDKSKNTFSLKLSSVTAADTAVYYCARRYYHSLY
VFYFDYWGQGTLVTVSS
Humanized 8F9A4A3 L-1.6 light chain variable domain sequence
(SEQ ID NO: 1103)
gatatccagatgacacagagccctagctccctgagcgccagcgtgggcgaccgggtcaccattacatgcagcgctt
ctcagggcatctccaactacctgaactggtaccagcagaaacccggcaaggcccctaagctgctgatcttctacaccagctctctgca
cagcggcgtgccatctagattcagcggatctggcagcggcaccgactacaccctgaccatcagctccctccagcctgaggacttcgc
cacctactactgtcagcaatacagcaagctgccttatacctttggcggcggaacaaaggtggaaatcaag
(SEQ ID NO: 1104)
DIQMTQSPSSLSASVGDRVTITCSASQGISNYLNWYQQKPGKAPKLLIFYT
SSLHSGVPSRFSGSGSGTDYTLTISSLQPEDFATYYCQQYSKLPYTFGGGTKVEIK
Humanized 8F9A4A3 L-3.15 light chain variable domain sequence
(SEQ ID NO: 1134)
Gagatcgtgatgacccagagcccagctacacttagtgtgagtccaggtgaacgggctaccctgtcctgcagcgcca
gccagggcatcagcaactacctgaactggtaccagcagaaacctggccaggcccctagactgctgatcttctacaccagcagcctgc
acagcggcatccccgccagattcagcggcagcggctctggaacagactacaccctgacaatctctagcctgcagtctgaagattttgc
cgtctactactgtcagcaatacagcaagctgccttataccttcggcggcggaaccaaggtggaaattaag
(SEQ ID NO: 1135)
EIVMTQSPATLSVSPGERATLSCSASQGISNYLNWYQQKPGQAPRLLIFYT
SSLHSGIPARFSGSGSGTDYTLTISSLQSEDFAVYYCQQYSKLPYTFGGGTKVEIK
Humanized 8F9A4A3 L-4.1 light chain variable domain sequence
(SEQ ID NO: 1107)
gatatcgtgatgacccagagcccagacagcctggcagtgagtctgggtgagcgtgctacaatcaactgcagcgcca
gccagggcatctccaactacctgaattggtatcagcagaaacctggccaggctcctaagctgctgatcttctacaccagcagcctgcac
agcggcgtgccagatagattcagcggcagcggatctggcaccgactacacactgaccatttcttctctccaggccgaggacgtggcc
gtctactactgtcagcaatacagcaagctgccttacacctttggcggaggcacaaaggtggaaatcaag
(SEQ ID NO: 1108)
DIVMTQSPDSLAVSLGERATINCSASQGISNYLNWYQQKPGQAPKLLIFYT
SSLHSGVPDRFSGSGSGTDYTLTISSLQAEDVAVYYCQQYSKLPYTFGGGTKVEIK
Monoclonal antibody 5F3A5D4
Heavy chain variable region sequence
H-2,3,4,13,15
(SEQ ID NO: 427)
gtccagctgcaacagtctggacctgatctggtgaagcctgggacttcagtgaagatatcctgtaagacttctggaaaca
cattcactgaatacaccatgcactgggtgaagcagagccatggaaagagccttgagtggattggaggttttaatcctaacaatggtgtta
ctaactacaaccagaagttcaagggcaaggccacattgactgtagacaagtcctccagcacagcctacatggagctccgcagcctga
catctgaggattctgcagtctattactgtgcaagacgttactaccatagtacctacgtgttctactttgactcctggggccaaggcaccact
ctcacagtctcctca
Translated protein, wherein the underlined sequence is the complementarity
determining region (CDR):
(SEQ ID NO: 428)
VQLQQSGPDLVKPGTSVKISCKTSGNTFTEYTMHWVKQSHGKSLEWIGGF
NPNNGVTNYNQKFKGKATLTVDKSSSTAYMELRSLTSEDSAVYYCARRYYHSTYVF
YFDSWGQGTTLTVSS
Heavy chain variable region CDR1:
(SEQ ID NO: 429)
NTFTEYTMH
Heavy chain variable region CDR2:
(SEQ ID NO: 430)
GFNPNNGVTNYNQKFKG
Heavy chain variable region CDR3:
(SEQ ID NO: 431)
RYYHSTYVFYFDS
Light chain variable region sequence
K-1,2,3,4,9
(SEQ ID NO: 432)
gatatccagatgacacagactacatcctccctgtctgcctctctgggagacagagtcaccatcagttgcagtgcaagtc
agggcattagcaattatttaaactggtttcagcagaaaccagatggaactattaagctcctgatctattacacatcaagtttacattcagga
gtcccatcaaggttcagtggcagtgggtctgggacagattattctctcaccatcagtaatgtggaacctgaagatattgccacttactattg
tcagcagtatagtaagcttccttacacgttcggaggggggaccaagctggagataaaacgg
Translated protein, wherein the underlined sequence is the complementarity
determining region (CDR):
(SEQ ID NO: 433)
DIQMTQTTSSLSASLGDRVTISCSASQGISNYLNWFQQKPDGTIKLLIYYTS
SLHSGVPSRFSGSGSGTDYSLTISNVEPEDIATYYCQQYSKLPYTFGGGTKLEIKR
Light chain variable region CDR 1:
(SEQ ID NO: 434)
SASQGISNYLN
Light chain variable region CDR2:
(SEQ ID NO: 435)
YTSSLHS
Light chain variable region CDR3:
(SEQ ID NO: 436)
QQYSKLPYT
Mouse 5F3A5D4-V2 heavy chain variable domain sequence:
(DNA)
(SEQ ID NO: 1155)
Gaggtccagctgcaacagtctggacctgatctggtgaagcctgggacttcagtgaagatatcctgtaagacttctgga
aacacattcactgaatacaccatgcactgggtgaagcagagccatggaaagagccttgagtggattggaggttttaatcctaacaatgg
tgttactaactacaaccagaagttcaagggcaaggccacattgactgtagacaagtcctccagcacagcctacatggagctccgcagc
ctgacatctgaggattctgcagtctattactgtgcaagacgttactaccatagtacctacgtgttctactttgactcctggggccaaggcac
cactctcacagtctcctca
(amino acids)
(SEQ ID NO: 1172)
EVQLQQSGPDLVKPGTSVKISCKTSGNTFTEYTMHWVKQSHGKSLEWIG
GFNPNNGVTNYNQKFKGKATLTVDKSSSTAYMELRSLTSEDSAVYYCARRYYHSTY
VFYFDSWGQGTTLTVSS
Mouse 5F3A5D4-V2 heavy chain variable domain framework 1 (FR1)
sequence
(DNA)
(SEQ ID NO: 1173)
Gaggtccagctgcaacagtctggacctgatctggtgaagcctgggacttcagtgaagatatcctgtaagacttctgga
(amino acids)
(SEQ ID NO: 1174)
EVQLQQSGPDLVKPGTSVKISCKTSG
Mouse 5F3A5D4-V2 heavy chain variable domain Complementarity-
determining region 1 (CDR1) sequence
(DNA)
(SEQ ID NO: 1187)
Aacacattcactgaatacaccatgcac
(amino acids)
(SEQ ID NO: 429)
NTFTEYTMH
Mouse 5F3A5D4-V2 heavy chain variable domain framework 2 (FR2)
sequence
(DNA)
(SEQ ID NO: 1188)
Tgggtgaagcagagccatggaaagagccttgagtggattgga
(amino acids)
(SEQ ID NO: 1189)
WVKQSHGKSLEWIG
Mouse 5F3A5D4-V2 heavy chain variable domain Complementarity-
determining region 2 (CDR2) sequence
(DNA)
(SEQ ID NO: 1190)
Ggttttaatcctaacaatggtgttactaactacaaccagaagttcaagggc
(amino acids)
(SEQ ID NO: 430)
GFNPNNGVTNYNQKFKG
Mouse 5F3A5D4-V2 heavy chain variable domain framework 3 (FR3)
sequence
(DNA)
(SEQ ID NO: 1191)
Aaggccacattgactgtagacaagtcctccagcacagcctacatggagctccgcagcctgacatctgaggattctgc
agtctattactgtgcaaga
(amino acids)
(SEQ ID NO: 1175)
KATLTVDKSSSTAYMELRSLTSEDSAVYYCAR
Mouse 5F3A5D4-V2 heavy chain variable domain Complementarity-
determining region 3 (CDR3) sequence
(DNA)
(SEQ ID NO: 1176)
Cgttactaccatagtacctacgtgttctactttgactcc
(amino acids)
(SEQ ID NO: 431)
RYYHSTYVFYFDS
Mouse 5F3A5D4-V2 heavy chain variable domain framework 4 (FR4)
sequence
(DNA)
(SEQ ID NO: 1177)
Tggggccaaggcaccactctcacagtctcctca
(amino acids)
(SEQ ID NO: 1178)
WGQGTTLTVSS
Mouse 5F3A5D4-2 light chain variable domain sequence
(DNA)
(SEQ ID NO: 1179)
gatatccagatgacacagactacatcctccctgtctgcctctctgggagacagagtcaccatcagttgcagtgcaagtc
agggcattagcaattatttaaactggtttcagcagaaaccagatggaactattaagctcctgatctattacacatcaagtttacattcagga
gtcccatcaaggttcagtggcagtgggtctgggacagattattctctcaccatcagtaatgtggaacctgaagatattgccacttactattg
tcagcagtatagtaagcttccttacacgttcggaggggggaccaagctggagataaaa
(amino acids)
(SEQ ID NO: 1180)
DIQMTQTTSSLSASLGDRVTISCSASQGISNYLNWFQQKPDGTIKLLIYYTS
SLHSGVPSRFSGSGSGTDYSLTISNVEPEDIATYYCQQYSKLPYTFGGGTKLEIK
Mouse 5F3A5D4-2 light chain variable domain framework 1 (FR1) sequence
(DNA)
(SEQ ID NO: 1171)
gatatccagatgacacagactacatcctccctgtctgcctctctgggagacagagtcaccatcagttgc
(amino acids)
(SEQ ID NO: 1147)
DIQMTQTTSSLSASLGDRVTISC
Mouse 5F3A5D4-2 light chain variable domain Complementarity-determining
region 1 (CDR1) sequence
(DNA)
(SEQ ID NO: 1193)
agtgcaagtcagggcattagcaattatttaaac
(amino acids)
(SEQ ID NO: 1208)
SASQGISNYLN
Mouse 5F3A5D4-2 light chain variable domain framework 2 (FR2) sequence
(DNA)
(SEQ ID NO: 1148)
tggtttcagcagaaaccagatggaactattaagctcctgatctat
(amino acids)
(SEQ ID NO: 1149)
WFQQKPDGTIKLLIY
Mouse 5F3A5D4-2 light chain variable domain Complementarity-determining
region 2 (CDR2) sequence
(DNA)
(SEQ ID NO: 1150)
tacacatcaagtttacattca
(amino acids)
(SEQ ID NO: 1210)
YTSSLHS
Mouse 5F3A5D4-2 light chain variable domain framework 3 (FR3) sequence
(DNA)
(SEQ ID NO: 1151)
ggagtcccatcaaggttcagtggcagtgggtctgggacagattattctctcaccatcagtaatgtggaacctgaagata
ttgccacttactattgt
(amino acids)
(SEQ ID NO: 1152)
GVPSRFSGSGSGTDYSLTISNVEPEDIATYYC
Mouse 5F3A5D4-2 light chain variable domain Complementarity-determining
region 3 (CDR3) sequence
(DNA)
(SEQ ID NO: 1200)
cagcagtatagtaagcttccttacacg
(amino acids)
(SEQ ID NO: 1212)
QQYSKLPYT
Mouse 5F3A5D4-2 light chain variable domain framework 4 (FR4) sequence
(DNA)
(SEQ ID NO: 1153)
ttcggaggggggaccaagctggagataaaa
(amino acids)
(SEQ ID NO: 1154)
FGGGTKLEIK
Mouse 5F3A5D4-3 light chain variable domain sequence
(DNA)
(SEQ ID NO: 1204)
gatatccagatgacacagactacatcctccctgtctgcctctctgggagacagagtcaccctcagttgcagtgcaagtc
agggcattagcaattatttaaactggtatcagcagaaaccagatggaactgttgaactcctgatcttttacacatcaagtttacactcagga
gtcccatcaaggttcagtggcagtgggtctgggacagattattctctcaccatcagcaacctggaacctgaagatattgccacttactatt
gtcagcagtatagtaagcttccttacacgttcggaggggggaccaagctggaaataaaa
(amino acids)
(SEQ ID NO: 1186)
DIQMTQTTSSLSASLGDRVTLSCSASQGISNYLNWYQQKPDGTVELLIFYT
SSLHSGVPSRFSGSGSGTDYSLTISNLEPEDIATYYCQQYSKLPYTFGGGTKLEIK
Mouse 5F3A5D4-3 light chain variable domain framework 1 (FR1) sequence
(DNA)
(SEQ ID NO: 1156)
gatatccagatgacacagactacatcctccctgtctgcctctctgggagacagagtcaccctcagttgc
(amino acids)
(SEQ ID NO: 1192)
DIQMTQTTSSLSASLGDRVTLSC
Mouse 5F3A5D4-3 light chain variable domain Complementarity-determining
region 1 (CDR1) sequence
(DNA)
(SEQ ID NO: 1194)
agtgcaagtcagggcattagcaattatttaaac
(amino acids)
(SEQ ID NO: 1209)
SASQGISNYLN
Mouse 5F3A5D4-3 light chain variable domain framework 2 (FR2) sequence
(DNA)
(SEQ ID NO: 1195)
tggtatcagcagaaaccagatggaactgttgaactcctgatcttt
(amino acids)
(SEQ ID NO: 1196)
WYQQKPDGTVELLIF
Mouse 5F3A5D4-3 light chain variable domain Complementarity-determining
region 2 (CDR2) sequence
(DNA)
(SEQ ID NO: 1197)
tacacatcaagtttacactca
(amino acids)
(SEQ ID NO: 1211)
YTSSLHS
Mouse 5F3A5D4-3 light chain variable domain framework 3 (FR3) sequence
(DNA)
(SEQ ID NO: 1198)
ggagtcccatcaaggttcagtggcagtgggtctgggacagattattctctcaccatcagcaacctggaacctgaagat
attgccacttactattgt
(amino acids)
(SEQ ID NO: 1199)
GVPSRFSGSGSGTDYSLTISNLEPEDIATYYC
Mouse 5F3A5D4-3 light chain variable domain Complementarity-determining
region 3 (CDR3) sequence
(DNA)
(SEQ ID NO: 1201)
cagcagtatagtaagcttccttacacg
(amino acids)
(SEQ ID NO: 1213)
QQYSKLPYT
Mouse 5F3A5D4-3 light chain variable domain framework 4 (FR4) sequence
(DNA)
(SEQ ID NO: 1157)
ttcggaggggggaccaagctggaaataaaa
(amino acids)
(SEQ ID NO: 1207)
FGGGTKLEIK
Humanized 5F3A5D4-V2 H-1.27 heavy chain variable domain sequence
(DNA)
(SEQ ID NO: 1158)
caagtgcagctggtccagagcggcgccgaggtgaaaaagcctggcgccagcgtgaaggtgtcctgcaaggtgtct
ggcaataccttcaccgagtacaccatgcactgggtgcggcaggcccctggaaaaggcctggaatggatcggcggatttaaccccaa
caacggcgtgaccaactacaaccagaagttcaagggcaaggttacactgaccgtggacaccagctcttctaccgcctacatggaact
gagcagcctgagaagcgaggatacagccgtgtactattgtgccagaagatactaccacagcacctacgtgttctacttcgacagctgg
ggccagggcacactggtgacagtgtccagc
(amnio acids)
(SEQ ID NO: 1181)
QVQLVQSGAEVKKPGASVKVSCKVSGNTFTEYTMHWVRQAPGKGLEWI
GGFNPNNGVTNYNQKFKGKVTLTVDTSSSTAYMELSSLRSEDTAVYYCARRYYHST
YVFYFDSWGQGTLVTVSS
Humanized 5F3A5D4-V2 H-1.46 heavy chain variable domain sequence
(DNA)
(SEQ ID NO: 1159)
caagtgcagctggtgcagagcggagccgaggtgaaaaagcccggcgcttctgtgaaggtgtcttgtaaagccagc
ggcaacaccttcaccgagtacaccatgcactgggtgcggcaggcccctggccagggcctggaatggatcggcggctttaatcctaac
aacggcgtgacaaactacaaccagaagttcaagggcaaggttacaatcaccagagataccagcagctctaccgtgtacatggaactg
agcagcctgagaagcgaggacaccgccgtgtattactgcgccagacggtactaccacagcacctacgtgttctacttcgacagctgg
ggccagggaacactggtcacagtgtcctcc
(amino acids)
(SEQ ID NO: 1160)
QVQLVQSGAEVKKPGASVKVSCKASGNTFTEYTMHWVRQAPGQGLEWI
GGFNPNNGVTNYNQKFKGKVTITRDTSSSTVYMELSSLRSEDTAVYYCARRYYHST
YVFYFDSWGQGTLVTVSS
Humanized 5F3A5D4-V2 H-3.15 heavy chain variable domain sequence
(DNA)
(SEQ ID NO: 1161)
gaggtgcagctggtggaaagcggaggaggcctggttaagcctggaggcagcctgagactgagctgtgccgcttct
ggcaataccttcaccgagtacaccatgcactgggtgcggcaggcccctggcaaaggcctggaatggatcggcggcttcaaccccaa
caacggcgtgacaaattacaaccagaaattcaagggcaagtttacaatcaccagagataagtctaagaacacactctatctgcaaatga
acagcctgaagtccgaggacaccgccgtgtactactgcgccagacggtactaccacagcacatacgtgttctacttcgacagctggg
gccagggcaccctggtcaccgtgtccagc
(amino acids)
(SEQ ID NO: 1162)
EVQLVESGGGLVKPGGSLRLSCAASGNTFTEYTMHWVRQAPGKGLEWIG
GFNPNNGVTNYNQKFKGKFTITRDKSKNTLYLQMNSLKSEDTAVYYCARRYYHSTY
VFYFDSWGQGTLVTVSS
Humanized 5F3A5D4-V2 H-4.4 heavy chain variable domain sequence
(DNA)
(SEQ ID NO: 1163)
caagtgcagctgcaggagagcggacctggcctggtcaagcctggcggcaccctgagcctcacctgtgctgtttctg
gcaataccttcaccgagtacaccatgcactgggtgcggcagcctccaggcaaaggcctggaatggatcggcggatttaaccccaaca
acggcgtgacaaattacaaccagaaattcaagggcaaggtgaccatcacagtggataagtccaagaacaccttcagcctgaagctgt
ctagcgtgacagccgccgacaccgccgtgtactactgcgccagaagatactatcacagcacctacgtgttctacttcgacagctgggg
acagggcacactggtgacagtgtccagc
(amino acids)
(SEQ ID NO: 1164)
QVQLQESGPGLVKPGGTLSLTCAVSGNTFTEYTMHWVRQPPGKGLEWIG
GFNPNNGVTNYNQKFKGKVTITVDKSKNTFSLKLSSVTAADTAVYYCARRYYHSTY
VFYFDSWGQGTLVTVSS
Humanized 5F3A5D4-2 L-1.6 light chain variable domain sequence
(DNA)
(SEQ ID NO: 1165)
gatatccagatgacacagagccctagctccctgagcgccagcgtgggcgaccgggtcaccattacatgcagcgctt
ctcagggcatctccaactacctgaactggtttcagcagaaacccggcaaggcccctaagctgctgatctattacaccagctctctgcac
agcggcgtgccatctagattcagcggatctggcagcggcaccgactacaccctgaccatcagctccctccagcctgaggacttcgcc
acctactactgtcagcaatacagcaagctgccttatacctttggcggcggaacaaaggtggaaatcaag
(amino acids)
(SEQ ID NO: 1166)
DIQMTQSPSSLSASVGDRVTITCSASQGISNYLNWFQQKPGKAPKLLIYYT
SSLHSGVPSRFSGSGSGTDYTLTISSLQPEDFATYYCQQYSKLPYTFGGGTKVEIK
Humanized 5F3A5D4-2 L-3.15 light chain variable domain sequence
(DNA)
(SEQ ID NO: 1167)
gagatcgtgatgacccagagcccagctacacttagtgtgagtccaggtgaacgggctaccctgtcctgcagcgcca
gccagggcatcagcaactacctgaactggtttcagcagaaacctggccaggcccctagactgctgatctattacaccagcagcctgca
cagcggcatccccgccagattcagcggcagcggctctggaacagactacaccctgacaatctctagcctgcagtctgaagattttgcc
gtctactactgtcagcaatacagcaagctgccttataccttcggtggcggaaccaaggtggaaattaag
(amino acids)
(SEQ ID NO: 1168)
EIVMTQSPATLSVSPGERATLSCSASQGISNYLNWFQQKPGQAPRLLIYYT
SSLHSGIPARFSGSGSGTDYTLTISSLQSEDFAVYYCQQYSKLPYTFGGGTKVEIK
Humanized 5F3A5D4-2 L-4.1 light chain variable domain sequence
(DNA)
(SEQ ID NO: 1169)
gatatcgtgatgacccagagcccagacagcctggcagtgagtctgggtgagcgtgctacaatcaactgcagcgcca
gccagggcatctccaactacctgaattggtttcagcagaaacctggccaggctcctaagctgctgatctattacaccagcagcctgcac
agcggcgtgccagatagattcagcggcagcggatctggcaccgactacacactgaccatttcttctctccaggccgaggacgtggcc
gtctactactgtcagcaatacagcaagctgccttacacctttggcggaggcacaaaggtggaaatcaag
(amino acids)
(SEQ ID NO: 1170)
DIVMTQSPDSLAVSLGERATINCSASQGISNYLNWFQQKPGQAPKLLIYYT
SSLHSGVPDRFSGSGSGTDYTLTISSLQAEDVAVYYCQQYSKLPYTFGGGTKVEIK
Humanized 5F3A5D4-3 L-1.6 light chain variable domain sequence
(DNA)
(SEQ ID NO: 1182)
gatatccagatgacacagagccctagctccctgagcgccagcgtgggcgaccgggtcaccattacatgcagcgctt
ctcagggcatctccaactacctgaactggtaccagcagaaacccggcaaggcccctaagctgctgatcttctacaccagctctctgca
cagcggcgtgccatctagattcagcggatctggcagcggcaccgactacaccctgaccatcagctccctccagcctgaggacttcgc
cacctactactgtcagcaatacagcaagctgccttatacctttggcggcggaacaaaggtggaaatcaag
(amino acids)
(SEQ ID NO: 1183)
DIQMTQSPSSLSASVGDRVTITCSASQGISNYLNWYQQKPGKAPKLLIFYT
SSLHSGVPSRFSGSGSGTDYTLTISSLQPEDFATYYCQQYSKLPYTFGGGTKVEIK
Humanized 5F3A5D4-3 L-3.15 light chain variable domain sequence
(DNA)
(SEQ ID NO: 1202)
gagatcgtgatgacccagagcccagctacacttagtgtgagtccaggtgaacgggctaccctgtcctgcagcgcca
gccagggcatcagcaactacctgaactggtaccagcagaaacctggccaggcccctagactgctgatcttctacaccagcagcctgc
acagcggcatccccgccagattcagcggcagcggctctggaacagactacaccctgacaatctctagcctgcagtctgaagattttgc
cgtctactactgtcagcaatacagcaagctgccttataccttcggcggcggaaccaaggtggaaattaag
(amino acids)
(SEQ ID NO: 1203)
EIVMTQSPATLSVSPGERATLSCSASQGISNYLNWYQQKPGQAPRLLIFYT
SSLHSGIPARFSGSGSGTDYTLTISSLQSEDFAVYYCQQYSKLPYTFGGGTKVEIK
Humanized 5F3A5D4-3 L-4.1 light chain variable domain sequence
(DNA)
(SEQ ID NO: 1184)
gatatcgtgatgacccagagcccagacagcctggcagtgagtctgggtgagcgtgctacaatcaactgcagcgcca
gccagggcatctccaactacctgaattggtatcagcagaaacctggccaggctcctaagctgctgatcttctacaccagcagcctgcac
agcggcgtgccagatagattcagcggcagcggatctggcaccgactacacactgaccatttcttctctccaggccgaggacgtggcc
gtctactactgtcagcaatacagcaagctgccttacacctttggcggaggcacaaaggtggaaatcaag
(amino acids)
(SEQ ID NO: 1185)
DIVMTQSPDSLAVSLGERATINCSASQGISNYLNWYQQKPGQAPKLLIFYT
SSLHSGVPDRFSGSGSGTDYTLTISSLQAEDVAVYYCQQYSKLPYTFGGGTKVEIK
Monoclonal antibody 8F9A5A1
Heavy chain variable region sequence
H-3,4,6,10,11
(SEQ ID NO: 437)
atccagttggtgcagtctggacctgagctgaagaagcctggagagacagtcaagatctcctgcaaggcttctgggtat
accttcacaaactatggaatgaactgggtgaagcaggctccaggaaagggtttaaagtggatgggctggataaacacctacactgga
gagccaacatatgttgatgacttcaagggacggtttgccttctctttggaaacctctgccaccactgcctatttgcagatcaacaacctca
aaaatgaggacacgtctacatatttctgtgcaagattgagggggatacgaccgggtcccttggcttactggggccaagggactctggtc
actgtctctgca
Translated protein, wherein the underlined sequence is the complementarity
determining region (CDR):
(SEQ ID NO: 438)
IQLVQSGPELKKPGETVKISCKASGYTFTNYGMNWVKQAPGKGLKWMG
WINTYTGEPTYVDDFKGRFAFSLETSATTAYLQINNLKNEDTSTYFCARLRGIRPGPL
AYWGQGTLVTVSA
Heavy chain variable region CDR1:
(SEQ ID NO: 439)
YTFTNYGMN
Heavy chain variable region CDR2:
(SEQ ID NO: 440)
WINTYTGEPTYVDDFKG
Heavy chain variable region CDR3:
(SEQ ID NO: 441)
LRGIRPGPLAY
Light chain variable region sequence
K-1,2,3,4,5
(SEQ ID NO: 442)
gaaattttgctcacccagtctccagcaatcatagctgcatctcctggggagaaggtcaccatcacctgcagtgccagct
caagtgtaagttacatgaactggtaccagcagaaaccaggatcctcccccaaaatatggatttatggtatatccaacctggcttctggag
ttcctgctcgcttcagtggcagtgggtctgggacatctttctctttcacaatcaacagcatggaggctgaagatgttgccacttattactgtc
agcaaaggagtagttacccacccacgttcggaggggggaccaagctggaaataaaacgg
Translated protein, wherein the underlined sequence is the complementarity
determining region (CDR):
(SEQ ID NO: 443)
EILLTQSPAIIAASPGEKVTITCSASSSVSYMNWYQQKPGSSPKIWIYGISNL
ASGVPARFSGSGSGTSFSFTINSMEAEDVATYYCQQRSSYPPTFGGGTKLEIKR
Light chain variable region CDR 1:
(SEQ ID NO: 444)
SASSSVSYMN
Light chain variable region CDR2:
(SEQ ID NO: 445)
GISNLAS
Light chain variable region CDR3:
(SEQ ID NO: 446)
QQRSSYPPT
8F9A4P3 Heavy chain variable region sequence mouse
(SEQ ID NO: 335)
Gtccagctgcaacagtctggacctgaactggtgaagcctggggcttcagtgaagatatcctgcaagacttctggaaa
cacattcactgaatacaccatgcactgggtgaagcagagccatggaaagagccttgagtggattggaggttttaatcctaacaatggtg
ttactaactacaaccagaagttcaagggcaaggccacattgactgtagacaagtcctccagcacagcctacatggagctccgcagcct
gacatctgaggattctgcagtctattactgtgcaagacggtactaccatagtctctacgtgttttactttgactactggggccaaggcacca
ctctcacagtctcctca
(SEQ ID NO: 1001)
VQLQQSGPELVKPGASVKISCKTSGNTFTEYTMHWVKQSHGKSLEWIGGF
NPNNGVTNYNQKFKGKATLTVDKSSSTAYMELRSLTSEDSAVYYCARRYYHSLYVF
YFDYWGQGTTLTVSS
IGHV1-24*01 V-REGION sequence human (closest match hu antibody
sequence)
(SEQ ID NO: 336)
Caggtccagctggtacagtctggggctgaggtgaagaagcctggggcctcagtgaaggtctcctgcaaggtttccg
gatacaccctcactgaattatccatgcactgggtgcgacaggctcctggaaaagggcttgagtggatgggaggttttgatcctgaagat
ggtgaaacaatctacgcacagaagttccagggcagagtcaccatgaccgaggacacatctacagacacagcctacatggagctgag
cagcctgagatctgaggacacggccgtgtattactgtgcaaca
(SEQ ID NO: 1002)
QVQLVQSGAEVKKPGASVKVSCKVSGYTLTELSMHWVRQAPGKGLEW
MGGFDPEDGETIYAQKFQGRVTMTEDTSTDTAYMELSSLRSEDTAVYYCAT
human (closest match hu antibody sequence)
IGHJ4*01 J-REGION sequence
(SEQ ID NO: 337)
tactttgactactggggccaaggaaccctggtcaccgtctcctca
(SEQ ID NO: 1003)
YFDYWGQGTLVTVSS
humanized heavy chain variable seq (SEQ ID NO: 1001 + SEQ ID NO: 1002 + SEQ ID NO: 1003)
humanized 8F9A4P3 Heavy chain variable region sequence
(DNA)
(SEQ ID NO: 338)
caggtccagctggtacagtctggggctgaggtgaagaagcctggggcctcagtgaaggtctcctgcaaggtttccg
gaaacacattcactgaatacaccatgcactgggtgcgacaggctcctggaaaagggcttgagtggatgggaggttttaatcctaacaat
ggtgttactaactacaaccagaagttcaagggcagagtcaccatgaccgaggacacatctacagacacagcctacatggagctgagc
agcctgagatctgaggacacggccgtgtattactgtgcaagacggtactaccatagtctctacgtgttttactttgactactggggccaag
gaaccctggtcaccgtctcctca
(SEQ ID NO: 1004)
QVQLVQSGAEVKKPGASVKVSCKVSGNTFTEYTMHWVRQAPGKGLEW
MGGFNPNNGVTNYNQKFKGRVTMTEDTSTDTAYMELSSLRSEDTAVYYCARRYYH
SLYVFYFDYWGQGTLVTVSS
humanized heavy chain variable seq (codon optimized version of 1004)
humanized 8F9A4P3 Heavy chain variable region sequence (codon optimized)
(DNA)
(SEQ ID NO: 339)
caggttcagctggttcagtctggcgccgaagtgaagaaacctggcgcctctgtgaaggtgtcctgcaaggtgtccgg
aaataccttcaccgagtacaccatgcactgggtccgacaggcccctggcaaaggacttgaatggatgggcggcttcaaccccaacaa
cggcgtgaccaactacaaccagaaattcaagggccgcgtgaccatgaccgaggacacaagcacagacaccgcctacatggaactg
agcagcctgagaagcgaggacaccgccgtgtactactgcgccagaaggtactaccacagcctgtacgtgttctacttcgactactggg
gccagggcaccctggtcacagtttcttct
(SEQ ID NO: 1005)
QVQLVQSGAEVKKPGASVKVSCKVSGNTFTEYTMHWVRQAPGKGLEW
MGGFNPNNGVTNYNQKFKGRVTMTEDTSTDTAYMELSSLRSEDTAVYYCARRYYH
SLYVFYFDYWGQGTLVTVSS
humanized heavy chain variable seq (“modified” SEQ ID NO: 1005 sequence,
where modified means certain amino acids that are thought to be critical for binding or
structure have been reverted to the mouse sequence).
Modified humanized 8F9A4P3 Heavy chain variable region sequence
(SEQ ID NO: 340)
caggtccagctggtacagtctggggctgaggtgaagaagcctggggcctcagtgaaggtctcctgcaaggtttccg
gaaacacattcactgaatacaccatgcactgggtgcgacaggctcctggaaaagggcttgagtggattggaggttttaatcctaacaat
ggtgttactaactacaaccagaagttcaagggcaaagtcaccctgaccgtggacacatctagcagcacagcctacatggagctgagc
agcctgagatctgaggacacggccgtgtattactgtgcaagacggtactaccatagtctctacgtgttttactttgactactggggccaag
gaaccctggtcaccgtctcctca
(SEQ ID NO: 1006)
QVQLVQSGAEVKKPGASVKVSCKVSGNTFTEYTMHWVRQAPGKGLEWI
GGFNPNNGVTNYNQKFKGKVTLTVDTSSSTAYMELSSLRSEDTAVYYCARRYYHSL
YVFYFDYWGQGTLVTVSS
humanized heavy chain variable seq (SEQ ID NO: 1006 codon optimized)
Modified humanized 8F9A4P3 Heavy chain variable region sequence (codon
optimized)
(SEQ ID NO: 341)
caggttcagctggttcagtctggcgccgaagtgaagaaacctggcgcctctgtgaaggtgtcctgcaaggtgtccgg
aaataccttcaccgagtacaccatgcactgggtccgacaggcccctggcaaaggactggaatggatcggcggcttcaaccccaaca
acggcgtgaccaactacaaccagaaattcaagggcaaagtgaccctgaccgtggacaccagcagcagcacagcctacatggaact
gagcagcctgagaagcgaggacaccgccgtgtactactgcgccagaaggtactaccacagcctgtacgtgttctacttcgactactgg
ggccagggcaccctggtcacagtttcttct
(SEQ ID NO: 1007)
QVQLVQSGAEVKKPGASVKVSCKVSGNTFTEYTMHWVRQAPGKGLEWI
GGFNPNNGVTNYNQKFKGKVTLTVDTSSSTAYMELSSLRSEDTAVYYCARRYYHSL
YVFYFDYWGQGTLVTVSS
8F9A4P3 Light chain variable region sequence mouse
(SEQ ID NO: 342)
gaaacaactgtgacccagtctccagcatccctgtccatggctataggagaaaaagtcaccatcagatgcataaccag
cactgatattgatgatgatatgaactggtaccagcagaagccaggggaacctcctaagctccttatttcagaaggcaatactcttcgtcct
ggagtcccatcccgattctccagcagtggctatggtacagattttgtttttacaattgaaaacatgctctcagaagatgttgcagattactac
tgtttgcaaagtgataacttgcctctcacgttcggctcggggacaaagttggaaataaaacgg
(SEQ ID NO: 1008)
ETTVTQSPASLSMAIGEKVTIRCITSTDIDDDMNWYQQKPGEPPKLLISEGN
TLRPGVPSRFSSSGYGTDFVFTIENMLSEDVADYYCLQSDNLPLTFGSGTKLEIKR
human (closest match hu antibody sequence)
IGKV5-2*01 V-REGION sequence
(SEQ ID NO: 343)
gaaacgacactcacgcagtctccagcattcatgtcagcgactccaggagacaaagtcaacatctcctgcaaagccag
ccaagacattgatgatgatatgaactggtaccaacagaaaccaggagaagctgctattttcattattcaagaagctactactctcgttcct
ggaatcccacctcgattcagtggcagcgggtatggaacagattttaccctcacaattaataacatagaatctgaggatgctgcatattact
tctgt
(SEQ ID NO: 1009)
ETTLTQSPAFMSATPGDKVNISCKASQDIDDDMNWYQQKPGEAAIFIIQEA
TTLVPGIPPRFSGSGYGTDFTLTINNIESEDAAYYFC
human (closest match hu antibody sequence)
IGKJ4*02 J-REGION sequence
(SEQ ID NO: 344)
ctcacgttcggcggagggaccaaggtggagatcaaa
(SEQ ID NO: 1010)
LTFGGGTKVEIK
humanized light chain variable seq (SEQ ID NO: 1008 + SEQ ID NO: 1009 + SEQ ID NO: 1)
humanized 8F9A4P3 Light chain variable region sequence
(SEQ ID NO: 345)
gaaacgacactcacgcagtctccagcattcatgtcagcgactccaggagacaaagtcaacatctcctgcataaccag
cactgatattgatgatgatatgaactggtaccaacagaaaccaggagaagctgctattttcattattcaagaaggcaatactcttcgtcctg
gaatcccacctcgattcagtggcagcgggtatggaacagattttaccctcacaattaataacatagaatctgaggatgctgcatattactt
ctgtttgcaaagtgataacttgcctctcacgttcggcggagggaccaaggtggagatcaaacgg
(SEQ ID NO: 1011)
ETTLTQSPAFMSATPGDKVNISCITSTDIDDDMNWYQQKPGEAAIFIIQEGN
TLRPGIPPRFSGSGYGTDFTLTINNIESEDAAYYFCLQSDNLPLTFGGGTKVEIKR
humanized light chain variable seq (codon optimized version of SEQ ID NO: 1011)
humanized 8F9A4P3 Light chain variable region sequence (codon optimized)
(SEQ ID NO: 346)
Gagacaaccctgacacagagccctgccttcatgtctgccacacctggcgacaaagtgaacatcagctgcatcacca
gcaccgacatcgacgacgacatgaactggtatcagcagaagcctggcgaggccgccatcttcatcatccaagagggcaacacactg
cggcctggcatccctcctagattttctggcagcggctacggcaccgacttcaccctgaccatcaacaacatcgagagcgaggacgcc
gcctactacttctgcctgcaaagcgacaacctgcctctgacctttggcggaggcaccaaggtggaaatcaagcgg
(SEQ ID NO: 1012)
ETTLTQSPAFMSATPGDKVNISCITSTDIDDDMNWYQQKPGEAAIFIIQEGN
TLRPGIPPRFSGSGYGTDFTLTINNIESEDAAYYFCLQSDNLPLTFGGGTKVEIKR
humanized light chain variable seq (“modified” SEQ ID NO: 1012 sequence,
where modified means certain amino acids that are thought to be critical for binding or
structure have been reverted to the mouse sequence).
Modified humanized 8F9A4P3 Light chain variable region sequence
(SEQ ID NO: 347)
gaaacgacagtgacgcagtctccagcattcatgtcagcgactccaggagacaaagtcaccatctcctgcataaccag
cactgatattgatgatgatatgaactggtaccaacagaaaccaggagaagctgctattctgctgattagcgaaggcaatactcttcgtcct
ggaatcccacctcgattcagtagcagcgggtatggaacagattttaccctcacaattaataacatagaatctgaggatgctgcatattact
tctgtttgcaaagtgataacttgcctctcacgttcggcggagggaccaaggtggagatcaaacgg
(SEQ ID NO: 1013)
ETTVTQSPAFMSATPGDKVTISCITSTDIDDDMNWYQQKPGEAAILLISEG
NTLRPGIPPRFSSSGYGTDFTLTINNIESEDAAYYFCLQSDNLPLTFGGGTKVEIKR
humanized light chain variable seq
Modified humanized 8F9A4P3 Light chain variable region sequence (codon
optimized)
(SEQ ID NO: 348)
gagacaaccgtgacacagagccctgccttcatgtctgccacacctggcgacaaagtgaccatcagctgcatcacca
gcaccgacatcgacgacgacatgaactggtatcagcagaagcctggcgaggccgccatcctgcttatctctgagggaaacacactgc
ggcctggcatccctcctagattttccagcagcggctacggcaccgacttcaccctgaccatcaacaacatcgagagcgaggacgccg
cctactacttctgcctgcaaagcgacaacctgcctctgacctttggcggaggcaccaaggtggaaatcaagcgg
(SEQ ID NO: 1014)
ETTVTQSPAFMSATPGDKVTISCITSTDIDDDMNWYQQKPGEAAILLISEG
NTLRPGIPPRFSSSGYGTDFTLTINNIESEDAAYYFCLQSDNLPLTFGGGTKVEIKR
humanized heavy and light chains joined via a flexible linker.
Modified humanized 8F9A4P3 sequence (codon optimized)
(SEQ ID NO: 349)
Caggttcagctggttcagtctggcgccgaagtgaagaaacctggcgcctctgtgaaggtgtcctgcaaggtgtccgg
aaataccttcaccgagtacaccatgcactgggtccgacaggcccctggcaaaggactggaatggatcggcggcttcaaccccaaca
acggcgtgaccaactacaaccagaaattcaagggcaaagtgaccctgaccgtggacaccagcagcagcacagcctacatggaact
gagcagcctgagaagcgaggacaccgccgtgtactactgcgccagaaggtactaccacagcctgtacgtgttctacttcgactactgg
ggccagggcaccctggtcacagtttcttctggcggtggcggaagcggaggcggtggctccggtggcggaggcagcgaaacgaca
gtgacgcagtctccagcattcatgtcagcgactccaggagacaaagtcaccatctcctgcataaccagcactgatattgatgatgatatg
aactggtaccaacagaaaccaggagaagctgctattctgctgattagcgaaggcaatactcttcgtcctggaatcccacctcgattcagt
agcagcgggtatggaacagattttaccctcacaattaataacatagaatctgaggatgctgcatattacttctgtttgcaaagtgataactt
gcctctcacgttcggcggagggaccaaggtggagatcaaacgg
(SEQ ID NO: 1015)
QVQLVQSGAEVKKPGASVKVSCKVSGNTFTEYTMHWVRQAPGKGLEWI
GGFNPNNGVTNYNQKFKGKVTLTVDTSSSTAYMELSSLRSEDTAVYYCARRYYHSL
YVFYFDYWGQGTLVTVSSGGGGSGGGGSGGGGSETTVTQSPAFMSATPGDKVTISCI
TSTDIDDDMNWYQQKPGEAAILLISEGNTLRPGIPPRFSSSGYGTDFTLTINNIESEDA
AYYFCLQSDNLPLTFGGGTKVEIKR
8F9A5A1 Heavy chain variable region sequence
(SEQ ID NO: 350)
atccagttggtgcagtctggacctgagctgaagaagcctggagagacagtcaagatctcctgcaaggcttctgggtat
accttcacaaactatggaatgaactgggtgaagcaggctccaggaaagggtttaaagtggatgggctggataaacacctacactgga
gagccaacatatgttgatgacttcaagggacggtttgccttctctttggaaacctctgccaccactgcctatttgcagatcaacaacctca
aaaatgaggacacgtctacatatttctgtgcaagattgagggggatacgaccgggtcccttggcttactggggccaagggactctggtc
actgtctctgca
(SEQ ID NO: 1016)
IQLVQSGPELKKPGETVKISCKASGYTFTNYGMNWVKQAPGKGLKWMG
WINTYTGEPTYVDDFKGRFAFSLETSATTAYLQINNLKNEDTSTYFCARLRGIRPGPL
AYWGQGTLVTVSA
IGHV7-81*01 V-REGION sequence
(SEQ ID NO: 351)
caggtgcagctggtgcagtctggccatgaggtgaagcagcctggggcctcagtgaaggtctcctgcaaggcttctg
gttacagtttcaccacctatggtatgaattgggtgccacaggcccctggacaagggcttgagtggatgggatggttcaacacctacact
gggaacccaacatatgcccagggcttcacaggacggtttgtcttctccatggacacctctgccagcacagcatacctgcagatcagca
gcctaaaggctgaggacatggccatgtattactgtgcgaga
(SEQ ID NO: 1017)
QVQLVQSGHEVKQPGASVKVSCKASGYSFTTYGMNWVPQAPGQGLEW
MGWFNTYTGNPTYAQGFTGRFVFSMDTSASTAYLQISSLKAEDMAMYYCAR
IGHJ4*03 J-REGION sequence
(SEQ ID NO: 352)
tactttgactactggggccaagggaccctggtcaccgtctcctca
(SEQ ID NO: 1018)
YFDYWGQGTLVTVSS
humanized 8F9A5A1 Heavy chain variable region sequence
(SEQ ID NO: 353)
Caggtgcagctggtgcagtctggccatgaggtgaagcagcctggggcctcagtgaaggtctcctgcaaggcttctg
ggtataccttcacaaactatggaatgaactgggtgccacaggcccctggacaagggcttgagtggatgggatggataaacacctaca
ctggagagccaacatatgttgatgacttcaagggacggtttgtcttctccatggacacctctgccagcacagcatacctgcagatcagc
agcctaaaggctgaggacatggccatgtattactgtgcaagattgagggggatacgaccgggtcccttggcttactggggccaaggg
accctggtcaccgtctcctca
(SEQ ID NO: 1019)
QVQLVQSGHEVKQPGASVKVSCKASGYTFTNYGMNWVPQAPGQGLEW
MGWINTYTGEPTYVDDFKGRFVFSMDTSASTAYLQISSLKAEDMAMYYCARLRGIR
PGPLAYWGQGTLVTVSS
humanized 8F9A5A1 Heavy chain variable region sequence (codon optimized)
(SEQ ID NO: 354)
caggttcagctggtgcagtctggccacgaagtgaaacagcctggcgcctctgtgaaggtgtcctgtaaagccagcg
gctacacctttaccaactacggcatgaactgggtgccccaggctcctggacaaggcttggaatggatgggctggatcaacacctacac
cggcgagcctacctacgtggacgacttcaagggcagattcgtgttcagcatggacaccagcgccagcacagcctacctgcagatca
gctctctgaaggccgaggatatggccatgtactactgcgccagactgagaggcatcagacctggacctctggcctattggggacagg
gcacactggtcacagtgtcctct
(SEQ ID NO: 1020)
QVQLVQSGHEVKQPGASVKVSCKASGYTFTNYGMNWVPQAPGQGLEW
MGWINTYTGEPTYVDDFKGRFVFSMDTSASTAYLQISSLKAEDMAMYYCARLRGIR
PGPLAYWGQGTLVTVSS
Modified humanized 8F9A5A1 Heavy chain variable region sequence
(SEQ ID NO: 355)
cagatccagctggtgcagtctggccccgaggtgaagcagcctggggcctcagtgaaggtctcctgcaaggcttctg
ggtataccttcacaaactatggaatgaactgggtgaagcaggcccctggacaagggcttgagtggatgggatggataaacacctaca
ctggagagccaacatatgttgatgacttcaagggacggtttgccttctccatggacacctctgccagcacagcatacctgcagatcagc
agcctaaaggctgaggacaccgccacctattactgtgcaagattgagggggatacgaccgggtcccttggcttactggggccaaggg
accctggtcaccgtctcctca
(SEQ ID NO: 1021)
QIQLVQSGPEVKQPGASVKVSCKASGYTFTNYGMNWVKQAPGQGLEWM
GWINTYTGEPTYVDDFKGRFAFSMDTSASTAYLQISSLKAEDTATYYCARLRGIRPG
PLAYWGQGTLVTVSS
Modified humanized 8F9A5A1 Heavy chain variable region sequence (codon
optimized)
(SEQ ID NO: 356)
cagattcagctggtgcagtctggccccgaagtgaaacaacctggcgcctctgtgaaggtgtcctgcaaggccagcg
gctacacctttaccaactacggcatgaactgggtcaagcaggcccctggacaaggcctggaatggatgggctggatcaacacctaca
ccggcgagcctacctacgtggacgacttcaagggcagattcgccttcagcatggacaccagcgccagcacagcctacctgcagatc
agctctctgaaggccgaggacaccgccacctactactgtgccagactgagaggcatcagacccggacctctggcctattggggaca
gggaacactggtcaccgtgtcctct
(SEQ ID NO: 1022)
QIQLVQSGPEVKQPGASVKVSCKASGYTFTNYGMNWVKQAPGQGLEWM
GWINTYTGEPTYVDDFKGRFAFSMDTSASTAYLQISSLKAEDTATYYCARLRGIRPG
PLAYWGQGTLVTVSS
8F9A5A1 Light chain variable region sequence
(SEQ ID NO: 357)
gaaattttgctcacccagtctccagcaatcatagctgcatctcctggggagaaggtcaccatcacctgcagtgccagct
caagtgtaagttacatgaactggtaccagcagaaaccaggatcctcccccaaaatatggatttatggtatatccaacctggcttctggag
ttcctgctcgcttcagtggcagtgggtctgggacatctttctctttcacaatcaacagcatggaggctgaagatgttgccacttattactgtc
agcaaaggagtagttacccacccacgttcggaggggggaccaagctggaaataaaacgg
(SEQ ID NO: 1023)
EILLTQSPANIAASPGEKVTITCSASSSVSYMNWYQQKPGSSPKIWIYGISNL
ASGVPARFSGSGSGTSFSFTINSMEAEDVATYYCQQRSSYPPTFGGGTKLEIKR
IGKV3D-15*02 V-REGION sequence
(SEQ ID NO: 358)
gaaatagtgatgatgcagtctccagccaccctgtctgtgtctccaggggaaagagccaccctctcctgcagggccag
tcagagtgttagcagcaacttagcctggtaccagcagaaacctggccaggctcccaggctcctcatctatggtgcatccaccagggcc
actggcatcccagccaggttcagtggcagtgggtctgggacagagttcactctcaccatcagcagcctgcagtctgaagattttgcagt
ttattactgtcagcagtataataac
(SEQ ID NO: 1024)
EIVMMQSPATLSVSPGERATLSCRASQSVSSNLAWYQQKPGQAPRLLIYG
ASTRATGIPARFSGSGSGTEFTLTISSLQSEDFAVYYCQQYNN
IGKJ4*02 J-REGION sequence
(SEQ ID NO: 359)
ctcacgttcggcggagggaccaaggtggagatcaaa
(SEQ ID NO: 1025)
LTFGGGTKVEIK
humanized 8F9A5A1 Light chain variable region sequence
(SEQ ID NO: 360)
gaaatagtgatgatgcagtctccagccaccctgtctgtgtctccaggggaaagagccaccctctcctgcagtgccagc
tcaagtgtaagttacatgaactggtaccagcagaaacctggccaggctcccaggctcctcatctatggtatatccaacctggcttctggc
atcccagccaggttcagtggcagtgggtctgggacagagttcactctcaccatcagcagcctgcagtctgaagattttgcagtttattact
gtcagcaaaggagtagttacccacccacgttcggcggagggaccaaggtggagatcaaacgg
(SEQ ID NO: 1026)
EIVMMQSPATLSVSPGERATLSCSASSSVSYMNWYQQKPGQAPRLLIYGIS
NLASGIPARFSGSGSGTEFTLTISSLQSEDFAVYYCQQRSSYPPTFGGGTKVEIKR
humanized 8F9A5A1 Light chain variable region sequence (codon optimized)
(SEQ ID NO: 361)
gagatcgtgatgatgcagagccccgccacactgagtgtgtctccaggcgaaagagccacactgtcctgtagcgcca
gcagcagcgtgtcctacatgaactggtatcagcagaagcccggacaggcccctagactgctgatctacggcatcagcaatctggcca
gcggcatccctgccagattttctggctctggctccggcaccgagttcaccctgacaatctctagcctgcagagcgaggacttcgccgtg
tactactgccagcagagaagcagctaccctcctacctttggcggaggcaccaaggtggaaatcaagcgg
(SEQ ID NO: 1027)
EIVMMQSPATLSVSPGERATLSCSASSSVSYMNWYQQKPGQAPRLLIYGIS
NLASGIPARFSGSGSGTEFTLTISSLQSEDFAVYYCQQRSSYPPTFGGGTKVEIKR
Modified humanized 8F9A5A1 Light chain variable region sequence
(SEQ ID NO: 362)
gaaatagtgctgacccagtctccagccaccctgtctgtgtctccaggggaaagagccaccctctcctgcagtgccag
ctcaagtgtaagttacatgaactggtaccagcagaaacctggccaggctcccaggctctggatctatggtatatccaacctggcttctgg
catcccagccaggttcagtggcagtgggtctgggacaagcttcagcctcaccatcagcagcctgcagtctgaagattttgcagtttatta
ctgtcagcaaaggagtagttacccacccacgttcggcggagggaccaaggtggagatcaaacgg
(SEQ ID NO: 1028)
EIVLTQSPATLSVSPGERATLSCSASSSVSYMNWYQQKPGQAPRLWIYGIS
NLASGIPARFSGSGSGTSFSLTISSLQSEDFAVYYCQQRSSYPPTFGGGTKVEIKR
Modified humanized 8F9A5A1 Light chain variable region sequence (codon
optimized)
(SEQ ID NO: 363)
gagatcgtgctgacacagtctcccgccacactgagtgtgtctccaggcgaaagagccacactgtcctgtagcgccag
cagcagcgtgtcctacatgaactggtatcagcagaagcccggacaggcccctagactgtggatctacggcatcagcaatctggccag
cggcatccctgccagattttctggctctggctccggcaccagcttcagcctgacaatcagcagcctgcagagcgaggacttcgccgtg
tactactgccagcagagaagcagctaccctcctacctttggcggaggcaccaaggtggaaatcaagcgg
(amino acids)
(SEQ ID NO: 1029)
EIVLTQSPATLSVSPGERATLSCSASSSVSYMNWYQQKPGQAPRLWIYGIS
NLASGIPARFSGSGSGTSFSLTISSLQSEDFAVYYCQQRSSYPPTFGGGTKVEIKR
Modified humanized 8F9A5A1 scFV sequence (codon optimized)
(SEQ ID NO: 364)
Cagattcagctggtgcagtctggccccgaagtgaaacaacctggcgcctctgtgaaggtgtcctgcaaggccagcg
gctacacctttaccaactacggcatgaactgggtcaagcaggcccctggacaaggcctggaatggatgggctggatcaacacctaca
ccggcgagcctacctacgtggacgacttcaagggcagattcgccttcagcatggacaccagcgccagcacagcctacctgcagatc
agctctctgaaggccgaggacaccgccacctactactgtgccagactgagaggcatcagacccggacctctggcctattggggaca
gggaacactggtcaccgtgtcctctggcggtggcggaagcggaggcggtggctccggtggcggaggcagcgagatcgtgctgac
acagtctcccgccacactgagtgtgtctccaggcgaaagagccacactgtcctgtagcgccagcagcagcgtgtcctacatgaactg
gtatcagcagaagcccggacaggcccctagactgtggatctacggcatcagcaatctggccagcggcatccctgccagattttctggc
tctggctccggcaccagcttcagcctgacaatcagcagcctgcagagcgaggacttcgccgtgtactactgccagcagagaagcag
ctaccctcctacctttggcggaggcaccaaggtggaaatcaagcgg
(SEQ ID NO: 1030)
QIQLVQSGPEVKQPGASVKVSCKASGYTFTNYGMNWVKQAPGQGLEWM
GWINTYTGEPTYVDDFKGRFAFSMDTSASTAYLQISSLKAEDTATYYCARLRGIRPG
PLAYWGQGTLVTVSSGGGGSGGGGSGGGGSEIVLTQSPATLSVSPGERATLSCSASS
SVSYMNWYQQKPGQAPRLWIYGISNLASGIPARFSGSGSGTSFSLTISSLQSEDFAVY
YCQQRSSYPPTFGGGTKVEIKR
8H5H5G4 Heavy chain variable region sequence
(SEQ ID NO: 365)
gtccagctgcaacagtctggacctgatctggtgaagcctgggacttcagtgaagatatcctgtaagacttctggaaaca
cattcactgaatacaccatgcactgggtgaagcagagccatggaaagagccttgagtggattggaggttttaatcctaacaatggtgtta
ctaactacaaccagaagttcaagggcaaggccacattgactgtagacaagtcctccagcacagcctacatggagctccgcagcctga
catctgaggattctgcagtctattactgtgcaagacgttactaccatagtacctacgtgttctactttgactcctggggccaaggcaccact
ctcacagtctcctca
(SEQ ID NO: 1031)
VQLQQSGPDLVKPGTSVKISCKTSGNTFTEYTMHWVKQSHGKSLEWIGGF
NPNNGVTNYNQKFKGKATLTVDKSSSTAYMELRSLTSEDSAVYYCARRYYHSTYVF
YFDSWGQGTTLTVSS
IGHV1-24*01 V-REGION sequence
(DNA)
(SEQ ID NO: 366)
caggtccagctggtacagtctggggctgaggtgaagaagcctggggcctcagtgaaggtctcctgcaaggtttccg
gatacaccctcactgaattatccatgcactgggtgcgacaggctcctggaaaagggcttgagtggatgggaggttttgatcctgaagat
ggtgaaacaatctacgcacagaagttccagggcagagtcaccatgaccgaggacacatctacagacacagcctacatggagctgag
cagcctgagatctgaggacacggccgtgtattactgtgcaaca
(amino acids)
(SEQ ID NO: 1032)
QVQLVQSGAEVKKPGASVKVSCKVSGYTLTELSMHWVRQAPGKGLEW
MGGFDPEDGETIYAQKFQGRVTMTEDTSTDTAYMELSSLRSEDTAVYYCAT
IGHJ4*03 J-REGION sequence
(DNA)
(SEQ ID NO: 367)
tactttgactactggggccaagggaccctggtcaccgtctcctca
(amino acids)
(SEQ ID NO: 1033)
YFDYWGQGTLVTVSS
Humanized 8H5H5G4 Heavy chain variable region sequence
(DNA)
(SEQ ID NO: 368)
caggtccagctggtacagtctggggctgaggtgaagaagcctggggcctcagtgaaggtctcctgcaaggtttccg
gaaacacattcactgaatacaccatgcacTgggtgcgacaggctcctggaaaagggcttgagtggatgggaggttttaatcctaacaa
tggtgttactaactacaaccagaagttcaagggcAgagtcaccatgaccgaggacacatctacagacacagcctacatggagctgag
cagcctgagatctgaggacacggccgtgtattactgtGcaagacgttactaccatagtacctacgtgttctactttgactcctggggcca
agggaccctggtcaccgtctcctca
(amino acids)
(SEQ ID NO: 1034)
QVQLVQSGAEVKKPGASVKVSCKVSGNTFTEYTMHWVRQAPGKGLEW
MGGFNPNNGVTNYNQKFKGRVTMTEDTSTDTAYMELSSLRSEDTAVYYCARRYYH
STYVFYFDSWGQGTLVTVSS
Humanized 8H5H5G4 Heavy chain variable region sequence (codon
optimized)
(DNA)
(SEQ ID NO: 369)
caggttcagctggttcagtctggcgccgaagtgaagaaacctggcgcctctgtgaaggtgtcctgcaaggtgtccgg
aaataccttcaccgagtacaccatgcactgggtccgacaggcccctggcaaaggacttgaatggatgggcggcttcaaccccaacaa
cggcgtgaccaactacaaccagaaattcaagggccgcgtgaccatgaccgaggacacaagcacagacaccgcctacatggaactg
agcagcctgagaagcgaggacaccgccgtgtactactgcgccagaaggtactaccacagcacctacgtgttctacttcgacagctgg
ggccagggcacactggtcacagtttcttct
(amino acids)
(SEQ ID NO: 1035)
QVQLVQSGAEVKKPGASVKVSCKVSGNTFTEYTMHWVRQAPGKGLEW
MGGFNPNNGVTNYNQKFKGRVTMTEDTSTDTAYMELSSLRSEDTAVYYCARRYYH
STYVFYFDSWGQGTLVTVSS
Modified Humanized 8H5H5G4 Heavy chain variable region sequence
(DNA)
(SEQ ID NO: 370)
caggtccagctggtacagtctggggctgaggtgaagaagcctggggcctcagtgaaggtctcctgcaaggtttccg
gaaacacattcactgaatacaccatgcactgggtgcgacaggctcctggaaaagggcttgagtggatcggaggttttaatcctaacaat
ggtgttactaactacaaccagaagttcaagggcaaggtcaccctgaccgtggacacatctagcagcacagcctacatggagctgagc
agcctgagatctgaggacacggccgtgtattactgtgcaagacgttactaccatagtacctacgtgttctactttgactcctggggccaa
gggaccctggtcaccgtctcctca
(amino acids)
(SEQ ID NO: 1036)
QVQLVQSGAEVKKPGASVKVSCKVSGNTFTEYTMHWVRQAPGKGLEWI
GGFNPNNGVTNYNQKFKGKVTLTVDTSSSTAYMELSSLRSEDTAVYYCARRYYHST
YVFYFDSWGQGTLVTVSS
Modified Humanized 8H5H5G4 Heavy chain variable region sequence (codon
optimized)
(DNA)
(SEQ ID NO: 371)
caggttcagctggttcagtctggcgccgaagtgaagaaacctggcgcctctgtgaaggtgtcctgcaaggtgtccgg
aaataccttcaccgagtacaccatgcactgggtccgacaggcccctggcaaaggactggaatggatcggcggcttcaaccccaaca
acggcgtgaccaactacaaccagaaattcaagggcaaagtgaccctgaccgtggacaccagcagcagcacagcctacatggaact
gagcagcctgagaagcgaggacaccgccgtgtactactgcgccagaaggtactaccacagcacctacgtgttctacttcgacagctg
gggccagggcacactggtcacagtttcttct
(amino acids)
(SEQ ID NO: 1037)
QVQLVQSGAEVKKPGASVKVSCKVSGNTFTEYTMHWVRQAPGKGLEWI
GGFNPNNGVTNYNQKFKGKVTLTVDTSSSTAYMELSSLRSEDTAVYYCARRYYHST
YVFYFDSWGQGTLVTVSS
8H5H5G4 Light chain variable region sequence
(DNA)
(SEQ ID NO: 372)
gatatccagatgacacagactacatcctccctgtctgcctctctgggagacagagtcaccatcagttgcagtgcaagtc
agggcattagcaattatttaaactggtttcagcagaaaccagatggaactattaagctcctgatctattacacatcaagtttacattcagga
gtcccatcaaggttcagtggcagtgggtctgggacagattattctctcaccatcagtaatgtggaacctgaagatattgccacttactattg
tcagcagtatagtaagcttccttacacgttcggaggggggaccaagctggagataaaacgg
(amino acids)
(SEQ ID NO: 1038)
DIQMTQTTSSLSASLGDRVTISCSASQGISNYLNWFQQKPDGTIKLLIYYTS
SLHSGVPSRFSGSGSGTDYSLTISNVEPEDIATYYCQQYSKLPYTFGGGTKLEIKR
IGKV1-27*01 V-REGION sequence
(DNA)
(SEQ ID NO: 373)
gacatccagatgacccagtctccatcctccctgtctgcatctgtaggagacagagtcaccatcacttgccgggcgagt
cagggcattagcaattatttagcctggtatcagcagaaaccagggaaagttcctaagctcctgatctatgctgcatccactttgcaatcag
gggtcccatctcggttcagtggcagtggatctgggacagatttcactctcaccatcagcagcctgcagcctgaagatgttgcaacttatt
actgtcaaaagtataacagtgcccct
(amino acids)
(SEQ ID NO: 1039)
DIQMTQSPSSLSASVGDRVTITCRASQGISNYLAWYQQKPGKVPKLLIYAA
STLQSGVPSRFSGSGSGTDFTLTISSLQPEDVATYYCQKYNSAP
IGKJ4*02 J-REGION sequence
(DNA)
(SEQ ID NO: 374)
ctcacgttcggcggagggaccaaggtggagatcaaa
(amino acids)
(SEQ ID NO: 1040)
LTFGGGTKVEIK
humanized 8H5H5G4 Light chain variable region sequence
(DNA)
(SEQ ID NO: 375)
gacatccagatgacccagtctccatcctccctgtctgcatctgtaggagacagagtcaccatcacttgcagtgcaagtc
agggcattagcaattatttaaacTggtatcagcagaaaccagggaaagttcctaagctcctgatctattacacatcaagtttacattcagg
ggtcccatctcggttcagtggcagtggatctgggacagatttcactctcaccatcagcagcctgcagcctgaagatgttgcaacttatta
ctgtcagcagtatagtaagcttccttacacgttcggcggagggaccaaggtggagatcaaacgg
(amino acids)
(SEQ ID NO: 1041)
DIQMTQSPSSLSASVGDRVTITCSASQGISNYLNWYQQKPGKVPKLLIYYT
SSLHSGVPSRFSGSGSGTDFTLTISSLQPEDVATYYCQQYSKLPYTFGGGTKVEIKR
humanized 8H5H5G4 Light chain variable region sequence (codon optimized)
(DNA)
(SEQ ID NO: 376)
gacatccagatgacacagagccctagcagcctgtctgccagcgtgggagacagagtgaccatcacatgtagcgcca
gccagggcatcagcaactacctgaactggtatcagcagaaacccggcaaggtgcccaagctgctgatctactacaccagcagcctg
cacagcggcgtgccaagcagattttctggcagcggctctggcaccgacttcaccctgaccatatctagcctgcagcctgaggacgtg
gccacctactactgtcagcagtacagcaagctgccctacacctttggcggaggcaccaaggtggaaatcaagcgg
(amino acids)
(SEQ ID NO: 1042)
DIQMTQSPSSLSASVGDRVTITCSASQGISNYLNWYQQKPGKVPKLLIYYT
SSLHSGVPSRFSGSGSGTDFTLTISSLQPEDVATYYCQQYSKLPYTFGGGTKVEIKR
Modified humanized 8H5H5G4 Light chain variable region sequence
(DNA)
(SEQ ID NO: 377)
gacatccagatgacccagtctccatcctccctgtctgcatctgtaggagacagagtcaccatcacttgcagtgcaagtc
agggcattagcaattatttaaactggtatcagcagaaaccagggaaagttcctaagctcctgatctattacacatcaagtttacattcagg
ggtcccatctcggttcagtggcagtggatctgggacagattacactctcaccatcagcagcctgcagcctgaagatgttgcaacttatta
ctgtcagcagtatagtaagcttccttacacgttcggcggagggaccaaggtggagatcaaacgg
(amino acids)
(SEQ ID NO: 1043)
DIQMTQSPSSLSASVGDRVTITCSASQGISNYLNWYQQKPGKVPKLLIYYT
SSLHSGVPSRFSGSGSGTDYTLTISSLQPEDVATYYCQQYSKLPYTFGGGTKVEIKR
Modified humanized 8H5H5G4 Light chain variable region sequence (codon
optimized)
(DNA)
(SEQ ID NO: 378)
gacatccagatgacacagagccctagcagcctgtctgccagcgtgggagacagagtgaccatcacatgtagcgcca
gccagggcatcagcaactacctgaactggtatcagcagaaacccggcaaggtgcccaagctgctgatctactacaccagcagcctg
cacagcggcgtgccaagcagattttctggcagcggctctggcaccgactacaccctgaccatatctagcctgcagcctgaggacgtg
gccacctactactgtcagcagtacagcaagctgccctacacctttggcggaggcaccaaggtggaaatcaagcgg
(amino acids)
(SEQ ID NO: 1044)
DIQMTQSPSSLSASVGDRVTITCSASQGISNYLNWYQQKPGKVPKLLIYYT
SSLHSGVPSRFSGSGSGTDYTLTISSLQPEDVATYYCQQYSKLPYTFGGGTKVEIKR
Modified humanized 8H5H5G4 scFV sequence (codon optimized)
(DNA)
(SEQ ID NO: 379)
Caggttcagctggttcagtctggcgccgaagtgaagaaacctggcgcctctgtgaaggtgtcctgcaaggtgtccgg
aaataccttcaccgagtacaccatgcactgggtccgacaggcccctggcaaaggactggaatggatcggcggcttcaaccccaaca
acggcgtgaccaactacaaccagaaattcaagggcaaagtgaccctgaccgtggacaccagcagcagcacagcctacatggaact
gagcagcctgagaagcgaggacaccgccgtgtactactgcgccagaaggtactaccacagcacctacgtgttctacttcgacagctg
gggccagggcacactggtcacagtttcttctggcggtggcggaagcggaggcggtggctccggtggcggaggcagcgacatcca
gatgacacagagccctagcagcctgtctgccagcgtgggagacagagtgaccatcacatgtagcgccagccagggcatcagcaact
acctgaactggtatcagcagaaacccggcaaggtgcccaagctgctgatctactacaccagcagcctgcacagcggcgtgccaagc
agattttctggcagcggctctggcaccgactacaccctgaccatatctagcctgcagcctgaggacgtggccacctactactgtcagca
gtacagcaagctgccctacacctttggcggaggcaccaaggtggaaatcaagcgg
(amino acids)
(SEQ ID NO: 1045)
QVQLVQSGAEVKKPGASVKVSCKVSGNTFTEYTMHWVRQAPGKGLEWI
GGFNPNNGVTNYNQKFKGKVTLTVDTSSSTAYMELSSLRSEDTAVYYCARRYYHST
YVFYFDSWGQGTLVTVSSGGGGSGGGGSGGGGSDIQMTQSPSSLSASVGDRVTITCS
ASQGISNYLNWYQQKPGKVPKLLIYYTSSLHSGVPSRFSGSGSGTDYTLTISSLQPED
VATYYCQQYSKLPYTFGGGTKVEIKR
Human IgG1 heavy chain constant region sequence: (for making full antibody -
pair with either kappa or lambda constant region; 2 plasmids, express together)
(DNA)
(SEQ ID NO: 380)
gctagcaccaagggcccatcggtcttccccctggcaccctcctccaagagcacctctgggggcacagcggccctgg
gctgcctggtcaaggactacttccccgaaccggtgacggtgtcgtggaactcaggcgccctgaccagcggcgtgcacaccttcccg
gctgtcctacagtcctcaggactctactccctcagcagcgtggtgacagtgccctccagcagcttgggcacccagacctacatctgca
acgtgaatcacaagcccagcaacaccaaggtggacaagaaagttgagcccaaatcttgtgacaaaactcacacatgcccaccgtgcc
cagcacctgaactcctggggggaccgtcagtcttcctcttccccccaaaacccaaggacaccctcatgatctcccggacccctgaggt
cacatgcgtggtggtggacgtgagccacgaagaccctgaggtcaagttcaactggtacgtggacggcgtggaggtgcataatgcca
agacaaagccgcgggaggagcagtacaacagcacgtaccgtgtggtcagcgtcctcaccgtcctgcaccaggactggctgaatgg
caaggagtacaagtgcaaggtctccaacaaagccctcccagcccccatcgagaaaaccatctccaaagccaaagggcagccccga
gaaccacaggtgtacaccctgcccccatcccgggaggagatgaccaagaaccaggtcagcctgacctgcctggtcaaaggcttctat
cccagcgacatcgccgtggagtgggagagcaatgggcagccggagaacaactacaagaccacgcctcccgtgctggactccgac
ggctccttcttcctctacagcaagctcaccgtggacaagagcaggtggcagcaggggaacgtcttctcatgctccgtgatgcatgagg
ctctgcacaaccactacacgcagaagagcctctccctgtctccgggtaaatga
(amino acids)
(SEQ ID NO: 1046)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGK
Human IgG2 heavy chain constant region sequence: (for making full antibody -
pair with either kappa or lambda constant region; 2 plasmids, express together)
(DNA)
(SEQ ID NO: 381)
gcctccaccaagggcccatcggtcttccccctggcgccctgctccaggagcacctccgagagcacagccgccctg
ggctgcctggtcaaggactacttccccgaaccggtgacggtgtcgtggaactcaggcgctctgaccagcggcgtgcacaccttccca
gctgtcctacagtcctcaggactctactccctcagcagcgtggtgaccgtgccctccagcaacttcggcacccagacctacacctgca
acgtagatcacaagcccagcaacaccaaggtggacaagacagttgagcgcaaatgttgtgtcgagtgcccaccgtgcccagcacca
cctgtggcaggaccgtcagtcttcctcttccccccaaaacccaaggacaccctcatgatctcccggacccctgaggtcacgtgcgtgg
tggtggacgtgagccacgaagaccccgaggtccagttcaactggtacgtggacggcgtggaggtgcataatgccaagacaaagcc
acgggaggagcagttcaacagcacgttccgtgtggtcagcgtcctcaccgttgtgcaccaggactggctgaacggcaaggagtaca
agtgcaaggtctccaacaaaggcctcccagcccccatcgagaaaaccatctccaaaaccaaagggcagccccgagaaccacaggt
gtacaccctgcccccatcccgggaggagatgaccaagaaccaggtcagcctgacctgcctggtcaaaggcttctaccccagcgaca
tcgccgtggagtgggagagcaatgggcagccggagaacaactacaagaccacacctcccatgctggactccgacggctccttcttc
ctctacagcaagctcaccgtggacaagagcaggtggcagcaggggaacgtcttctcatgctccgtgatgcatgaggctctgcacaac
cactacacgcagaagagcctctccctgtctccgggtaaatag
(amino acids)
(SEQ ID NO: 1047)
ASTKGPSVFPLAPCSRSTSESTAALGCLVKDYFPEPVTVSWNSGALTSGVH
TFPAVLQSSGLYSLSSVVTVPSSNFGTQTYTCNVDHKPSNTKVDKTVERKCCVECPP
CPAPPVAGPSVFLFPPKPKDTLMISRTPEVTCVVVDVSHEDPEVQFNWYVDGVEVHN
AKTKPREEQFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDS
DGSFFLYSKLTVDKSRWQQGNVFSCSVMHEALHNHYTQKSLSLSPGK
Human Kappa light chain constant region sequence:
(DNA)
(SEQ ID NO: 382)
aggacggtggctgcaccatctgtcttcatcttcccgccatctgatgagcagttgaaatctggaactgcctctgttgtgtg
cctgctgaataacttctatcccagagaggccaaagtacagtggaaggtggataacgccctccaatcgggtaactcccaggagagtgtc
acagagcaggacagcaaggacagcacctacagcctcagcagcaccctgacgctgagcaaagcagactacgagaaacacaaagtc
tacgcctgcgaagtcacccatcagggcctgagctcgcccgtcacaaagagcttcaacaggggagagtgttag
(amino acids)
(SEQ ID NO: 1048)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
Human Lambda light chain constant region sequence:
(DNA)
(SEQ ID NO: 383)
ggtcagcccaaggctgccccctcggtcactctgttcccgccctcctctgaggagcttcaagccaacaaggccacact
ggtgtgtctcataagtgacttctacccgggagccgtgacagtggcctggaaggcagatagcagccccgtcaaggcgggagtggaga
ccaccacaccctccaaacaaagcaacaacaagtacgcggccagcagctatctgagcctgacgcctgagcagtggaagtcccacag
aagctacagctgccaggtcacgcatgaagggagcaccgtggagaagacagtggcccctacagaatgttcatag
(amino acids)
(SEQ ID NO: 1049)
GQPKAAPSVTLFPPSSEELQANKATLVCLISDFYPGAVTVAWKADSSPVK
AGVETTTPSKQSNNKYAASSYLSLTPEQWKSHRSYSCQVTHEGSTVEKTVAPTECS
Human IgG1 Fc region sequence: (to be fused to scFv for homo-dimerizes)
(DNA)
(SEQ ID NO: 384)
gagcccaaatcttgtgacaaaactcacacatgcccaccgtgcccagcacctgaactcctggggggaccgtcagtctt
cctcttccccccaaaacccaaggacaccctcatgatctcccggacccctgaggtcacatgcgtggtggtggacgtgagccacgaaga
ccctgaggtcaagttcaactggtacgtggacggcgtggaggtgcataatgccaagacaaagccgcgggaggagcagtacaacagc
acgtaccgtgtggtcagcgtcctcaccgtcctgcaccaggactggctgaatggcaaggagtacaagtgcaaggtctccaacaaagcc
ctcccagcccccatcgagaaaaccatctccaaagccaaagggcagccccgagaaccacaggtgtacaccctgcccccatcccggg
aggagatgaccaagaaccaggtcagcctgacctgcctggtcaaaggcttctatcccagcgacatcgccgtggagtgggagagcaat
gggcagccggagaacaactacaagaccacgcctcccgtgctggactccgacggctccttcttcctctacagcaagctcaccgtggac
aagagcaggtggcagcaggggaacgtcttctcatgctccgtgatgcatgaggctctgcacaaccactacacgcagaagagcctctcc
ctgtctccgggtaaatga
(amino acids)
(SEQ ID NO: 1050)
EPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEALHNH
YTQKSLSLSPGK*
Human IgG2 Fc region sequence:
(DNA)
(SEQ ID NO: 385)
gagcgcaaatgttgtgtcgagtgcccaccgtgcccagcaccacctgtggcaggaccgtcagtcttcctcttcccccca
aaacccaaggacaccctcatgatctcccggacccctgaggtcacgtgcgtggtggtggacgtgagccacgaagaccccgaggtcca
gttcaactggtacgtggacggcgtggaggtgcataatgccaagacaaagccacgggaggagcagttcaacagcacgttccgtgtgg
tcagcgtcctcaccgttgtgcaccaggactggctgaacggcaaggagtacaagtgcaaggtctccaacaaaggcctcccagccccc
atcgagaaaaccatctccaaaaccaaagggcagccccgagaaccacaggtgtacaccctgcccccatcccgggaggagatgacca
agaaccaggtcagcctgacctgcctggtcaaaggcttctaccccagcgacatcgccgtggagtgggagagcaatgggcagccgga
gaacaactacaagaccacacctcccatgctggactccgacggctccttcttcctctacagcaagctcaccgtggacaagagcaggtg
gcagcaggggaacgtcttctcatgctccgtgatgcatgaggctctgcacaaccactacacgcagaagagcctctccctgtctccgggt
aaatag
(amino acids)
(SEQ ID NO: 1051)
ERKCCVECPPCPAPPVAGPSVFLFPPKPKDTLMISRTPEVTCVVVDVSHED
PEVQFNWYVDGVEVHNAKTKPREEQFNSTFRVVSVLTVVHQDWLNGKEYKCKVSN
KGLPAPIEKTISKTKGQPREPQVYTLPPSREEMTKNQVSLTCLVKGFYPSDIAVEWES
NGQPENNYKTTPPMLDSDGSFFLYSKLTVDKSRWQQGNVFSCSVMHEALHNHYTQ
KSLSLSPGK*

In another aspect of the invention, an immune cell engineered to express a CAR is administered to a patient diagnosed with or at risk of developing a cancer or cancer metastasis, wherein the immune cell is also engineered to express an anti-NME7 antibody or antibody fragment, which may be expressed off of an inducible promoter. In one aspect, the CAR is guided by an anti-MUC1* antibody fragment. In one case, the CAR is huMNC2-CAR44. In one aspect, the anti-NME7 antibody or antibody fragment binds to an NME peptide listed under sections “Homologous peptides to A1, A2, B1, B2 or B3 peptides” and the “Homologous extended peptides to A1, A2, B1, B2 or B3 peptides” above. In another aspect, the antibody or antibody fragment binds to an NME7 derived peptide chosen from among A1, A2, B1, B2 or B3 (SEQ ID NOS: 141-145). In yet another aspect, the antibody, antibody fragment or antibody mimic binds to an NME7 peptide comprising the B3 peptide. In one aspect of the invention, the anti-NME7 antibody, antibody fragment or antibody mimic comprises sequences derived from the variable domains of anti-NME7 antibodies 8F9A4A3, 8F9A5A1, or 8H5H5G4.

Such antibodies may be human or humanized. Such antibodies may be polyclonal, monoclonal, bispecific, bivalent, monovalent, single chain, scFv, or may be an antibody mimic such as protein scaffolds that present recognition regions that bind to a specific target. As is appreciated by those skilled in the art, antibodies can be of non-human origin, human or humanized. Methods for humanizing antibodies include fusing all or some of the mouse variable regions to V- and J-regions of a closest match human antibody sequence, for example, as shown in sequences listed as SEQ ID NOS: 1001-1045. Full antibodies, rather than single chain constructs, can also be made. For example, the heavy chain variable mouse sequence is fused to human V- and J-regions then fused to the human heavy chain constant regions of IgG1, IgG2 or IgG3. Similarly, the light chain variable mouse sequences are fused to human V- and J-regions then fused to either the human Kappa or Lambda constant regions of IgG1, IgG2 or IgG3. Plasmids are expressed together and associate to form the full antibody (SEQ ID NOS: 1047-1051).

In another aspect of the invention, small molecules are anti-cancer agents that are selected for their ability to inhibit the tumorigenic effects of NME7, NME7_{AB} or NME7-X1. For example, a high throughput screen identifies small molecules that will treat cancer. In a multi-well plate, small molecules are separately added to wells in which cancer cells are cultured in a medium containing NME7_{AB}. If the small molecule diminishes the amount of cells that become floaters and/or reduces the expression of metastatic markers such as CXCR4, CHD1 or pluripotent stem cell markers, then that small molecule is an anti-cancer drug candidate. Another method of identifying small molecules that are anti-cancer agents is to select those small molecules that bind to NME7, NME7_{AB} or NME7-X1 or suppresses expression of the NME7 species. Yet another high throughput screen is to select for small molecules that inhibit the binding of NME7_{AB} to the PSMGFR peptide of the MUC1* extracellular domain and those small molecules will be anti-cancer agents.

The sequences of NME7_{AB} and NME7-X1 differ only in that NME7-X1 is missing some of the N-terminal sequence that NME7_{AB} has. Experiments show that there is a naturally occurring NME7 species that is nearly identical to NME7_{AB}, which we call NME7_{AB}-like species. Antibodies that bind to NME7-X1 may also bind to the naturally occurring species that mimics NME7_{AB}, unless there are conformational differences that an antibody can differentiate. Therefore, if it is desired to inhibit NME7-X1 but not NME7_{AB}-like species, or vice versa, siRNA, anti-sense nucleic acids, or genetic editing techniques can be used to inhibit expression of one but not the other.

In one case, the anti-cancer therapeutic agent is a nucleic acid that directly or indirectly suppresses specific expression of NME7, NME7-X1 or NME7_{AB}-like species. Such nucleic acids can be siRNA, RNAi, anti-sense nucleic acids and the like that directly suppress the NME7 species. In another aspect of the invention, the nucleic acid can indirectly suppress the NME7 species for example by altering the expression of a molecule that regulates it. For example, the super enhancer BRD4 suppresses expression of NME7. Therefore, an effective therapeutic for the treatment or prevention of cancer is an agent that increases expression of BRD4. An effective therapeutic may be an agent that increases expression of BRD4's co-factor, JMJD6.

Peptides derived from NME7_{AB} or NME7-X1, or the entire protein, are used to generate anti-NME7 or anti-NME7-X1 antibodies in animals that we have demonstrated inhibit cancer growth and inhibit transition of cancer cells to metastatic cancer cells. Similarly, NME7 derived peptides can be administered to a human such that they generate antibodies that treat or prevent cancer or inhibit transition of cancer cells to metastatic cancer cells. NME7 peptides or proteins are administered to a person as a type of vaccine to stimulate the production of anti-NME7, anti-NME7_{AB} or anti-NME7-X1 antibodies in the recipient. The results shown in FIG. 12 and FIG. 13 indicate that immunizing a person with a collection of peptides derived from NME7, especially in the NME7-X1 or NME7_{AB} sequences may be a more effective vaccine than immunizing with a single peptide. Said peptides or proteins may further be conjugated to a carrier protein or other adjuvant, known to those skilled in the art to aid in the stimulation of an immune response.

NME7 peptides that lie outside of the DM10 domain are preferred to generate antibodies for the treatment or prevention of cancer. Peptides that can be administered to a patient for the prevention of cancer or metastasis contain sequences of the peptides listed in FIG. 6-FIG. 9. A1, A2, B1, B2 and B3 are examples of peptides that generate antibodies that bind to NME7_{AB} and NME7-X1 and are administered to a patient for the treatment or prevention of cancer. The invention is not limited to peptides of the exact sequence as is naturally occurring in NME7 or NME7-X1. As is known to those skilled in the art, substitution of several amino acids of a peptide sequence can still give rise to antibodies that specifically recognize the natural protein sequence. It is not intended that the invention be limited to the peptides demonstrated herein to inhibit cancer growth or inhibit the transition of regular cancer cells to metastatic cancer cells. The methods used here to identify peptides A1, A2, B1, B2 and B3 can also be used to identify other peptide sequences that could be equally or more effective than the peptides demonstrated here.

Chimeric antigen receptor molecules comprising portions of human NME7_{AB} or NME7-X1 or comprising an antibody fragment that binds to NME7_{AB} or NME7-X1 are anti-cancer therapeutics and are administered to a patient for the treatment or prevention of cancers or cancer metastases.

In one instance, the recognition units or variable regions of anti-NME7 antibodies are fused to molecules of T cells using the technology known as CAR (chimeric antigen receptor) technology or CAR T technology. The salient feature of antibodies or fragments thereof that can be used therapeutically to treat or prevent cancers is the identification of antibody-like variable regions that recognize NME7 and prevent its interaction with targets that promote cancers. In one case, the target is the PSMGFR region of MUC1*.

Antibodies, antibody fragments or single chain antibodies can be engineered into chimeric molecules, including chimeric antigen receptors, also known as CARs, which molecules are then transfected or transduced into an immune system cell, such as a T cell, and administered to a patient. The humanized antibodies or antibody fragments, typically an scFv, comprises much of the extracellular domain of a CAR. The antibody fragment is biochemically fused to immune system signaling molecules, such as CD8 as the transmembrane domain and cytoplasmic signaling motifs such as T cell receptor signaling molecules also called activation domains, or co-stimulatory domains including but not limited to CD3-zeta, CD28, 41bb, OX40. CARs can be transfected into T cells or other cells, preferably immune system cells and administered to a patient. Here we describe CARs in which the extracellular portion contains an anti-NME7, anti-NME7_{AB} or anti-NME7-X1 antibody, antibody fragment or single chain, scFv antibody fragment. In a preferred embodiment, the antibody or antibody fragment is human or humanized.

Effective anti-NME7 or anti-NME7-X1 antibodies or fragments will have the ability to bind to native NME7, NME7_{AB} or NME7-X1. In practice, the parent antibody, from which the extracellular domain of the CAR is engineered, is generated by immunizing an animal with an NME7, NME7_{AB} or NME7-X1 derived peptide. In one aspect of the invention, the immunizing peptide is comprised of NME7 amino acids 1-376. In one aspect of the invention, the immunizing peptide is comprised of NME7 amino acids 92-376. In another aspect of the invention, the immunizing peptide is comprised of NME7 amino acids 125-376. In yet another aspect of the invention, the immunizing peptide is made up of sequences listed in FIG. 6-FIG. 8. In another aspect of the invention, the immunizing peptide is made up of sequences listed in FIG. 9. Alternatively, the parent antibody or the antibody fragment is selected from a library or pool of antibodies, which may be natural, synthetic or fragments of either, wherein they are selected for their ability to bind to NME7, NME7_{AB} or NME7-X1, peptides listed in FIG. 6-FIG. 8, or peptides listed in FIG. 9.

The targeting portion of a CAR need not be an antibody or antibody fragment. Here we describe a CAR wherein the extracellular domain contains an NME7 fragment. NME7-derived peptide(s) are engineered into a different sort of CAR wherein the targeting portion of the extracellular domain is a protein fragment or peptide rather than an antibody or antibody fragment. The peptide CARs are transfected or transduced into an immune system cell, typically a T cell. The NME7 fragments or NME7 derived peptides are selected for their ability to bind to their cognate binding partners but should not be able to function as intact NME7, NME7_{AB} or NME7-X1 and confer tumorigenic activity. NME7 fragments or NME7 derived peptides are biochemically fused to immune system signaling molecules, such as CD8 as the transmembrane domain and cytoplasmic signaling motifs such as T cell receptor signaling molecules also called activation domains, or co-stimulatory domains including but not limited to CD3-zeta, CD28, 41bb, OX40.

In one aspect of the invention, the NME7 fragment is most or all of the NME7 NDPK B domain. In another aspect of the invention, the NME7 fragment is an NME7 peptide that contains one or more of the peptide sequences listed in FIG. 6-FIG. 9. Experiments indicate that, for strategies that use NME7 or fragments of NME7, NME7_{AB}, or NME7-X1 as the targeting portion of a chimeric antigen receptor (CAR) for engineered immune cell therapeutics, fairly large fragments of NME7_{AB} or NME7-X1 would be more effective than shorter peptides, for example peptides less than 15 amino acids in length. Alternatively, a collection of CARs, each bearing a different NME7_{AB} derived peptide can collectively be transfected or transduced into an immune system cell and administered to a patient for the treatment or prevention of cancers. Experiments shown in FIG. 12-FIG. 13 support the validity of this approach.

CARs that contain an NME7 fragment in its extracellular domain are transfected or transduced into an immune system cell, typically a T cell, and administered to a patient for the treatment or prevention of cancers. In one aspect, the cancer is a MUC1*-positive cancer. In another aspect, the cancer is a metastatic cancer.

Agents that inhibit an enzyme that cleaves NME7 can be used to treat or prevent cancers. Some forms of NME7 are sequestered within the cell and therefore are not secreted from the cell whereupon they can act as growth factors to promote cancers. Full-length NME7 is 42 kDa. However, we found that a ~33 kDa NME7 species that is devoid of the DM10 domain and appears to be essentially identical to the recombinant NME7_{AB} that we generated, is secreted from cancer cells and stem cells. This ~33 kDa NME7 species and another ~25 kDa NME7 species may be cleavage products that would be eliminated by an agent that inhibited cleavage of NME7.

The detection of elevated levels of NME7, or an ~33 kDa NME7 species, which we call NME7_{AB}-like species, or NME7-X1 in a patient sample is diagnostic of the presence of cancer or its progression to a more aggressive or metastatic state. The inventors have discovered that both early stage, naïve stem cells and cancer cells, especially MUC1*-positive cancer cells, express high levels of a ~33 kDa NME7 that is devoid of the DM10 domain and NME7-X1.

NME7-X1 was recently listed in a protein database as being a theoretical alternative isoform of NME7, however, it had never been detected in tissues or cells. We designed primers that differentiate NME7-X1 from NME7 by PCR. The expression levels of human NME7, NME7a, NME7b and NME7-X1 were measured by PCR in a panel of cells that included fibroblast cells, human embryonic stem cells, human iPS cells, T47D human breast cancer cells, DU145 human prostate cancer cells, PC3 human prostate cancer cells, HEK295 human fetal liver cells, and other human stem cell lines. NME7 is expressed at higher levels in cancer cells than in stem cells. Particularly, NME7-X1 is expressed 10-fold higher in prostate cancer cells and 3-fold higher in breast cancer cells, than it is in fibroblast cells or stem cells. NME7-X1 is expressed ~5-fold higher in HEK293 fetal liver cells than it is in fibroblast cells or stem cells and therefore predicts that NME7-X1 is elevated in liver cancers. NME7b is expressed 17-25-times higher in prostate cancer cells than in stem cells.

Detection of elevated levels of NME7 species in a patient sample will be indicators that the patient has a cancer or is at risk of developing a cancer. Levels of NME7 species levels can be measured or assessed by PCR, hybridization schemes, cycling probe technologies, FISH, immunocytochemistry, IHC, Western blot, immunoprecipitation, sandwich assays, ELISA assays and the like. The patient sample may be a fluid sample, a blood sample, milk, urine, cells, liquid biopsy, biopsy and the like. In a patient diagnosed with cancer, elevated levels of NME7 species are indicators of increased metastatic potential. Elevated levels of NME7-X1 are indicators of prostate cancer. Antibodies of the invention are used to detect and distinguish NME7 species and are used as a diagnostic tool.

Because adult cells and tissues do not express significant levels of NME7 or secrete NME7, an effective way to diagnose cancer or to diagnose a more aggressive or metastatic form, or a shift to a more aggressive form, is to measure levels of NME7 in a sample from a patient, from a collection of cells or tissues or from cultured cells, compared to NME7 levels in a healthy sample or compared to levels of NME7 known to exist in healthy adult cells or tissues. Increased levels of NME7 indicate the presence of cancer, the presence of a metastatic cancer or the onset of metastasis. Increased levels of NME7 is also indicative of a MUC1*-positive cancer. The sample assayed for the presence of NME7 may be a collection of cells that may be cultured cell lines or cells from a patient, a bodily fluid, a blood sample, a tissue specimen, or a biopsy specimen. Therefore, a diagnostic assay that will detect the presence of cancer or the progression of cancer, comprises the steps of: 1) obtaining a sample from a patient having cancer or at risk of developing a cancer; 2) subjecting that sample to an assay capable of detecting or measuring levels of NME7, or levels of nucleic acids encoding NME7; 3) comparing levels of the measured NME7 protein or NME7-encoding nucleic acids in the test sample to levels in control patients or control cells; 4) determining that the levels of NME7 or nucleic acids encoding NME7 are elevated compared to the controls; and 5) concluding that the donor of the test sample has cancer or has had a progression of cancer if the control to which the test was compared came from a donor previously diagnosed with a cancer.

In this assay, the control sample to which the test sample is compared can be non-cancerous cells, cultured cells, a sample from a healthy donor, a non-cancerous sample from the donor, or a sample from the donor of the test sample wherein the control sample was taken from the donor at a previous point in time. The source of such samples may be any specimen taken from the patient being tested for the presence or progression of cancer, including bodily fluids, cerebrospinal fluid, bone marrow samples, blood, tissues, cells, biopsy tissues or cells, cultured cells derived from a patient's cells and the like. The source of the sample to which the test sample is compared can be bodily fluids, cerebrospinal fluid, bone marrow samples, blood, tissues, cells, biopsy tissues or cells, or cultured cells that may be derived from a healthy donor or the test patient wherein the samples were taken at a previous point in time. The measured levels to which the test sample is compared may be from previously recorded data and compiled into lists for comparison to test samples.

### Theranostics

Patients diagnosed with elevated levels of NME7 protein or nucleic acids encoding NME7 are then treated with therapeutic agents that suppress expression of NME7, inhibit cleavage of NME7 or inhibit NME7 binding to its targets, wherein such interaction promotes cancers. An important target of NME7 or a cleavage product of NME7, is MUC1*. NME7 binds to and dimerizes the extracellular domain of MUC1*. Therefore, patients diagnosed with elevated levels of NME7 will benefit from treatment with therapeutic agents that inhibit NME7 and/or therapeutic agents that inhibit the dimerization of a cleaved form of MUC1, whose extracellular domain is comprised of some or all of the PSMGFR sequence. Thus assessing suitability of cancer treatments and administration of an effective amount of a therapeutic for the treatment or prevention of cancers would consists of the steps of: 1) obtaining a sample from a patient suspected of having a cancer or at risk of developing a cancer or at risk of developing a metastatic cancer; 2) measuring an amount of NME7 or a cleavage product thereof or an NME7 encoding nucleic acid wherein the measured levels are significantly above those measured in a control sample; 3) determining that the patient has a cancer or has developed a more aggressive or a metastatic cancer; 4) administering to the patient an effective amount of a therapeutic agent that suppresses expression of NME7, inhibits cleavage of NME7 or inhibits NME7 binding to its targets and/or administering to the patient an effective amount of a therapeutic agent that suppresses expression of MUC1, inhibits cleavage of MUC1 to MUC1* or inhibits MUC1* binding to its targets. In a preferred embodiment, the therapeutic agent that inhibits NME7 binding to its targets, inhibits its interaction with MUC1*. In a more preferred embodiments, it inhibits its interaction with the extracellular domain of MUC1* comprised essentially of the PSMGFR sequence. In a preferred embodiment, the therapeutic agent that inhibits MUC1* binding to its targets, inhibits the interaction between MUC1* and NME7. In a more preferred embodiment, the therapeutic agent that inhibits the interaction between MUC1* and NME7 inhibits the binding of MUC1* to the portion of NME7 that is comprised essentially of the sequence of NME7_{AB}.

### Chemically Modified Peptides

Polypeptide or antibody therapeutics may suffer from short circulating half-life, and proteolytic degradation and low solubility. To improve the pharmacokinetics and pharmacodynamics properties of the inventive biopharmaceuticals, methods such as manipulation of the amino acid sequence may be made to decrease or increase immunogenicity and decrease proteolytic cleavage; fusion or conjugation of the peptides to immunoglobulins and serum proteins, such as albumin may be made; incorporation into drug delivery vehicles for the biopharmaceuticals such as the inventive peptides and antibodies for protection and slow release may also be made; and conjugating to natural or synthetic polymers are also contemplated. In particular, for synthetic polymer conjugation, pegylation or acylation, such as N-acylation, S-acylation and so forth are also contemplated.

### Nucleic Acid Constructs

Also provided is an expression vector comprising a nucleic acid molecule of the invention as described herein, wherein the nucleic acid molecule is operatively linked to an expression control sequence. Also provided is a host-vector system for the production of a polypeptide which comprises the expression vector of the invention which has been introduced into a host cell suitable for expression of the polypeptide. The suitable host cell may be a bacterial cell such as E. coli, a yeast cell, such as Pichia pastoris, an insect cell, such as Spodoptera frugiperda, or a mammalian cell, such as a COS, HEK or CHO cell.

The present invention also provides for methods of producing the polypeptides of the invention by growing cells of the host-vector system described herein, under conditions permitting production of the polypeptide and recovering the polypeptide so produced. The polypeptides useful for practicing the present invention may be prepared by expression in a prokaryotic or eukaryotic expression system.

The recombinant gene may be expressed and the polypeptide purified utilizing any number of methods. The gene may be subcloned into a bacterial expression vector, such as for example, but not by way of limitation, pZErO.

The polypeptides may be purified by any technique which allows for the subsequent formation of a stable, biologically active protein. For example, and not by way of limitation, the factors may be recovered from cells either as soluble proteins or as inclusion bodies, from which they may be extracted quantitatively by 8M guanidinium hydrochloride and dialysis. In order to further purify the factors, any number of purification methods may be used, including but not limited to conventional ion exchange chromatography, affinity chromatography, different sugar chromatography, hydrophobic interaction chromatography, reverse phase chromatography or gel filtration.

When used herein, polypeptide includes functionally equivalent molecules in which amino acid residues are substituted for residues within the sequence resulting in a silent or conservative change. For example, one or more amino acid residues within the sequence can be substituted by another amino acid of a similar polarity, which acts as a functional equivalent, resulting in a silent or conservative alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. The potential glycosylation amino acids include serine, threonine, and asparagine. Also included within the scope of the invention are proteins or fragments or derivatives thereof which exhibit the same or similar biological activity and derivatives which are differentially modified during or after translation, e.g., by glycosylation, proteolytic cleavage, linkage to an antibody molecule or other cellular ligand, etc.

Any of the methods known to one skilled in the art for the insertion of DNA fragments into a vector may be used to construct expression vectors encoding the polypeptides of the invention using appropriate transcriptional/translational control signals and protein coding sequences. These methods may include in vitro recombinant DNA and synthetic techniques and in vivo recombinations (genetic recombination). Expression of nucleic acid sequence encoding the polypeptides of the invention may be regulated by a second nucleic acid sequence so that the polypeptide is expressed in a host transformed with the recombinant DNA molecule. For example, expression of the polypeptides described herein may be controlled by any promoter/enhancer element known in the art. Promoters which may be used to control expression of the polypeptide include, but are not limited to the long terminal repeat as described in Squinto et al., (1991, Cell 65:1-20); the SV40 early promoter region (Bernoist and Chambon, 1981, Nature 290:304-310), the CMV promoter, the M-MuLV 5′ terminal repeat the promoter contained in the 3′long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:144-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25), see also “Useful proteins from recombinant bacteria” in Scientific American, 1980, 242:74-94; promoter elements from yeast or other fungi such as the Gal 4 promoter, the ADH (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), Sendai virus, lenti virus, albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58); alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94); myelin basic protein gene control region which is active in oligodendrocyte cells in the brain (Readhead et al., 1987, Cell 48:703-712); myosin light chain-2 gene control region which is active in skeletal muscle (Shani, 1985, Nature 314:283-286), and gonadotropic releasing hormone gene control region which is active in the hypothalamus (Mason et al., 1986, Science 234:1372-1378).

Thus, according to the invention, expression vectors capable of being replicated in a bacterial or eukaryotic host comprising nucleic acids encoding a polypeptide as described herein, are used to transfect the host and thereby direct expression of such nucleic acid to produce polypeptides which may then be recovered in biologically active form. As used herein, a biologically active form includes a form capable of binding to the relevant receptor and causing a differentiated function and/or influencing the phenotype of the cell expressing the receptor.

Expression vectors containing the nucleic acid inserts can be identified by without limitation, at least three general approaches: (a) DNA-DNA hybridization, (b) presence or absence of “marker” gene functions, and (c) expression of inserted sequences. In the first approach, the presence of foreign nucleic acids inserted in an expression vector can be detected by DNA-DNA hybridization using probes comprising sequences that are homologous to an inserted nucleic acid sequences. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of foreign nucleic acid sequences in the vector. For example, if an efl nucleic acid sequence is inserted within the marker gene sequence of the vector, recombinants containing the insert can be identified by the absence of the marker gene function. In the third approach, recombinant expression vectors can be identified by assaying the foreign nucleic acid product expressed by the recombinant constructs. Such assays can be based, for example, on the physical or functional properties of the nucleic acid product of interest, for example, by binding of a ligand to a receptor or portion thereof which may be tagged with, for example, a detectable antibody or portion thereof or binding to antibodies produced against the protein of interest or a portion thereof.

The polypeptide, in particular modified of the present invention, may be expressed in the host cells transiently, constitutively or permanently.

Effective doses useful for treating the diseases or disorders indicated in the present application may be determined using methods known to one skilled in the art (see, for example, Fingl, et al., The Pharmacological Basis of Therapeutics, Goodman and Gilman, eds. Macmillan Publishing Co, New York, pp. 1-46 (1975). Pharmaceutical compositions for use according to the invention include the polypeptides described above in a pharmacologically acceptable liquid, solid or semi-solid carrier, linked to a carrier or targeting molecule (e.g., antibody, hormone, growth factor, etc.) and/or incorporated into liposomes, microcapsules, and controlled release preparation prior to administration in vivo. For example, the pharmaceutical composition may comprise a polypeptide in an aqueous solution, such as sterile water, saline, phosphate buffer or dextrose solution. Alternatively, the active agents may be comprised in a solid (e.g. wax) or semi-solid (e.g. gelatinous) formulation that may be implanted into a patient in need of such treatment. The administration route may be any mode of administration known in the art, including but not limited to intravenously, intrathecally, subcutaneously, intrauterinely, by injection into involved tissue, intraarterially, intranasally, orally, or via an implanted device.

Administration may result in the distribution of the active agent of the invention throughout the body or in a localized area. For example, in some conditions, which involve distant regions of the nervous system, intravenous or intrathecal administration of agent may be desirable. In some situations, an implant containing active agent may be placed in or near the lesioned area. Suitable implants include, but are not limited to, gelfoam, wax, spray, or microparticle-based implants.

The present invention also provides for pharmaceutical compositions comprising the polypeptides described herein, in a pharmacologically acceptable vehicle. The compositions may be administered systemically or locally. Any appropriate mode of administration known in the art may be used, including, but not limited to, intravenous, intrathecal, intraarterial, intranasal, oral, subcutaneous, intraperitoneal, or by local injection or surgical implant. Sustained release formulations are also provided for.

### Gene Therapy

Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11 (5): 155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors, or by direct injection of naked DNA, or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors) and so on. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor. Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contain nucleic acid sequences encoding the polypeptide are used. The nucleic acid sequences encoding the polypeptide to be used in gene therapy are cloned into one or more vectors, which facilitates delivery of the gene into a patient. Lentiviral vectors, such as retroviral vectors, and other vectors such as adenoviral vectors and adeno-associated viruses are examples of viral vectors that may be used. Retroviral vectors contain the components necessary for the correct packaging of the viral genome and integration into the host cell DNA.

Adenoviruses are especially attractive vehicles for delivering genes to respiratory epithelia because they naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. In addition, adeno-associated virus (AAV) has also been proposed for use in gene therapy.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion and so on. Numerous techniques are known in the art for the introduction of foreign genes into cells and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T-lymphocytes, B-lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, and so on.

In a preferred embodiment, the cell used for gene therapy is autologous to the patient.

In an embodiment in which recombinant cells are used in gene therapy, nucleic acid sequences encoding the polypeptide are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention.

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### Therapeutic Composition

The formulation of therapeutic compounds is generally known in the art and reference can conveniently be made to Remington's Pharmaceutical Sciences, 17th ed., Mack Publishing Co., Easton, Pa., USA. For example, from about 0.05 ng to about 20 mg per kilogram of body weight per day may be administered. Dosage regime may be adjusted to provide the optimum therapeutic response. For example, several divided doses may be administered daily or the dose may be proportionally reduced as indicated by the exigencies of the therapeutic situation. The active compound may be administered in a convenient manner such as by the oral, intravenous (where water soluble), intramuscular, subcutaneous, intra nasal, intra ocular, intradermal or suppository routes or implanting (eg using slow release molecules by the intraperitoneal route or by using cells e.g. monocytes or dendrite cells sensitized in vitro and adoptively transferred to the recipient). Depending on the route of administration, the peptide may be required to be coated in a material to protect it from the action of enzymes, acids and other natural conditions which may inactivate said ingredients.

For example, the low lipophilicity of the peptides will allow them to be destroyed in the gastrointestinal tract by enzymes capable of cleaving peptide bonds and in the stomach by acid hydrolysis. In order to administer peptides by other than parenteral administration, they will be coated by, or administered with, a material to prevent its inactivation. For example, peptides may be administered in an adjuvant, co-administered with enzyme inhibitors or in liposomes. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEP) and trasylol. Liposomes include water-in-oil-in-water CGF emulsions as well as conventional liposomes.

The active compounds may also be administered parenterally or intraperitoneally. Dispersions can also be prepared in glycerol liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, chlorobutanol, phenol, sorbic acid, theomersal and the like. In many cases, it will be preferable to include isotonic agents, for example, sugars or sodium chloride. Prolonged absorption of the injectable compositions can be brought about by the use in the composition of agents delaying absorption, for example, aluminium monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterile active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from a previously sterile-filtered solution thereof.

When the peptides are suitably protected as described above, the active compound may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations.

### Delivery Systems

Various delivery systems are known and can be used to administer a compound of the invention, e.g., encapsulation in liposomes, microparticles, microcapsules, recombinant cells capable of expressing the compound, receptor-mediated endocytosis, construction of a nucleic acid as part of a retroviral or other vector, etc. Methods of introduction include but are not limited to intradermal, intramuscular, intraperitoneal, intravenous, subcutaneous, intranasal, intra ocular, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody or a peptide of the invention, care must be taken to use materials to which the protein does not absorb. In another embodiment, the compound or composition can be delivered in a vesicle, in particular a liposome. In yet another embodiment, the compound or composition can be delivered in a controlled release system. In one embodiment, a pump may be used. In another embodiment, polymeric materials can be used. In yet another embodiment, a controlled release system can be placed in proximity of the therapeutic target, thus requiring only a fraction of the systemic dose.

### Sequence Listing Free Text

As regards the use of nucleotide symbols other than a, g, c, t, they follow the convention set forth in WIPO Standard ST.25, Appendix 2, Table 1, wherein k represents t or g; n represents a, c, t or g; m represents a or c; r represents a or g; s represents c or g; w represents a or t and y represents c or t.

(SEQ ID NO: 1)
MTPGTQSPFF LLLLLTVLTV VTGSGHASST PGGEKETSAT
QRSSVPSSTE KNAVSMTSSV LSSHSPGSGS STTQGQDVTL
APATEPASGS AATWGQDVTS VPVTRPALGS TTPPAHDVTS
APDNKPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDNRPALGS TAPPVHNVTS
ASGSASGSAS TLVHNGTSAR ATTTPASKST PFSIPSHHSD
TPTTLASHST KTDASSTHHS SVPPLTSSNH STSPQLSTGV
SFFFLSFHIS NLQFNSSLED PSTDYYQELQ RDISEMFLQI
YKQGGFLGLS NIKFRPGSVV VQLTLAFREG TINVHDVETQ
FNQYKTEAAS RYNLTISDVS VSDVPFPFSA QSGAGVPGWG
IALLVLVCVL VALAIVYLIA LAVCQCRRKN YGQLDIFPAR
DTYHPMSEYP TYHTHGRYVP PSSTDRSPYE KVSAGNGGSS
LSYTNPAVAA ASANL describes full-length MUC1
Receptor (Mucin 1 precursor, Genbank Accession
number: P15941).
(SEQ ID NO: 2)
MTPGTQSPFFLLLLLTVLT
(SEQ ID NO: 3)
MTPGTQSPFFLLLLLTVLT VVTA
(SEQ ID NO: 4)
MTPGTQSPFFLLLLLTVLT VVTG

SEQ ID NOS: 2, 3 and 4 describe N-terminal MUC-1 signaling sequence for directing MUC1 receptor and truncated isoforms to cell membrane surface. Up to 3 amino acid residues may be absent at C-terminal end as indicated by variants in SEQ ID NOS: 2, 3 and 4.

GTINVHDVETQFNQYKTEAASRYNLTISDVSVSDVPFPFSAQSGAGVPGW GIALLVLVCVLVALAIVYLIALAVCQCRRKNYGQLDIFPARDTYHPMSEYPTYHTHG RYVPPSSTDRSPYEKVSAGNGGSSLSYTNPAVAAASANL (SEQ ID NO:5) describes a truncated MUC1 receptor isoform having nat-PSMGFR at its N-terminus and including the transmembrane and cytoplasmic sequences of a full-length MUC1 receptor.

GTINVHDVETQFNQYKTEAASRYNLTISDVSVSDVPFPFSAQSGA (SEQ ID NO: 6) describes the extracellular domain of Native Primary Sequence of the MUC1 Growth Factor Receptor (nat-PSMGFR—an example of “PSMGFR”):

TINVHDVETQFNQYKTEAASRYNLTISDVSVSDVPFPFSAQSGA (SEQ ID NO: 7) describes the extracellular domain of Native Primary Sequence of the MUC1 Growth Factor Receptor (nat-PSMGFR—An example of “PSMGFR”), having a single amino acid deletion at the N-terminus of SEQ ID NO:6).

GTINVHDVETQFNQYKTEAASPYNLTISDVSVSDVPFPFSAQSGA (SEQ ID NO: 8) describes the extracellular domain of “SPY” functional variant of the native Primary Sequence of the MUC1 Growth Factor Receptor having enhanced stability (var-PSMGFR—An example of “PSMGFR”).

TINVHDVETQFNQYKTEAASPYNLTISDVSVSDVPFPFSAQSGA (SEQ ID NO: 9) describes the extracellular domain of “SPY” functional variant of the native Primary Sequence of the MUC1 Growth Factor Receptor having enhanced stability (var-PSMGFR—An example of “PSMGFR”), having a single amino acid deletion at the C-terminus of SEQ ID NO: 8).

describes MUC1 cytoplasmic domain nucleotide sequence
(SEQ ID NO: 10)
tgtcagtgccgccgaaagaactacgggcagctggacatctttccagcccgggatacctaccatcctatgagcgagta
ccccacctaccacacccatgggcgctatgtgccccctagcagtaccgatcgtagcccctatgagaaggtttctgcaggtaacggtggc
agcagcctctcttacacaaacccagcagtggcagccgcttctgccaacttg describes MUCI cytoplasmic
domain amino acid sequence.
(SEQ ID NO: 11)
CQCRRKNYGQLDIFPARDTYHPMSEYPTYHTHGRYVPPSSTDRSPYEKVS
AGNGGSSLSYTNPAVAAASANL describes NME7 nucleotide sequence (NME7: GENBANK
ACCESSION AB209049).
(SEQ ID NO: 12)
gagatcctgagacaatgaatcatagtgaaagattcgttttcattgcagagtggtatgatccaaatgcttcacttcttcgac
gttatgagcttttattttacccaggggatggatctgttgaaatgcatgatgtaaagaatcatcgcacctttttaaagcggaccaaatatgata
acctgcacttggaagatttatttataggcaacaaagtgaatgtcttttctcgacaactggtattaattgactatggggatcaatatacagctc
gccagctgggcagtaggaaagaaaaaacgctagccctaattaaaccagatgcaatatcaaaggctggagaaataattgaaataataa
acaaagctggatttactataaccaaactcaaaatgatgatgctttcaaggaaagaagcattggattttcatgtagatcaccagtcaagacc
ctttttcaatgagctgatccagtttattacaactggtcctattattgccatggagattttaagagatgatgctatatgtgaatggaaaagactg
ctgggacctgcaaactctggagtggcacgcacagatgcttctgaaagcattagagccctctttggaacagatggcataagaaatgcag
cgcatggccctgattcttttgcttctgcggccagagaaatggagttgttttttccttcaagtggaggttgtgggccggcaaacactgctaa
atttactaattgtacctgttgcattgttaaaccccatgctgtcagtgaaggtatgttgaatacactatattcagtacattttgttaataggagag
caatgtttattttcttgatgtactttatgtatagaaaataa describes NME7 amino acid sequence (NME7:
GENBANK ACCESSION AB209049).
(SEQ ID NO: 13)
DPETMNHSERFVFIAEWYDPNASLLRRYELLFYPGDGSVEMHDVKNHRT
FLKRTKYDNLHLEDLFIGNKVNVFSRQLVLIDYGDQYTARQLGSRKEKTLALIKPDAI
SKAGEIIEIINKAGFTITKLKMMMLSRKEALDFHVDHQSRPFFNELIQFITTGPIIAMEIL
RDDAICEWKRLLGPANSGVARTDASESIRALFGTDGIRNAAHGPDSFASAAREMELF
FPSSGGCGPANTAKFTNCTCCIVKPHAVSEGMLNTLYSVHFVNRRAMFIFLMYFMY
RK describes NM23-H1 nucleotide sequence (NM23-H1: GENBANK
ACCESSION AF487339).
(SEQ ID NO: 14)
atggtgctactgtctactttagggatcgtctttcaaggcgaggggcctcctatctcaagctgtgatacaggaaccatggc
caactgtgagcgtaccttcattgcgatcaaaccagatggggtccagcggggtcttgtgggagagattatcaagcgttttgagcagaaag
gattccgccttgttggtctgaaattcatgcaagcttccgaagatcttctcaaggaacactacgttgacctgaaggaccgtccattctttgcc
ggcctggtgaaatacatgcactcagggccggtagttgccatggtctgggaggggctgaatgtggtgaagacgggccgagtcatgctc
ggggagaccaaccctgcagactccaagcctgggaccatccgtggagacttctgcatacaagttggcaggaacattatacatggcagt
gattctgtggagagtgcagagaaggagatcggcttgtggtttcaccctgaggaactggtagattacacgagctgtgctcagaactggat
ctatgaatga NM23-H1 describes amino acid sequence (NM23-H1:
GENBANK ACCESSION AF487339).
(SEQ ID NO: 15)
MVLLSTLGIVFQGEGPPISSCDTGTMANCERTFIAIKPDGVQRGLVGEIIKR
FEQKGFRLVGLKFMQASEDLLKEHYVDLKDRPFFAGLVKYMHSGPVVAMVWEGL
NVVKTGRVMLGETNPADSKPGTIRGDFCIQVGRNIIHGSDSVESAEKEIGLWFHPEEL
VDYTSCAQNWIYE describes NM23-H1 S120G mutant nucleotide sequence (NM23-
H1: GENBANK ACCESSION AF487339).
(SEQ ID NO: 16)
atggtgctactgtctactttagggatcgtctttcaaggcgaggggcctcctatctcaagctgtgatacaggaaccatggc
caactgtgagcgtaccttcattgcgatcaaaccagatggggtccagcggggtcttgtgggagagattatcaagcgttttgagcagaaag
gattccgccttgttggtctgaaattcatgcaagcttccgaagatcttctcaaggaacactacgttgacctgaaggaccgtccattctttgcc
ggcctggtgaaatacatgcactcagggccggtagttgccatggtctgggaggggctgaatgtggtgaagacgggccgagtcatgctc
ggggagaccaaccctgcagactccaagcctgggaccatccgtggagacttctgcatacaagttggcaggaacattatacatggcggt
gattctgtggagagtgcagagaaggagatcggcttgtggtttcaccctgaggaactggtagattacacgagctgtgctcagaactggat
ctatgaatga describes NM23-H1 S120G mutant amino acid
sequence (NM23-H1: GENBANK ACCESSION AF487339).
(SEQ ID NO: 17)
MVLLSTLGIVFQGEGPPISSCDTGTMANCERTFIAIKPDGVQRGLVGEIIKR
FEQKGFRLVGLKFMQASEDLLKEHYVDLKDRPFFAGLVKYMHSGPVVAMVWEGL
NVVKTGRVMLGETNPADSKPGTIRGDFCIQVGRNIIHGGDSVESAEKEIGLWFHPEEL
VDYTSCAQNWIYE describes NM23-H2 nucleotide sequence
(NM23-H2: GENBANK ACCESSION AK313448).
(SEQ ID NO: 18)
atggccaacctggagcgcaccttcatcgccatcaagccggacggcgtgcagcgcggcctggtgggcgagatcatc
aagcgcttcgagcagaagggattccgcctcgtggccatgaagttcctccgggcctctgaagaacacctgaagcagcactacattgac
ctgaaagaccgaccattcttccctgggctggtgaagtacatgaactcagggccggttgtggccatggtctgggaggggctgaacgtg
gtgaagacaggccgagtgatgcttggggagaccaatccagcagattcaaagccaggcaccattcgtggggacttctgcattcaggtt
ggcaggaacatcattcatggcagtgattcagtaaaaagtgctgaaaaagaaatcagcctatggtttaagcctgaagaactggttgacta
caagtcttgtgctcatgactgggtctatgaataa describes NM23-H2 amino acid sequence (NM23-H2:
GENBANK ACCESSION AK313448).
(SEQ ID NO: 19)
MANLERTFIAIKPDGVQRGLVGEIIKRFEQKGFRLVAMKFLRASEEHLKQH
YIDLKDRPFFPGLVKYMNSGPVVAMVWEGLNVVKTGRVMLGETNPADSKPGTIRG
DFCIQVGRNIIHGSDSVKSAEKEISLWFKPEELVDYKSCAHDWVYE describes NM23-H2 amino acid
sequence (NM23-H2: GENBANK ACCESSION AK313448).
Human NM23-H7-2 sequence optimized for E. coli expression:
(DNA)
(SEQ ID NO: 20)
atgcatgacgttaaaaatcaccgtacctttctgaaacgcacgaaatatgataatctgcatctggaagacctgtttattggc
aacaaagtcaatgtgttctctcgtcagctggtgctgatcgattatggcgaccagtacaccgcgcgtcaactgggtagtcgcaaagaaaa
aacgctggccctgattaaaccggatgcaatctccaaagctggcgaaattatcgaaattatcaacaaagcgggtttcaccatcacgaaac
tgaaaatgatgatgctgagccgtaaagaagccctggattttcatgtcgaccaccagtctcgcccgtttttcaatgaactgattcaattcatc
accacgggtccgattatcgcaatggaaattctgcgtgatgacgctatctgcgaatggaaacgcctgctgggcccggcaaactcaggtg
ttgcgcgtaccgatgccagtgaatccattcgcgctctgtttggcaccgatggtatccgtaatgcagcacatggtccggactcattcgcat
cggcagctcgtgaaatggaactgtttttcccgagctctggcggttgcggtccggcaaacaccgccaaatttaccaattgtacgtgctgta
ttgtcaaaccgcacgcagtgtcagaaggcctgctgggtaaaattctgatggcaatccgtgatgctggctttgaaatctcggccatgcag
atgttcaacatggaccgcgttaacgtcgaagaattctacgaagtttacaaaggcgtggttaccgaatatcacgatatggttacggaaatg
tactccggtccgtgcgtcgcgatggaaattcagcaaaacaatgccaccaaaacgtttcgtgaattctgtggtccggcagatccggaaat
cgcacgtcatctgcgtccgggtaccctgcgcgcaatttttggtaaaacgaaaatccagaacgctgtgcactgtaccgatctgccggaa
gacggtctgctggaagttcaatactttttcaaaattctggataattga
(amino acids)
(SEQ ID NO: 21)
MHDVKNHRTFLKRTKYDNLHLEDLFIGNKVNVFSRQLVLIDYGDQYTAR
QLGSRKEKTLALIKPDAISKAGEIIEIINKAGFTITKLKMMMLSRKEALDFHVDHQSRP
FFNELIQFITTGPIIAMEILRDDAICEWKRLLGPANSGVARTDASESIRALFGTDGIRNA
AHGPDSFASAAREMELFFPSSGGCGPANTAKFTNCTCCIVKPHAVSEGLLGKILMAIR
DAGFEISAMQMFNMDRVNVEEFYEVYKGVVTEYHDMVTEMYSGPCVAMEIQQNN
ATKTFREFCGPADPEIARHLRPGTLRAIFGKTKIQNAVHCTDLPEDGLLEVQYFFKILD
N-
Human NME7-A:
(DNA)
(SEQ ID NO: 22)
atggaaaaaacgctagccctaattaaaccagatgcaatatcaaaggctggagaaataattgaaataataaacaaagct
ggatttactataaccaaactcaaaatgatgatgctttcaaggaaagaagcattggattttcatgtagatcaccagtcaagaccctttttcaat
gagctgatccagtttattacaactggtcctattattgccatggagattttaagagatgatgctatatgtgaatggaaaagactgctgggacc
tgcaaactctggagtggcacgcacagatgcttctgaaagcattagagccctctttggaacagatggcataagaaatgcagcgcatggc
cctgattcttttgcttctgcggccagagaaatggagttgtttttttga
(amino acids)
(SEQ ID NO: 23)
MEKTLALIKPDAISKAGEIIEIINKAGFTITKLKMMMLSRKEALDFHVDHQS
RPFFNELIQFITTGPIIAMEILRDDAICEWKRLLGPANSGVARTDASESIRALFGTDGIR
NAAHGPDSFASAAREMELFF-
Human NME7-A1:
(DNA)
(SEQ ID NO: 24)
atggaaaaaacgctagccctaattaaaccagatgcaatatcaaaggctggagaaataattgaaataataaacaaagct
ggatttactataaccaaactcaaaatgatgatgctttcaaggaaagaagcattggattttcatgtagatcaccagtcaagaccctttttcaat
gagctgatccagtttattacaactggtcctattattgccatggagattttaagagatgatgctatatgtgaatggaaaagactgctgggacc
tgcaaactctggagtggcacgcacagatgcttctgaaagcattagagccctctttggaacagatggcataagaaatgcagcgcatggc
cctgattcttttgcttctgcggccagagaaatggagttgttttttccttcaagtggaggttgtgggccggcaaacactgctaaatttacttga
(amino acids)
(SEQ ID NO: 25)
MEKTLALIKPDAISKAGEIIEIINKAGFTITKLKMMMLSRKEALDFHVDHQS
RPFFNELIQFITTGPIIAMEILRDDAICEWKRLLGPANSGVARTDASESIRALFGTDGIR
NAAHGPDSFASAAREMELFFPSSGGCGPANTAKFT-
Human NME7-A2:
(DNA)
(SEQ ID NO: 26)
atgaatcatagtgaaagattcgttttcattgcagagtggtatgatccaaatgcttcacttcttcgacgttatgagcttttatttt
acccaggggatggatctgttgaaatgcatgatgtaaagaatcatcgcacctttttaaagcggaccaaatatgataacctgcacttggaag
atttatttataggcaacaaagtgaatgtcttttctcgacaactggtattaattgactatggggatcaatatacagctcgccagctgggcagta
ggaaagaaaaaacgctagccctaattaaaccagatgcaatatcaaaggctggagaaataattgaaataataaacaaagctggatttact
ataaccaaactcaaaatgatgatgctttcaaggaaagaagcattggattttcatgtagatcaccagtcaagaccctttttcaatgagctgat
ccagtttattacaactggtcctattattgccatggagattttaagagatgatgctatatgtgaatggaaaagactgctgggacctgcaaact
ctggagtggcacgcacagatgcttctgaaagcattagagccctctttggaacagatggcataagaaatgcagcgcatggccctgattct
tttgcttctgcggccagagaaatggagttgtttttttga
(amino acids)
(SEQ ID NO: 27)
MNHSERFVFIAEWYDPNASLLRRYELLFYPGDGSVEMHDVKNHRTFLKR
TKYDNLHLEDLFIGNKVNVFSRQLVLIDYGDQYTARQLGSRKEKTLALIKPDAISKA
GEIIEIINKAGFTITKLKMMMLSRKEALDFHVDHQSRPFFNELIQFITTGPIIAMEILRDD
AICEWKRLLGPANSGVARTDASESIRALFGTDGIRNAAHGPDSFASAAREMELFF-
Human NME7-A3:
(DNA)
(SEQ ID NO: 28)
atgaatcatagtgaaagattcgttttcattgcagagtggtatgatccaaatgcttcacttcttcgacgttatgagcttttatttt
acccaggggatggatctgttgaaatgcatgatgtaaagaatcatcgcacctttttaaagcggaccaaatatgataacctgcacttggaag
atttatttataggcaacaaagtgaatgtcttttctcgacaactggtattaattgactatggggatcaatatacagctcgccagctgggcagta
ggaaagaaaaaacgctagccctaattaaaccagatgcaatatcaaaggctggagaaataattgaaataataaacaaagctggatttact
ataaccaaactcaaaatgatgatgctttcaaggaaagaagcattggattttcatgtagatcaccagtcaagaccctttttcaatgagctgat
ccagtttattacaactggtcctattattgccatggagattttaagagatgatgctatatgtgaatggaaaagactgctgggacctgcaaact
ctggagtggcacgcacagatgcttctgaaagcattagagccctctttggaacagatggcataagaaatgcagcgcatggccctgattct
tttgcttctgcggccagagaaatggagttgttttttccttcaagtggaggttgtgggccggcaaacactgctaaatttacttga
(amino acids)
(SEQ ID NO: 29)
MNHSERFVFIAEWYDPNASLLRRYELLFYPGDGSVEMHDVKNHRTFLKR
TKYDNLHLEDLFIGNKVNVFSRQLVLIDYGDQYTARQLGSRKEKTLALIKPDAISKA
GEIIEIINKAGFTITKLKMMMLSRKEALDFHVDHQSRPFFNELIQFITTGPIIAMEILRDD
AICEWKRLLGPANSGVARTDASESIRALFGTDGIRNAAHGPDSFASAAREMELFFPSS
GGCGPANTAKFT-
Human NME7-B:
(DNA)
(SEQ ID NO: 30)
atgaattgtacctgttgcattgttaaaccccatgctgtcagtgaaggactgttgggaaagatcctgatggctatccgaga
tgcaggttttgaaatctcagctatgcagatgttcaatatggatcgggttaatgttgaggaattctatgaagtttataaaggagtagtgaccg
aatatcatgacatggtgacagaaatgtattctggcccttgtgtagcaatggagattcaacagaataatgctacaaagacatttcgagaattt
tgtggacctgctgatcctgaaattgcccggcatttacgccctggaactctcagagcaatctttggtaaaactaagatccagaatgctgttc
actgtactgatctgccagaggatggcctattagaggttcaatacttcttctga
(amino acids)
(SEQ ID NO: 31)
MNCTCCIVKPHAVSEGLLGKILMAIRDAGFEISAMQMFNMDRVNVEEFY
EVYKGVVTEYHDMVTEMYSGPCVAMEIQQNNATKTFREFCGPADPEIARHLRPGTL
RAIFGKTKIQNAVHCTDLPEDGLLEVQYFF-
Human NME7-B1:
(DNA)
(SEQ ID NO: 32)
atgaattgtacctgttgcattgttaaaccccatgctgtcagtgaaggactgttgggaaagatcctgatggctatccgaga
tgcaggttttgaaatctcagctatgcagatgttcaatatggatcgggttaatgttgaggaattctatgaagtttataaaggagtagtgaccg
aatatcatgacatggtgacagaaatgtattctggcccttgtgtagcaatggagattcaacagaataatgctacaaagacatttcgagaattt
tgtggacctgctgatcctgaaattgcccggcatttacgccctggaactctcagagcaatctttggtaaaactaagatccagaatgctgttc
actgtactgatctgccagaggatggcctattagaggttcaatacttcttcaagatcttggataattagtga
(amino acids)
(SEQ ID NO: 33)
MNCTCCIVKPHAVSEGLLGKILMAIRDAGFEISAMQMFNMDRVNVEEFY
EVYKGVVTEYHDMVTEMYSGPCVAMEIQQNNATKTFREFCGPADPEIARHLRPGTL
RAIFGKTKIQNAVHCTDLPEDGLLEVQYFFKILDN-
Human NME7-B2:
(DNA)
(SEQ ID NO: 34)
atgccttcaagtggaggttgtgggccggcaaacactgctaaatttactaattgtacctgttgcattgttaaaccccatgct
gtcagtgaaggactgttgggaaagatcctgatggctatccgagatgcaggttttgaaatctcagctatgcagatgttcaatatggatcgg
gttaatgttgaggaattctatgaagtttataaaggagtagtgaccgaatatcatgacatggtgacagaaatgtattctggcccttgtgtagc
aatggagattcaacagaataatgctacaaagacatttcgagaattttgtggacctgctgatcctgaaattgcccggcatttacgccctgga
actctcagagcaatctttggtaaaactaagatccagaatgctgttcactgtactgatctgccagaggatggcctattagaggttcaatactt
cttctga
(amino acids)
(SEQ ID NO: 35)
MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTFREFC
GPADPEIARHLRPGTLRAIFGKTKIQNAVHCTDLPEDGLLEVQYFF-
Human NME7-B3:
(DNA)
(SEQ ID NO: 36)
atgccttcaagtggaggttgtgggccggcaaacactgctaaatttactaattgtacctgttgcattgttaaaccccatgct
gtcagtgaaggactgttgggaaagatcctgatggctatccgagatgcaggttttgaaatctcagctatgcagatgttcaatatggatcgg
gttaatgttgaggaattctatgaagtttataaaggagtagtgaccgaatatcatgacatggtgacagaaatgtattctggcccttgtgtagc
aatggagattcaacagaataatgctacaaagacatttcgagaattttgtggacctgctgatcctgaaattgcccggcatttacgccctgga
actctcagagcaatctttggtaaaactaagatccagaatgctgttcactgtactgatctgccagaggatggcctattagaggttcaatactt
cttcaagatcttggataattagtga
(amino acids)
(SEQ ID NO: 37)
MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTFREFC
GPADPEIARHLRPGTLRAIFGKTKIQNAVHCTDLPEDGLLEVQYFFKILDN--
Human NME7_{AB}, also known as NME7_{AB}:
(DNA)
(SEQ ID NO: 38)
atggaaaaaacgctagccctaattaaaccagatgcaatatcaaaggctggagaaataattgaaataataaacaaagct
ggatttactataaccaaactcaaaatgatgatgctttcaaggaaagaagcattggattttcatgtagatcaccagtcaagaccctttttcaat
gagctgatccagtttattacaactggtcctattattgccatggagattttaagagatgatgctatatgtgaatggaaaagactgctgggacc
tgcaaactctggagtggcacgcacagatgcttctgaaagcattagagccctctttggaacagatggcataagaaatgcagcgcatggc
cctgattcttttgcttctgcggccagagaaatggagttgttttttccttcaagtggaggttgtgggccggcaaacactgctaaatttactaatt
gtacctgttgcattgttaaaccccatgctgtcagtgaaggactgttgggaaagatcctgatggctatccgagatgcaggttttgaaatctc
agctatgcagatgttcaatatggatcgggttaatgttgaggaattctatgaagtttataaaggagtagtgaccgaatatcatgacatggtga
cagaaatgtattctggcccttgtgtagcaatggagattcaacagaataatgctacaaagacatttcgagaattttgtggacctgctgatcct
gaaattgcccggcatttacgccctggaactctcagagcaatctttggtaaaactaagatccagaatgctgttcactgtactgatctgccag
aggatggcctattagaggttcaatacttcttcaagatcttggataattagtga
(amino acids)
(SEQ ID NO: 39)
MEKTLALIKPDAISKAGEIIEIINKAGFTITKLKMMMLSRKEALDFHVDHQS
RPFFNELIQFITTGPIIAMEILRDDAICEWKRLLGPANSGVARTDASESIRALFGTDGIR
NAAHGPDSFASAAREMELFFPSSGGCGPANTAKFTNCTCCIVKPHAVSEGLLGKILM
AIRDAGFEISAMQMFNMDRVNVEEFYEVYKGVVTEYHDMVTEMYSGPCVAMEIQQ
NNATKTFREFCGPADPEIARHLRPGTLRAIFGKTKIQNAVHCTDLPEDGLLEVQYFFK
ILDN--
Human NME7_{AB}1:
(DNA)
(SEQ ID NO: 40)
atggaaaaaacgctagccctaattaaaccagatgcaatatcaaaggctggagaaataattgaaataataaacaaagct
ggatttactataaccaaactcaaaatgatgatgctttcaaggaaagaagcattggattttcatgtagatcaccagtcaagaccctttttcaat
gagctgatccagtttattacaactggtcctattattgccatggagattttaagagatgatgctatatgtgaatggaaaagactgctgggacc
tgcaaactctggagtggcacgcacagatgcttctgaaagcattagagccctctttggaacagatggcataagaaatgcagcgcatggc
cctgattcttttgcttctgcggccagagaaatggagttgttttttccttcaagtggaggttgtgggccggcaaacactgctaaatttactaatt
gtacctgttgcattgttaaaccccatgctgtcagtgaaggactgttgggaaagatcctgatggctatccgagatgcaggttttgaaatctc
agctatgcagatgttcaatatggatcgggttaatgttgaggaattctatgaagtttataaaggagtagtgaccgaatatcatgacatggtga
cagaaatgtattctggcccttgtgtagcaatggagattcaacagaataatgctacaaagacatttcgagaattttgtggacctgctgatcct
gaaattgcccggcatttacgccctggaactctcagagcaatctttggtaaaactaagatccagaatgctgttcactgtactgatctgccag
aggatggcctattagaggttcaatacttcttctga
(amino acids)
(SEQ ID NO: 41)
MEKTLALIKPDAISKAGEIIEIINKAGFTITKLKMMMLSRKEALDFHVDHQS
RPFFNELIQFITTGPIIAMEILRDDAICEWKRLLGPANSGVARTDASESIRALFGTDGIR
NAAHGPDSFASAAREMELFFPSSGGCGPANTAKFTNCTCCIVKPHAVSEGLLGKILM
AIRDAGFEISAMQMFNMDRVNVEEFYEVYKGVVTEYHDMVTEMYSGPCVAMEIQQ
NNATKTFREFCGPADPEIARHLRPGTLRAIFGKTKIQNAVHCTDLPEDGLLEVQYFF-
Human NME7-A sequence optimized for E. coli expression:
(DNA)
(SEQ ID NO: 42)
atggaaaaaacgctggccctgattaaaccggatgcaatctccaaagctggcgaaattatcgaaattatcaacaaagcg
ggtttcaccatcacgaaactgaaaatgatgatgctgagccgtaaagaagccctggattttcatgtcgaccaccagtctcgcccgtttttca
atgaactgattcaattcatcaccacgggtccgattatcgcaatggaaattctgcgtgatgacgctatctgcgaatggaaacgcctgctgg
gcccggcaaactcaggtgttgcgcgtaccgatgccagtgaatccattcgcgctctgtttggcaccgatggtatccgtaatgcagcacat
ggtccggactcattcgcatcggcagctcgtgaaatggaactgtttttctga
(amino acids)
(SEQ ID NO: 43)
MEKTLALIKPDAISKAGEIIEIINKAGFTITKLKMMMLSRKEALDFHVDHQS
RPFFNELIQFITTGPIIAMEILRDDAICEWKRLLGPANSGVARTDASESIRALFGTDGIR
NAAHGPDSFASAAREMELFF-
Human NME7-Al sequence optimized for E. coli expression:
(DNA)
(SEQ ID NO: 44)
atggaaaaaacgctggccctgattaaaccggatgcaatctccaaagctggcgaaattatcgaaattatcaacaaagcg
ggtttcaccatcacgaaactgaaaatgatgatgctgagccgtaaagaagccctggattttcatgtcgaccaccagtctcgcccgtttttca
atgaactgattcaattcatcaccacgggtccgattatcgcaatggaaattctgcgtgatgacgctatctgcgaatggaaacgcctgctgg
gcccggcaaactcaggtgttgcgcgtaccgatgccagtgaatccattcgcgctctgtttggcaccgatggtatccgtaatgcagcacat
ggtccggactcattcgcatcggcagctcgtgaaatggaactgtttttcccgagctctggcggttgcggtccggcaaacaccgccaaatt
tacctga
(amino acids)
(SEQ ID NO: 45)
MEKTLALIKPDAISKAGEIIEIINKAGFTITKLKMMMLSRKEALDFHVDHQS
RPFFNELIQFITTGPIIAMEILRDDAICEWKRLLGPANSGVARTDASESIRALFGTDGIR
NAAHGPDSFASAAREMELFFPSSGGCGPANTAKFT-
Human NME7-A2 sequence optimized for E. coli expression:
(DNA)
(SEQ ID NO: 46)
atgaatcactccgaacgctttgtttttatcgccgaatggtatgacccgaatgcttccctgctgcgccgctacgaactgct
gttttatccgggcgatggtagcgtggaaatgcatgacgttaaaaatcaccgtacctttctgaaacgcacgaaatatgataatctgcatctg
gaagacctgtttattggcaacaaagtcaatgtgttctctcgtcagctggtgctgatcgattatggcgaccagtacaccgcgcgtcaactg
ggtagtcgcaaagaaaaaacgctggccctgattaaaccggatgcaatctccaaagctggcgaaattatcgaaattatcaacaaagcgg
gtttcaccatcacgaaactgaaaatgatgatgctgagccgtaaagaagccctggattttcatgtcgaccaccagtctcgcccgtttttcaa
tgaactgattcaattcatcaccacgggtccgattatcgcaatggaaattctgcgtgatgacgctatctgcgaatggaaacgcctgctggg
cccggcaaactcaggtgttgcgcgtaccgatgccagtgaatccattcgcgctctgtttggcaccgatggtatccgtaatgcagcacatg
gtccggactcattcgcatcggcagctcgtgaaatggaactgtttttctga
(amino acids)
(SEQ ID NO: 47)
MNHSERFVFIAEWYDPNASLLRRYELLFYPGDGSVEMHDVKNHRTFLKR
TKYDNLHLEDLFIGNKVNVFSRQLVLIDYGDQYTARQLGSRKEKTLALIKPDAISKA
GEIIEIINKAGFTITKLKMMMLSRKEALDFHVDHQSRPFFNELIQFITTGPIIAMEILRDD
AICEWKRLLGPANSGVARTDASESIRALFGTDGIRNAAHGPDSFASAAREMELFF-
Human NME7-A3 sequence optimized for E. coli expression:
(DNA)
(SEQ ID NO: 48)
atgaatcactccgaacgctttgtttttatcgccgaatggtatgacccgaatgcttccctgctgcgccgctacgaactgct
gttttatccgggcgatggtagcgtggaaatgcatgacgttaaaaatcaccgtacctttctgaaacgcacgaaatatgataatctgcatctg
gaagacctgtttattggcaacaaagtcaatgtgttctctcgtcagctggtgctgatcgattatggcgaccagtacaccgcgcgtcaactg
ggtagtcgcaaagaaaaaacgctggccctgattaaaccggatgcaatctccaaagctggcgaaattatcgaaattatcaacaaagcgg
gtttcaccatcacgaaactgaaaatgatgatgctgagccgtaaagaagccctggattttcatgtcgaccaccagtctcgcccgtttttcaa
tgaactgattcaattcatcaccacgggtccgattatcgcaatggaaattctgcgtgatgacgctatctgcgaatggaaacgcctgctggg
cccggcaaactcaggtgttgcgcgtaccgatgccagtgaatccattcgcgctctgtttggcaccgatggtatccgtaatgcagcacatg
gtccggactcattcgcatcggcagctcgtgaaatggaactgtttttcccgagctctggcggttgcggtccggcaaacaccgccaaattt
acctga
(amino acids)
(SEQ ID NO: 49)
MNHSERFVFIAEWYDPNASLLRRYELLFYPGDGSVEMHDVKNHRTFLKR
TKYDNLHLEDLFIGNKVNVFSRQLVLIDYGDQYTARQLGSRKEKTLALIKPDAISKA
GEIIEIINKAGFTITKLKMMMLSRKEALDFHVDHQSRPFFNELIQFITTGPIIAMEILRDD
AICEWKRLLGPANSGVARTDASESIRALFGTDGIRNAAHGPDSFASAAREMELFFPSS
GGCGPANTAKFT-
Human NME7-B sequence optimized for E. coli expression:
(DNA)
(SEQ ID NO: 50)
atgaattgtacgtgctgtattgtcaaaccgcacgcagtgtcagaaggcctgctgggtaaaattctgatggcaatccgtg
atgctggctttgaaatctcggccatgcagatgttcaacatggaccgcgttaacgtcgaagaattctacgaagtttacaaaggcgtggtta
ccgaatatcacgatatggttacggaaatgtactccggtccgtgcgtcgcgatggaaattcagcaaaacaatgccaccaaaacgtttcgt
gaattctgtggtccggcagatccggaaatcgcacgtcatctgcgtccgggtaccctgcgcgcaatttttggtaaaacgaaaatccagaa
cgctgtgcactgtaccgatctgccggaagacggtctgctggaagttcaatactttttctga
(amino acids)
(SEQ ID NO: 51)
MNCTCCIVKPHAVSEGLLGKILMAIRDAGFEISAMQMFNMDRVNVEEFY
EVYKGVVTEYHDMVTEMYSGPCVAMEIQQNNATKTFREFCGPADPEIARHLRPGTL
RAIFGKTKIQNAVHCTDLPEDGLLEVQYFF-
Human NME7-B1 sequence optimized for E. coli expression:
(DNA)
(SEQ ID NO: 52)
atgaattgtacgtgctgtattgtcaaaccgcacgcagtgtcagaaggcctgctgggtaaaattctgatggcaatccgtg
atgctggctttgaaatctcggccatgcagatgttcaacatggaccgcgttaacgtcgaagaattctacgaagtttacaaaggcgtggtta
ccgaatatcacgatatggttacggaaatgtactccggtccgtgcgtcgcgatggaaattcagcaaaacaatgccaccaaaacgtttcgt
gaattctgtggtccggcagatccggaaatcgcacgtcatctgcgtccgggtaccctgcgcgcaatttttggtaaaacgaaaatccagaa
cgctgtgcactgtaccgatctgccggaagacggtctgctggaagttcaatactttttcaaaattctggataattga
(amino acids)
(SEQ ID NO: 53)
MNCTCCIVKPHAVSEGLLGKILMAIRDAGFEISAMQMFNMDRVNVEEFY
EVYKGVVTEYHDMVTEMYSGPCVAMEIQQNNATKTFREFCGPADPEIARHLRPGTL
RAIFGKTKIQNAVHCTDLPEDGLLEVQYFFKILDN-
Human NME7-B2 sequence optimized for E. coli expression:
(DNA)
(SEQ ID NO: 54)
atgccgagctctggcggttgcggtccggcaaacaccgccaaatttaccaattgtacgtgctgtattgtcaaaccgcac
gcagtgtcagaaggcctgctgggtaaaattctgatggcaatccgtgatgctggctttgaaatctcggccatgcagatgttcaacatggac
cgcgttaacgtcgaagaattctacgaagtttacaaaggcgtggttaccgaatatcacgatatggttacggaaatgtactccggtccgtgc
gtcgcgatggaaattcagcaaaacaatgccaccaaaacgtttcgtgaattctgtggtccggcagatccggaaatcgcacgtcatctgcg
tccgggtaccctgcgcgcaatttttggtaaaacgaaaatccagaacgctgtgcactgtaccgatctgccggaagacggtctgctggaa
gttcaatactttttctga
(amino acids)
(SEQ ID NO: 55)
MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTFREFC
GPADPEIARHLRPGTLRAIFGKTKIQNAVHCTDLPEDGLLEVQYFF-
Human NME7-B3 sequence optimized for E. coli expression:
(DNA)
(SEQ ID NO: 56)
atgccgagctctggcggttgcggtccggcaaacaccgccaaatttaccaattgtacgtgctgtattgtcaaaccgcac
gcagtgtcagaaggcctgctgggtaaaattctgatggcaatccgtgatgctggctttgaaatctcggccatgcagatgttcaacatggac
cgcgttaacgtcgaagaattctacgaagtttacaaaggcgtggttaccgaatatcacgatatggttacggaaatgtactccggtccgtgc
gtcgcgatggaaattcagcaaaacaatgccaccaaaacgtttcgtgaattctgtggtccggcagatccggaaatcgcacgtcatctgcg
tccgggtaccctgcgcgcaatttttggtaaaacgaaaatccagaacgctgtgcactgtaccgatctgccggaagacggtctgctggaa
gttcaatactttttcaaaattctggataattga
(amino acids)
(SEQ ID NO: 57)
MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTFREFC
GPADPEIARHLRPGTLRAIFGKTKIQNAVHCTDLPEDGLLEVQYFFKILDN-
Human NME7_{AB}, also known as NME7_{AB} sequence optimized for E. coli
expression:
(DNA)
(SEQ ID NO: 58)
atggaaaaaacgctggccctgattaaaccggatgcaatctccaaagctggcgaaattatcgaaattatcaacaaagcg
ggtttcaccatcacgaaactgaaaatgatgatgctgagccgtaaagaagccctggattttcatgtcgaccaccagtctcgcccgtttttca
atgaactgattcaattcatcaccacgggtccgattatcgcaatggaaattctgcgtgatgacgctatctgcgaatggaaacgcctgctgg
gcccggcaaactcaggtgttgcgcgtaccgatgccagtgaatccattcgcgctctgtttggcaccgatggtatccgtaatgcagcacat
ggtccggactcattcgcatcggcagctcgtgaaatggaactgtttttcccgagctctggcggttgcggtccggcaaacaccgccaaatt
taccaattgtacgtgctgtattgtcaaaccgcacgcagtgtcagaaggcctgctgggtaaaattctgatggcaatccgtgatgctggcttt
gaaatctcggccatgcagatgttcaacatggaccgcgttaacgtcgaagaattctacgaagtttacaaaggcgtggttaccgaatatca
cgatatggttacggaaatgtactccggtccgtgcgtcgcgatggaaattcagcaaaacaatgccaccaaaacgtttcgtgaattctgtgg
tccggcagatccggaaatcgcacgtcatctgcgtccgggtaccctgcgcgcaatttttggtaaaacgaaaatccagaacgctgtgcact
gtaccgatctgccggaagacggtctgctggaagttcaatactttttcaaaattctggataattga
(amino acids)
(SEQ ID NO: 59)
MEKTLALIKPDAISKAGEIIEIINKAGFTITKLKMMMLSRKEALDFHVDHQS
RPFFNELIQFITTGPIIAMEILRDDAICEWKRLLGPANSGVARTDASESIRALFGTDGIR
NAAHGPDSFASAAREMELFFPSSGGCGPANTAKFTNCTCCIVKPHAVSEGLLGKILM
AIRDAGFEISAMQMFNMDRVNVEEFYEVYKGVVTEYHDMVTEMYSGPCVAMEIQQ
NNATKTFREFCGPADPEIARHLRPGTLRAIFGKTKIQNAVHCTDLPEDGLLEVQYFFK
ILDN-
Human NME7_{AB}1, also known as NME7_{AB}1 sequence optimized for E. coli
expression:
(DNA)
(SEQ ID NO: 60)
Atggaaaaaacgctggccctgattaaaccggatgcaatctccaaagctggcgaaattatcgaaattatcaacaaagc
gggtttcaccatcacgaaactgaaaatgatgatgctgagccgtaaagaagccctggattttcatgtcgaccaccagtctcgcccgtttttc
aatgaactgattcaattcatcaccacgggtccgattatcgcaatggaaattctgcgtgatgacgctatctgcgaatggaaacgcctgctg
ggcccggcaaactcaggtgttgcgcgtaccgatgccagtgaatccattcgcgctctgtttggcaccgatggtatccgtaatgcagcaca
tggtccggactcattcgcatcggcagctcgtgaaatggaactgtttttcccgagctctggcggttgcggtccggcaaacaccgccaaat
ttaccaattgtacgtgctgtattgtcaaaccgcacgcagtgtcagaaggcctgctgggtaaaattctgatggcaatccgtgatgctggctt
tgaaatctcggccatgcagatgttcaacatggaccgcgttaacgtcgaagaattctacgaagtttacaaaggcgtggttaccgaatatca
cgatatggttacggaaatgtactccggtccgtgcgtcgcgatggaaattcagcaaaacaatgccaccaaaacgtttcgtgaattctgtgg
tccggcagatccggaaatcgcacgtcatctgcgtccgggtaccctgcgcgcaatttttggtaaaacgaaaatccagaacgctgtgcact
gtaccgatctgccggaagacggtctgctggaagttcaatactttttctga
(amino acids)
(SEQ ID NO: 61)
MEKTLALIKPDAISKAGEIIEIINKAGFTITKLKMMMLSRKEALDFHVDHQS
RPFFNELIQFITTGPIIAMEILRDDAICEWKRLLGPANSGVARTDASESIRALFGTDGIR
NAAHGPDSFASAAREMELFFPSSGGCGPANTAKFTNCTCCIVKPHAVSEGLLGKILM
AIRDAGFEISAMQMFNMDRVNVEEFYEVYKGVVTEYHDMVTEMYSGPCVAMEIQQ
NNATKTFREFCGPADPEIARHLRPGTLRAIFGKTKIQNAVHCTDLPEDGLLEVQYFF-
Mouse NME6
(DNA)
(SEQ ID NO: 62)
Atgacctccatcttgcgaagtccccaagctcttcagctcacactagccctgatcaagcctgatgcagttgcccaccca
ctgatcctggaggctgttcatcagcagattctgagcaacaagttcctcattgtacgaacgagggaactgcagtggaagctggaggact
gccggaggttttaccgagagcatgaagggcgttttttctatcagcggctggtggagttcatgacaagtgggccaatccgagcctatatc
cttgcccacaaagatgccatccaactttggaggacactgatgggacccaccagagtatttcgagcacgctatatagccccagattcaat
tcgtggaagtttgggcctcactgacacccgaaatactacccatggctcagactccgtggtttccgccagcagagagattgcagccttctt
ccctgacttcagtgaacagcgctggtatgaggaggaggaaccccagctgcggtgtggtcctgtgcactacagtccagaggaaggtat
ccactgtgcagctgaaacaggaggccacaaacaacctaacaaaacctag
(amino acids)
(SEQ ID NO: 63)
MTSILRSPQALQLTLALIKPDAVAHPLILEAVHQQILSNKFLIVRTRELQWK
LEDCRRFYREHEGRFFYQRLVEFMTSGPIRAYILAHKDAIQLWRTLMGPTRVFRARY
IAPDSIRGSLGLTDTRNTTHGSDSVVSASREIAAFFPDFSEQRWYEEEEPQLRCGPVHY
SPEEGIHCAAETGGHKQPNKT-
Human NME6:
(DNA)
(SEQ ID NO: 64)
Atgacccagaatctggggagtgagatggcctcaatcttgcgaagccctcaggctctccagctcactctagccctgat
caagcctgacgcagtcgcccatccactgattctggaggctgttcatcagcagattctaagcaacaagttcctgattgtacgaatgagag
aactactgtggagaaaggaagattgccagaggttttaccgagagcatgaagggcgttttttctatcagaggctggtggagttcatggcc
agcgggccaatccgagcctacatccttgcccacaaggatgccatccagctctggaggacgctcatgggacccaccagagtgttccga
gcacgccatgtggccccagattctatccgtgggagtttcggcctcactgacacccgcaacaccacccatggttcggactctgtggtttc
agccagcagagagattgcagccttcttccctgacttcagtgaacagcgctggtatgaggaggaagagccccagttgcgctgtggccct
gtgtgctatagcccagagggaggtgtccactatgtagctggaacaggaggcctaggaccagcctga
(amino acids)
(SEQ ID NO: 65)
MTQNLGSEMASILRSPQALQLTLALIKPDAVAHPLILEAVHQQILSNKFLIV
RMRELLWRKEDCQRFYREHEGRFFYQRLVEFMASGPIRAYILAHKDAIQLWRTLMG
PTRVFRARHVAPDSIRGSFGLTDTRNTTHGSDSVVSASREIAAFFPDFSEQRWYEEEE
PQLRCGPVCYSPEGGVHYVAGTGGLGPA-
Human NME6 1:
(DNA)
(SEQ ID NO: 66)
Atgacccagaatctggggagtgagatggcctcaatcttgcgaagccctcaggctctccagctcactctagccctgat
caagcctgacgcagtcgcccatccactgattctggaggctgttcatcagcagattctaagcaacaagttcctgattgtacgaatgagag
aactactgtggagaaaggaagattgccagaggttttaccgagagcatgaagggcgttttttctatcagaggctggtggagttcatggcc
agcgggccaatccgagcctacatccttgcccacaaggatgccatccagctctggaggacgctcatgggacccaccagagtgttccga
gcacgccatgtggccccagattctatccgtgggagtttcggcctcactgacacccgcaacaccacccatggttcggactctgtggtttc
agccagcagagagattgcagccttcttccctgacttcagtgaacagcgctggtatgaggaggaagagccccagttgcgctgtggccct
gtgtga
(amino acids)
(SEQ ID NO: 67)
MTQNLGSEMASILRSPQALQLTLALIKPDAVAHPLILEAVHQQILSNKFLIV
RMRELLWRKEDCQRFYREHEGRFFYQRLVEFMASGPIRAYILAHKDAIQLWRTLMG
PTRVFRARHVAPDSIRGSFGLTDTRNTTHGSDSVVSASREIAAFFPDFSEQRWYEEEE
PQLRCGPV-
Human NME6 2:
(DNA)
(SEQ ID NO: 68)
Atgctcactctagccctgatcaagcctgacgcagtcgcccatccactgattctggaggctgttcatcagcagattctaa
gcaacaagttcctgattgtacgaatgagagaactactgtggagaaaggaagattgccagaggttttaccgagagcatgaagggcgtttt
ttctatcagaggctggtggagttcatggccagcgggccaatccgagcctacatccttgcccacaaggatgccatccagctctggagga
cgctcatgggacccaccagagtgttccgagcacgccatgtggccccagattctatccgtgggagtttcggcctcactgacacccgcaa
caccacccatggttcggactctgtggtttcagccagcagagagattgcagccttcttccctgacttcagtgaacagcgctggtatgagg
aggaagagccccagttgcgctgtggccctgtgtga
(amino acids)
(SEQ ID NO: 69)
MLTLALIKPDAVAHPLILEAVHQQILSNKFLIVRMRELLWRKEDCQRFYRE
HEGRFFYQRLVEFMASGPIRAYILAHKDAIQLWRTLMGPTRVFRARHVAPDSIRGSF
GLTDTRNTTHGSDSVVSASREIAAFFPDFSEQRWYEEEEPQLRCGPV-
Human NME6 3:
(DNA)
(SEQ ID NO: 70)
Atgctcactctagccctgatcaagcctgacgcagtcgcccatccactgattctggaggctgttcatcagcagattctaa
gcaacaagttcctgattgtacgaatgagagaactactgtggagaaaggaagattgccagaggttttaccgagagcatgaagggcgtttt
ttctatcagaggctggtggagttcatggccagcgggccaatccgagcctacatccttgcccacaaggatgccatccagctctggagga
cgctcatgggacccaccagagtgttccgagcacgccatgtggccccagattctatccgtgggagtttcggcctcactgacacccgcaa
caccacccatggttcggactctgtggtttcagccagcagagagattgcagccttcttccctgacttcagtgaacagcgctggtatgagg
aggaagagccccagttgcgctgtggccctgtgtgctatagcccagagggaggtgtccactatgtagctggaacaggaggcctagga
ccagcctga
(amino acids)
(SEQ ID NO: 71)
MLTLALIKPDAVAHPLILEAVHQQILSNKFLIVRMRELLWRKEDCQRFYRE
HEGRFFYQRLVEFMASGPIRAYILAHKDAIQLWRTLMGPTRVFRARHVAPDSIRGSF
GLTDTRNTTHGSDSVVSASREIAAFFPDFSEQRWYEEEEPQLRCGPVCYSPEGGVHY
VAGTGGLGPA-
Human NME6 sequence optimized for E. coli expression:
(DNA)
(SEQ ID NO: 72)
Atgacgcaaaatctgggctcggaaatggcaagtatcctgcgctccccgcaagcactgcaactgaccctggctctgat
caaaccggacgctgttgctcatccgctgattctggaagcggtccaccagcaaattctgagcaacaaatttctgatcgtgcgtatgcgcg
aactgctgtggcgtaaagaagattgccagcgtttttatcgcgaacatgaaggccgtttcttttatcaacgcctggttgaattcatggcctct
ggtccgattcgcgcatatatcctggctcacaaagatgcgattcagctgtggcgtaccctgatgggtccgacgcgcgtctttcgtgcacgt
catgtggcaccggactcaatccgtggctcgttcggtctgaccgatacgcgcaataccacgcacggtagcgactctgttgttagtgcgtc
ccgtgaaatcgcggcctttttcccggacttctccgaacagcgttggtacgaagaagaagaaccgcaactgcgctgtggcccggtctgtt
attctccggaaggtggtgtccattatgtggcgggcacgggtggtctgggtccggcatga
(amino acids)
(SEQ ID NO: 73)
MTQNLGSEMASILRSPQALQLTLALIKPDAVAHPLILEAVHQQILSNKFLIV
RMRELLWRKEDCQRFYREHEGRFFYQRLVEFMASGPIRAYILAHKDAIQLWRTLMG
PTRVFRARHVAPDSIRGSFGLTDTRNTTHGSDSVVSASREIAAFFPDFSEQRWYEEEE
PQLRCGPVCYSPEGGVHYVAGTGGLGPA-
Human NME6 1 sequence optimized for E. coli expression:
(DNA)
(SEQ ID NO: 74)
Atgacgcaaaatctgggctcggaaatggcaagtatcctgcgctccccgcaagcactgcaactgaccctggctctgat
caaaccggacgctgttgctcatccgctgattctggaagcggtccaccagcaaattctgagcaacaaatttctgatcgtgcgtatgcgcg
aactgctgtggcgtaaagaagattgccagcgtttttatcgcgaacatgaaggccgtttcttttatcaacgcctggttgaattcatggcctct
ggtccgattcgcgcatatatcctggctcacaaagatgcgattcagctgtggcgtaccctgatgggtccgacgcgcgtctttcgtgcacgt
catgtggcaccggactcaatccgtggctcgttcggtctgaccgatacgcgcaataccacgcacggtagcgactctgttgttagtgcgtc
ccgtgaaatcgcggcctttttcccggacttctccgaacagcgttggtacgaagaagaagaaccgcaactgcgctgtggcccggtctga
(amino acids)
(SEQ ID NO: 75)
MTQNLGSEMASILRSPQALQLTLALIKPDAVAHPLILEAVHQQILSNKFLIV
RMRELLWRKEDCQRFYREHEGRFFYQRLVEFMASGPIRAYILAHKDAIQLWRTLMG
PTRVFRARHVAPDSIRGSFGLTDTRNTTHGSDSVVSASREIAAFFPDFSEQRWYEEEE
PQLRCGPV-
Human NME6 2 sequence optimized for E. coli expression:
(DNA)
(SEQ ID NO: 76)
Atgctgaccctggctctgatcaaaccggacgctgttgctcatccgctgattctggaagcggtccaccagcaaattctg
agcaacaaatttctgatcgtgcgtatgcgcgaactgctgtggcgtaaagaagattgccagcgtttttatcgcgaacatgaaggccgtttct
tttatcaacgcctggttgaattcatggcctctggtccgattcgcgcatatatcctggctcacaaagatgcgattcagctgtggcgtaccctg
atgggtccgacgcgcgtctttcgtgcacgtcatgtggcaccggactcaatccgtggctcgttcggtctgaccgatacgcgcaataccac
gcacggtagcgactctgttgttagtgcgtcccgtgaaatcgcggcctttttcccggacttctccgaacagcgttggtacgaagaagaag
aaccgcaactgcgctgtggcccggtctga
(amino acids)
(SEQ ID NO: 77)
MLTLALIKPDAVAHPLILEAVHQQILSNKFLIVRMRELLWRKEDCQRFYRE
HEGRFFYQRLVEFMASGPIRAYILAHKDAIQLWRTLMGPTRVFRARHVAPDSIRGSF
GLTDTRNTTHGSDSVVSASREIAAFFPDFSEQRWYEEEEPQLRCGPV-
Human NME6 3 sequence optimized for E. coli expression:
(DNA)
(SEQ ID NO: 78)
Atgctgaccctggctctgatcaaaccggacgctgttgctcatccgctgattctggaagcggtccaccagcaaattctg
agcaacaaatttctgatcgtgcgtatgcgcgaactgctgtggcgtaaagaagattgccagcgtttttatcgcgaacatgaaggccgtttct
tttatcaacgcctggttgaattcatggcctctggtccgattcgcgcatatatcctggctcacaaagatgcgattcagctgtggcgtaccctg
atgggtccgacgcgcgtctttcgtgcacgtcatgtggcaccggactcaatccgtggctcgttcggtctgaccgatacgcgcaataccac
gcacggtagcgactctgttgttagtgcgtcccgtgaaatcgcggcctttttcccggacttctccgaacagcgttggtacgaagaagaag
aaccgcaactgcgctgtggcccggtctgttattctccggaaggtggtgtccattatgtgggggcacgggtggtctgggtccggcatg
a
(amino acids)
(SEQ ID NO: 79)
MLTLALIKPDAVAHPLILEAVHQQILSNKFLIVRMRELLWRKEDCQRFYRE
HEGRFFYQRLVEFMASGPIRAYILAHKDAIQLWRTLMGPTRVFRARHVAPDSIRGSF
GLTDTRNTTHGSDSVVSASREIAAFFPDFSEQRWYEEEEPQLRCGPVCYSPEGGVHY
VAGTGGLGPA-
OriGene-NME7-1 full length
(DNA)
(SEQ ID NO: 80)
gacgttgtatacgactcctatagggcggccgggaattcgtcgactggatccggtaccgaggagatctgccgccgcg
atcgccatgaatcatagtgaaagattcgttttcattgcagagtggtatgatccaaatgcttcacttcttcgacgttatgagcttttattttaccc
aggggatggatctgttgaaatgcatgatgtaaagaatcatcgcacctttttaaagcggaccaaatatgataacctgcacttggaagattta
tttataggcaacaaagtgaatgtcttctctcgacaactggtattaattgactatggggatcaatatacagctcgccagctgggcagtagga
aagaaaaaacgctagccctaattaaaccagatgcaatatcaaaggctggagaaataattgaaataataaacaaagctggatttactataa
ccaaactcaaaatgatgatgctttcaaggaaagaagcattggattttcatgtagatcaccagtcaagaccctttttcaatgagctgatcca
gtttattacaactggtcctattattgccatggagattttaagagatgatgctatatgtgaatggaaaagactgctgggacctgcaaactctg
gagtggcacgcacagatgcttctgaaagcattagagccctctttggaacagatggcataagaaatgcagcgcatggccctgattctttt
gcttctgcggccagagaaatggagttgttttttccttcaagtggaggttgtgggccggcaaacactgctaaatttactaattgtacctgttg
cattgttaaaccccatgctgtcagtgaaggactgttgggaaagatcctgatggctatccgagatgcaggttttgaaatctcagctatgcag
atgttcaatatggatcgggttaatgttgaggaattctatgaagtttataaaggagtagtgaccgaatatcatgacatggtgacagaaatgta
ttctggcccttgtgtagcaatggagattcaacagaataatgctacaaagacatttcgagaattttgtggacctgctgatcctgaaattgccc
ggcatttacgccctggaactctcagagcaatctttggtaaaactaagatccagaatgctgttcactgtactgatctgccagaggatggcct
attagaggttcaatacttcttcaagatcttggataatacgcgtacgcggccgctcgagcagaaactcatctcagaagaggatctggcag
caaatgatatcctggattacaaggatgacgacgataaggtttaa
(amino acids)
(SEQ ID NO: 81)
MNHSERFVFIAEWYDPNASLLRRYELLFYPGDGSVEMHDVKNHRTFLKR
TKYDNLHLEDLFIGNKVNVFSRQLVLIDYGDQYTARQLGSRKEKTLALIKPDAISKA
GEIIEIINKAGFTITKLKMMMLSRKEALDFHVDHQSRPFFNELIQFITTGPIIAMEILRDD
AICEWKRLLGPANSGVARTDASESIRALFGTDGIRNAAHGPDSFASAAREMELFFPSS
GGCGPANTAKFTNCTCCIVKPHAVSEGLLGKILMAIRDAGFEISAMQMFNMDRVNV
EEFYEVYKGVVTEYHDMVTEMYSGPCVAMEIQQNNATKTFREFCGPADPEIARHLR
PGTLRAIFGKTKIQNAVHCTDLPEDGLLEVQYFFKILDNTRTRRLEQKLISEEDLAAN
DILDYKDDDDKV
Abnova NME7-1 Full length
(amino acids)
(SEQ ID NO: 82)
MNHSERFVFIAEWYDPNASLLRRYELLFYPGDGSVEMHDVKNHRTFLKR
TKYDNLHLEDLFIGNKVNVFSRQLVLIDYGDQYTARQLGSRKEKTLALIKPDAISKA
GEIIEIINKAGFTITKLKMMMLSRKEALDFHVDHQSRPFFNELIQFITTGPIIAMEILRDD
AICEWKRLLGPANSGVARTDASESIRALFGTDGIRNAAHGPDSFASAAREMELFFPSS
GGCGPANTAKFTNCTCCIVKPHAVSEGLLGKILMAIRDAGFEISAMQMFNMDRVNV
EEFYEVYKGVVTEYHDMVTEMYSGPCVAMEIQQNNATKTFREFCGPADPEIARHLR
PGTLRAIFGKTKIQNAVHCTDLPEDGLLEVQYFFKILDN
Abnova Partial NME7-B
(amino acids)
(SEQ ID NO: 83)
DRVNVEEFYEVYKGVVTEYHDMVTEMYSGPCVAMEIQQNNATKTFREF
CGPADPEIARHLRPGTLRAIFGKTKIQNAVHCTDLPEDGLLEVQYFFKIL
Histidine Tag
(SEQ ID NO: 84)
(ctcgag)caccaccaccaccaccactga
Strept II Tag
(SEQ ID NO: 85)
(accggt)tggagccatcctcagttcgaaaagtaatga
N-10 peptide:
(SEQ ID NO: 86)
QFNQYKTEAASRYNLTISDVSVSDVPFPFSAQSGA
C-10 peptide
(SEQ ID NO: 87)
GTINVHDVETQFNQYKTEAASRYNLTISDVSVSDV
(SEQ ID NO: 88)
LALIKPDA
(SEQ ID NO: 89)
MMMLSRKEALDFHVDHQS
(SEQ ID NO: 90)
ALDFHVDHQS
(SEQ ID NO: 91)
EILRDDAICEWKRL
(SEQ ID NO: 92)
FNELIQFITTGP
(SEQ ID NO: 93)
RDDAICEW
(SEQ ID NO: 94)
SGVARTDASESIRALFGTDGIRNAA
(SEQ ID NO: 95)
ELFFPSSGG
(SEQ ID NO: 96)
KFTNCTCCIVKPHAVSEGLLGKILMA
(SEQ ID NO: 97)
LMAIRDAGFEISAMQMFNMDRVNVEEFYEVYKGVVT
(SEQ ID NO: 98)
EFYEVYKGVVTEYHD
(SEQ ID NO: 99)
EIQQNNATKTFREFCGPADPEIARHLRPGTLRAIFGKTKIQNA
(SEQ ID NO: 100)
YSGPCVAM
(SEQ ID NO: 101)
FREFCGP
(SEQ ID NO: 102)
VHCTDLPEDGLLEVQYFFKILDN
(SEQ ID NO: 103)
IQNAVHCTD
(SEQ ID NO: 104)
TDLPEDGLLEVQYFFKILDN
(SEQ ID NO: 105)
PEDGLLEVQYFFK
(SEQ ID NO: 106)
EIINKAGFTITK
(SEQ ID NO: 107)
MLSRKEALDFHVDHQS
(SEQ ID NO: 108)
NELIQFITT
(SEQ ID NO: 109)
EILRDDAICEWKRL
(SEQ ID NO: 110)
SGVARTDASESIRALFGTDGI
(SEQ ID NO: 111)
SGVARTDASES
(SEQ ID NO: 112)
ALFGTDGI
(SEQ ID NO: 113)
NCTCCIVKPHAVSE
(SEQ ID NO: 114)
LGKILMAIRDA
(SEQ ID NO: 115)
EISAMQMFNMDRVNVE
(SEQ ID NO: 116)
EVYKGVVT
(SEQ ID NO: 117)
EYHDMVTE
(SEQ ID NO: 118)
EFCGPADPEIARHLR
(SEQ ID NO: 119)
AIFGKTKIQNAV
(SEQ ID NO: 120)
LPEDGLLEVQYFFKILDN
(SEQ ID NO: 121)
GPDSFASAAREMELFFP
Immunizing peptides derived from human NME7
(SEQ ID NO: 122)
ICEWKRL
(SEQ ID NO: 123)
LGKILMAIRDA
(SEQ ID NO: 124)
HAVSEGLLGK
(SEQ ID NO: 125)
VTEMYSGP
(SEQ ID NO: 126)
NATKTFREF
(SEQ ID NO: 127)
AIRDAGFEI
(SEQ ID NO: 128)
AICEWKRLLGPAN
(SEQ ID NO: 129)
DHQSRPFF
(SEQ ID NO: 130)
AICEWKRLLGPAN
(SEQ ID NO: 131)
VDHQSRPF
(SEQ ID NO: 132)
PDSFAS
(SEQ ID NO: 133)
KAGEIIEIINKAGFTITK
Immunizing peptides derived from human NME1
(SEQ ID NO: 134)
MANCERTFIAIKPDGVQRGLVGEIIKRFE
(SEQ ID NO: 135)
VDLKDRPF
(SEQ ID NO: 136)
HGSDSVESAEKEIGLWF
(SEQ ID NO: 137)
ERTFIAIKPDGVQRGLVGEIIKRFE
(SEQ ID NO: 138)
VDLKDRPFFAGLVKYMHSGPVVAMVWEGLN
(SEQ ID NO: 139)
NIIHGSDSVESAEKEIGLWFHPEELV
(SEQ ID NO: 140)
KPDGVQRGLVGEII
Immunizing peptide derived from human NME7, but which does not bind NME1
peptide A1
(SEQ ID NO: 141)
MLSRKEALDFHVDHQS
peptide A2
(SEQ ID NO: 142)
SGVARTDASES
peptide B1
(SEQ ID NO: 143)
DAGFEISAMQMFNMDRVNVE
peptide B2
(SEQ ID NO: 144)
EVYKGVVTEYHDMVTE
peptide B3
(SEQ ID NO: 145)
AIFGKTKIQNAVHCTDLPEDGLLEVQYFF
Human NME7 a
(DNA)
(SEQ ID NO: 146)
atgaatcatagtgaaagattcgttttcattgcagagtggtatgatccaaatgcttcacttcttcgacgttatgagcttttatttt
acccaggggatggatctgttgaaatgcatgatgtaaagaatcatcgcacctttttaaagcggaccaaatatgataacctgcacttggaag
atttatttataggcaacaaagtgaatgtcttttctcgacaactggtattaattgactatggggatcaatatacagctcgccagctgggcagta
ggaaagaaaaaacgctagccctaattaaaccagatgcaatatcaaaggctggagaaataattgaaataataaacaaagctggatttact
ataaccaaactcaaaatgatgatgctttcaaggaaagaagcattggattttcatgtagatcaccagtcaagaccctttttcaatgagctgat
ccagtttattacaactggtcctattattgccatggagattttaagagatgatgctatatgtgaatggaaaagactgctgggacctgcaaact
ctggagtggcacgcacagatgcttctgaaagcattagagccctctttggaacagatggcataagaaatgcagcgcatggccctgattct
tttgcttctgcggccagagaaatggagttgttttttccttcaagtggaggttgtgggccggcaaacactgctaaatttactaattgtacctgtt
gcattgttaaaccccatgctgtcagtgaaggactgttgggaaagatcctgatggctatccgagatgcaggttttgaaatctcagctatgca
gatgttcaatatggatcgggttaatgttgaggaattctatgaagtttataaaggagtagtgaccgaatatcatgacatggtgacagaaatgt
attctggcccttgtgtagcaatggagattcaacagaataatgctacaaagacatttcgagaattttgtggacctgctgatcctgaaattgcc
cggcatttacgccctggaactctcagagcaatctttggtaaaactaagatccagaatgctgttcactgtactgatctgccagaggatggc
ctattagaggttcaatacttcttcaagatcttggataattag
(amino acids)
(SEQ ID NO: 147)
MNHSERFVFIAEWYDPNASLLRRYELLFYPGDGSVEMHDVKNHRTFLKR
TKYDNLHLEDLFIGNKVNVFSRQLVLIDYGDQYTARQLGSRKEKTLALIKPDAISKA
GEIIEIINKAGFTITKLKMMMLSRKEALDFHVDHQSRPFFNELIQFITTGPIIAMEILRDD
AICEWKRLLGPANSGVARTDASESIRALFGTDGIRNAAHGPDSFASAAREMELFFPSS
GGCGPANTAKFTNCTCCIVKPHAVSEGLLGKILMAIRDAGFEISAMQMFNMDRVNV
EEFYEVYKGVVTEYHDMVTEMYSGPCVAMEIQQNNATKTFREFCGPADPEIARHLR
PGTLRAIFGKTKIQNAVHCTDLPEDGLLEVQYFFKILDN
Human NME7 b
(DNA)
(SEQ ID NO: 148)
atgcatgatgtaaagaatcatcgcacctttttaaagcggaccaaatatgataacctgcacttggaagatttatttataggc
aacaaagtgaatgtcttttctcgacaactggtattaattgactatggggatcaatatacagctcgccagctgggcagtaggaaagaaaaa
acgctagccctaattaaaccagatgcaatatcaaaggctggagaaataattgaaataataaacaaagctggatttactataaccaaactc
aaaatgatgatgctttcaaggaaagaagcattggattttcatgtagatcaccagtcaagaccctttttcaatgagctgatccagtttattaca
actggtcctattattgccatggagattttaagagatgatgctatatgtgaatggaaaagactgctgggacctgcaaactctggagtggca
cgcacagatgcttctgaaagcattagagccctctttggaacagatggcataagaaatgcagcgcatggccctgattcttttgcttctgcg
gccagagaaatggagttgttttttccttcaagtggaggttgtgggccggcaaacactgctaaatttactaattgtacctgttgcattgttaaa
ccccatgctgtcagtgaaggactgttgggaaagatcctgatggctatccgagatgcaggttttgaaatctcagctatgcagatgttcaata
tggatcgggttaatgttgaggaattctatgaagtttataaaggagtagtgaccgaatatcatgacatggtgacagaaatgtattctggccct
tgtgtagcaatggagattcaacagaataatgctacaaagacatttcgagaattttgtggacctgctgatcctgaaattgcccggcatttac
gccctggaactctcagagcaatctttggtaaaactaagatccagaatgctgttcactgtactgatctgccagaggatggcctattagagg
ttcaatacttcttcaagatcttggataattag
(amino acids)
(SEQ ID NO: 149)
MHDVKNHRTFLKRTKYDNLHLEDLFIGNKVNVFSRQLVLIDYGDQYTAR
QLGSRKEKTLALIKPDAISKAGEIIEIINKAGFTITKLKMMMLSRKEALDFHVDHQSRP
FFNELIQFITTGPIIAMEILRDDAICEWKRLLGPANSGVARTDASESIRALFGTDGIRNA
AHGPDSFASAAREMELFFPSSGGCGPANTAKFTNCTCCIVKPHAVSEGLLGKILMAIR
DAGFEISAMQMFNMDRVNVEEFYEVYKGVVTEYHDMVTEMYSGPCVAMEIQQNN
ATKTFREFCGPADPEIARHLRPGTLRAIFGKTKIQNAVHCTDLPEDGLLEVQYFFKILD
N
Human NME7_{AB} also known as NME7_{AB}
(DNA)
(SEQ ID NO: 150)
atggaaaaaacgctagccctaattaaaccagatgcaatatcaaaggctggagaaataattgaaataataaacaaagct
ggatttactataaccaaactcaaaatgatgatgctttcaaggaaagaagcattggattttcatgtagatcaccagtcaagaccctttttcaat
gagctgatccagtttattacaactggtcctattattgccatggagattttaagagatgatgctatatgtgaatggaaaagactgctgggacc
tgcaaactctggagtggcacgcacagatgcttctgaaagcattagagccctctttggaacagatggcataagaaatgcagcgcatggc
cctgattcttttgcttctgcggccagagaaatggagttgttttttccttcaagtggaggttgtgggccggcaaacactgctaaatttactaatt
gtacctgttgcattgttaaaccccatgctgtcagtgaaggactgttgggaaagatcctgatggctatccgagatgcaggttttgaaatctc
agctatgcagatgttcaatatggatcgggttaatgttgaggaattctatgaagtttataaaggagtagtgaccgaatatcatgacatggtga
cagaaatgtattctggcccttgtgtagcaatggagattcaacagaataatgctacaaagacatttcgagaattttgtggacctgctgatcct
gaaattgcccggcatttacgccctggaactctcagagcaatctttggtaaaactaagatccagaatgctgttcactgtactgatctgccag
aggatggcctattagaggttcaatacttcttcaagatcttggataattag
(amino acids)
(SEQ ID NO: 151)
MEKTLALIKPDAISKAGEIIEIINKAGFTITKLKMMMLSRKEALDFHVDHQS
RPFFNELIQFITTGPIIAMEILRDDAICEWKRLLGPANSGVARTDASESIRALFGTDGIR
NAAHGPDSFASAAREMELFFPSSGGCGPANTAKFTNCTCCIVKPHAVSEGLLGKILM
AIRDAGFEISAMQMFNMDRVNVEEFYEVYKGVVTEYHDMVTEMYSGPCVAMEIQQ
NNATKTFREFCGPADPEIARHLRPGTLRAIFGKTKIQNAVHCTDLPEDGLLEVQYFFK
ILDN
Human NME7-X1
(DNA)
(SEQ ID NO: 152)
atgatgatgctttcaaggaaagaagcattggattttcatgtagatcaccagtcaagaccctttttcaatgagctgatccag
tttattacaactggtcctattattgccatggagattttaagagatgatgctatatgtgaatggaaaagactgctgggacctgcaaactctgg
agtggcacgcacagatgcttctgaaagcattagagccctctttggaacagatggcataagaaatgcagcgcatggccctgattcttttgc
ttctgcggccagagaaatggagttgttttttccttcaagtggaggttgtgggccggcaaacactgctaaatttactaattgtacctgttgcat
tgttaaaccccatgctgtcagtgaaggactgttgggaaagatcctgatggctatccgagatgcaggttttgaaatctcagctatgcagat
gttcaatatggatcgggttaatgttgaggaattctatgaagtttataaaggagtagtgaccgaatatcatgacatggtgacagaaatgtatt
ctggcccttgtgtagcaatggagattcaacagaataatgctacaaagacatttcgagaattttgtggacctgctgatcctgaaattgcccg
gcatttacgccctggaactctcagagcaatctttggtaaaactaagatccagaatgctgttcactgtactgatctgccagaggatggccta
ttagaggttcaatacttcttcaagatcttggataattag
(amino acids)
(SEQ ID NO: 153)
MMMLSRKEALDFHVDHQSRPFFNELIQFITTGPIIAMEILRDDAICEWKRL
LGPANSGVARTDASESIRALFGTDGIRNAAHGPDSFASAAREMELFFPSSGGCGPANT
AKFTNCTCCIVKPHAVSEGLLGKILMAIRDAGFEISAMQMFNMDRVNVEEFYEVYK
GVVTEYHDMVTEMYSGPCVAMEIQQNNATKTFREFCGPADPEIARHLRPGTLRAIFG
KTKIQNAVHCTDLPEDGLLEVQYFFKILDN*
Human NME7 a (optimized for E coli expression)
(DNA)
(SEQ ID NO: 154)
atgaatcactccgaacgctttgtttttatcgccgaatggtatgacccgaatgcttccctgctgcgccgctacgaactgct
gttttatccgggcgatggtagcgtggaaatgcatgacgttaaaaatcaccgtacctttctgaaacgcacgaaatatgataatctgcatctg
gaagacctgtttattggcaacaaagtcaatgtgttctctcgtcagctggtgctgatcgattatggcgaccagtacaccgcgcgtcaactg
ggtagtcgcaaagaaaaaacgctggccctgattaaaccggatgcaatctccaaagctggcgaaattatcgaaattatcaacaaagcgg
gtttcaccatcacgaaactgaaaatgatgatgctgagccgtaaagaagccctggattttcatgtcgaccaccagtctcgcccgtttttcaa
tgaactgattcaattcatcaccacgggtccgattatcgcaatggaaattctgcgtgatgacgctatctgcgaatggaaacgcctgctggg
cccggcaaactcaggtgttgcgcgtaccgatgccagtgaatccattcgcgctctgtttggcaccgatggtatccgtaatgcagcacatg
gtccggactcattcgcatcggcagctcgtgaaatggaactgtttttcccgagctctggcggttgcggtccggcaaacaccgccaaattt
accaattgtacgtgctgtattgtcaaaccgcacgcagtgtcagaaggcctgctgggtaaaattctgatggcaatccgtgatgctggcttt
gaaatctcggccatgcagatgttcaacatggaccgcgttaacgtcgaagaattctacgaagtttacaaaggcgtggttaccgaatatca
cgatatggttacggaaatgtactccggtccgtgcgtcgcgatggaaattcagcaaaacaatgccaccaaaacgtttcgtgaattctgtgg
tccggcagatccggaaatcgcacgtcatctgcgtccgggtaccctgcgcgcaatttttggtaaaacgaaaatccagaacgctgtgcact
gtaccgatctgccggaagacggtctgctggaagttcaatactttttcaaaattctggataat
(amino acids)
(SEQ ID NO: 155)
MNHSERFVFIAEWYDPNASLLRRYELLFYPGDGSVEMHDVKNHRTFLKR
TKYDNLHLEDLFIGNKVNVFSRQLVLIDYGDQYTARQLGSRKEKTLALIKPDAISKA
GEIIEIINKAGFTITKLKMMMLSRKEALDFHVDHQSRPFFNELIQFITTGPIIAMEILRDD
AICEWKRLLGPANSGVARTDASESIRALFGTDGIRNAAHGPDSFASAAREMELFFPSS
GGCGPANTAKFTNCTCCIVKPHAVSEGLLGKILMAIRDAGFEISAMQMFNMDRVNV
EEFYEVYKGVVTEYHDMVTEMYSGPCVAMEIQQNNATKTFREFCGPADPEIARHLR
PGTLRAIFGKTKIQNAVHCTDLPEDGLLEVQYFFKILDNTG
Human NME7 b (optimized for E coli expression)
(DNA)
(SEQ ID NO: 156)
atgcatgacgttaaaaatcaccgtacctttctgaaacgcacgaaatatgataatctgcatctggaagacctgtttattggc
aacaaagtcaatgtgttctctcgtcagctggtgctgatcgattatggcgaccagtacaccgcgcgtcaactgggtagtcgcaaagaaaa
aacgctggccctgattaaaccggatgcaatctccaaagctggcgaaattatcgaaattatcaacaaagcgggtttcaccatcacgaaac
tgaaaatgatgatgctgagccgtaaagaagccctggattttcatgtcgaccaccagtctcgcccgtttttcaatgaactgattcaattcatc
accacgggtccgattatcgcaatggaaattctgcgtgatgacgctatctgcgaatggaaacgcctgctgggcccggcaaactcaggtg
ttgcgcgtaccgatgccagtgaatccattcgcgctctgtttggcaccgatggtatccgtaatgcagcacatggtccggactcattcgcat
cggcagctcgtgaaatggaactgtttttcccgagctctggcggttgcggtccggcaaacaccgccaaatttaccaattgtacgtgctgta
ttgtcaaaccgcacgcagtgtcagaaggcctgctgggtaaaattctgatggcaatccgtgatgctggctttgaaatctcggccatgcag
atgttcaacatggaccgcgttaacgtcgaagaattctacgaagtttacaaaggcgtggttaccgaatatcacgatatggttacggaaatg
tactccggtccgtgcgtcgcgatggaaattcagcaaaacaatgccaccaaaacgtttcgtgaattctgtggtccggcagatccggaaat
cgcacgtcatctgcgtccgggtaccctgcgcgcaatttttggtaaaacgaaaatccagaacgctgtgcactgtaccgatctgccggaa
gacggtctgctggaagttcaatactttttcaaaattctggataat
(amino acids)
(SEQ ID NO: 157)
MHDVKNHRTFLKRTKYDNLHLEDLFIGNKVNVFSRQLVLIDYGDQYTAR
QLGSRKEKTLALIKPDAISKAGEIIEIINKAGFTITKLKMMMLSRKEALDFHVDHQSRP
FFNELIQFITTGPIIAMEILRDDAICEWKRLLGPANSGVARTDASESIRALFGTDGIRNA
AHGPDSFASAAREMELFFPSSGGCGPANTAKFTNCTCCIVKPHAVSEGLLGKILMAIR
DAGFEISAMQMFNMDRVNVEEFYEVYKGVVTEYHDMVTEMYSGPCVAMEIQQNN
ATKTFREFCGPADPEIARHLRPGTLRAIFGKTKIQNAVHCTDLPEDGLLEVQYFFKILD
NTG
Human NME7_{AB} also known as NME7_{AB} (optimized for E coli expression)
(DNA)
(SEQ ID NO: 158)
atggaaaaaacgctggccctgattaaaccggatgcaatctccaaagctggcgaaattatcgaaattatcaacaaagcg
ggtttcaccatcacgaaactgaaaatgatgatgctgagccgtaaagaagccctggattttcatgtcgaccaccagtctcgcccgtttttca
atgaactgattcaattcatcaccacgggtccgattatcgcaatggaaattctgcgtgatgacgctatctgcgaatggaaacgcctgctgg
gcccggcaaactcaggtgttgcgcgtaccgatgccagtgaatccattcgcgctctgtttggcaccgatggtatccgtaatgcagcacat
ggtccggactcattcgcatcggcagctcgtgaaatggaactgtttttcccgagctctggcggttgcggtccggcaaacaccgccaaatt
taccaattgtacgtgctgtattgtcaaaccgcacgcagtgtcagaaggcctgctgggtaaaattctgatggcaatccgtgatgctggcttt
gaaatctcggccatgcagatgttcaacatggaccgcgttaacgtcgaagaattctacgaagtttacaaaggcgtggttaccgaatatca
cgatatggttacggaaatgtactccggtccgtgcgtcgcgatggaaattcagcaaaacaatgccaccaaaacgtttcgtgaattctgtgg
tccggcagatccggaaatcgcacgtcatctgcgtccgggtaccctgcgcgcaatttttggtaaaacgaaaatccagaacgctgtgcact
gtaccgatctgccggaagacggtctgctggaagttcaatactttttcaaaattctggataat
(amino acids)
(SEQ ID NO: 159)
MEKTLALIKPDAISKAGEIIEIINKAGFTITKLKMMMLSRKEALDFHVDHQS
RPFFNELIQFITTGPIIAMEILRDDAICEWKRLLGPANSGVARTDASESIRALFGTDGIR
NAAHGPDSFASAAREMELFFPSSGGCGPANTAKFTNCTCCIVKPHAVSEGLLGKILM
AIRDAGFEISAMQMFNMDRVNVEEFYEVYKGVVTEYHDMVTEMYSGPCVAMEIQQ
NNATKTFREFCGPADPEIARHLRPGTLRAIFGKTKIQNAVHCTDLPEDGLLEVQYFFK
ILDNTG
Human NME7-X1 (optimized for E coli expression)
(DNA)
(SEQ ID NO: 160)
atgatgatgctgagccgtaaagaagccctggattttcatgtcgaccaccagtctcgcccgtttttcaatgaactgattcaa
ttcatcaccacgggtccgattatcgcaatggaaattctgcgtgatgacgctatctgcgaatggaaacgcctgctgggcccggcaaactc
aggtgttgcgcgtaccgatgccagtgaatccattcgcgctctgtttggcaccgatggtatccgtaatgcagcacatggtccggactcatt
cgcatcggcagctcgtgaaatggaactgtttttcccgagctctggcggttgcggtccggcaaacaccgccaaatttaccaattgtacgt
gctgtattgtcaaaccgcacgcagtgtcagaaggcctgctgggtaaaattctgatggcaatccgtgatgctggctttgaaatctcggcca
tgcagatgttcaacatggaccgcgttaacgtcgaagaattctacgaagtttacaaaggcgtggttaccgaatatcacgatatggttacgg
aaatgtactccggtccgtgcgtcgcgatggaaattcagcaaaacaatgccaccaaaacgtttcgtgaattctgtggtccggcagatccg
gaaatcgcacgtcatctgcgtccgggtaccctgcgcgcaatttttggtaaaacgaaaatccagaacgctgtgcactgtaccgatctgcc
ggaagacggtctgctggaagttcaatactttttcaaaattctggataat
(amino acids)
(SEQ ID NO: 161)
MMMLSRKEALDFHVDHQSRPFFNELIQFITTGPIIAMEILRDDAICEWKRL
LGPANSGVARTDASESIRALFGTDGIRNAAHGPDSFASAAREMELFFPSSGGCGPANT
AKFTNCTCCIVKPHAVSEGLLGKILMAIRDAGFEISAMQMFNMDRVNVEEFYEVYK
GVVTEYHDMVTEMYSGPCVAMEIQQNNATKTFREFCGPADPEIARHLRPGTLRAIFG
KTKIQNAVHCTDLPEDGLLEVQYFFKILDNTG
DM10 domain of NME7
(amino acids)
(SEQ ID NO: 162)
MNHSERFVFIAEWYDPNASLLRRYELLFYPGDGSVEMHDVKNHRTFLKR
TKYDNLHLEDLFIGNKVNVFSRQLVLIDYGDQYTARQLGSRK
a fragment or variation of PSMGFR peptide
(SEQ ID NO: 163)
SNIKFRPGSVVVQLTLAFREGTINVHDVETQFNQYKTEAASRY;
a fragment or variation of PSMGFR peptide
(SEQ ID NO: 164)
SVVVQLTLAFREGTINVHDVETQFNQYKTEAASRY;
a fragment or variation of PSMGFR peptide
(SEQ ID NO: 165)
VQLTLAFREGTINVHDVETQFNQY;
a fragment or variation of PSMGFR peptide
(SEQ ID NO: 166)
SNIKFRPGSVVVQLTLAFREGTIN;
a fragment or variation of PSMGFR peptide
(SEQ ID NO: 167)
SNIKFRPGSVVVQLTLAFREGTINVHDVETQFNQYKTE;
a fragment or variation of PSMGFR peptide
(SEQ ID NO: 168)
VQLTLAFREGTINVHDVETQFNQYKTEAASRYNLTISDVSVSDVP.
Cys at residue 14 is mutated to Ser of NME7B peptide 3 (B domain):
(SEQ ID NO: 169)
AIFGKTKIQNAVHSTDLPEDGLLEVQYFF
N-10 peptide
(SEQ ID NO: 170)
QFNQYKTEAASRYNLTISDVSVSDVPFPFSAQSGA
C-10 peptide
(SEQ ID NO: 171)
GTINVHDVETQFNQYKTEAASRYNLTISDVSVSDV

### EXAMPLES

### Example 1—Components of Minimal Serum-Free Base (“MM”) (500 Mls)

400 ml DME/F12/GlutaMAX I (Invitrogen #10565-018)

100 ml Knockout Serum Replacement (KO-SR, Invitrogen #10828-028)

5 ml 100×MEM Non-essential Amino Acid Solution (Invitrogen #11140-050)

0.9 ml (0.1 mM) β-mercaptoethanol (55 mM stock, Invitrogen #21985-023.

### Example 2—Generation of Protein Constructs

For generating recombinant NME7, first, constructs were made to make a recombinant NME7 that could be expressed efficiently and in soluble form. The first approach was to make a construct that would encode the native NME7 (a) or an alternative splice variant NME7 (b), which has an N-terminal deletion. In some cases, the constructs carried a histidine tag or a strep tag to aid in purification. NME7-a, full-length NME7 expressed poorly in E. coli and NME7-b did not express at all in E. coli. However, a novel construct was made in which the DM10 sequence was deleted and the NME7 comprised essentially the NDPK A and B domains having a calculated molecular weight of 33 kDa.

This novel NME7_{AB} expressed very well in E. coli and existed as the soluble protein. NME7_{AB} was first purified over an NTA-Ni column and then further purified by size exclusion chromatography (FPLC) over a Sephadex 200 column. Fractions were collected and tested by SDS-PAGE to identify fractions with the highest and purest expression of NME7_{AB}. The FPLC trace for the combined fractions that were the most pure were combined. The purified NME7_{AB} protein was then tested and shown to fully support the growth of human stem cells and further reverts them to the most naïve, pre-X-inactivation state. The purified NME7_{AB} was also shown to accelerate the growth of cancer cells.

### Example 3—ELISA Assay Showing NME7_{AB} Simultaneously Binds to Two MUC1* Extra Cellular Domain Peptides

Results are shown in FIG. 1. The PSMGFR peptide bearing a C-terminal Cysteine (PSMGFR-Cys) was covalently coupled to BSA using Imject Maleimide activated BSA kit (Thermo Fisher). PSMGFR-Cys coupled BSA was diluted to 10 ug/mL in 0.1M carbonate/bicarbonate buffer pH 9.6 and 50 uL was added to each well of a 96 well plate. After overnight incubation at 4° C., the plate was washed twice with PBS-T and a 3% BSA solution was added to block remaining binding site on the well. After 1 h at RT the plate was washed twice with PBS-T and NME7, diluted in PBS-T+1% BSA, was added at different concentrations. After 1 h at RT the plate was washed 3× with PBS-T and anti-NM23-H7 (B-9, Santa Cruz Biotechnology), diluted in PBS-T+1% BSA, was added at 1/500 dilution. After 1 h at RT the plate was washed 3× with PBS-T and goat anti mouse-HRP, diluted in PBS-T+1% BSA, was added at 1/3333 dilution. After 1 h at RT the plate was washed 3× with PBS-T and binding of NME7 was measured at 415 nm using ABTS solution (Pierce).

ELISA MUC1* dimerization: The protocol for NME7 binding was used, and NME7 was used at 11.6 ug/mL.

After 1 h at RT the plate was washed 3× with PBS-T and His-Tagged PSMGFR peptide (PSMGFR-His) or biotinylated PSMGFR peptide (PSMGFR-biotin), diluted in PBS-T+1% BSA, was added at different concentration. After 1 h at RT the plate was washed 3× with PBS-T and anti-Histag-HRP (Abcam) or streptavidin-HRP (Pierce), diluted in PBS-T+1% BSA, was added at a concentration of 1/5000. After 1 h at RT the plate was washed 3× with PBS-T and binding of PSMGFR peptide to NME7 already bound to another PSMGFR peptide (which could not signal by anti-His antibody or by streptavidin) coupled BSA was measured at 415 nm using a ABTS solution (Pierce).

### Example 4—Functional Testing of Human Recombinant NME7_{AB}

For testing recombinant NME7_{AB} for ability to maintain pluripotency and inhibit differentiation, a soluble variant of NME7, NME7_{AB}, was generated and purified. Human stem cells (iPS cat #SC101a-1, System Biosciences) were grown per the manufacturer's directions in 4 ng/ml bFGF over a layer of mouse fibroblast feeder cells for four passages. These source stem cells were then plated into 6-well cell culture plates (Vita™, Thermo Fisher) that had been coated with 12.5 μg/well of a monoclonal anti-MUC1* antibody, MN-C3. Cells were plated at a density of 300,000 cells per well. The base media was Minimal Stem Cell Media consisting of: 400 ml DME/F12/GlutaMAX I (Invitrogen #10565-018), 100 ml Knockout Serum Replacement (KO-SR, Invitrogen #10828-028), 5 ml 100×MEM Non-essential Amino Acid Solution (Invitrogen #11140-050) and 0.9 ml (0.1 mM) β-mercaptoethanol (55 mM stock, Invitrogen #21985-023). The base media can be any media. In a preferred embodiment, the base media is free of other growth factors and cytokines. To the base media was added either 8 nM of NME7_{AB} or 8 nM NM23-H1 refolded and purified as stable dimers. Media was changed every 48 hours and due to accelerated growth, had to be harvested and passaged at Day 3 post-plating. Comparable pluripotent stem cell growth was achieved when stem cells were grown in NM23-H1 dimers or in NME7 monomers.

NME7 and NM23-H1 (NME1) dimers both grew pluripotently and had no differentiation even when 100% confluent. As can be seen in the photos, NME7 cells grew faster than the cells grown in NM23-H1 dimers. Cell counts at the first harvest verified that culture in NME7 produced 1.4-times more cells than culture in NM23-H1 dimers. ICC staining for the typical pluripotent markers confirmed that NME7_{AB} fully supported human stem cell growth, pluripotency, and resisted differentiation.

The NME7 species of ~30-33 kDa may be an alternative splice isoform or a post translational modification such as cleavage, which may enable secretion from the cell.

### Example 5—Inducing Transition of Cancer Cells to Metastatic Cancer Cells by Culturing Cells Under Conditions that Revert Stem Cells to a More Naïve State

Cancer cells are normally cultured in a serum-containing media such as RPMI. We discovered that culturing cancer cells in the presence of reagents that make stem cells revert to a more naïve state, makes the cancer cells transform to a more metastatic state.

We demonstrated that NME7_{AB}, human NME1 dimers, bacterial NME1 dimers, NME7-X1 and “2i” inhibitors were each able to transform regular cancer cells into metastatic cancer cells, which are also called cancer stem cells “CSCs” or tumor initiating cells “TICs”. 2i is the name given to two biochemical inhibitors that researchers found made human stem cells revert to a more naïve state. 2i are MEK and GSK3-beta inhibitors PD0325901 and CHIR99021, which are added to culture medium to final concentrations of about 1 mM and 3 mM, respectively.

NME7_{AB} and NME7-X1 are at a final concentration of about 4 nM when added to separate batches of minimal medium to make cancer cells transform to metastatic cells, although lower and higher concentrations also work well in the range of about 1 nM to 16 nM. Human or bacterial NME1 dimers are used at a final concentration of 4 nM to 32 nM, with 16 nM typically used in these experiments, wherein the human NME bears the S120G mutation. Lower concentrations may be required if using wild type. It is not intended that these exact concentrations are important. It is important that the NME1 proteins are dimers and the range of concentrations over which this happens is in the low nanomolar range although certain mutations allow higher concentrations to remain as dimers.

Similarly, the concentrations of NME7 proteins can vary. NME7_{AB} and NME7-X1 are monomers and concentrations used to transform cancer cells to metastatic cells should allow the proteins to remain as monomers. Various molecular markers have been proposed as being indicators of metastatic cancer cells. Different cancer types may have different molecules that are up-regulated. For example, the receptor CXCR4 is up-regulated in metastatic breast cancers while E-cadherin, also known as CHD1, is up-regulated more in metastatic prostate cancers.

In addition to these specific metastasis markers, typical markers of pluripotency such as OCT4, SOX2, NANOG, and KLF4 are up-regulated as cancers become metastatic. The starting cancer cells and the later metastatic cancer cells can be assayed by PCR to measure expression levels of these genes.

FIG. 2 shows a graph of RT-PCR measurements of T47D breast cancer cells that were cultured in a media that contained NME7_{AB}. A rho I kinase inhibitor, ROCi, ROCKi or Ri, was added to prevent the transformed cells from floating off the plate. Expression levels of various metastatic markers as well as pluripotent stem cell markers were measured for the parent cells and for the NME7_{AB} cultured cells. The results show that the floater cells express higher amounts of metastatic and pluripotency markers compared to the cells that received ROCi. We reasoned it was because those measurements were the average of cells that did not transform and those that did but the ROCi made them remain adherent. This can clearly be seen in figures wherein “—Ri” means adherent cells that did not receive ROCi and so were not mixed with the highly metastatic cells that float.

Prostate cancer cells also transitioned to a more metastatic state when cultured in media containing NM23, aka NME1, or NME7_{AB}. Here we show that for every cell line tested so far, culture in NME7_{AB}, human NME1 dimers, or bacterial NMEs that have high sequence homology to human, induces transition to a more metastatic state.

FIG. 4 shows a graph of RT-PCR measurements of expression levels of metastatic and pluripotency markers for breast cancer cells that are cultured in media containing either 2i inhibitors, NME7_{AB} or both. As can be seen, 2i inhibitors are also able to induce the transition of cancer cells to a more metastatic state. Ovarian cancer cell lines SK-OV3, OV-90, pancreatic cancer cell lines CAPAN-2 and PANC-1, breast cancer cell line MDA-MB all displayed the morphological transition of going from adherent to non-adherent when cultured in NME7_{AB} and or 2i inhibitors.

FIG. 20 shows graphs of RT-PCR measurement of metastatic or pluripotency markers for various cancer cell lines cultured for 72 or 144 hours in NME7_{AB}. FIG. 20A shows that SK-OV3 cells increase expression of metastatic markers CHD1, SOX2 and NME7-X1 when cultured in NME7_{AB}. FIG. 20B shows that OV-90 cells increase expression of metastatic markers CXCR4 and NME7-X1 after culture in NME7_{AB}.

### Example 6—Demonstration that Cancer Cells Cultured in NME7 Become Metastatic

A functional test of whether or not a population of cancer cells is metastatic is to implant very low numbers, e.g. 200, of the cells in immuno-compromised mice and see if they develop into a tumor. Typically 5-6 million cancer cells are required to form a tumor in an immuno-compromised mouse. We showed that as few as 50 of the NME-induced metastatic cancer cells formed tumors in mice. In addition, mice that were injected throughout the test period with human NME7_{AB}, NME1, or NME7-X1 developed remote metastases.

T47D human breast cancer cells were cultured in standard RPMI media for 14 days with media changes every 48 hours and passed by trypsinization when approximately 75% confluent. The cells were then plated into 6-well plates and cultured in minimal stem cell media (see Example 1) that was supplemented with 4 nM NME7_{AB}. Media was changed every 48 hours. By about Day 4, some cells become detached from the surface and float. Media is carefully changed so as to retain the “floaters” as these are the cells that have the highest metastatic potential as evidenced by RT-PCR measurement of metastatic markers. On Day 7 or 8, the floaters are harvested and counted. Samples are retained for RT-PCR measurement. The key marker measured is CXCR4 which is up-regulated by 40-200 times after being briefly cultured in NME7_{AB}.

The freshly harvested floater metastatic cells are xenografted into the flank of female nu/nu athymic mice that have been implanted with 90-day slow release estrogen pellets. Floater cells were xenografted as 10,000, 1,000, 100 or 50 cells each. Half of the mice in each group of 6 were also injected daily with 32 nM NME7_{AB} near the original implantation site. The parent T47D cells that were cultured in RPMI media without NME7_{AB} were also implanted into mice as 6 million, 10,000 or 100 as controls. Mice implanted with the NME7-induced floater cells developed tumors even when as few as 50 cells were implanted. Mice that were implanted with the floater cells and that received daily injections of NME7_{AB} also developed remote tumors or remote metastases in various organs. 11 out of the 12 mice, or 92%, that were injected with human NME7_{AB} after implantation of the NME7_{AB} cultured cancer cells, developed tumors at the injection site. Only 7 out of the 12 mice, or 58%, that were not injected with human NME7_{AB} after implantation developed tumors. 9 out of the 11 mice, or 82%, that got tumors and were injected with human NME7_{AB} developed multiple tumors remote from the injection site. None of the mice that were not injected with NME7_{AB} developed multiple, visible tumors.

After sacrifice, RT-PCR and Western blots showed that the remote bumps on the mice injected with NME7_{AB} were indeed human breast tumors. Similar analysis of their organs showed that in addition to remote bumps, mice had randomly metastasized to the liver and lung with human breast cancer characteristic of the human breast cancer cells that were implanted. As expected, only the mice implanted with 6 million cells grew tumors.

Several experiments like the one described above were performed with essentially the same results. In each experiment, there were either 24 or 52 mice, including all proper controls.

### Example 7—Peptides Selected Because their Sequence is Unique to NME7, A1, A2, B1, B2 and B3, Inhibit the Binding of NME7 Species to MUC1* Extracellular Domain Peptide

NME7 peptides were selected as immunizing agents for antibody production. NME7 peptides A1, A2, B1, B2 and B3 (FIG. 9) were chosen using a process of sequence alignment among human NME1, human NME7 and several bacterial NMEs that were homologous to human NME1 or human NME7. Five regions that had high sequence homology among all were identified. However, to prevent selecting peptides that would give rise to antibodies that would inhibit human NME1 as well as human NME7, we chose NME7 sequences that were adjacent to the homologous regions wherein those peptides had sequences that were different from human NME1. We did ELISA assays to see if the peptides on their own could bind to a synthetic MUC1* peptide on the surface and inhibit the binding of human NME7 or human NME1 to the immobilized peptide (FIG. 11). FIG. 11 shows that the peptides inhibited the binding of NME7 and NME1 to the immobilized PSMGFR peptide. Recall that each of the NME7 A domain and B domain can bind to a PSMGFR peptide. Therefore complete inhibition of NME7_{AB} binding to a PSMGFR peptide cannot be accomplished with a single antibody or peptide that is derived from just one domain. This showed that those regions from which the peptides were derived were the regions that interacted with MUC1* and would give rise to antibodies that would bind to those regions of NME7 and inhibit its binding to MUC1* receptor.

In another experiment, the free peptides A1, A2, B1, B2 and B3 were added to cancer cells in culture that were undergoing transition to a more metastatic state by culturing in either NME7_{AB} or 2i. FIG. 14 shows a table of scientist observations when cancer cells are grown in either NME7_{AB} or 2i inhibitors, and shows that the free peptides inhibited the morphological change from adherent cells to floaters, which for breast cancer cells is directly correlated to increased expression of metastatic markers, especially CXCR4. RT-PCR measurements confirm that the NME7_{AB} peptides inhibited the increase in expression of metastasis marker CXCR4.

FIG. 15 shows a graph of RT-PCR measurements of CXCR4 expression in T47D breast cancer cells that were grown in either NME7_{AB} or 2i inhibitors, each of which transform cancer cells to a more metastatic state, and the inhibitory effect of NME7-derived peptides, A1, A2, B1, B2 and B3, on the metastatic transformation. FIG. 32 shows a table of recorded RNA levels in samples that were used for RT-PCR measurement of CXCR4 in FIG. 15 as well as the threshold cycle number for CXCR4 expression as well as for the control housekeeping gene.

### Example 8—Anti-NME7 Antibodies Specifically Bind to Human NME7 but not to Human NME1

A standard ELISA assay was performed to determine whether or not the NME7 antibodies we generated by immunization with NME7_{AB} peptides A1, A2, B1, B2, and B3 would bind specifically to NME7_{AB}, but not to human NME1 as it has healthy functions and it may be detrimental to a human to block it with an antibody. The ELISAs of FIG. 24-25 show that all of the NME7 antibodies that were generated from peptides A1, A2, B1, B2, and B3 bind to human NME7_{AB} (FIG. 24) but not to human NME1 (FIG. 25). The peptides used to generate these antibodies are common to both NME7_{AB} and NME7-X1. This assays show that the antibodies generated from peptides A1, A2, B1, B2, and B3 specifically bind to NME7_{AB} and by extension will bind to NME7-X1.

NME7A peptide 1 (A domain):
(SEQ ID NO: 141)
MLSRKEALDFHVDHQS
NME7A peptide 2 (A domain):
(SEQ ID NO: 142)
SGVARTDASES
NME7B peptide 1 (B domain):
(SEQ ID NO: 143)
DAGFEISAMQMFNMDRVNVE
NME7B peptide 2 (B domain):
(SEQ ID NO: 144)
EVYKGVVTEYHDMVTE
NME7B peptide 3 (B domain):
(SEQ ID NO: 145)
AIFGKTKIQNAVHCTDLPEDGLLEVQYFF

### Example 9—Anti-NME7 Specific Antibodies and the Peptides that Generated them Inhibit Cancer Cell Growth

Rabbits were immunized with NME7 peptides A1, A2, B1, B2, and B3 and antibodies were generated, collected and purified over a column to which the immunizing peptide had been conjugated. T47D breast cancer cells were plated and cultured according to ATCC protocols in RPMI media supplemented with serum. Antibodies generated from immunization with peptides A1, A2, B1, B2, and B3 were added at the concentrations indicated in FIG. 12. Immunizing peptides A1, A2, B1, B2, and B3, and the PSMGFR extracellular domain peptide of MUC1*, “FLR” here, were also added separately to growing T47D breast cancer cells. Taxol and the E6 anti-MUC1* Fab were added as controls. The graph of FIG. 12 shows that the antibodies generated, as well as the free peptides, potently inhibited the growth of the cancer cells. Note the comparison to inhibition using Taxol, which is a chemotherapy agent that kills healthy and cancer cells alike. Also, for comparison, a polyclonal antibody generated using a large stretch of NME7 from amino acid 100 to 376 is shown. Although this antibody is a potent inhibitor of cancer growth it could have non-specific effects since it can bind to NME1 as well as to NME7.

In a similar experiment, combinations of the antibodies generated from immunization with peptides A1, A2, B1, B2, and B3 as well as the peptides themselves were added to growing cancer cells at the concentrations indicated. The graphs of cell growth shown in FIG. 13 show that the combinations of antibodies and peptides potently inhibited the growth of cancer cells. In these two experiments, the cells were MUC1* positive breast cancer cells.

### Example 10—Anti-NME7 Antibodies Inhibit the Transition of Cancer Cells to Metastatic Cancer Cells

Cancer cells transform to a more metastatic state when cultured in the presence of agents that revert stem cells to a more naïve state. We have demonstrated that culturing cancer cells in NME7_{AB}, human NME1 dimers, bacterial NME1 dimers or MEK and GSK3-beta inhibitors, called “2i”, causes the cells to become more metastatic. As the cells transition to a more metastatic state, they become non-adherent and float off of the culture plate. These floating cells, “floaters” were collected separately from those that were adherent and were shown to: a) express much higher levels of metastatic genes; and b) when xenografted into mice, the floater cells were able to generate tumors when implanted at very low numbers. RT-PCR measurement of specific metastatic markers such as CXCR4 in breast cancers, CHD1 in prostate cancer, and other pluripotent stem cell markers such as OCT4, SOX2, NANOG, KLF4, c-Myc and others were dramatically over-expressed in cancer cells that were cultured in NME7_{AB} and most over-expressed in the cells that became non-adherent, called “floaters” here and in figures.

Here we show that the NME7-specific antibodies, generated by immunization with NME7-derived peptides A1, A2, B1, B2 and B3, as well as the peptides themselves, inhibit the transition from cancer cell to metastatic cancer cells. In the first of these experiments, the antibodies generated by immunization with A1, A2, B1, B2 and B3 were tested for their ability to inhibit the metastatic transition induced by culture of T47D breast cancer cells in NME7_{AB} or in 2i inhibitors. The most striking observation was that the antibodies and the peptides dramatically reduced the number of floater cells, which was the first indication that the antibodies and peptides had inhibited the transformation to metastatic cancer cells. In particular, cells to which the antibody generated from immunization with the B3 peptide barely generated any floater cells.

FIG. 14 shows the recorded observations of the percentage of floater cells visible for each antibody relative to the control wells that did not receive any antibody treatment. mRNA was extracted from both the floater cells and the adherent cells. RT-PCR was used to measure expression levels of metastatic markers, including CXCR4. Treatment with the anti-NME7 antibodies greatly reduced the amount of metastatic markers, such as CXCR4, indicating the antibodies inhibited the transition to metastatic cancer. (See FIG. 15). Notably, the antibody generated by immunization with peptide B3, aka antibody #61, essentially completely inhibited the transition to a more metastatic state. FIG. 15B shows that breast cancer cells that were treated with the NME7_{AB} peptides, A1, A2, B1, B2 and B3, alone were able to potently inhibit the transition to a more metastatic state induced by culturing the cells in a media containing the 2i inhibitors. Peptide B3 was especially effective as was antibody #61 that it generated. FIG. 15C shows the same graph but with the Y-axis expanded to show the peptide inhibition of metastatic markers. The amount of mRNA, which indicates cell viability and growth, was measured. Cells that were treated with antibody had much less mRNA, indicating that in addition to inhibiting the transition to a more metastatic state, the anti-NME7_{AB} antibodies inhibited the growth of the cancer cells. FIG. 16 shows a table of the amounts of RNA recovered for the inhibition experiment shown in FIG. 15A.

### Example 11—Anti-NME7 Antibodies Generated with NME7-Derived Peptides A1, A2, B1, B2 and B3 Identify Novel NME7 Species not Detectable Using any Commercially Available Antibodies

As is known to those skilled in the art, some antibodies recognize a linear portion of the target protein and can be used in Western blot assays while other antibodies recognize a non-linear conformational motif and can be used in pull-down or immunoprecipitation assays. Previous to this application, cleaved NME7 or isoform NME7-X1 was not known to exist. Using antibodies that were commercially available at the time of filing shows that existing antibodies could not specifically detect these important NME7 species. B9 (Santa Cruz Biotechnology) is a monoclonal antibody raised against NME7 amino acids 100-376. FIG. 19D-19F shows that it only detects full-length 42 kDa NME7. Another commercially available antibody, H278, is a rabbit polyclonal raised against NME7 amino acids 100-376, which includes amino acid sequences that are not unique to NME7. FIG. 19D-19F shows that this antibody also stains NME1, which is 17 kDa as well as full-length NME7 and other bands that do not appear to be specific to NME7_{AB}.

NME7 antibodies generated by immunization with NME7_{AB} peptides A1, A2, B1, B2 or B3 identify new NME7 species including the full-length 42 kDa protein, a ~33 kDa NME7 species that may be a cleavage product or alternative isoform, a ~30 kDa NME7 species that may be a cleavage product or alternative isoform, wherein the ~30 kDa species appears to be NME7-X1. FIG. 19A-C shows that antibodies generated by peptides A1, B1 and B3 identify the secreted forms of NME7, NME7_{AB} and NME7-X1 in a wide range of cancer cell lines, including T47D breast cancer cells, PC3 and DU145 prostate cancer cells, HEK293 fetal liver cells, and leukemia cells IM-9, K562, and MV411.

### Example 12—Generation of Anti-NME7 Antibodies

A synthetic peptide having the sequence of the B3 region of NME7, AIFGKTKIQNAVHCTDLPEDGLLEVQYFFC (SEQ ID NO: 1142), was used to immunize rabbits. Antibodies that resulted from immunization with NME7 peptide B3 inhibited the growth of MUC1* positive cancer cells and also inhibited the formation of cancer stem cells, which are characterized by upregulation of metastatic markers, ability to grow anchorage independently, and are able to form tumors in animals from as few as 200 cells, whereas regular cancer cells typically require implantation of about 4 million cells for tumor engraftment.

In some cases, the NME7 B3 peptide was made with a C14A or C14V mutation. This sequence more reproducibly generated anti-NME7 antibodies.

Monoclonal antibodies were generated in mice according to standard methods by immunizing with NME7 B3, B3 with C14A mutation, or B3 with C14V mutation. The antibodies listed were selected because of their ability to bind to NME7, NME7-X1, NME7_{AB}, but importantly did not bind to NME1, which is thought to be required for some normal cellular functions. These antibodies also bind to the NME7 derived peptides B3, B3 with C14A mutation, and B3 with C14V mutation.

Experiments showed that these anti-NME7 antibodies inhibited the binding of NME7 to the MUC1* extra cellular domain, but did not block the binding of NME1 to the MUC1* extra cellular domain peptide. Further, the antibodies inhibited the formation of cancer stem cells.

### CITED REFERENCES LIST

Al-Hajj et al. (2003) Prospective identification of tumorigenic breast cancer cells. PNAS. Aprl 1; 100 (7): 3983-3988.
Clarke M F, Dick J E, Dirks P B, Eaves C J, Jamieson C H, Jones D L, Visvader J, Weissman I L, Wahl G M. (2006) Cancer stem cells—perspectives on current status and future directions: AACR Workshop on cancer stem cell. Cancer Res. October 1; 66 (19): 9339-44. Epub 2006 Sep. 21.
Chen K, Huang Y H, Chen J L. (2013) Understanding and targeting cancer stem cells: therapeutic implications and challenges. Acta Pharmacologica Sinica 34:732-740; Review
Darash-Yahana M, Pikarsky E, Abramovitch R, Zeira E, Pal B, Karplus R, Beider K, Avniel S, Kasem S, Galun E, Peled A (2004) Role of high expression levels of CXCR4 in tumor growth, vascularization, and metastasis. FASEB J 18 (11): 1240-1242
Mahanta S, Fessler S, Park J, Bamdad C. A Minimal Fragment of MUC1 Mediates Growth of Cancer Cells, 2008 PloS ONE 3: e2054-2065.
Hikita S, Clegg O, Kosik K, Bamdad C. MUC1* Mediates the Growth of Human Pluripotent Stem Cells, 2008 PloS ONE 3: e3312-3325.
Kumar S M, Liu S, Lu H, Zhang H, Zhang P J, Gimotty P A, Guerra M, Guo W, Xu X. (2012) Acquired cancer stem cell phenotypes through Oct4-mediated dedifferentiation. Oncogene. November 22; 31 (47): 4898-911.
Liu K, Lin B, Zhao M, Yang X, Chen M, Gao A, Liu F, Que J, Lan X. (2013) The multiple roles for Sox2 in stem cell maintenance and tumorigenesis. Cellular Signaling May; 25 (5): 1264-71. Review
Wang M L, Chiou S H, Wu C W. (2013) Targeting cancer stem cells: emerging role of Nanog transcription factor. Onco targets and Therapy. September 4; 6:1207-20. Review.
Xu C, Rosler E, Jiang J, Lebkowski J S, Gold J D, et al. (2005) Basic Fibroblast Growth Factor Supports Undifferentiated Human Embryonic Stem Cell Growth Without Conditioned Medium. STEM CELLS 23:315-323.
Fessler S, Wotkowicz M, Mahanta S, Bamdad C (2009) MUC1* is a determinant of trastuzumab (Herceptin) resistance in breast cancer cells, Breast Cancer Res Treat 118:113-124 DOI 10.1007/s10549-009-0412-3
Miki J, Furusato B, Li H, Gu Y, Takahashi H, Egawa S, Sesterhenn I A, McLeod D G, Srivastava S, Rhim J S. Identification of putative stem cell markers, CD133 and CXCR4, in hTERT-immortalized primary nonmalignant and malignant tumor-derived human prostate epithelial cell lines and in prostate cancer specimens. Cancer Res. 2007 Apr. 1; 67 (7): 3153-61.
Jeter C R, Liu B, Liu X, Chen X, Liu C, Calhoun-Davis T, Repass J, Zaehres H, Shen J J, Tang D G. NANOG promotes cancer stem cell characteristics and prostate cancer resistance to androgen deprivation. Oncogene. 2011 Sep. 8; 30 (36): 3833-45. PMCID:
Faber A, Goessler U R, Hoermann K, Schultz J D, Umbreit C, Stern-Straeter J. SDF-1-CXCR4 axis: cell trafficking in the cancer stem cell niche of head and neck squamous cell carcinoma. Oncol. Rep. 2013 June; 29 (6): 2325-31.
Mukherjee D, Zhao J. The Role of chemokine receptor CXCR4 in breast cancer metastasis. Am J Cancer Res. 2013; 3 (1): 46-57. PMCID: PMC3555200
Herreros-Villanueva M, Zhang J-S, Koenig A, Abel E V, Smyrk T C, Bamlet W R, de Narvajas A A-M, Gomez T S, Simeone D M, Bujanda L, Billadeau D D. SOX2 promotes dedifferentiation and imparts stem cell-like features to pancreatic cancer cells. Oncogenesis. 2013; 2: e61. PMCID: PMC3759123
Hanna J, Cheng A W, Saha K, Kim J, Lengner C J, et al. (2010) Human embryonic stem cells with biological and epigenetic characteristics similar to those of mouse ESCs. Proc Natl Acad Sci USA 107:9222-9227.
Smagghe, B. J. Stewart A. K., Carter M. G., Shelton L. S., Bernier K. J., Hartman E. J., Calhoun A. K., Hatziioannou V. M., Lillacci G., Kirk B. A., DiNardo B. A., Kosik K. S., Bamdad C. (2013) MUC1* Ligand, NM23-H1, Is a Novel Growth Factor That Maintains Human Stem Cells in a More Naïve State. PloS ONE 8 (3): e58601
Theunissen T W, Powell B E, Wang H, Mitalipova M, Faddah D A, Reddy J, Fan Z P, Maetzel D, Ganz K, Shi L, Lungjangwa T, Imsoonthornruksa S, Stelzer Y, Rangarajan S, D'Alessio A, Zhang J, Gao Q, Dawlaty M M, Young R A, Gray N S, Jaenisch R. (2014) Systematic Identification of Culture Conditions for Induction and Maintenance of Naive Human Pluripotency. Cell Stem Cell. 2014 Jul. 24, S1934-5909 (14) 00298-7.
Rais Y1, Zviran A, Geula S, Gafni O, Chomsky E, Viukov S, Mansour A A, Caspi I, Krupalnik V, Zerbib M, Maza I, Mor N, Baran D, Weinberger L, Jaitin D A, Lara-Astiaso D, Blecher-Gonen R, Shipony Z, Mukamel Z, Hagai T, Gilad S, Amann-Zalcenstein D, Tanay A, Amit I, Novershtern N, Hanna J H (2013). Deterministic direct reprogramming of somatic cells to pluripotency., 502 (7469): 65-70.
Xu R H, Peck R M, Li D S, Feng X, Ludwig T, et al. (2005) Basic FGF and suppression of BMP signaling sustain undifferentiated proliferation of human E S cells. Nat Methods 2:185-190.
Liu W, Ma Q, Wong K, Li W, Ohgi K, Zhang J, Aggarwal A K, Rosenfeld M G. Brd4 and JMJD6-Associated Anti-Pause Enhancers in Regulation of Transcriptional Pause Release. Cell. 2013 Dec. 19; 155 (7): 1581-95. PMCID: PMC3886918.
Silva J, Barrandon O, Nichols J, Kawaguchi J, Theunissen T W, Smith A. Promotion of reprogramming to ground state pluripotency by signal inhibition. PLOS Biol. 2008 Oct. 21; 6 (10): e253. PMCID: PMC2570424
Takahashi K and Yamanaka S (2006) Induction of pluripotent stem cells from mouse embryonic and adult fibroblast cultures by defined factors. Cell 126 (4): 663-676.
Porter D et al. (2011) Chimeric antigen receptor-modified T cells in chronic lymphoid leukemia. N Engl J Med 365:725-733 DOI: 10.1056/NEJMoal103849
Tiller T et al. (2013) A fully synthetic human Fab antibody library based on fixed V H/V L framework pairings with favorable biophysical properties. MABs 9: 5 (3) PMID: 23571156
Webb P A, Perisic O, Mendola C E, Backer J M and Williams R L. The crystal structure of a human nucleoside diphosphate kinase, NM23-H2. J Mol Biol. 1995, 251:574-587.
Min K, Song H K, Chang C, Kim S Y, Lee K J and Suh S W. Crystal structure of human nucleoside diphosphate kinase A, a metastasis suppressor. Proteins. 2002, 46:340-342.
Okabe-Kado et al., “A new function of Nm23/NDP kinase as a differentiation inhibitory factor, which does not require it's kinase activity”, FEBS Letters 363:311-315, 1995
Lombardi et al., “nm23: Unraveling Its Biological Function in Cell Differentiation” JOURNAL O F CELLULAR PHYSIOLOGY 182:144-149 (2000)
Harrell et al., Estrogen Receptor Positive Breast Cancer Metastasis: Altered Hormonal Sensitivity and Tumor Aggressiveness in Lymphatic Vessels and Lymph Nodes. Cancer Res 2006; 66: (18). Sep. 15, 2006.
Suzuki et al., Combined effect of dehydroxymethylepoxyquinomicin and gemcitabine in a mouse model of liver metastasis of pancreatic cancer. Clin Exp Metastasis (2013) 30:381-392.

Those skilled in the art will recognize, or be able to ascertain using no more than routine experimentation, many equivalents to the specific embodiments of the invention specifically described herein. Such equivalents are intended to be encompassed in the scope of the claims.

1. An antibody that binds to NME7, comprising:
(a) a heavy chain variable region (HCV) comprising the sequence of any one of SEQ ID NOs: 1160, 1162, 1164, and 1181; and a light chain variable region (LCV) comprising complementarity determining region (LC-CDR) 1 comprising the sequence of SEQ ID NO: 434, an LC-CDR2 comprising the sequence of SEQ ID NO: 435, and an LC-CDR3 comprising the sequence of SEQ ID NO: 436; or
(b) an HCV comprising complementarity determining region (HC-CDR) 1 comprising the sequence of SEQ ID NO: 429, an HC-CDR2 comprising the sequence of SEQ ID NO: 430, and an HC-CDR3 comprising the sequence of SEQ ID NO: 431; and an LCV comprising the sequence of any one of SEQ ID NOs: 1166, 1168, 1170, 1183, 1185, and 1203.

2. The antibody of claim 1, wherein the antibody comprises a monoclonal antibody.

3. The antibody of claim 1, wherein the antibody comprises a humanized antibody.

4. The antibody of claim 1, wherein the antibody binds to the NME7 B3 peptide of SEQ ID NO: 145 or SEQ ID NO: 169.

5. The antibody of claim 1, wherein the HCV comprises the sequence of any one of SEQ ID NOs: 1160, 1162, 1164, and 1181; and the LCV comprises the sequence of any one of SEQ ID NOs: 1166, 1168, 1170, 1183, 1185, and 1203.

6. The antibody of claim 1, wherein the HCV comprises the sequence of SEQ ID NO: 1164, and the LCV comprises the sequence of any one of SEQ ID NOs: 1166, 1168, 1170, 1183, 1185, and 1203.

7. The antibody of claim 1, wherein the HCV comprises the sequence of any one of SEQ ID NOs: 1160, 1162, 1164, and 1181; and the LCV comprises the sequence of SEQ ID NO: 1185.

8. The antibody of claim 1, wherein the HCV comprises the sequence of SEQ ID NO: 1164.

9. The antibody of claim 1, wherein the LCV comprises the sequence of SEQ ID NO: 1185.

10. The antibody of claim 1, wherein the antibody is monovalent.

11. The antibody of claim 1, wherein the antibody is bivalent.

12. The antibody of claim 1, wherein the antibody comprises a fragment antigen-binding (Fab) region.

13. The antibody of claim 1, wherein the antibody comprises a single chain variable fragment (scFv) antibody.

14. The antibody of claim 1, wherein the antibody comprises a bispecific antibody.

15. A bispecific T cell engager comprising the antibody of claim 1.

16. An antibody drug conjugate (ADC) comprising the antibody of claim 1.

17. The ADC of claim 16, further comprising a toxin or a pro-toxin that is linked to the antibody.

18. A chimeric antigen receptor (CAR) comprising the antibody of claim 1.

19. An immune cell comprising the CAR of claim 18.

20. The immune cell of claim 19, wherein the immune cell comprises a T cell or NK cell.

21. A method of administering the antibody of claim 1 to a patient having a cancer or at risk of developing a cancer.

22. A method of detecting a cancer, the method comprising measuring a level of an NME7 protein and/or a level of a nucleic acid encoding an NME7 protein or a fragment thereof in a patient sample.

23.-36. (canceled)


SEQUENCE LISTING (1213 sequences)

SEQ ID NO: 1 - protein, 1255 aa, synthetic construct
  REGION 1..1255: note = MUC1 Receptor - Mucin 1 precursor, Genbank Accession number: P15941
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 2 - protein, 19 aa, synthetic construct
  REGION 1..19: note = N-terminal MUC-1 signaling sequence
MTPGTQSPFFLLLLLTVLT

SEQ ID NO: 3 - protein, 23 aa, synthetic construct
  REGION 1..23: note = N-terminal MUC-1 signaling sequence
MTPGTQSPFFLLLLLTVLTVVTA

SEQ ID NO: 4 - protein, 23 aa, synthetic construct
  REGION 1..23: note = N-terminal MUC-1 signaling sequence
MTPGTQSPFFLLLLLTVLTVVTG

SEQ ID NO: 5 - protein, 146 aa, synthetic construct
  REGION 1..146: note = Truncated MUC1 receptor isoform having nat-PSMGFR at its N-terminus and including the transmembrane and cytoplasmic sequences of a full-length MUC1 receptor
GTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 6 - protein, 45 aa, synthetic construct
  REGION 1..45: note = PSMGFR sequence
GTINVHDVETQFNQYKTEAASRYNLTISDVSVSDVPFPFSAQSGA

SEQ ID NO: 7 - protein, 44 aa, synthetic construct
  REGION 1..44: note = PSMGFR sequence
TINVHDVETQFNQYKTEAASRYNLTISDVSVSDVPFPFSAQSGA

SEQ ID NO: 8 - protein, 45 aa, synthetic construct
  REGION 1..45: note = PSMGFR sequence
GTINVHDVETQFNQYKTEAASPYNLTISDVSVSDVPFPFSAQSGA

SEQ ID NO: 9 - protein, 44 aa, synthetic construct
  REGION 1..44: note = PSMGFR sequence
TINVHDVETQFNQYKTEAASPYNLTISDVSVSDVPFPFSAQSGA

SEQ ID NO: 10 - DNA, 216 bp, synthetic construct
  misc_feature 1..216: note = MUC1 cytoplasmic domain nucleotide sequence
  source 1..216: mol_type = other DNA
tgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaacggtggcagcagcctctcttacacaaacccagcagtggcagccgcttctgccaacttg

SEQ ID NO: 11 - protein, 72 aa, synthetic construct
  REGION 1..72: note = MUC1 cytoplasmic domain amino acid sequence
CQCRRKNYGQLDIFPARDTYHPMSEYPTYHTHGRYVPPSSTDRSPYEKVSAGNGGSSLSYTNPAVAAASANL

SEQ ID NO: 12 - DNA, 854 bp, synthetic construct
  misc_feature 1..854: note = NME7 nucleotide sequence (NME7: GENBANK ACCESSION AB209049)
  source 1..854: mol_type = other DNA
gagatcctgagacaatgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggtatgttgaatacactatattcagtacattttgttaataggagagcaatgtttattttcttgatgtactttatgtatagaaaataa

SEQ ID NO: 13 - protein, 283 aa, synthetic construct
  REGION 1..283: note = NME7 amino acid sequence (NME7: GENBANK ACCESSION AB209049)
DPETMNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGMLNTLYSVHFVNRRAMFIFLMYFMYRK

SEQ ID NO: 14 - DNA, 534 bp, synthetic construct
  misc_feature 1..534: note = NM23-H1 nucleotide sequence (NM23-H1: GENBANK ACCESSION AF487339)
  source 1..534: mol_type = other DNA
atggtgctactgtctactttagggatcgtctttcaaggcgaggggcctcctatctcaagctgtgatacaggaaccatggccaactgtgagcgtaccttcattgcgatcaaaccagatggggtccagcggggtcttgtgggagagattatcaagcgttttgagcagaaaggattccgccttgttggtctgaaattcatgcaagcttccgaagatcttctcaaggaacactacgttgacctgaaggaccgtccattctttgccggcctggtgaaatacatgcactcagggccggtagttgccatggtctgggaggggctgaatgtggtgaagacgggccgagtcatgctcggggagaccaaccctgcagactccaagcctgggaccatccgtggagacttctgcatacaagttggcaggaacattatacatggcagtgattctgtggagagtgcagagaaggagatcggcttgtggtttcaccctgaggaactggtagattacacgagctgtgctcagaactggatctatgaatga

SEQ ID NO: 15 - protein, 177 aa, synthetic construct
  REGION 1..177: note = NM23-H1 describes amino acid sequence (NM23-H1: GENBANK ACCESSION AF487339)
MVLLSTLGIVFQGEGPPISSCDTGTMANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 16 - DNA, 534 bp, synthetic construct
  misc_feature 1..534: note = NM23-H1 S120G mutant nucleotide sequence (NM23-H1: GENBANK ACCESSION AF487339)
  source 1..534: mol_type = other DNA
atggtgctactgtctactttagggatcgtctttcaaggcgaggggcctcctatctcaagctgtgatacaggaaccatggccaactgtgagcgtaccttcattgcgatcaaaccagatggggtccagcggggtcttgtgggagagattatcaagcgttttgagcagaaaggattccgccttgttggtctgaaattcatgcaagcttccgaagatcttctcaaggaacactacgttgacctgaaggaccgtccattctttgccggcctggtgaaatacatgcactcagggccggtagttgccatggtctgggaggggctgaatgtggtgaagacgggccgagtcatgctcggggagaccaaccctgcagactccaagcctgggaccatccgtggagacttctgcatacaagttggcaggaacattatacatggcggtgattctgtggagagtgcagagaaggagatcggcttgtggtttcaccctgaggaactggtagattacacgagctgtgctcagaactggatctatgaatga

SEQ ID NO: 17 - protein, 177 aa, synthetic construct
  REGION 1..177: note = NM23-H1 S120G mutant amino acid sequence (NM23-H1: GENBANK ACCESSION AF487339)
MVLLSTLGIVFQGEGPPISSCDTGTMANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGGDSVESAEKEIGLWFHPEELVDYTSCAQNWIYE

SEQ ID NO: 18 - DNA, 459 bp, synthetic construct
  misc_feature 1..459: note = NM23-H2 nucleotide sequence (NM23-H2: GENBANK ACCESSION AK313448)
  source 1..459: mol_type = other DNA
atggccaacctggagcgcaccttcatcgccatcaagccggacggcgtgcagcgcggcctggtgggcgagatcatcaagcgcttcgagcagaagggattccgcctcgtggccatgaagttcctccgggcctctgaagaacacctgaagcagcactacattgacctgaaagaccgaccattcttccctgggctggtgaagtacatgaactcagggccggttgtggccatggtctgggaggggctgaacgtggtgaagacaggccgagtgatgcttggggagaccaatccagcagattcaaagccaggcaccattcgtggggacttctgcattcaggttggcaggaacatcattcatggcagtgattcagtaaaaagtgctgaaaaagaaatcagcctatggtttaagcctgaagaactggttgactacaagtcttgtgctcatgactgggtctatgaataa

SEQ ID NO: 19 - protein, 152 aa, synthetic construct
  REGION 1..152: note = NM23-H2 amino acid sequence (NM23-H2: GENBANK ACCESSION AK313448)
MANLERTFIAIKPDGVQRGLVGEIIKRFEQKGFRLVAMKFLRASEEHLKQHYIDLKDRPFFPGLVKYMNSGPVVAMVWEGLNVVKTGRVMLGETNPADSKPGTIRGDFCIQVGRNIIHGSDSVKSAEKEISLWFKPEELVDYKSCAHDWVYE

SEQ ID NO: 20 - DNA, 1023 bp, synthetic construct
  misc_feature 1..1023: note = Human NM23-H7-2 sequence optimized for E. coli expression
  source 1..1023: mol_type = other DNA
atgcatgacgttaaaaatcaccgtacctttctgaaacgcacgaaatatgataatctgcatctggaagacctgtttattggcaacaaagtcaatgtgttctctcgtcagctggtgctgatcgattatggcgaccagtacaccgcgcgtcaactgggtagtcgcaaagaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataattga

SEQ ID NO: 21 - protein, 340 aa, synthetic construct
  REGION 1..340: note = Human NM23-H7-2 sequence optimized for E. coli expression
MHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 22 - DNA, 399 bp, synthetic construct
  misc_feature 1..399: note = Human NME7-A
  source 1..399: mol_type = other DNA
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgtttttttga

SEQ ID NO: 23 - protein, 132 aa, synthetic construct
  REGION 1..132: note = Human NME7-A
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFF

SEQ ID NO: 24 - DNA, 444 bp, synthetic construct
  misc_feature 1..444: note = Human NME7-A1
  source 1..444: mol_type = other DNA
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttacttga

SEQ ID NO: 25 - protein, 147 aa, synthetic construct
  REGION 1..147: note = Human NME7-A1
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFT

SEQ ID NO: 26 - DNA, 669 bp, synthetic construct
  misc_feature 1..669: note = Human NME7-A2
  source 1..669: mol_type = other DNA
atgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgtttttttga

SEQ ID NO: 27 - protein, 222 aa, synthetic construct
  REGION 1..222: note = Human NME7-A2
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFF

SEQ ID NO: 28 - DNA, 714 bp, synthetic construct
  misc_feature 1..714: note = Human NME7-A3
  source 1..714: mol_type = other DNA
atgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttacttga

SEQ ID NO: 29 - protein, 237 aa, synthetic construct
  REGION 1..237: note = Human NME7-A3
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFT

SEQ ID NO: 30 - DNA, 408 bp, synthetic construct
  misc_feature 1..408: note = Human NME7-B
  source 1..408: mol_type = other DNA
atgaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttctga

SEQ ID NO: 31 - protein, 135 aa, synthetic construct
  REGION 1..135: note = Human NME7-B
MNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 32 - DNA, 426 bp, synthetic construct
  misc_feature 1..426: note = Human NME7-B1
  source 1..426: mol_type = other DNA
atgaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattagtga

SEQ ID NO: 33 - protein, 140 aa, synthetic construct
  REGION 1..140: note = Human NME7-B1
MNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 34 - DNA, 453 bp, synthetic construct
  misc_feature 1..453: note = Human NME7-B2
  source 1..453: mol_type = other DNA
atgccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttctga

SEQ ID NO: 35 - protein, 150 aa, synthetic construct
  REGION 1..150: note = Human NME7-B2
MPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 36 - DNA, 471 bp, synthetic construct
  misc_feature 1..471: note = Human NME7-B3
  source 1..471: mol_type = other DNA
atgccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattagtga

SEQ ID NO: 37 - protein, 155 aa, synthetic construct
  REGION 1..155: note = Human NME7-B3
MPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 38 - DNA, 864 bp, synthetic construct
  misc_feature 1..864: note = Human NME7-AB, also known as NME7AB
  source 1..864: mol_type = other DNA
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattagtga

SEQ ID NO: 39 - protein, 286 aa, synthetic construct
  REGION 1..286: note = Human NME7-AB, also known as NME7AB
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 40 - DNA, 846 bp, synthetic construct
  misc_feature 1..846: note = Human NME7-AB1
  source 1..846: mol_type = other DNA
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttctga

SEQ ID NO: 41 - protein, 281 aa, synthetic construct
  REGION 1..281: note = Human NME7-AB1
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 42 - DNA, 399 bp, synthetic construct
  misc_feature 1..399: note = Human NME7-A sequence optimized for E. coli expression
  source 1..399: mol_type = other DNA
atggaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttctga

SEQ ID NO: 43 - protein, 132 aa, synthetic construct; identical to SEQ ID NO: 23
  REGION 1..132: note = Human NME7-A sequence optimized for E. coli expression

SEQ ID NO: 44 - DNA, 444 bp, synthetic construct
  misc_feature 1..444: note = Human NME7-A1 sequence optimized for E. coli expression
  source 1..444: mol_type = other DNA
atggaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttacctga

SEQ ID NO: 45 - protein, 147 aa, synthetic construct; identical to SEQ ID NO: 25
  REGION 1..147: note = Human NME7-A1 sequence optimized for E. coli expression

SEQ ID NO: 46 - DNA, 669 bp, synthetic construct
  misc_feature 1..669: note = Human NME7-A2 sequence optimized for E. coli expression
  source 1..669: mol_type = other DNA
atgaatcactccgaacgctttgtttttatcgccgaatggtatgacccgaatgcttccctgctgcgccgctacgaactgctgttttatccgggcgatggtagcgtggaaatgcatgacgttaaaaatcaccgtacctttctgaaacgcacgaaatatgataatctgcatctggaagacctgtttattggcaacaaagtcaatgtgttctctcgtcagctggtgctgatcgattatggcgaccagtacaccgcgcgtcaactgggtagtcgcaaagaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttctga

SEQ ID NO: 47 - protein, 222 aa, synthetic construct; identical to SEQ ID NO: 27
  REGION 1..222: note = Human NME7-A2 sequence optimized for E. coli expression

SEQ ID NO: 48 - DNA, 714 bp, synthetic construct
  misc_feature 1..714: note = Human NME7-A3 sequence optimized for E. coli expression
  source 1..714: mol_type = other DNA
atgaatcactccgaacgctttgtttttatcgccgaatggtatgacccgaatgcttccctgctgcgccgctacgaactgctgttttatccgggcgatggtagcgtggaaatgcatgacgttaaaaatcaccgtacctttctgaaacgcacgaaatatgataatctgcatctggaagacctgtttattggcaacaaagtcaatgtgttctctcgtcagctggtgctgatcgattatggcgaccagtacaccgcgcgtcaactgggtagtcgcaaagaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttacctga

SEQ ID NO: 49 - protein, 237 aa, synthetic construct; identical to SEQ ID NO: 29
  REGION 1..237: note = Human NME7-A3 sequence optimized for E. coli expression

SEQ ID NO: 50 - DNA, 408 bp, synthetic construct
  misc_feature 1..408: note = Human NME7-B sequence optimized for E. coli expression
  source 1..408: mol_type = other DNA
atgaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttctga

SEQ ID NO: 51 - protein, 135 aa, synthetic construct; identical to SEQ ID NO: 31
  REGION 1..135: note = Human NME7-B sequence optimized for E. coli expression

SEQ ID NO: 52 - DNA, 423 bp, synthetic construct
  misc_feature 1..423: note = Human NME7-B1 sequence optimized for E. coli expression
  source 1..423: mol_type = other DNA
atgaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataattga

SEQ ID NO: 53 - protein, 140 aa, synthetic construct; identical to SEQ ID NO: 33
  REGION 1..140: note = Human NME7-B1 sequence optimized for E. coli expression

SEQ ID NO: 54 - DNA, 453 bp, synthetic construct
  misc_feature 1..453: note = Human NME7-B2 sequence optimized for E. coli expression
  source 1..453: mol_type = other DNA
atgccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttctga

SEQ ID NO: 55 - protein, 150 aa, synthetic construct; identical to SEQ ID NO: 35
  REGION 1..150: note = Human NME7-B2 sequence optimized for E. coli expression

SEQ ID NO: 56 - DNA, 468 bp, synthetic construct
  misc_feature 1..468: note = Human NME7-B3 sequence optimized for E. coli expression
  source 1..468: mol_type = other DNA
atgccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataattga

SEQ ID NO: 57 - protein, 155 aa, synthetic construct; identical to SEQ ID NO: 37
  REGION 1..155: note = Human NME7-B3 sequence optimized for E. coli expression

SEQ ID NO: 58 - DNA, 861 bp, synthetic construct
  misc_feature 1..861: note = Human NME7-AB, also known as NME7AB sequence optimized for E. coli expression
  source 1..861: mol_type = other DNA
atggaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataattga

SEQ ID NO: 59 - protein, 286 aa, synthetic construct; identical to SEQ ID NO: 39
  REGION 1..286: note = Human NME7-AB, also known as NME7AB sequence optimized for E. coli expression

SEQ ID NO: 60 - DNA, 846 bp, synthetic construct
  misc_feature 1..846: note = Human NME7-AB1, also known as NME7AB1 sequence optimized for E. coli expression
  source 1..846: mol_type = other DNA
atggaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttctga

SEQ ID NO: 61 - protein, 281 aa, synthetic construct; identical to SEQ ID NO: 41
  REGION 1..281: note = Human NME7-AB1, also known as NME7AB1 sequence optimized for E. coli expression

SEQ ID NO: 62 - DNA, 570 bp, synthetic construct
  misc_feature 1..570: note = Mouse NME6
  source 1..570: mol_type = other DNA
atgacctccatcttgcgaagtccccaagctcttcagctcacactagccctgatcaagcctgatgcagttgcccacccactgatcctggaggctgttcatcagcagattctgagcaacaagttcctcattgtacgaacgagggaactgcagtggaagctggaggactgccggaggttttaccgagagcatgaagggcgttttttctatcagcggctggtggagttcatgacaagtgggccaatccgagcctatatccttgcccacaaagatgccatccaactttggaggacactgatgggacccaccagagtatttcgagcacgctatatagccccagattcaattcgtggaagtttgggcctcactgacacccgaaatactacccatggctcagactccgtggtttccgccagcagagagattgcagccttcttccctgacttcagtgaacagcgctggtatgaggaggaggaaccccagctgcggtgtggtcctgtgcactacagtccagaggaaggtatccactgtgcagctgaaacaggaggccacaaacaacctaacaaaacctag

SEQ ID NO: 63 - protein, 189 aa, synthetic construct
  REGION 1..189: note = Mouse NME6
MTSILRSPQALQLTLALIKPDAVAHPLILEAVHQQILSNKFLIVRTRELQWKLEDCRRFYREHEGRFFYQRLVEFMTSGPIRAYILAHKDAIQLWRTLMGPTRVFRARYIAPDSIRGSLGLTDTRNTTHGSDSVVSASREIAAFFPDFSEQRWYEEEEPQLRCGPVHYSPEEGIHCAAETGGHKQPNKT

SEQ ID NO: 64 - DNA, 585 bp, synthetic construct
  misc_feature 1..585: note = Human NME6
  source 1..585: mol_type = other DNA
atgacccagaatctggggagtgagatggcctcaatcttgcgaagccctcaggctctccagctcactctagccctgatcaagcctgacgcagtcgcccatccactgattctggaggctgttcatcagcagattctaagcaacaagttcctgattgtacgaatgagagaactactgtggagaaaggaagattgccagaggttttaccgagagcatgaagggcgttttttctatcagaggctggtggagttcatggccagcgggccaatccgagcctacatccttgcccacaaggatgccatccagctctggaggacgctcatgggacccaccagagtgttccgagcacgccatgtggccccagattctatccgtgggagtttcggcctcactgacacccgcaacaccacccatggttcggactctgtggtttcagccagcagagagattgcagccttcttccctgacttcagtgaacagcgctggtatgaggaggaagagccccagttgcgctgtggccctgtgtgctatagcccagagggaggtgtccactatgtagctggaacaggaggcctaggaccagcctga

SEQ ID NO: 65 - protein, 194 aa, synthetic construct
  REGION 1..194: note = Human NME6
MTQNLGSEMASILRSPQALQLTLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFFPDFSEQRWYEEEEPQLRCGPVCYSPEGGVHYVAGTGGLGPA

SEQ ID NO: 66 - DNA, 525 bp, synthetic construct
  misc_feature 1..525: note = Human NME6 1
  source 1..525: mol_type = other DNA
atgacccagaatctggggagtgagatggcctcaatcttgcgaagccctcaggctctccagctcactctagccctgatcaagcctgacgcagtcgcccatccactgattctggaggctgttcatcagcagattctaagcaacaagttcctgattgtacgaatgagagaactactgtggagaaaggaagattgccagaggttttaccgagagcatgaagggcgttttttctatcagaggctggtggagttcatggccagcgggccaatccgagcctacatccttgcccacaaggatgccatccagctctggaggacgctcatgggacccaccagagtgttccgagcacgccatgtggccccagattctatccgtgggagtttcggcctcactgacacccgcaacaccacccatggttcggactctgtggtttcagccagcagagagattgcagccttcttccctgacttcagtgaacagcgctggtatgaggaggaagagccccagttgcgctgtggccctgtgtga

SEQ ID NO: 67 - protein, 174 aa, synthetic construct
  REGION 1..174: note = Human NME6 1
MTQNLGSEMASILRSPQALQLTLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFFPDFSEQRWYEEEEPQLRCGPV

SEQ ID NO: 68 - DNA, 468 bp, synthetic construct
  misc_feature 1..468: note = Human NME6 2
  source 1..468: mol_type = other DNA
atgctcactctagccctgatcaagcctgacgcagtcgcccatccactgattctggaggctgttcatcagcagattctaagcaacaagttcctgattgtacgaatgagagaactactgtggagaaaggaagattgccagaggttttaccgagagcatgaagggcgttttttctatcagaggctggtggagttcatggccagcgggccaatccgagcctacatccttgcccacaaggatgccatccagctctggaggacgctcatgggacccaccagagtgttccgagcacgccatgtggccccagattctatccgtgggagtttcggcctcactgacacccgcaacaccacccatggttcggactctgtggtttcagccagcagagagattgcagccttcttccctgacttcagtgaacagcgctggtatgaggaggaagagccccagttgcgctgtggccctgtgtga

SEQ ID NO: 69 - protein, 155 aa, synthetic construct
  REGION 1..155: note = Human NME6 2
MLTLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFFPDFSEQRWYEEEEPQLRCGPV

SEQ ID NO: 70 - DNA, 528 bp, synthetic construct
  misc_feature 1..528: note = Human NME6 3
  source 1..528: mol_type = other DNA
atgctcactctagccctgatcaagcctgacgcagtcgcccatccactgattctggaggctgttcatcagcagattctaagcaacaagttcctgattgtacgaatgagagaactactgtggagaaaggaagattgccagaggttttaccgagagcatgaagggcgttttttctatcagaggctggtggagttcatggccagcgggccaatccgagcctacatccttgcccacaaggatgccatccagctctggaggacgctcatgggacccaccagagtgttccgagcacgccatgtggccccagattctatccgtgggagtttcggcctcactgacacccgcaacaccacccatggttcggactctgtggtttcagccagcagagagattgcagccttcttccctgacttcagtgaacagcgctggtatgaggaggaagagccccagttgcgctgtggccctgtgtgctatagcccagagggaggtgtccactatgtagctggaacaggaggcctaggaccagcctga

SEQ ID NO: 71 - protein, 175 aa, synthetic construct
  REGION 1..175: note = Human NME6 3
MLTLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFFPDFSEQRWYEEEEPQLRCGPVCYSPEGGVHYVAGTGGLGPA

SEQ ID NO: 72 - DNA, 585 bp, synthetic construct
  misc_feature 1..585: note = Human NME6 sequence optimized for E. coli expression
  source 1..585: mol_type = other DNA
atgacgcaaaatctgggctcggaaatggcaagtatcctgcgctccccgcaagcactgcaactgaccctggctctgatcaaaccggacgctgttgctcatccgctgattctggaagcggtccaccagcaaattctgagcaacaaatttctgatcgtgcgtatgcgcgaactgctgtggcgtaaagaagattgccagcgtttttatcgcgaacatgaaggccgtttcttttatcaacgcctggttgaattcatggcctctggtccgattcgcgcatatatcctggctcacaaagatgcgattcagctgtggcgtaccctgatgggtccgacgcgcgtctttcgtgcacgtcatgtggcaccggactcaatccgtggctcgttcggtctgaccgatacgcgcaataccacgcacggtagcgactctgttgttagtgcgtcccgtgaaatcgcggcctttttcccggacttctccgaacagcgttggtacgaagaagaagaaccgcaactgcgctgtggcccggtctgttattctccggaaggtggtgtccattatgtggcgggcacgggtggtctgggtccggcatga

SEQ ID NO: 73 - protein, 194 aa, synthetic construct; identical to SEQ ID NO: 65
  REGION 1..194: note = Human NME6 sequence optimized for E. coli expression

SEQ ID NO: 74 - DNA, 525 bp, synthetic construct
  misc_feature 1..525: note = Human NME6 1 sequence optimized for E. coli expression
  source 1..525: mol_type = other DNA
atgacgcaaaatctgggctcggaaatggcaagtatcctgcgctccccgcaagcactgcaactgaccctggctctgatcaaaccggacgctgttgctcatccgctgattctggaagcggtccaccagcaaattctgagcaacaaatttctgatcgtgcgtatgcgcgaactgctgtggcgtaaagaagattgccagcgtttttatcgcgaacatgaaggccgtttcttttatcaacgcctggttgaattcatggcctctggtccgattcgcgcatatatcctggctcacaaagatgcgattcagctgtggcgtaccctgatgggtccgacgcgcgtctttcgtgcacgtcatgtggcaccggactcaatccgtggctcgttcggtctgaccgatacgcgcaataccacgcacggtagcgactctgttgttagtgcgtcccgtgaaatcgcggcctttttcccggacttctccgaacagcgttggtacgaagaagaagaaccgcaactgcgctgtggcccggtctga

SEQ ID NO: 75 - protein, 174 aa, synthetic construct; identical to SEQ ID NO: 67
  REGION 1..174: note = Human NME6 1 sequence optimized for E. coli expression

SEQ ID NO: 76 - DNA, 468 bp, synthetic construct
  misc_feature 1..468: note = Human NME6 2 sequence optimized for E. coli expression
  source 1..468: mol_type = other DNA
atgctgaccctggctctgatcaaaccggacgctgttgctcatccgctgattctggaagcggtccaccagcaaattctgagcaacaaatttctgatcgtgcgtatgcgcgaactgctgtggcgtaaagaagattgccagcgtttttatcgcgaacatgaaggccgtttcttttatcaacgcctggttgaattcatggcctctggtccgattcgcgcatatatcctggctcacaaagatgcgattcagctgtggcgtaccctgatgggtccgacgcgcgtctttcgtgcacgtcatgtggcaccggactcaatccgtggctcgttcggtctgaccgatacgcgcaataccacgcacggtagcgactctgttgttagtgcgtcccgtgaaatcgcggcctttttcccggacttctccgaacagcgttggtacgaagaagaagaaccgcaactgcgctgtggcccggtctga

SEQ ID NO: 77 - protein, 155 aa, synthetic construct; identical to SEQ ID NO: 69
  REGION 1..155: note = Human NME6 2 sequence optimized for E. coli expression

SEQ ID NO: 78 - DNA, 528 bp, synthetic construct
  misc_feature 1..528: note = Human NME6 3 sequence optimized for E. coli expression
  source 1..528: mol_type = other DNA
atgctgaccctggctctgatcaaaccggacgctgttgctcatccgctgattctggaagcggtccaccagcaaattctgagcaacaaatttctgatcgtgcgtatgcgcgaactgctgtggcgtaaagaagattgccagcgtttttatcgcgaacatgaaggccgtttcttttatcaacgcctggttgaattcatggcctctggtccgattcgcgcatatatcctggctcacaaagatgcgattcagctgtggcgtaccctgatgggtccgacgcgcgtctttcgtgcacgtcatgtggcaccggactcaatccgtggctcgttcggtctgaccgatacgcgcaataccacgcacggtagcgactctgttgttagtgcgtcccgtgaaatcgcggcctttttcccggacttctccgaacagcgttggtacgaagaagaagaaccgcaactgcgctgtggcccggtctgttattctccggaaggtggtgtccattatgtggcgggcacgggtggtctgggtccggcatga

SEQ ID NO: 79 - protein, 175 aa, synthetic construct; identical to SEQ ID NO: 71
  REGION 1..175: note = Human NME6 3 sequence optimized for E. coli expression

SEQ ID NO: 80 - DNA, 1306 bp, synthetic construct
  misc_feature 1..1306: note = OriGene-NME7-1 full length
  source 1..1306: mol_type = other DNA
gacgttgtatacgactcctatagggcggccgggaattcgtcgactggatccggtaccgaggagatctgccgccgcgatcgccatgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttctctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataatacgcgtacgcggccgctcgagcagaaactcatctcagaagaggatctggcagcaaatgatatcctggattacaaggatgacgacgataaggtttaa

SEQ ID NO: 81 - protein, 407 aa, synthetic construct
  REGION 1..407: note = OriGene-NME7-1 full length
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDNTRTRRLEQKLISEEDLAANDILDYKDDDDKV

SEQ ID NO: 82 - protein, 376 aa, synthetic construct
  REGION 1..376: note = Abnova NME7-1 Full length
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 83 - protein, 98 aa, synthetic construct
  REGION 1..98: note = Abnova Partial NME7-B
DRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKIL

SEQ ID NO: 84 - DNA, 27 bp, synthetic construct
  misc_feature 1..27: note = Histidine Tag
  source 1..27: mol_type = other DNA
ctcgagcaccaccaccaccaccactga

SEQ ID NO: 85 - DNA, 36 bp, synthetic construct
  misc_feature 1..36: note = Strept II Tag
  source 1..36: mol_type = other DNA
accggttggagccatcctcagttcgaaaagtaatga

SEQ ID NO: 86 - protein, 35 aa, synthetic construct
  REGION 1..35: note = N-10 peptide
QFNQYKTEAASRYNLTISDVSVSDVPFPFSAQSGA

SEQ ID NO: 87 - protein, 35 aa, synthetic construct
  REGION 1..35: note = C-10 peptide
GTINVHDVETQFNQYKTEAASRYNLTISDVSVSDV

SEQ ID NO: 88 - protein, 8 aa, synthetic construct
  REGION 1..8: note = Peptide of NME7AB
LALIKPDA

SEQ ID NO: 89 - protein, 18 aa, synthetic construct
  REGION 1..18: note = Peptide of NME7AB
MMMLSRKEALDFHVDHQS

SEQ ID NO: 90 - protein, 10 aa, synthetic construct
  REGION 1..10: note = Peptide of NME7AB
ALDFHVDHQS

SEQ ID NO: 91 - protein, 14 aa, synthetic construct
  REGION 1..14: note = Peptide of NME7AB
EILRDDAICEWKRL

SEQ ID NO: 92 - protein, 12 aa, synthetic construct
  REGION 1..12: note = Peptide of NME7AB
FNELIQFITTGP

SEQ ID NO: 93 - protein, 8 aa, synthetic construct
  REGION 1..8: note = Peptide of NME7AB
RDDAICEW

SEQ ID NO: 94 - protein, 25 aa, synthetic construct
  REGION 1..25: note = Peptide of NME7AB
SGVARTDASESIRALFGTDGIRNAA

SEQ ID NO: 95 - protein, 9 aa, synthetic construct
  REGION 1..9: note = Peptide of NME7AB
ELFFPSSGG

SEQ ID NO: 96 - protein, 26 aa, synthetic construct
  REGION 1..26: note = Peptide of NME7AB
KFTNCTCCIVKPHAVSEGLLGKILMA

SEQ ID NO: 97 - protein, 36 aa, synthetic construct
  REGION 1..36: note = Peptide of NME7AB
LMAIRDAGFEISAMQMFNMDRVNVEEFYEVYKGVVT

SEQ ID NO: 98 - protein, 15 aa, synthetic construct
  REGION 1..15: note = Peptide of NME7AB
EFYEVYKGVVTEYHD

SEQ ID NO: 99 - protein, 43 aa, synthetic construct
  REGION 1..43: note = Peptide of NME7AB
EIQQNNATKTFREFCGPADPEIARHLRPGTLRAIFGKTKIQNA

SEQ ID NO: 100 - protein, 8 aa, synthetic construct
  REGION 1..8: note = Peptide of NME7AB
YSGPCVAM

SEQ ID NO: 101 - protein, 7 aa, synthetic construct
  REGION 1..7: note = Peptide of NME7AB
FREFCGP

SEQ ID NO: 102 - protein, 23 aa, synthetic construct
  REGION 1..23: note = Peptide of NME7AB
VHCTDLPEDGLLEVQYFFKILDN

SEQ ID NO: 103 - protein, 9 aa, synthetic construct
  REGION 1..9: note = Peptide of NME7AB
IQNAVHCTD

SEQ ID NO: 104 - protein, 20 aa, synthetic construct
  REGION 1..20: note = Peptide of NME7AB
TDLPEDGLLEVQYFFKILDN

SEQ ID NO: 105 - protein, 13 aa, synthetic construct
  REGION 1..13: note = Peptide of NME7AB
PEDGLLEVQYFFK

SEQ ID NO: 106 - protein, 12 aa, synthetic construct
  REGION 1..12: note = Peptide of NME7AB
EIINKAGFTITK

SEQ ID NO: 107 - protein, 16 aa, synthetic construct
  REGION 1..16: note = Peptide of NME7AB
MLSRKEALDFHVDHQS

SEQ ID NO: 108 - protein, 9 aa, synthetic construct
  REGION 1..9: note = Peptide of NME7AB
NELIQFITT

SEQ ID NO: 109 - protein, 14 aa, synthetic construct
  REGION 1..14: note = Peptide of NME7AB
EILRDDAICEWKRL

SEQ ID NO: 110 - protein, 21 aa, synthetic construct
  REGION 1..21: note = Peptide of NME7AB
SGVARTDASESIRALFGTDGI

SEQ ID NO: 111 - protein, 11 aa, synthetic construct
  REGION 1..11: note = Peptide of NME7AB
SGVARTDASES

SEQ ID NO: 112 - protein, 8 aa, synthetic construct
  REGION 1..8: note = Peptide of NME7AB
ALFGTDGI

SEQ ID NO: 113 - protein, 14 aa, synthetic construct
  REGION 1..14: note = Peptide of NME7AB
NCTCCIVKPHAVSE

SEQ ID NO: 114 - protein, 11 aa, synthetic construct
  REGION 1..11: note = Peptide of NME7AB
LGKILMAIRDA

SEQ ID NO: 115 - protein, 16 aa, synthetic construct
  REGION 1..16: note = Peptide of NME7AB
EISAMQMFNMDRVNVE

SEQ ID NO: 116 - protein, 8 aa, synthetic construct
  REGION 1..8: note = Peptide of NME7AB
EVYKGVVT

SEQ ID NO: 117 - protein, 8 aa, synthetic construct
  REGION 1..8: note = Peptide of NME7AB
EYHDMVTE

SEQ ID NO: 118 - protein, 15 aa, synthetic construct
  REGION 1..15: note = Peptide of NME7AB
EFCGPADPEIARHLR

SEQ ID NO: 119 - protein, 12 aa, synthetic construct
  REGION 1..12: note = Peptide of NME7AB
AIFGKTKIQNAV

SEQ ID NO: 120 - protein, 18 aa, synthetic construct
  REGION 1..18: note = Peptide of NME7AB
LPEDGLLEVQYFFKILDN

SEQ ID NO: 121 - protein, 17 aa, synthetic construct
  REGION 1..17: note = Peptide of NME7AB
GPDSFASAAREMELFFP

SEQ ID NO: 122 - protein, 7 aa, synthetic construct
  REGION 1..7: note = Immunizing peptides derived from human NME7
ICEWKRL

SEQ ID NO: 123 - protein, 11 aa, synthetic construct
  REGION 1..11: note = Immunizing peptides derived from human NME7
LGKILMAIRDA

SEQ ID NO: 124 - protein, 10 aa, synthetic construct
  REGION 1..10: note = Immunizing peptides derived from human NME7
HAVSEGLLGK

SEQ ID NO: 125 - protein, 8 aa, synthetic construct
  REGION 1..8: note = Immunizing peptides derived from human NME7
VTEMYSGP

SEQ ID NO: 126 - protein, 9 aa, synthetic construct
  REGION 1..9: note = Immunizing peptides derived from human NME7
NATKTFREF

SEQ ID NO: 127 - protein, 9 aa, synthetic construct
  REGION 1..9: note = Immunizing peptides derived from human NME7
AIRDAGFEI

SEQ ID NO: 128 - protein, 13 aa, synthetic construct
  REGION 1..13: note = Immunizing peptides derived from human NME7
AICEWKRLLGPAN

SEQ ID NO: 129 - protein, 8 aa, synthetic construct
  REGION 1..8: note = Immunizing peptides derived from human NME7
DHQSRPFF

SEQ ID NO: 130 - protein, 13 aa, synthetic construct
  REGION 1..13: note = Immunizing peptides derived from human NME7
AICEWKRLLGPAN

SEQ ID NO: 131 - protein, 8 aa, synthetic construct
  REGION 1..8: note = Immunizing peptides derived from human NME7
VDHQSRPF

SEQ ID NO: 132 - protein, 6 aa, synthetic construct
  REGION 1..6: note = Immunizing peptides derived from human NME7
PDSFAS

SEQ ID NO: 133 - protein, 18 aa, synthetic construct
  REGION 1..18: note = Immunizing peptides derived from human NME7
KAGEIIEIINKAGFTITK

SEQ ID NO: 134 - protein, 29 aa, synthetic construct
  REGION 1..29: note = Immunizing peptides derived from human NME1
MANCERTFIAIKPDGVQRGLVGEIIKRFE

SEQ ID NO: 135 - protein, 8 aa, synthetic construct
  REGION 1..8: note = Immunizing peptides derived from human NME1
VDLKDRPF

SEQ ID NO: 136 - protein, 17 aa, synthetic construct
  REGION 1..17: note = Immunizing peptides derived from human NME1
HGSDSVESAEKEIGLWF

SEQ ID NO: 137 - protein, 25 aa, synthetic construct
  REGION 1..25: note = Immunizing peptides derived from human NME1
ERTFIAIKPDGVQRGLVGEIIKRFE

SEQ ID NO: 138 - protein, 30 aa, synthetic construct
  REGION 1..30: note = Immunizing peptides derived from human NME1
VDLKDRPFFAGLVKYMHSGPVVAMVWEGLN

SEQ ID NO: 139 - protein, 26 aa, synthetic construct
  REGION 1..26: note = Immunizing peptides derived from human NME1
NIIHGSDSVESAEKEIGLWFHPEELV

SEQ ID NO: 140 - protein, 14 aa, synthetic construct
  REGION 1..14: note = Immunizing peptides derived from human NME1
KPDGVQRGLVGEII

SEQ ID NO: 141 - protein, 16 aa, synthetic construct
  REGION 1..16: note = NME7A peptide 1 (A domain)
MLSRKEALDFHVDHQS

SEQ ID NO: 142 - protein, 11 aa, synthetic construct
  REGION 1..11: note = NME7A peptide 2 (A domain)
SGVARTDASES

SEQ ID NO: 143 - protein, 20 aa, synthetic construct
  REGION 1..20: note = NME7B peptide 1 (B domain)
DAGFEISAMQMFNMDRVNVE

SEQ ID NO: 144 - protein, 16 aa, synthetic construct
  REGION 1..16: note = NME7B peptide 2 (B domain)
EVYKGVVTEYHDMVTE

SEQ ID NO: 145 - protein, 29 aa, synthetic construct
  REGION 1..29: note = NME7B peptide 3 (B domain)
AIFGKTKIQNAVHCTDLPEDGLLEVQYFF

SEQ ID NO: 146 - DNA, 1131 bp, synthetic construct
  misc_feature 1..1131: note = Human NME7 a
  source 1..1131: mol_type = other DNA
atgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattag

SEQ ID NO: 147 - protein, 376 aa, synthetic construct; identical to SEQ ID NO: 82
  REGION 1..376: note = Human NME7 a

SEQ ID NO: 148 - DNA, 1023 bp, synthetic construct
  misc_feature 1..1023: note = Human NME7 b
  source 1..1023: mol_type = other DNA
atgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattag

SEQ ID NO: 149 - protein, 340 aa, synthetic construct; identical to SEQ ID NO: 21
  REGION 1..340: note = Human NME7 b

SEQ ID NO: 150 - DNA, 861 bp, synthetic construct
  misc_feature 1..861: note = Human NME7-AB also known as NME7AB
  source 1..861: mol_type = other DNA
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattag

SEQ ID NO: 151 - protein, 286 aa, synthetic construct; identical to SEQ ID NO: 39
  REGION 1..286: note = Human NME7-AB also known as NME7AB

SEQ ID NO: 152 - DNA, 759 bp, synthetic construct
  misc_feature 1..759: note = Human NME7-X1
  source 1..759: mol_type = other DNA
atgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattag

SEQ ID NO: 153 - protein, 252 aa, synthetic construct [sequence omitted]

SEQ ID NO: 154 - DNA, 1128 bp, synthetic construct
  misc_feature 1..1128: note = Human NME7 a (optimized for E coli expression)
  source 1..1128: mol_type = other DNA
atgaatcactccgaacgctttgtttttatcgccgaatggtatgacccgaatgcttccctgctgcgccgctacgaactgctgttttatccgggcgatggtagcgtggaaatgcatgacgttaaaaatcaccgtacctttctgaaacgcacgaaatatgataatctgcatctggaagacctgtttattggcaacaaagtcaatgtgttctctcgtcagctggtgctgatcgattatggcgaccagtacaccgcgcgtcaactgggtagtcgcaaagaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataat

SEQ ID NO: 155 - protein, 378 aa, synthetic construct
  REGION 1..378: note = Human NME7 a (optimized for E coli expression)
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDNTG

SEQ ID NO: 156 - DNA, 1020 bp, synthetic construct
  misc_feature 1..1020: note = Human NME7 b (optimized for E coli expression)
  source 1..1020: mol_type = other DNA
atgcatgacgttaaaaatcaccgtacctttctgaaacgcacgaaatatgataatctgcatctggaagacctgtttattggcaacaaagtcaatgtgttctctcgtcagctggtgctgatcgattatggcgaccagtacaccgcgcgtcaactgggtagtcgcaaagaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataat

SEQ ID NO: 157 - protein, 342 aa, synthetic construct
  REGION 1..342: note = Human NME7 b (optimized for E coli expression)
MHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDNTG

SEQ ID NO: 158 - DNA, 858 bp, synthetic construct
  misc_feature 1..858: note = Human NME7-AB also known as NME7AB (optimized for E coli expression)
  source 1..858: mol_type = other DNA
atggaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataat

SEQ ID NO: 159 - protein, 288 aa, synthetic construct
  REGION 1..288: note = Human NME7-AB also known as NME7AB (optimized for E coli expression)
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDNTG

SEQ ID NO: 160 - DNA, 756 bp, synthetic construct
  misc_feature 1..756: note = Human NME7-X1 (optimized for E coli expression)
  source 1..756: mol_type = other DNA
atgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataat

SEQ ID NO: 161 - protein, 254 aa, synthetic construct
  REGION 1..254: note = Human NME7-X1 (optimized for E coli expression)
MMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDNTG

SEQ ID NO: 162 - protein, 91 aa, synthetic construct
  REGION 1..91: note = DM10 domain of NME7
MNHSERFVFIAEWYDPNASLLRRYELLFYPGDGSVEMHDVKNHRTFLKRTKYDNLHLEDLFIGNKVNVFSRQLVLIDYGDQYTARQLGSRK

SEQ ID NO: 163 - protein, 43 aa, synthetic construct
  REGION 1..43: note = Fragment or variation of PSMGFR peptide
SNIKFRPGSVVVQLTLAFREGTINVHDVETQFNQYKTEAASRY

SEQ ID NO: 164 - protein, 35 aa, synthetic construct
  REGION 1..35: note = Fragment or variation of PSMGFR peptide
SVVVQLTLAFREGTINVHDVETQFNQYKTEAASRY

SEQ ID NO: 165 - protein, 24 aa, synthetic construct
  REGION 1..24: note = Fragment or variation of PSMGFR peptide
VQLTLAFREGTINVHDVETQFNQY

SEQ ID NO: 166 - protein, 24 aa, synthetic construct
  REGION 1..24: note = Fragment or variation of PSMGFR peptide
SNIKFRPGSVVVQLTLAFREGTIN

SEQ ID NO: 167 - protein, 38 aa, synthetic construct
  REGION 1..38: note = Fragment or variation of PSMGFR peptide
SNIKFRPGSVVVQLTLAFREGTINVHDVETQFNQYKTE

SEQ ID NO: 168 - protein, 45 aa, synthetic construct
  REGION 1..45: note = Fragment or variation of PSMGFR peptide
VQLTLAFREGTINVHDVETQFNQYKTEAASRYNLTISDVSVSDVP

SEQ ID NO: 169 - protein, 29 aa, synthetic construct
  REGION 1..29: note = Ser of NME7B peptide 3 (B domain)
AIFGKTKIQNAVHSTDLPEDGLLEVQYFF

SEQ ID NO: 170 - protein, 35 aa, synthetic construct; identical to SEQ ID NO: 86
  REGION 1..35: note = N-10 peptide

SEQ ID NO: 171 - protein, 35 aa, synthetic construct; identical to SEQ ID NO: 87
  REGION 1..35: note = C-10 peptide

SEQ ID NO: 172 - protein, 119 aa, synthetic construct
  REGION 1..119: note = 8F9A5A1H heavy chain
IQLVQSGPELKKPGETVKISCKASGYTFTNYGMNWVKQAPGKGLKWMGWINTYTGEPTYVDDFKGRFAFSLETSATTAYLQINNLKNEDTSTYFCARLRGIRPGPLAYWGQGTLVTVSA

SEQ ID NO: 173 - protein, 121 aa, synthetic construct
  REGION 1..121: note = 8F9A4P3H heavy chain
VQLQQSGPELVKPGASVKISCKTSGNTFTEYTMHWVKQSHGKSLEWIGGFNPNNGVTNYNQKFKGKATLTVDKSSSTAYMELRSLTSEDSAVYYCARRYYHSLYVFYFDYWGQGTTLTVSS

SEQ ID NO: 174 - protein, 116 aa, synthetic construct
  REGION 1..116: note = 5D9E2B11H
VQLQQSGPDLVKPGTSVKISCKTSGNTFTEYTMHWVKQSHGKSLEWIGGFNPNNGVTNYNQKFKGKATLTVDKSSSTAYMELRSLTSEDSAVYYCARRYYHSTYVFYFDSWGQGTT

SEQ ID NO: 175 - protein, 121 aa, synthetic construct
  REGION 1..121: note = 5D9E10E4H
VQLQQSGPDLVKPGTSVKISCKTSGNTFTEYTMHWVKQSHGKSLEWIGGFNPNNGVTNYNQKFKGKATLTVDKSSSTAYMELRSLTSEDSAVYYCARRYYHSTYVFYFDSWGQGTTLTVSS

SEQ ID NO: 176 - protein, 121 aa, synthetic construct; identical to SEQ ID NO: 175
  REGION 1..121: note = 5D9G2C4H

SEQ ID NO: 177 - protein, 121 aa, synthetic construct; identical to SEQ ID NO: 175
  REGION 1..121: note = 5F3A5D4H

SEQ ID NO: 178 - protein, 121 aa, synthetic construct; identical to SEQ ID NO: 175
  REGION 1..121: note = 8H5H5G4H

SEQ ID NO: 179 - protein, 121 aa, synthetic construct; identical to SEQ ID NO: 173
  REGION 1..121: note = 8F9A4A3H

SEQ ID NO: 180 - protein, 116 aa, synthetic construct; identical to SEQ ID NO: 174
  REGION 1..116: note = 5D9E2B11H

SEQ ID NO: 181 - protein, 121 aa, synthetic construct; identical to SEQ ID NO: 175
  REGION 1..121: note = 5D9E10E4H

SEQ ID NO: 182 - protein, 121 aa, synthetic construct; identical to SEQ ID NO: 175
  REGION 1..121: note = 5D9G2C4H

SEQ ID NO: 183 - protein, 121 aa, synthetic construct; identical to SEQ ID NO: 175
  REGION 1..121: note = 5F3A5D4H

SEQ ID NO: 184 - protein, 121 aa, synthetic construct; identical to SEQ ID NO: 175
  REGION 1..121: note = 8H5H5G4H

SEQ ID NO: 185 - protein, 108 aa, synthetic construct
  REGION 1..108: note = 8F9A4P3L
ETTVTQSPASLSMAIGEKVTIRCITSTDIDDDMNWYQQKPGEPPKLLISEGNTLRPGVPSRFSSSGYGTDFVFTIENMLSEDVADYYCLQSDNLPLTFGSGTKLEIKR

SEQ ID NO: 186 - protein, 108 aa, synthetic construct
  REGION 1..108: note = 5D9E2B11L
DIQMTQTTSSLSASLGDRVTISCSASQGISNYLNWFQQKPDGTIKLLIYYTSSLHSGVPSRFSGSGSGTDYSLTISNVEPEDIATYYCQQYSKLPYTFGGGTKLEIKR

SEQ ID NO: 187 - protein, 108 aa, synthetic construct; identical to SEQ ID NO: 186
  REGION 1..108: note = 5D9E10E4L

SEQ ID NO: 188 - protein, 108 aa, synthetic construct; identical to SEQ ID NO: 186
  REGION 1..108: note = 5D9G2C4L

SEQ ID NO: 189 - protein, 108 aa, synthetic construct; identical to SEQ ID NO: 186
  REGION 1..108: note = 5F3A5D4L

SEQ ID NO: 190 - protein, 108 aa, synthetic construct; identical to SEQ ID NO: 186
  REGION 1..108: note = 8H5H5G4L

SEQ ID NO: 191 - protein, 107 aa, synthetic construct
  REGION 1..107: note = 8F9A5A1L
EILLTQSPAIIAASPGEKVTITCSASSSVSYMNWYQQKPGSSPKIWIYGISNLASGVPARFSGSGSGTSFSFTINSMEAEDVATYYCQQRSSYPPTFGGGTKLEIKR

SEQ ID NO: 192 - protein, 108 aa, synthetic construct; identical to SEQ ID NO: 186
  REGION 1..108: note = 5D9E2B11L

SEQ ID NO: 193 - protein, 108 aa, synthetic construct; identical to SEQ ID NO: 186
  REGION 1..108: note = 5D9E10E4L

SEQ ID NO: 194 - protein, 108 aa, synthetic construct; identical to SEQ ID NO: 186
  REGION 1..108: note = 5D9G2C4L

SEQ ID NO: 195 - protein, 108 aa, synthetic construct; identical to SEQ ID NO: 186
  REGION 1..108: note = 5F3A5D4L

SEQ ID NO: 196 - protein, 108 aa, synthetic construct; identical to SEQ ID NO: 186
  REGION 1..108: note = 8H5H5G4L

SEQ ID NO: 197 - protein, 108 aa, synthetic construct; identical to SEQ ID NO: 185
  REGION 1..108: note = 8F9A4P3L

SEQ ID NO: 198 - protein, 107 aa, synthetic construct; identical to SEQ ID NO: 191
  REGION 1..107: note = 8F9A5A1L

SEQ ID NO: 199 - protein, 131 aa, synthetic construct
  REGION 1..131: note = NME7A partial sequence
EKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFF

SEQ ID NO: 200 - protein, 152 aa, synthetic construct; identical to SEQ ID NO: 19
  REGION 1..152: note = NME2 partial sequence

SEQ ID NO: 201 - protein, 134 aa, synthetic construct
  REGION 1..134: note = NME7B partial sequence
NCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 202 - protein, 152 aa, synthetic construct; identical to SEQ ID NO: 19
  REGION 1..152: note = NME2 partial sequence

SEQ ID NO: 203 - protein, 131 aa, synthetic construct; identical to SEQ ID NO: 199
  REGION 1..131: note = NME7A partial sequence

SEQ ID NO: 204 - protein, 130 aa, synthetic construct
  REGION 1..130: note = NME3 partial sequence
ERTFLAVKPDGVQRRLVGEIVRRFERKGFKLVALKLVQASEELLREHYVELRERPFYSRLVKYMGSGPVVAMVWQGLDVVRASRALIGATDPGDATPGTIRGDFCVEVGKNVIHGSDSVESAQREIALWF

SEQ ID NO: 205 - protein, 134 aa, synthetic construct; identical to SEQ ID NO: 201
  REGION 1..134: note = NME7B partial sequence

SEQ ID NO: 206 - protein, 169 aa, synthetic construct
  REGION 1..169: note = NME3 partial sequence
MICLVLTIFANLFPSAYSGVNERTFLAVKPDGVQRRLVGEIVRRFERKGFKLVALKLVQASEELLREHYVELRERPFYSRLVKYMGSGPVVAMVWQGLDVVRASRALIGATDPGDATPGTIRGDFCVEVGKNVIHGSDSVESAQREIALWFREDELLCWEDSAGHWLYE

SEQ ID NO: 207 - protein, 131 aa, synthetic construct; identical to SEQ ID NO: 199
  REGION 1..131: note = NME7A partial sequence

SEQ ID NO: 208 - protein, 130 aa, synthetic construct
  REGION 1..130: note = NME4 partial sequence
ERTLVAVKPDGVQRRLVGDVIQRFERRGFTLVGMKMLQAPESVLAEHYQDLRRKPFYPALIRYMSSGPVVAMVWEGYNVVRASRAMIGHTDSAEAAPGTIRGDFSVHISRNVIHASDSVEGAQREIQLWF

SEQ ID NO: 209 - protein, 132 aa, synthetic construct
  REGION 1..132: note = NME7B partial sequence
TCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 210 - protein, 128 aa, synthetic construct
  REGION 1..128: note = NME4 partial sequence
TLVAVKPDGVQRRLVGDVIQRFERRGFTLVGMKMLQAPESVLAEHYQDLRRKPFYPALIRYMSSGPVVAMVWEGYNVVRASRAMIGHTDSAEAAPGTIRGDFSVHISRNVIHASDSVEGAQREIQLWF

SEQ ID NO: 211 - protein, 131 aa, synthetic construct; identical to SEQ ID NO: 199
  REGION 1..131: note = NME7A partial sequence

SEQ ID NO: 212 - protein, 131 aa, synthetic construct
  REGION 1..131: note = NME5 partial sequence
EKTLAIIKPDIVDKEEEIQDIILRSGFTIVQRRKLRLSPEQCSNFYVEKYGKMFFPNLTAYMSSGPLVAMILARHKAISYWLELLGPNNSLVAKETHPDSLRAIYGTDDLRNALHGSNDFAAAEREIRFMF

SEQ ID NO: 213 - protein, 132 aa, synthetic construct; identical to SEQ ID NO: 209
  REGION 1..132: note = NME7B partial sequence

SEQ ID NO: 214 - protein, 129 aa, synthetic construct
  REGION 1..129: note = NME5 partial sequence
TLAIIKPDIVDKEEEIQDIILRSGFTIVQRRKLRLSPEQCSNFYVEKYGKMFFPNLTAYMSSGPLVAMILARHKAISYWLELLGPNNSLVAKETHPDSLRAIYGTDDLRNALHGSNDFAAAEREIRFMF

SEQ ID NO: 215 - protein, 129 aa, synthetic construct
  REGION 1..129: note = NME7A partial sequence
TLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFF

SEQ ID NO: 216 - protein, 132 aa, synthetic construct
  REGION 1..132: note = NME6 partial sequence
TLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFF

SEQ ID NO: 217 - protein, 132 aa, synthetic construct; identical to SEQ ID NO: 209
  REGION 1..132: note = NME7B partial sequence

SEQ ID NO: 218 - protein, 132 aa, synthetic construct; identical to SEQ ID NO: 216
  REGION 1..132: note = NME6 partial sequence

SEQ ID NO: 219 - protein, 131 aa, synthetic construct; identical to SEQ ID NO: 199
  REGION 1..131: note = NME7A partial sequence

SEQ ID NO: 220 - protein, 133 aa, synthetic construct
  REGION 1..133: note = NME8 partial sequence
EKTLALLRPNLFHERKDDVLRIIKDEDFKILEQRQVVLSEKEAQALCKEYENEDYFNKLIENMTSGPSLALVLLRDNGLQYWKQLLGPRTVEEAIEYFPESLCAQFAMDSLPVNQLYGSDSLETAEREIQHFF

SEQ ID NO: 221 - protein, 127 aa, synthetic construct
  REGION 1..127: note = NME7A partial sequence
EKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREM

SEQ ID NO: 222 - protein, 127 aa, synthetic construct
  REGION 1..127: note = NME8 partial sequence
QSTLGLIKPHATSEQREQILKIVKEAGFDLTQVKKMFLTPEQIEKIYPKVTGKDFYKDLLEMLSVGPSMVMILTKWNAVAEWRRLMGPTDPEEAKLLSPDSIRAQFGISKLKNIVHGASNAYEAKEV

SEQ ID NO: 223 - protein, 63 aa, synthetic construct
  REGION 1..63: note = NME7A partial sequence
TLALIKPDAISKAGEIIEIINKAGFTITKLKMMMLSRKEALDFHVDHQSRPFFNELIQFITTG

SEQ ID NO: 224 - protein, 65 aa, synthetic construct
  REGION 1..65: note = NME8 partial sequence
SIAIIKPDAVISKKVLEIKRKITKAGFIIEAEHKTVLTEEQVVNFYSRIADQCDFEEFVSFMTSG

SEQ ID NO: 225 - protein, 116 aa, synthetic construct
  REGION 1..116: note = NME7B partial sequence
TCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVH

SEQ ID NO: 226 - protein, 114 aa, synthetic construct
  REGION 1..114: note = NME8 partial sequence
TLGLIKPHATSEQREQILKIVKEAGFDLTQVKKMFLTPEQIEKIYPKVTGKDFYKDLLEMLSVGPSMVMILTKWNAVAEWRRLMGPTDPEEAKLLSPDSIRAQFGISKLKNIVH

SEQ ID NO: 227 - protein, 115 aa, synthetic construct
  REGION 1..115: note = NME7B partial sequence
ILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 228 - protein, 115 aa, synthetic construct
  REGION 1..115: note = NME8 partial sequence
VLRIIKDEDFKILEQRQVVLSEKEAQALCKEYENEDYFNKLIENMTSGPSLALVLLRDNGLQYWKQLLGPRTVEEAIEYFPESLCAQFAMDSLPVNQLYGSDSLETAEREIQHFF

SEQ ID NO: 229 - protein, 100 aa, synthetic construct
  REGION 1..100: note = NME7B partial sequence
IVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLR

SEQ ID NO: 230 - protein, 110 aa, synthetic construct
  REGION 1..110: note = NME8 partial sequence
IIKPDAVISKKVLEIKRKITKAGFIIEAEHKTVLTEEQVVNFYSRIADQCDFEEFVSFMTSGLSYILVVSQGSKHNPPSEETEPQTDTEPNERSEDQPEVEAQVTPGMMK

SEQ ID NO: 231 - protein, 174 aa, synthetic construct
  REGION 1..174: note = NME9
MLSSKGLTVVDVYQGWCGPCKPVVSLFQKMRIEVGLDLLHFALAEADRLDVLEKYRGKCEPTFLFYAIKDEALSDEDECVSHGKNNGEDEDMVSSERTCTLAIIKPDAVAHGKTDEIIMKIQEAGFEILTNEERTMTEAEVRLFYQHKAGESPSSVRHRNALQCRPWKPGQRRC

SEQ ID NO: 232 - protein, 58 aa, synthetic construct
  REGION 1..58: note = NME7A partial sequence
TLALIKPDAISKAGEIIEIINKAGFTITKLKMMMLSRKEALDFHAMEILRDDAICEWK

SEQ ID NO: 233 - protein, 60 aa, synthetic construct
  REGION 1..60: note = NME9 partial sequence
TLAIIKPDAVAHGKTDEIIMKIQEAGFEILTNEERTMTEAEVRLFYTLAIIKPDAVAHGK

SEQ ID NO: 234 - protein, 31 aa, synthetic construct
  REGION 1..31: note = NME7A partial sequence
ISKAGEIIEIINKAGFTITKLKMMMLSRKEA

SEQ ID NO: 235 - protein, 29 aa, synthetic construct
  REGION 1..29: note = NME9 partial sequence
IQEAGFEILTNEERTMTEAEVRLFYQHKA

SEQ ID NO: 236 - protein, 53 aa, synthetic construct
  REGION 1..53: note = NME7B partial sequence
NCTCCIVKPHAVSEGLLGKILMAIRDAGFEISAMQMFNMDRVNVEEFYEVYKG

SEQ ID NO: 237 - protein, 53 aa, synthetic construct
  REGION 1..53: note = NME9 partial sequence
TCTLAIIKPDAVAHGKTDEIIMKIQEAGFEILTNEERTMTEAEVRLFYQHKAG

SEQ ID NO: 238 - protein, 350 aa, synthetic construct
  REGION 1..350: note = NME10
MGCFFSKRRKADKESRPENEEERPKQYSWDQREKVDPKDYMFSGLKDETVGRLPGTVAGQQFLIQDCENCNIYIFDHSATVTIDDCTNCIIFLGPVKGSVFFRNCRDCKCTLACQQFRVRDCRKLEVFLCCATQPIIESSSNIKFGCFQWYYPELAFQFKDAGLSIFNNTWSNIHDFTPVSGELNWSLLPEDAVVQDYVPIPTTEELKAVRVSTEANRSIVPISRGQRQKSSDESCLVVLFAGDYTIANARKLIDEMVGKGFFLVQTKEVSMKAEDAQRVFREKAPDFLPLLNKGPVIALEFNGDGAVEVCQLIVNEIFNGTKMFVSESKETASGDVDSFYNFADIQMGI

SEQ ID NO: 239 - protein, 68 aa, synthetic construct
  REGION 1..68: note = NME7A partial sequence
AISKAGEIIEIINKAGFTITKLKMMMLSRKEALDFHVDHQSRPFFNELIQFITTGPIIAMEILRDDAI

SEQ ID NO: 240 - protein, 63 aa, synthetic construct
  REGION 1..63: note = NME10 partial sequence
TIANARKLIDEMVGKGFFLVQTKEVSMKAEDAQRVFREKAPDFLPLLNKGPVIALEFNGDGAV

SEQ ID NO: 241 - protein, 43 aa, synthetic construct
  REGION 1..43: note = NME7A partial sequence
PIIAMEILRDDAICEWKRLLGPANSGVARTDASESIRALFGTD

SEQ ID NO: 242 - protein, 45 aa, synthetic construct
  REGION 1..45: note = NME10 partial sequence
PIPTTEELKAVRVSTEANRSIVPISRGQRQKSSDESCLVVLFAGD

SEQ ID NO: 243 - protein, 74 aa, synthetic construct
  REGION 1..74: note = NME7A partial sequence
IKPDAISKAGEIIEIINKAGFTITKLKMMMLSRKEALDFHVDHQSRPFFNELIQFITTGPIIAMEILRDDAICE

SEQ ID NO: 244 - protein, 75 aa, synthetic construct
  REGION 1..75: note = NME10 partial sequence
VDPKDYMFSGLKDETVGRLPGTVAGQQFLIQDCENCNIYIFDHSATVTIDDCTNCIIFLGPVKGSVFFRNCRDCK

SEQ ID NO: 245 - protein, 47 aa, synthetic construct
  REGION 1..47: note = NME7B partial sequence
CCIVKPHAVSEGLLGKILMAIRDAGFEISAMQMFNMDRVNVEEFYEV

SEQ ID NO: 246 - protein, 51 aa, synthetic construct
  REGION 1..51: note = NME10 partial sequence
CCATQPIIESSSNIKFGCFQWYYPELAFQFKDAGLSIFNNTWSNIHDFTPV

SEQ ID NO: 247 - protein, 16 aa, synthetic construct
  REGION 1..16: note = NME2A1
RASEEHLKQHYIDLKD

SEQ ID NO: 248 - protein, 8 aa, synthetic construct
  REGION 1..8: note = NME2A2
PADSKPGT

SEQ ID NO: 249 - protein, 20 aa, synthetic construct
  REGION 1..20: note = NME2B1
QKGFRLVAMKFLRASEEHLK

SEQ ID NO: 250 - protein, 14 aa, synthetic construct
  REGION 1..14: note = NME2B2
IDLKDRPFPGLVKY

SEQ ID NO: 251 - protein, 29 aa, synthetic construct
  REGION 1..29: note = NME2B3
GDFCIQVGRNIIHGSDSVKSAEKEISLWF

SEQ ID NO: 252 - protein, 16 aa, synthetic construct
  REGION 1..16: note = NME3A1
QASEELLREHYVELRE

SEQ ID NO: 253 - protein, 8 aa, synthetic construct
  REGION 1..8: note = NME3A1
PGDATPGT

SEQ ID NO: 254 - protein, 20 aa, synthetic construct
  REGION 1..20: note = NME3B1
RKGFKLVALKLVQASEELLR

SEQ ID NO: 255 - protein, 15 aa, synthetic construct
  REGION 1..15: note = NME3B2
VELRERPFYSRLVKY

SEQ ID NO: 256 - protein, 29 aa, synthetic construct
  REGION 1..29: note = NME3B3
GDFCVEVGKNVIHGSDSVESAQREIALWF

SEQ ID NO: 257 - protein, 16 aa, synthetic construct
  REGION 1..16: note = NME4A1
QAPESVLAEHYQDLRR

SEQ ID NO: 258 - protein, 8 aa, synthetic construct
  REGION 1..8: note = NME4A2
SAEAAPGT

SEQ ID NO: 259 - protein, 20 aa, synthetic construct
  REGION 1..20: note = NME4B1
RRGFTLVGMKMLQAPESVLA

SEQ ID NO: 260 - protein, 15 aa, synthetic construct
  REGION 1..15: note = NME4B2
QDLRRKPFYPALIRY

SEQ ID NO: 261 - protein, 29 aa, synthetic construct
  REGION 1..29: note = NME4B3
GDFSVHISRNVIHASDSVEGAQREIQLWF

SEQ ID NO: 262 - protein, 16 aa, synthetic construct
  REGION 1..16: note = NME5A1
RLSPEQCSNFYVEKYG

SEQ ID NO: 263 - protein, 11 aa, synthetic construct
  REGION 1..11: note = NME5A2
SLVAKETHPDS

SEQ ID NO: 264 - protein, 20 aa, synthetic construct
  REGION 1..20: note = NME5B1
RSGFTIVQRRKLRLSPEQCS

SEQ ID NO: 265 - protein, 15 aa, synthetic construct
  REGION 1..15: note = NME5B2
VEKYGKMFFPNLTAY

SEQ ID NO: 266 - protein, 29 aa, synthetic construct
  REGION 1..29: note = NME5B3
AIYGTDDLRNALHGSNDFAAAEREIRFMF

SEQ ID NO: 267 - protein, 16 aa, synthetic construct
  REGION 1..16: note = NME6A1
LWRKEDCQRFYREHEG

SEQ ID NO: 268 - protein, 11 aa, synthetic construct
  REGION 1..11: note = NME6A2
VFRARHVAPDS

SEQ ID NO: 269 - protein, 20 aa, synthetic construct
  REGION 1..20: note = NME6B1
SNKFLIVRMRELLWRKEDCQ

SEQ ID NO: 270 - protein, 15 aa, synthetic construct
  REGION 1..15: note = NME6B2
REHEGRFFYQRLVEF

SEQ ID NO: 271 - protein, 29 aa, synthetic construct
  REGION 1..29: note = NME6B3
GSFGLTDTRNTTHGSDSVVSASREIAAFF

SEQ ID NO: 272 - protein, 16 aa, synthetic construct
  REGION 1..16: note = NME8A1
VLSEKEAQALCKEYEN

SEQ ID NO: 273 - protein, 11 aa, synthetic construct
  REGION 1..11: note = NME8A2
VEEAIEYFPES

SEQ ID NO: 274 - protein, 16 aa, synthetic construct
  REGION 1..16: note = NME8A3
FLTPEQIEKIYPKVTG

SEQ ID NO: 275 - protein, 11 aa, synthetic construct
  REGION 1..11: note = NME8A4
PEEAKLLSPDS

SEQ ID NO: 276 - protein, 16 aa, synthetic construct
  REGION 1..16: note = NME8A5
VLTEEQVVNFYSRIAD

SEQ ID NO: 277 - protein, 20 aa, synthetic construct
  REGION 1..20: note = NME8B1
EAGFDLTQVKKMFLTPEQIE

SEQ ID NO: 278 - protein, 15 aa, synthetic construct
  REGION 1..15: note = NME8B2
PKVTGKDFYKDLLEM

SEQ ID NO: 279 - protein, 13 aa, synthetic construct
  REGION 1..13: note = NME8B3
AQFGISKLKNIVH

SEQ ID NO: 280 - protein, 20 aa, synthetic construct
  REGION 1..20: note = NME8B4
DEDFKILEQRQVVLSEKEAQ

SEQ ID NO: 281 - protein, 15 aa, synthetic construct
  REGION 1..15: note = NME8B5
KEYENEDYFNKLIEN

SEQ ID NO: 282 - protein, 30 aa, synthetic construct
  REGION 1..30: note = NME8B6
AQFAMDSLPVNQLYGSDSLETAEREIQHFF

SEQ ID NO: 283 - protein, 20 aa, synthetic construct
  REGION 1..20: note = NME8B7
KAGFIIEAEHKTVLTEEQVV

SEQ ID NO: 284 - protein, 15 aa, synthetic construct
  REGION 1..15: note = NME8B8
SRIADQCDFEEFVSF

SEQ ID NO: 285 - protein, 11 aa, synthetic construct
  REGION 1..11: note = NME9A1
TMTEAEVRLFY

SEQ ID NO: 286 - protein, 20 aa, synthetic construct
  REGION 1..20: note = NME9B1
EAGFEILTNEERTMTEAEVR

SEQ ID NO: 287 - protein, 14 aa, synthetic construct
  REGION 1..14: note = NME10A1
SMKAEDAQRVFREK

SEQ ID NO: 288 - protein, 10 aa, synthetic construct
  REGION 1..10: note = NME10A2
GQRQKSSDES

SEQ ID NO: 289 - protein, 16 aa, synthetic construct
  REGION 1..16: note = NME10A3
IQDCENCNIYIFDHSA

SEQ ID NO: 290 - protein, 22 aa, synthetic construct
  REGION 1..22: note = NME10B1
ELAFQFKDAGLSIFNNTWSNIH

SEQ ID NO: 291 - protein, 23 aa, synthetic construct
  REGION 1..23: note = NME2A1
RASEEHLKQHYIDLKDRPFFPGL

SEQ ID NO: 292 - protein, 18 aa, synthetic construct
  REGION 1..18: note = NME2A2
LGETNPADSKPGTIRGDF

SEQ ID NO: 293 - protein, 34 aa, synthetic construct
  REGION 1..34: note = NME2B1
GLVGEIIKRFEQKGFRLVAMKFLRASEEHLKQHY

SEQ ID NO: 294 - protein, 25 aa, synthetic construct
  REGION 1..25: note = NME2B2
YIDLKDRPFFPGLVKYMNSGPVVAM

SEQ ID NO: 295 - protein, 34 aa, synthetic construct
  REGION 1..34: note = NME2B3
PGTIRGDFCIQVGRNIIHGSDSVKSAEKEISLWF

SEQ ID NO: 296 - protein, 27 aa, synthetic construct
  REGION 1..27: note = NME3A1
LKLVQASEELLREHYVELRERPFYSRL

SEQ ID NO: 297 - protein, 19 aa, synthetic construct
  REGION 1..19: note = NME3A1
LIGATDPGDATPGTIRGDF

SEQ ID NO: 298 - protein, 31 aa, synthetic construct
  REGION 1..31: note = NME3B1
LVGEIVRRFERKGFKLVALKLVQASEELLRE

SEQ ID NO: 299 - protein, 27 aa, synthetic construct
  REGION 1..27: note = NME3B2
EHYVELRERPFYSRLVKYMGSGPVVAM

SEQ ID NO: 300 - protein, 34 aa, synthetic construct
  REGION 1..34: note = NME3B3
PGTIRGDFCVEVGKNVIHGSDSVESAQREIALWF

SEQ ID NO: 301 - protein, 29 aa, synthetic construct
  REGION 1..29: note = NME4A1
GFTLVGMKMLQAPESVLAEHYQDLRRKPF

SEQ ID NO: 302 - protein, 17 aa, synthetic construct
  REGION 1..17: note = NME4A2
GHTDSAEAAPGTIRGDF

SEQ ID NO: 303 - protein, 33 aa, synthetic construct
  REGION 1..33: note = NME4B1
LVGDVIQRFERRGFTLVGMKMLQAPESVLAEHY

SEQ ID NO: 304 - protein, 27 aa, synthetic construct
  REGION 1..27: note = NME4B2
EHYQDLRRKPFYPALIRYMSSGPVVAM

SEQ ID NO: 305 - protein, 34 aa, synthetic construct
  REGION 1..34: note = NME4B3
PGTIRGDFSVHISRNVIHASDSVEGAQREIQLWF

SEQ ID NO: 306 - protein, 30 aa, synthetic construct
  REGION 1..30: note = NME5A1
GFTIVQRRKLRLSPEQCSNFYVEKYGKMFF

SEQ ID NO: 307 - protein, 25 aa, synthetic construct
  REGION 1..25: note = NME5A2
LLGPNNSLVAKETHPDSLRAIYGTD

SEQ ID NO: 308 - protein, 29 aa, synthetic construct
  REGION 1..29: note = NME5B1
IQDIILRSGFTIVQRRKLRLSPEQCSNFY

SEQ ID NO: 309 - protein, 26 aa, synthetic construct
  REGION 1..26: note = NME5B2
FYVEKYGKMFFPNLTAYMSSGPLVAM

SEQ ID NO: 310 - protein, 34 aa, synthetic construct
  REGION 1..34: note = NME5B3
PDSLRAIYGTDDLRNALHGSNDFAAAEREIRFMF

SEQ ID NO: 311 - protein, 32 aa, synthetic construct
  REGION 1..32: note = NME6A1
FLIVRMRELLWRKEDCQRFYREHEGRFFYQRL

SEQ ID NO: 312 - protein, 23 aa, synthetic construct
  REGION 1..23: note = NME6A2
LMGPTRVFRARHVAPDSIRGSFG

SEQ ID NO: 313 - protein, 25 aa, synthetic construct
  REGION 1..25: note = NME6B1
ILSNKFLIVRMRELLWRKEDCQRFY

SEQ ID NO: 314 - protein, 25 aa, synthetic construct
  REGION 1..25: note = NME6B2
FYREHEGRFFYQRLVEFMASGPIRA

SEQ ID NO: 315 - protein, 39 aa, synthetic construct
  REGION 1..39: note = NME6B3
ARHVAPDSIRGSFGLTDTRNTTHGSDSVVSASREIAAFF

SEQ ID NO: 316 - protein, 33 aa, synthetic construct
  REGION 1..33: note = NME8A1
FKILEQRQVVLSEKEAQALCKEYENEDYFNKLI

SEQ ID NO: 317 - protein, 28 aa, synthetic construct
  REGION 1..28: note = NME8A2
WKQLLGPRTVEEAIEYFPESLCAQFAMD

SEQ ID NO: 318 - protein, 34 aa, synthetic construct
  REGION 1..34: note = NME8A3
AGFDLTQVKKMFLTPEQIEKIYPKVTGKDFYKDL

SEQ ID NO: 319 - protein, 27 aa, synthetic construct
  REGION 1..27: note = NME8A4
EWRRLMGPTDPEEAKLLSPDSIRAQFG

SEQ ID NO: 320 - protein, 34 aa, synthetic construct
  REGION 1..34: note = NME8A5
KAGFIIEAEHKTVLTEEQVVNFYSRIADQCDFEE

SEQ ID NO: 321 - protein, 29 aa, synthetic construct
  REGION 1..29: note = NME8B1
ILKIVKEAGFDLTQVKKMFLTPEQIEKIY

SEQ ID NO: 322 - protein, 21 aa, synthetic construct
  REGION 1..21: note = NME8B2
YPKVTGKDFYKDLLEMLSVGP

SEQ ID NO: 323 - protein, 27 aa, synthetic construct
  REGION 1..27: note = NME8B3
DPEEAKLLSPDSIRAQFGISKLKNIVH

SEQ ID NO: 324 - protein, 25 aa, synthetic construct
  REGION 1..25: note = NME8B4
LRIIKDEDFKILEQRQVVLSEKEAQ

SEQ ID NO: 325 - protein, 23 aa, synthetic construct
  REGION 1..23: note = NME8B5
KEYENEDYFNKLIENMTSGPSLA

SEQ ID NO: 326 - protein, 35 aa, synthetic construct
  REGION 1..35: note = NME8B6
PESLCAQFAMDSLPVNQLYGSDSLETAEREIQHFF

SEQ ID NO: 327 - protein, 29 aa, synthetic construct
  REGION 1..29: note = NME8B7
IKRKITKAGFIIEAEHKTVLTEEQVVNFY

SEQ ID NO: 328 - protein, 21 aa, synthetic construct
  REGION 1..21: note = NME8B8
FYSRIADQCDFEEFVSFMTSG

SEQ ID NO: 329 - protein, 22 aa, synthetic construct
  REGION 1..22: note = NME9A1
AGFEILTNEERTMTEAEVRLFY

SEQ ID NO: 330 - protein, 29 aa, synthetic construct
  REGION 1..29: note = NME9B1
IIMKIQEAGFEILTNEERTMTEAEVRLFY

SEQ ID NO: 331 - protein, 26 aa, synthetic construct
  REGION 1..26: note = NME10A1
GFFLVQTKEVSMKAEDAQRVFREKAP

SEQ ID NO: 332 - protein, 30 aa, synthetic construct
  REGION 1..30: note = NME10A2
EANRSIVPISRGQRQKSSDESCLVVLFAGD

SEQ ID NO: 333 - protein, 16 aa, synthetic construct
  REGION 1..16: note = NME10A3
IQDCENCNIYIFDHSA

SEQ ID NO: 334 - protein, 30 aa, synthetic construct
  REGION 1..30: note = NME10B1
ELAFQFKDAGLSIFNNTWSNIHDFTPVDCT

SEQ ID NO: 335 - DNA, 363 bp, synthetic construct
  misc_feature 1..363: note = 8F9A4P3 Heavy chain variable region sequence mouse
  source 1..363: mol_type = other DNA
gtccagctgcaacagtctggacctgaactggtgaagcctggggcttcagtgaagatatcctgcaagacttctggaaacacattcactgaatacaccatgcactgggtgaagcagagccatggaaagagccttgagtggattggaggttttaatcctaacaatggtgttactaactacaaccagaagttcaagggcaaggccacattgactgtagacaagtcctccagcacagcctacatggagctccgcagcctgacatctgaggattctgcagtctattactgtgcaagacggtactaccatagtctctacgtgttttactttgactactggggccaaggcaccactctcacagtctcctca

SEQ ID NO: 336 - DNA, 294 bp, synthetic construct
  misc_feature 1..294: note = IGHV1-24*01 V-REGION sequence human (closest match hu antibody sequence)
  source 1..294: mol_type = other DNA
caggtccagctggtacagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaaca

SEQ ID NO: 337 - DNA, 45 bp, synthetic construct
  misc_feature 1..45: note = IGHJ4*01 J-REGION sequence human (closest match hu antibody sequence)
  source 1..45: mol_type = other DNA
tactttgactactggggccaaggaaccctggtcaccgtctcctca

SEQ ID NO: 338 - DNA, 366 bp, synthetic construct
  misc_feature 1..366: note = humanized 8F9A4P3 Heavy chain variable region sequence
  source 1..366: mol_type = other DNA
caggtccagctggtacagtctggggctgaggtgaagaagcctggggcctcagtgaaggtctcctgcaaggtttccggaaacacattcactgaatacaccatgcactgggtgcgacaggctcctggaaaagggcttgagtggatgggaggttttaatcctaacaatggtgttactaactacaaccagaagttcaagggcagagtcaccatgaccgaggacacatctacagacacagcctacatggagctgagcagcctgagatctgaggacacggccgtgtattactgtgcaagacggtactaccatagtctctacgtgttttactttgactactggggccaaggaaccctggtcaccgtctcctca

SEQ ID NO: 339 - DNA, 366 bp, synthetic construct
  misc_feature 1..366: note = humanized 8F9A4P3 Heavy chain variable region sequence (codon optimized)
  source 1..366: mol_type = other DNA
caggttcagctggttcagtctggcgccgaagtgaagaaacctggcgcctctgtgaaggtgtcctgcaaggtgtccggaaataccttcaccgagtacaccatgcactgggtccgacaggcccctggcaaaggacttgaatggatgggcggcttcaaccccaacaacggcgtgaccaactacaaccagaaattcaagggccgcgtgaccatgaccgaggacacaagcacagacaccgcctacatggaactgagcagcctgagaagcgaggacaccgccgtgtactactgcgccagaaggtactaccacagcctgtacgtgttctacttcgactactggggccagggcaccctggtcacagtttcttct

SEQ ID NO: 340 - DNA, 366 bp, synthetic construct
  misc_feature 1..366: note = Modified humanized 8F9A4P3 Heavy chain variable region sequence
  source 1..366: mol_type = other DNA
caggtccagctggtacagtctggggctgaggtgaagaagcctggggcctcagtgaaggtctcctgcaaggtttccggaaacacattcactgaatacaccatgcactgggtgcgacaggctcctggaaaagggcttgagtggattggaggttttaatcctaacaatggtgttactaactacaaccagaagttcaagggcaaagtcaccctgaccgtggacacatctagcagcacagcctacatggagctgagcagcctgagatctgaggacacggccgtgtattactgtgcaagacggtactaccatagtctctacgtgttttactttgactactggggccaaggaaccctggtcaccgtctcctca

SEQ ID NO: 341 - DNA, 366 bp, synthetic construct
  misc_feature 1..366: note = Modified humanized 8F9A4P3 Heavy chain variable region sequence (codon optimized)
  source 1..366: mol_type = other DNA
caggttcagctggttcagtctggcgccgaagtgaagaaacctggcgcctctgtgaaggtgtcctgcaaggtgtccggaaataccttcaccgagtacaccatgcactgggtccgacaggcccctggcaaaggactggaatggatcggcggcttcaaccccaacaacggcgtgaccaactacaaccagaaattcaagggcaaagtgaccctgaccgtggacaccagcagcagcacagcctacatggaactgagcagcctgagaagcgaggacaccgccgtgtactactgcgccagaaggtactaccacagcctgtacgtgttctacttcgactactggggccagggcaccctggtcacagtttcttct

SEQ ID NO: 342 - DNA, 324 bp, synthetic construct
  misc_feature 1..324: note = 8F9A4P3 Light chain variable region sequence mouse
  source 1..324: mol_type = other DNA
gaaacaactgtgacccagtctccagcatccctgtccatggctataggagaaaaagtcaccatcagatgcataaccagcactgatattgatgatgatatgaactggtaccagcagaagccaggggaacctcctaagctccttatttcagaaggcaatactcttcgtcctggagtcccatcccgattctccagcagtggctatggtacagattttgtttttacaattgaaaacatgctctcagaagatgttgcagattactactgtttgcaaagtgataacttgcctctcacgttcggctcggggacaaagttggaaataaaacgg

SEQ ID NO: 343 - DNA, 264 bp, synthetic construct
  misc_feature 1..264: note = IGKV5-2*01 V-REGION sequence human (closest match hu antibody sequence)
  source 1..264: mol_type = other DNA
gaaacgacactcacgcagtctccagcattcatgtcagcgactccaggagacaaagtcaacatctcctgcaaagccagccaagacattgatgatgatatgaactggtaccaacagaaaccaggagaagctgctattttcattattcaagaagctactactctcgttcctggaatcccacctcgattcagtggcagcgggtatggaacagattttaccctcacaattaataacatagaatctgaggatgctgcatattacttctgt

SEQ ID NO: 344 - DNA, 36 bp, synthetic construct
  misc_feature 1..36: note = IGKJ4*02 J-REGION sequence human (closest match hu antibody sequence)
  source 1..36: mol_type = other DNA
ctcacgttcggcggagggaccaaggtggagatcaaa

SEQ ID NO: 345 - DNA, 324 bp, synthetic construct
  misc_feature 1..324: note = humanized 8F9A4P3 Light chain variable region sequence
  source 1..324: mol_type = other DNA
gaaacgacactcacgcagtctccagcattcatgtcagcgactccaggagacaaagtcaacatctcctgcataaccagcactgatattgatgatgatatgaactggtaccaacagaaaccaggagaagctgctattttcattattcaagaaggcaatactcttcgtcctggaatcccacctcgattcagtggcagcgggtatggaacagattttaccctcacaattaataacatagaatctgaggatgctgcatattacttctgtttgcaaagtgataacttgcctctcacgttcggcggagggaccaaggtggagatcaaacgg

SEQ ID NO: 346 - DNA, 324 bp, synthetic construct
  misc_feature 1..324: note = humanized 8F9A4P3 Light chain variable region sequence (codon optimized)
  source 1..324: mol_type = other DNA
gagacaaccctgacacagagccctgccttcatgtctgccacacctggcgacaaagtgaacatcagctgcatcaccagcaccgacatcgacgacgacatgaactggtatcagcagaagcctggcgaggccgccatcttcatcatccaagagggcaacacactgcggcctggcatccctcctagattttctggcagcggctacggcaccgacttcaccctgaccatcaacaacatcgagagcgaggacgccgcctactacttctgcctgcaaagcgacaacctgcctctgacctttggcggaggcaccaaggtggaaatcaagcgg

SEQ ID NO: 347 - DNA, 324 bp, synthetic construct
  misc_feature 1..324: note = Modified humanized 8F9A4P3 Light chain variable region sequence
  source 1..324: mol_type = other DNA
gaaacgacagtgacgcagtctccagcattcatgtcagcgactccaggagacaaagtcaccatctcctgcataaccagcactgatattgatgatgatatgaactggtaccaacagaaaccaggagaagctgctattctgctgattagcgaaggcaatactcttcgtcctggaatcccacctcgattcagtagcagcgggtatggaacagattttaccctcacaattaataacatagaatctgaggatgctgcatattacttctgtttgcaaagtgataacttgcctctcacgttcggcggagggaccaaggtggagatcaaacgg

SEQ ID NO: 348 - DNA, 324 bp, synthetic construct
  misc_feature 1..324: note = Modified humanized 8F9A4P3 Light chain variable region sequence (codon optimized)
  source 1..324: mol_type = other DNA
gagacaaccgtgacacagagccctgccttcatgtctgccacacctggcgacaaagtgaccatcagctgcatcaccagcaccgacatcgacgacgacatgaactggtatcagcagaagcctggcgaggccgccatcctgcttatctctgagggaaacacactgcggcctggcatccctcctagattttccagcagcggctacggcaccgacttcaccctgaccatcaacaacatcgagagcgaggacgccgcctactacttctgcctgcaaagcgacaacctgcctctgacctttggcggaggcaccaaggtggaaatcaagcgg

SEQ ID NO: 349 - DNA, 735 bp, synthetic construct
  misc_feature 1..735: note = Modified humanized 8F9A4P3 sequence (codon optimized) - humanized heavy and light chains joined via a flexible linker
  source 1..735: mol_type = other DNA
caggttcagctggttcagtctggcgccgaagtgaagaaacctggcgcctctgtgaaggtgtcctgcaaggtgtccggaaataccttcaccgagtacaccatgcactgggtccgacaggcccctggcaaaggactggaatggatcggcggcttcaaccccaacaacggcgtgaccaactacaaccagaaattcaagggcaaagtgaccctgaccgtggacaccagcagcagcacagcctacatggaactgagcagcctgagaagcgaggacaccgccgtgtactactgcgccagaaggtactaccacagcctgtacgtgttctacttcgactactggggccagggcaccctggtcacagtttcttctggcggtggcggaagcggaggcggtggctccggtggcggaggcagcgaaacgacagtgacgcagtctccagcattcatgtcagcgactccaggagacaaagtcaccatctcctgcataaccagcactgatattgatgatgatatgaactggtaccaacagaaaccaggagaagctgctattctgctgattagcgaaggcaatactcttcgtcctggaatcccacctcgattcagtagcagcgggtatggaacagattttaccctcacaattaataacatagaatctgaggatgctgcatattacttctgtttgcaaagtgataacttgcctctcacgttcggcggagggaccaaggtggagatcaaacgg

SEQ ID NO: 350 - DNA, 357 bp, synthetic construct
  misc_feature 1..357: note = 8F9A5A1 Heavy chain variable region sequence
  source 1..357: mol_type = other DNA
atccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgttgatgacttcaagggacggtttgccttctctttggaaacctctgccaccactgcctatttgcagatcaacaacctcaaaaatgaggacacgtctacatatttctgtgcaagattgagggggatacgaccgggtcccttggcttactggggccaagggactctggtcactgtctctgca

SEQ ID NO: 351 - DNA, 294 bp, synthetic construct
  misc_feature 1..294: note = IGHV7-81*01 V-REGION sequence
  source 1..294: mol_type = other DNA
caggtgcagctggtgcagtctggccatgaggtgaagcagcctggggcctcagtgaaggtctcctgcaaggcttctggttacagtttcaccacctatggtatgaattgggtgccacaggcccctggacaagggcttgagtggatgggatggttcaacacctacactgggaacccaacatatgcccagggcttcacaggacggtttgtcttctccatggacacctctgccagcacagcatacctgcagatcagcagcctaaaggctgaggacatggccatgtattactgtgcgaga

SEQ ID NO: 352 - DNA, 45 bp, synthetic construct
  misc_feature 1..45: note = IGHJ4*03 J-REGION sequence
  source 1..45: mol_type = other DNA
tactttgactactggggccaagggaccctggtcaccgtctcctca

SEQ ID NO: 353 - DNA, 360 bp, synthetic construct
  misc_feature 1..360: note = humanized 8F9A5A1 Heavy chain variable region sequence
  source 1..360: mol_type = other DNA
caggtgcagctggtgcagtctggccatgaggtgaagcagcctggggcctcagtgaaggtctcctgcaaggcttctgggtataccttcacaaactatggaatgaactgggtgccacaggcccctggacaagggcttgagtggatgggatggataaacacctacactggagagccaacatatgttgatgacttcaagggacggtttgtcttctccatggacacctctgccagcacagcatacctgcagatcagcagcctaaaggctgaggacatggccatgtattactgtgcaagattgagggggatacgaccgggtcccttggcttactggggccaagggaccctggtcaccgtctcctca

SEQ ID NO: 354 - DNA, 360 bp, synthetic construct
  misc_feature 1..360: note = humanized 8F9A5A1 Heavy chain variable region sequence (codon optimized)
  source 1..360: mol_type = other DNA
caggttcagctggtgcagtctggccacgaagtgaaacagcctggcgcctctgtgaaggtgtcctgtaaagccagcggctacacctttaccaactacggcatgaactgggtgccccaggctcctggacaaggcttggaatggatgggctggatcaacacctacaccggcgagcctacctacgtggacgacttcaagggcagattcgtgttcagcatggacaccagcgccagcacagcctacctgcagatcagctctctgaaggccgaggatatggccatgtactactgcgccagactgagaggcatcagacctggacctctggcctattggggacagggcacactggtcacagtgtcctct

SEQ ID NO: 355 - DNA, 360 bp, synthetic construct
  misc_feature 1..360: note = Modified humanized 8F9A5A1 Heavy chain variable region sequence
  source 1..360: mol_type = other DNA
cagatccagctggtgcagtctggccccgaggtgaagcagcctggggcctcagtgaaggtctcctgcaaggcttctgggtataccttcacaaactatggaatgaactgggtgaagcaggcccctggacaagggcttgagtggatgggatggataaacacctacactggagagccaacatatgttgatgacttcaagggacggtttgccttctccatggacacctctgccagcacagcatacctgcagatcagcagcctaaaggctgaggacaccgccacctattactgtgcaagattgagggggatacgaccgggtcccttggcttactggggccaagggaccctggtcaccgtctcctca

SEQ ID NO: 356 - DNA, 360 bp, synthetic construct
  misc_feature 1..360: note = Modified humanized 8F9A5A1 Heavy chain variable region sequence (codon optimized)
  source 1..360: mol_type = other DNA
cagattcagctggtgcagtctggccccgaagtgaaacaacctggcgcctctgtgaaggtgtcctgcaaggccagcggctacacctttaccaactacggcatgaactgggtcaagcaggcccctggacaaggcctggaatggatgggctggatcaacacctacaccggcgagcctacctacgtggacgacttcaagggcagattcgccttcagcatggacaccagcgccagcacagcctacctgcagatcagctctctgaaggccgaggacaccgccacctactactgtgccagactgagaggcatcagacccggacctctggcctattggggacagggaacactggtcaccgtgtcctct

SEQ ID NO: 357 - DNA, 321 bp, synthetic construct
  misc_feature 1..321: note = 8F9A5A1 Light chain variable region sequence
  source 1..321: mol_type = other DNA
gaaattttgctcacccagtctccagcaatcatagctgcatctcctggggagaaggtcaccatcacctgcagtgccagctcaagtgtaagttacatgaactggtaccagcagaaaccaggatcctcccccaaaatatggatttatggtatatccaacctggcttctggagttcctgctcgcttcagtggcagtgggtctgggacatctttctctttcacaatcaacagcatggaggctgaagatgttgccacttattactgtcagcaaaggagtagttacccacccacgttcggaggggggaccaagctggaaataaaacgg

SEQ ID NO: 358 - DNA, 279 bp, synthetic construct
  misc_feature 1..279: note = IGKV3D-15*02 V-REGION sequence
  source 1..279: mol_type = other DNA
gaaatagtgatgatgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggcatcccagccaggttcagtggcagtgggtctgggacagagttcactctcaccatcagcagcctgcagtctgaagattttgcagtttattactgtcagcagtataataac

SEQ ID NO: 359 - DNA, 36 bp, synthetic construct; identical to SEQ ID NO: 344
  misc_feature 1..36: note = IGKJ4*02 J-REGION sequence
  source 1..36: mol_type = other DNA

SEQ ID NO: 360 - DNA, 321 bp, synthetic construct
  misc_feature 1..321: note = humanized 8F9A5A1 Light chain variable region sequence
  source 1..321: mol_type = other DNA
gaaatagtgatgatgcagtctccagccaccctgtctgtgtctccaggggaaagagccaccctctcctgcagtgccagctcaagtgtaagttacatgaactggtaccagcagaaacctggccaggctcccaggctcctcatctatggtatatccaacctggcttctggcatcccagccaggttcagtggcagtgggtctgggacagagttcactctcaccatcagcagcctgcagtctgaagattttgcagtttattactgtcagcaaaggagtagttacccacccacgttcggcggagggaccaaggtggagatcaaacgg

SEQ ID NO: 361 - DNA, 321 bp, synthetic construct
  misc_feature 1..321: note = humanized 8F9A5A1 Light chain variable region sequence (codon optimized)
  source 1..321: mol_type = other DNA
gagatcgtgatgatgcagagccccgccacactgagtgtgtctccaggcgaaagagccacactgtcctgtagcgccagcagcagcgtgtcctacatgaactggtatcagcagaagcccggacaggcccctagactgctgatctacggcatcagcaatctggccagcggcatccctgccagattttctggctctggctccggcaccgagttcaccctgacaatctctagcctgcagagcgaggacttcgccgtgtactactgccagcagagaagcagctaccctcctacctttggcggaggcaccaaggtggaaatcaagcgg

SEQ ID NO: 362 - DNA, 321 bp, synthetic construct
  misc_feature 1..321: note = Modified humanized 8F9A5A1 Light chain variable region sequence
  source 1..321: mol_type = other DNA
gaaatagtgctgacccagtctccagccaccctgtctgtgtctccaggggaaagagccaccctctcctgcagtgccagctcaagtgtaagttacatgaactggtaccagcagaaacctggccaggctcccaggctctggatctatggtatatccaacctggcttctggcatcccagccaggttcagtggcagtgggtctgggacaagcttcagcctcaccatcagcagcctgcagtctgaagattttgcagtttattactgtcagcaaaggagtagttacccacccacgttcggcggagggaccaaggtggagatcaaacgg

SEQ ID NO: 363 - DNA, 321 bp, synthetic construct
  misc_feature 1..321: note = Modified humanized 8F9A5A1 Light chain variable region sequence (codon optimized)
  source 1..321: mol_type = other DNA
gagatcgtgctgacacagtctcccgccacactgagtgtgtctccaggcgaaagagccacactgtcctgtagcgccagcagcagcgtgtcctacatgaactggtatcagcagaagcccggacaggcccctagactgtggatctacggcatcagcaatctggccagcggcatccctgccagattttctggctctggctccggcaccagcttcagcctgacaatcagcagcctgcagagcgaggacttcgccgtgtactactgccagcagagaagcagctaccctcctacctttggcggaggcaccaaggtggaaatcaagcgg

SEQ ID NO: 364 - DNA, 726 bp, synthetic construct
  misc_feature 1..726: note = Modified humanized 8F9A5A1 scFV sequence (codon optimized)
  source 1..726: mol_type = other DNA
cagattcagctggtgcagtctggccccgaagtgaaacaacctggcgcctctgtgaaggtgtcctgcaaggccagcggctacacctttaccaactacggcatgaactgggtcaagcaggcccctggacaaggcctggaatggatgggctggatcaacacctacaccggcgagcctacctacgtggacgacttcaagggcagattcgccttcagcatggacaccagcgccagcacagcctacctgcagatcagctctctgaaggccgaggacaccgccacctactactgtgccagactgagaggcatcagacccggacctctggcctattggggacagggaacactggtcaccgtgtcctctggcggtggcggaagcggaggcggtggctccggtggcggaggcagcgagatcgtgctgacacagtctcccgccacactgagtgtgtctccaggcgaaagagccacactgtcctgtagcgccagcagcagcgtgtcctacatgaactggtatcagcagaagcccggacaggcccctagactgtggatctacggcatcagcaatctggccagcggcatccctgccagattttctggctctggctccggcaccagcttcagcctgacaatcagcagcctgcagagcgaggacttcgccgtgtactactgccagcagagaagcagctaccctcctacctttggcggaggcaccaaggtggaaatcaagcgg

SEQ ID NO: 365 - DNA, 363 bp, synthetic construct
  misc_feature 1..363: note = 8H5H5G4 Heavy chain variable region sequence
  source 1..363: mol_type = other DNA
gtccagctgcaacagtctggacctgatctggtgaagcctgggacttcagtgaagatatcctgtaagacttctggaaacacattcactgaatacaccatgcactgggtgaagcagagccatggaaagagccttgagtggattggaggttttaatcctaacaatggtgttactaactacaaccagaagttcaagggcaaggccacattgactgtagacaagtcctccagcacagcctacatggagctccgcagcctgacatctgaggattctgcagtctattactgtgcaagacgttactaccatagtacctacgtgttctactttgactcctggggccaaggcaccactctcacagtctcctca

SEQ ID NO: 366 - DNA, 294 bp, synthetic construct; identical to SEQ ID NO: 336
  misc_feature 1..294: note = IGHV1-24*01 V-REGION sequence
  source 1..294: mol_type = other DNA

SEQ ID NO: 367 - DNA, 45 bp, synthetic construct; identical to SEQ ID NO: 352
  misc_feature 1..45: note = IGHJ4*03 J-REGION sequence
  source 1..45: mol_type = other DNA

SEQ ID NO: 368 - DNA, 366 bp, synthetic construct
  misc_feature 1..366: note = Humanized 8H5H5G4 Heavy chain variable region sequence
  source 1..366: mol_type = other DNA
caggtccagctggtacagtctggggctgaggtgaagaagcctggggcctcagtgaaggtctcctgcaaggtttccggaaacacattcactgaatacaccatgcactgggtgcgacaggctcctggaaaagggcttgagtggatgggaggttttaatcctaacaatggtgttactaactacaaccagaagttcaagggcagagtcaccatgaccgaggacacatctacagacacagcctacatggagctgagcagcctgagatctgaggacacggccgtgtattactgtgcaagacgttactaccatagtacctacgtgttctactttgactcctggggccaagggaccctggtcaccgtctcctca

SEQ ID NO: 369 - DNA, 366 bp, synthetic construct
  misc_feature 1..366: note = Humanized 8H5H5G4 Heavy chain variable region sequence (codon optimized)
  source 1..366: mol_type = other DNA
caggttcagctggttcagtctggcgccgaagtgaagaaacctggcgcctctgtgaaggtgtcctgcaaggtgtccggaaataccttcaccgagtacaccatgcactgggtccgacaggcccctggcaaaggacttgaatggatgggcggcttcaaccccaacaacggcgtgaccaactacaaccagaaattcaagggccgcgtgaccatgaccgaggacacaagcacagacaccgcctacatggaactgagcagcctgagaagcgaggacaccgccgtgtactactgcgccagaaggtactaccacagcacctacgtgttctacttcgacagctggggccagggcacactggtcacagtttcttct

SEQ ID NO: 370 - DNA, 366 bp, synthetic construct
  misc_feature 1..366: note = Modified Humanized 8H5H5G4 Heavy chain variable region sequence
  source 1..366: mol_type = other DNA
caggtccagctggtacagtctggggctgaggtgaagaagcctggggcctcagtgaaggtctcctgcaaggtttccggaaacacattcactgaatacaccatgcactgggtgcgacaggctcctggaaaagggcttgagtggatcggaggttttaatcctaacaatggtgttactaactacaaccagaagttcaagggcaaggtcaccctgaccgtggacacatctagcagcacagcctacatggagctgagcagcctgagatctgaggacacggccgtgtattactgtgcaagacgttactaccatagtacctacgtgttctactttgactcctggggccaagggaccctggtcaccgtctcctca

SEQ ID NO: 371 - DNA, 366 bp, synthetic construct
  misc_feature 1..366: note = Modified Humanized 8H5H5G4 Heavy chain variable region sequence (codon optimized)
  source 1..366: mol_type = other DNA
caggttcagctggttcagtctggcgccgaagtgaagaaacctggcgcctctgtgaaggtgtcctgcaaggtgtccggaaataccttcaccgagtacaccatgcactgggtccgacaggcccctggcaaaggactggaatggatcggcggcttcaaccccaacaacggcgtgaccaactacaaccagaaattcaagggcaaagtgaccctgaccgtggacaccagcagcagcacagcctacatggaactgagcagcctgagaagcgaggacaccgccgtgtactactgcgccagaaggtactaccacagcacctacgtgttctacttcgacagctggggccagggcacactggtcacagtttcttct

SEQ ID NO: 372 - DNA, 324 bp, synthetic construct
  misc_feature 1..324: note = 8H5H5G4 Light chain variable region sequence
  source 1..324: mol_type = other DNA
gatatccagatgacacagactacatcctccctgtctgcctctctgggagacagagtcaccatcagttgcagtgcaagtcagggcattagcaattatttaaactggtttcagcagaaaccagatggaactattaagctcctgatctattacacatcaagtttacattcaggagtcccatcaaggttcagtggcagtgggtctgggacagattattctctcaccatcagtaatgtggaacctgaagatattgccacttactattgtcagcagtatagtaagcttccttacacgttcggaggggggaccaagctggagataaaacgg

SEQ ID NO: 373 - DNA, 285 bp, synthetic construct
  misc_feature 1..285: note = IGKV1-27*01 V-REGION sequence
  source 1..285: mol_type = other DNA
gacatccagatgacccagtctccatcctccctgtctgcatctgtaggagacagagtcaccatcacttgccgggcgagtcagggcattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgcccct

SEQ ID NO: 374 - DNA, 36 bp, synthetic construct; identical to SEQ ID NO: 344
  misc_feature 1..36: note = IGKJ4*02 J-REGION sequence
  source 1..36: mol_type = other DNA

SEQ ID NO: 375 - DNA, 324 bp, synthetic construct
  misc_feature 1..324: note = humanized 8H5H5G4 Light chain variable region sequence
  source 1..324: mol_type = other DNA
gacatccagatgacccagtctccatcctccctgtctgcatctgtaggagacagagtcaccatcacttgcagtgcaagtcagggcattagcaattatttaaactggtatcagcagaaaccagggaaagttcctaagctcctgatctattacacatcaagtttacattcaggggtcccatctcggttcagtggcagtggatctgggacagatttcactctcaccatcagcagcctgcagcctgaagatgttgcaacttattactgtcagcagtatagtaagcttccttacacgttcggcggagggaccaaggtggagatcaaacgg

SEQ ID NO: 376 - DNA, 324 bp, synthetic construct
  misc_feature 1..324: note = humanized 8H5H5G4 Light chain variable region sequence (codon optimized)
  source 1..324: mol_type = other DNA
gacatccagatgacacagagccctagcagcctgtctgccagcgtgggagacagagtgaccatcacatgtagcgccagccagggcatcagcaactacctgaactggtatcagcagaaacccggcaaggtgcccaagctgctgatctactacaccagcagcctgcacagcggcgtgccaagcagattttctggcagcggctctggcaccgacttcaccctgaccatatctagcctgcagcctgaggacgtggccacctactactgtcagcagtacagcaagctgccctacacctttggcggaggcaccaaggtggaaatcaagcgg

SEQ ID NO: 377 - DNA, 324 bp, synthetic construct
  misc_feature 1..324: note = Modified humanized 8H5H5G4 Light chain variable region sequence
  source 1..324: mol_type = other DNA
gacatccagatgacccagtctccatcctccctgtctgcatctgtaggagacagagtcaccatcacttgcagtgcaagtcagggcattagcaattatttaaactggtatcagcagaaaccagggaaagttcctaagctcctgatctattacacatcaagtttacattcaggggtcccatctcggttcagtggcagtggatctgggacagattacactctcaccatcagcagcctgcagcctgaagatgttgcaacttattactgtcagcagtatagtaagcttccttacacgttcggcggagggaccaaggtggagatcaaacgg

SEQ ID NO: 378 - DNA, 324 bp, synthetic construct
  misc_feature 1..324: note = Modified humanized 8H5H5G4 Light chain variable region sequence (codon optimized)
  source 1..324: mol_type = other DNA
gacatccagatgacacagagccctagcagcctgtctgccagcgtgggagacagagtgaccatcacatgtagcgccagccagggcatcagcaactacctgaactggtatcagcagaaacccggcaaggtgcccaagctgctgatctactacaccagcagcctgcacagcggcgtgccaagcagattttctggcagcggctctggcaccgactacaccctgaccatatctagcctgcagcctgaggacgtggccacctactactgtcagcagtacagcaagctgccctacacctttggcggaggcaccaaggtggaaatcaagcgg

SEQ ID NO: 379 - DNA, 735 bp, synthetic construct
  misc_feature 1..735: note = Modified humanized 8H5H5G4 scFV sequence (codon optimized)
  source 1..735: mol_type = other DNA
caggttcagctggttcagtctggcgccgaagtgaagaaacctggcgcctctgtgaaggtgtcctgcaaggtgtccggaaataccttcaccgagtacaccatgcactgggtccgacaggcccctggcaaaggactggaatggatcggcggcttcaaccccaacaacggcgtgaccaactacaaccagaaattcaagggcaaagtgaccctgaccgtggacaccagcagcagcacagcctacatggaactgagcagcctgagaagcgaggacaccgccgtgtactactgcgccagaaggtactaccacagcacctacgtgttctacttcgacagctggggccagggcacactggtcacagtttcttctggcggtggcggaagcggaggcggtggctccggtggcggaggcagcgacatccagatgacacagagccctagcagcctgtctgccagcgtgggagacagagtgaccatcacatgtagcgccagccagggcatcagcaactacctgaactggtatcagcagaaacccggcaaggtgcccaagctgctgatctactacaccagcagcctgcacagcggcgtgccaagcagattttctggcagcggctctggcaccgactacaccctgaccatatctagcctgcagcctgaggacgtggccacctactactgtcagcagtacagcaagctgccctacacctttggcggaggcaccaaggtggaaatcaagcgg

SEQ ID NO: 380 - DNA, 993 bp, synthetic construct
  misc_feature 1..993: note = Human IgG1 heavy chain constant region sequence: (for making full antibody - pair with either kappa or lambda constant region; 2 plasmids, express together)
  source 1..993: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgacagtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 381 - DNA, 981 bp, synthetic construct
  misc_feature 1..981: note = Human IgG2 heavy chain constant region sequence: (for making full antibody - pair with either kappa or lambda constant region; 2 plasmids, express together)
  source 1..981: mol_type = other DNA
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 382 - DNA, 324 bp, synthetic construct
  misc_feature 1..324: note = Human Kappa light chain constant region sequence
  source 1..324: mol_type = other DNA
aggacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 383 - DNA, 321 bp, synthetic construct
  misc_feature 1..321: note = Human Lambda light chain constant region sequence
  source 1..321: mol_type = other DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 384 - DNA, 699 bp, synthetic construct
  misc_feature 1..699: note = Human IgG1 Fc region sequence: (to be fused to scFv for homo-dimerizes)
  source 1..699: mol_type = other DNA
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 385 - DNA, 687 bp, synthetic construct
  misc_feature 1..687: note = Human IgG2 Fc region sequence
  source 1..687: mol_type = other DNA
gagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 386 - DNA, 363 bp, synthetic construct; identical to SEQ ID NO: 335
  misc_feature 1..363: note = Monoclonal antibody 8F9A4A3 - Heavy chain variable region sequence - H-1,8,9,10,11
  source 1..363: mol_type = other DNA

SEQ ID NO: 387 - protein, 121 aa, synthetic construct; identical to SEQ ID NO: 173
  REGION 1..121: note = Monoclonal antibody 8F9A4A3 Translated protein, wherein the underlined sequence is the complementarity determining region (CDR)

SEQ ID NO: 388 - protein, 9 aa, synthetic construct
  REGION 1..9: note = Monoclonal antibody 8F9A4A3 Heavy chain variable region CDR1
NTFTEYTMH

SEQ ID NO: 389 - protein, 17 aa, synthetic construct
  REGION 1..17: note = Monoclonal antibody 8F9A4A3 Heavy chain variable region CDR2
GFNPNNGVTNYNQKFKG

SEQ ID NO: 390 - protein, 13 aa, synthetic construct
  REGION 1..13: note = Monoclonal antibody 8F9A4A3 Heavy chain variable region CDR3
RYYHSLYVFYFDY

SEQ ID NO: 391 - DNA, 324 bp, synthetic construct; identical to SEQ ID NO: 342
  misc_feature 1..324: note = Monoclonal antibody 8F9A4A3 Light chain variable region sequence - K-3,4,9,10,11
  source 1..324: mol_type = other DNA

SEQ ID NO: 392 - protein, 108 aa, synthetic construct; identical to SEQ ID NO: 185
  REGION 1..108: note = Monoclonal antibody 8F9A4A3 Translated protein, wherein the underlined sequence is the complementarity determining region (CDR)

SEQ ID NO: 393 - protein, 11 aa, synthetic construct
  REGION 1..11: note = Monoclonal antibody 8F9A4A3 Light chain variable region CDR1
SASQGISNYLN

SEQ ID NO: 394 - protein, 7 aa, synthetic construct
  REGION 1..7: note = Monoclonal antibody 8F9A4A3 Light chain variable region CDR2
YTSSLHS

SEQ ID NO: 395 - protein, 9 aa, synthetic construct
  REGION 1..9: note = Monoclonal antibody 8F9A4A3 Light chain variable region CDR3
QQYSKLPYT

SEQ ID NO: 396 - DNA, 363 bp, synthetic construct; identical to SEQ ID NO: 365
  misc_feature 1..363: note = Monoclonal antibody 5D9E2B11 - Heavy chain variable region sequence - H-1,4,7,8,12
  source 1..363: mol_type = other DNA

SEQ ID NO: 397 - protein, 121 aa, synthetic construct; identical to SEQ ID NO: 175
  REGION 1..121: note = Monoclonal antibody 5D9E2B11 Translated protein, wherein the underlined sequence is the complementarity determining region (CDR)

SEQ ID NO: 398 - protein, 9 aa, synthetic construct
  REGION 1..9: note = Monoclonal antibody 5D9E2B11 Heavy chain variable region CDR1
NTFTEYTMH

SEQ ID NO: 399 - protein, 17 aa, synthetic construct
  REGION 1..17: note = Monoclonal antibody 5D9E2B11 Heavy chain variable region CDR2
GFNPNNGVTNYNQKFKG

SEQ ID NO: 400 - protein, 13 aa, synthetic construct
  REGION 1..13: note = Monoclonal antibody 5D9E2B11 Heavy chain variable region CDR3
RYYHSTYVFYFDS

SEQ ID NO: 401 - DNA, 324 bp, synthetic construct; identical to SEQ ID NO: 372
  misc_feature 1..324: note = Monoclonal antibody 5D9E2B11 Light chain variable region sequence - K-3,4,5,6,12
  source 1..324: mol_type = other DNA

SEQ ID NO: 402 - protein, 108 aa, synthetic construct; identical to SEQ ID NO: 186
  REGION 1..108: note = Monoclonal antibody 5D9E2B11 Translated protein

SEQ ID NO: 403 - protein, 108 aa, synthetic construct; identical to SEQ ID NO: 186
  REGION 1..108: note = Monoclonal antibody 5D9E2B11 Translated protein, wherein the underlined sequence is the complementarity determining region (CDR)

SEQ ID NO: 404 - protein, 11 aa, synthetic construct
  REGION 1..11: note = Monoclonal antibody 5D9E2B11 Light chain variable region CDR 1
SASQGISNYLN

SEQ ID NO: 405 - protein, 7 aa, synthetic construct
  REGION 1..7: note = Monoclonal antibody 5D9E2B11 Light chain variable region CDR2
YTSSLHS

SEQ ID NO: 406 - protein, 9 aa, synthetic construct
  REGION 1..9: note = Monoclonal antibody 5D9E2B11 Light chain variable region CDR3
QQYSKLPYT

SEQ ID NO: 407 - DNA, 363 bp, synthetic construct; identical to SEQ ID NO: 365
  misc_feature 1..363: note = Monoclonal antibody 5D9E10E4 - Heavy chain variable region sequence - H-2,4,7,10,12
  source 1..363: mol_type = other DNA

SEQ ID NO: 408 - protein, 121 aa, synthetic construct; identical to SEQ ID NO: 175
  REGION 1..121: note = Monoclonal antibody 5D9E10E4 Translated protein, wherein the underlined sequence is the complementarity determining region (CDR)

SEQ ID NO: 409 - protein, 9 aa, synthetic construct
  REGION 1..9: note = Monoclonal antibody 5D9E10E4 Heavy chain variable region CDR1
NTFTEYTMH

SEQ ID NO: 410 - protein, 17 aa, synthetic construct
  REGION 1..17: note = Monoclonal antibody 5D9E10E4 Heavy chain variable region CDR2
GFNPNNGVTNYNQKFKG

SEQ ID NO: 411 - protein, 13 aa, synthetic construct
  REGION 1..13: note = Monoclonal antibody 5D9E10E4 Heavy chain variable region CDR3
RYYHSTYVFYFDS

SEQ ID NO: 412 - DNA, 324 bp, synthetic construct; identical to SEQ ID NO: 372
  misc_feature 1..324: note = Monoclonal antibody 5D9E10E4 Light chain variable region sequence - K-2,6,8,14,15
  source 1..324: mol_type = other DNA

SEQ ID NO: 413 - protein, 108 aa, synthetic construct; identical to SEQ ID NO: 186
  REGION 1..108: note = Monoclonal antibody 5D9E10E4 Translated protein, wherein the underlined sequence is the complementarity determining region (CDR)

SEQ ID NO: 414 - protein, 11 aa, synthetic construct
  REGION 1..11: note = Monoclonal antibody 5D9E10E4 Light chain variable region CDR 1
SASQGISNYLN

SEQ ID NO: 415 - protein, 7 aa, synthetic construct
  REGION 1..7: note = Monoclonal antibody 5D9E10E4 Light chain variable region CDR2
YTSSLHS

SEQ ID NO: 416 - protein, 9 aa, synthetic construct
  REGION 1..9: note = Monoclonal antibody 5D9E10E4 Light chain variable region CDR3
QQYSKLPYT

SEQ ID NO: 417 - DNA, 363 bp, synthetic construct; identical to SEQ ID NO: 365
  misc_feature 1..363: note = Monoclonal antibody 5D9G2C4 - Heavy chain variable region sequence - H-4,9,10,11,13
  source 1..363: mol_type = other DNA

SEQ ID NO: 418 - protein, 121 aa, synthetic construct; identical to SEQ ID NO: 175
  REGION 1..121: note = Monoclonal antibody 5D9G2C4 Translated protein, wherein the underlined sequence is the complementarity determining region (CDR)

SEQ ID NO: 419 - protein, 9 aa, synthetic construct
  REGION 1..9: note = Monoclonal antibody 5D9G2C4 Heavy chain variable region CDR1
NTFTEYTMH

SEQ ID NO: 420 - protein, 17 aa, synthetic construct
  REGION 1..17: note = Monoclonal antibody 5D9G2C4 Heavy chain variable region CDR2
GFNPNNGVTNYNQKFKG

SEQ ID NO: 421 - protein, 13 aa, synthetic construct
  REGION 1..13: note = Monoclonal antibody 5D9G2C4 Heavy chain variable region CDR3
RYYHSTYVFYFDS

SEQ ID NO: 422 - DNA, 324 bp, synthetic construct; identical to SEQ ID NO: 372
  misc_feature 1..324: note = Monoclonal antibody 5D9G2C4 Light chain variable region sequence - K-4,6,7,8,10
  source 1..324: mol_type = other DNA

SEQ ID NO: 423 - protein, 108 aa, synthetic construct; identical to SEQ ID NO: 186
  REGION 1..108: note = Monoclonal antibody 5D9G2C4 Translated protein, wherein the underlined sequence is the complementarity determining region (CDR)

SEQ ID NO: 424 - protein, 11 aa, synthetic construct
  REGION 1..11: note = Monoclonal antibody 5D9G2C4 Light chain variable region CDR 1
SASQGISNYLN

SEQ ID NO: 425 - protein, 7 aa, synthetic construct
  REGION 1..7: note = Monoclonal antibody 5D9G2C4 Light chain variable region CDR2
YTSSLHS

SEQ ID NO: 426 - protein, 9 aa, synthetic construct
  REGION 1..9: note = Monoclonal antibody 5D9G2C4 Light chain variable region CDR3
QQYSKLPYT

SEQ ID NO: 427 - DNA, 363 bp, synthetic construct; identical to SEQ ID NO: 365
  misc_feature 1..363: note = Monoclonal antibody 5F3A5D4 - Heavy chain variable region sequence - H-2,3,4,13,15
  source 1..363: mol_type = other DNA

SEQ ID NO: 428 - protein, 121 aa, synthetic construct; identical to SEQ ID NO: 175
  REGION 1..121: note = Monoclonal antibody 5F3A5D4 Translated protein, wherein the underlined sequence is the complementarity determining region (CDR)

SEQ ID NO: 429 - protein, 9 aa, synthetic construct
  REGION 1..9: note = Monoclonal antibody 5F3A5D4 Heavy chain variable region CDR1
NTFTEYTMH

SEQ ID NO: 430 - protein, 17 aa, synthetic construct
  REGION 1..17: note = Monoclonal antibody 5F3A5D4 Heavy chain variable region CDR2
GFNPNNGVTNYNQKFKG

SEQ ID NO: 431 - protein, 13 aa, synthetic construct
  REGION 1..13: note = Monoclonal antibody 5F3A5D4 Heavy chain variable region CDR3
RYYHSTYVFYFDS

SEQ ID NO: 432 - DNA, 324 bp, synthetic construct; identical to SEQ ID NO: 372
  misc_feature 1..324: note = Monoclonal antibody 5F3A5D4 Light chain variable region sequence - K-1,2,3,4,9
  source 1..324: mol_type = other DNA

SEQ ID NO: 433 - protein, 108 aa, synthetic construct; identical to SEQ ID NO: 186
  REGION 1..108: note = Monoclonal antibody 5F3A5D4 Translated protein, wherein the underlined sequence is the complementarity determining region (CDR)

SEQ ID NO: 434 - protein, 11 aa, synthetic construct
  REGION 1..11: note = Monoclonal antibody 5F3A5D4 Light chain variable region CDR1
SASQGISNYLN

SEQ ID NO: 435 - protein, 7 aa, synthetic construct
  REGION 1..7: note = Monoclonal antibody 5F3A5D4 Light chain variable region CDR2
YTSSLHS

SEQ ID NO: 436 - protein, 9 aa, synthetic construct
  REGION 1..9: note = Monoclonal antibody 5F3A5D4 Light chain variable region CDR3
QQYSKLPYT

SEQ ID NO: 437 - DNA, 357 bp, synthetic construct; identical to SEQ ID NO: 350
  misc_feature 1..357: note = Monoclonal antibody 8F9A5A1 - Heavy chain variable region sequence - H-3,4,6,10,11
  source 1..357: mol_type = other DNA

SEQ ID NO: 438 - protein, 119 aa, synthetic construct; identical to SEQ ID NO: 172
  REGION 1..119: note = Monoclonal antibody 8F9A5A1 Translated protein, wherein the underlined sequence is the complementarity determining region (CDR)

SEQ ID NO: 439 - protein, 9 aa, synthetic construct
  REGION 1..9: note = Monoclonal antibody 8F9A5A1 Heavy chain variable region CDR1
YTFTNYGMN

SEQ ID NO: 440 - protein, 17 aa, synthetic construct
  REGION 1..17: note = Monoclonal antibody 8F9A5A1 Heavy chain variable region CDR2
WINTYTGEPTYVDDFKG

SEQ ID NO: 441 - protein, 11 aa, synthetic construct
  REGION 1..11: note = Monoclonal antibody 8F9A5A1 Heavy chain variable region CDR3
LRGIRPGPLAY

SEQ ID NO: 442 - DNA, 321 bp, synthetic construct; identical to SEQ ID NO: 357
  misc_feature 1..321: note = Monoclonal antibody 8F9A5A1 Light chain variable region sequence - K-1,2,3,4,5
  source 1..321: mol_type = other DNA

SEQ ID NO: 443 - protein, 107 aa, synthetic construct; identical to SEQ ID NO: 191
  REGION 1..107: note = Monoclonal antibody 8F9A5A1 Translated protein, wherein the underlined sequence is the complementarity determining region (CDR)

SEQ ID NO: 444 - protein, 10 aa, synthetic construct
  REGION 1..10: note = Monoclonal antibody 8F9A5A1 Light chain variable region CDR 1
SASSSVSYMN

SEQ ID NO: 445 - protein, 7 aa, synthetic construct
  REGION 1..7: note = Monoclonal antibody 8F9A5A1 Light chain variable region CDR2
GISNLAS

SEQ ID NO: 446 - protein, 9 aa, synthetic construct
  REGION 1..9: note = Monoclonal antibody 8F9A5A1 Light chain variable region CDR3
QQRSSYPPT

SEQ ID NO: 447 - DNA, 363 bp, synthetic construct; identical to SEQ ID NO: 365
  misc_feature 1..363: note = Monoclonal antibody 8H5H5G4 - Heavy chain variable region sequence - H-1,3,5,6,10
  source 1..363: mol_type = other DNA

SEQ ID NO: 448 - protein, 121 aa, synthetic construct; identical to SEQ ID NO: 175
  REGION 1..121: note = Monoclonal antibody 8H5H5G4 Translated protein, wherein the underlined sequence is the complementarity determining region (CDR)

SEQ ID NO: 449 - protein, 9 aa, synthetic construct
  REGION 1..9: note = Monoclonal antibody 8H5H5G4 Heavy chain variable region CDR1
NTFTEYTMH

SEQ ID NO: 450 - protein, 17 aa, synthetic construct
  REGION 1..17: note = Monoclonal antibody 8H5H5G4 Heavy chain variable region CDR2
GFNPNNGVTNYNQKFKG

SEQ ID NO: 451 - protein, 13 aa, synthetic construct
  REGION 1..13: note = Monoclonal antibody 8H5H5G4 Heavy chain variable region CDR3
RYYHSTYVFYFDS

SEQ ID NO: 452 - DNA, 324 bp, synthetic construct; identical to SEQ ID NO: 372
  misc_feature 1..324: note = Monoclonal antibody 8H5H5G4 Light chain variable region sequence - K-2,5,8,9,15
  source 1..324: mol_type = other DNA

SEQ ID NO: 453 - protein, 108 aa, synthetic construct; identical to SEQ ID NO: 186
  REGION 1..108: note = Monoclonal antibody 8H5H5G4 Translated protein, wherein the underlined sequence is the complementarity determining region (CDR)

SEQ ID NO: 454 - protein, 11 aa, synthetic construct
  REGION 1..11: note = Monoclonal antibody 8H5H5G4 Light chain variable region CDR 1
SASQGISNYLN

SEQ ID NO: 455 - protein, 7 aa, synthetic construct
  REGION 1..7: note = Monoclonal antibody 8H5H5G4 Light chain variable region CDR2
YTSSLHS

SEQ ID NO: 456 - protein, 9 aa, synthetic construct
  REGION 1..9: note = Monoclonal antibody 8H5H5G4 Light chain variable region CDR3
QQYSKLPYT

SEQ ID NO: 457-1000 - skipped (544 entries)

SEQ ID NO: 1001 - protein, 121 aa, synthetic construct; identical to SEQ ID NO: 173
  REGION 1..121: note = Anti-NME7 B3 peptide monoclonal antibodies - 8F9A4P3 Heavy chain variable region sequence mouse

SEQ ID NO: 1002 - protein, 98 aa, synthetic construct
  REGION 1..98: note = IGHV1-24*01 V-REGION sequence human (closest match hu antibody sequence)
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAT

SEQ ID NO: 1003 - protein, 15 aa, synthetic construct
  REGION 1..15: note = IGHJ4*01 J-REGION sequence human (closest match hu antibody sequence)
YFDYWGQGTLVTVSS

SEQ ID NO: 1004 - protein, 122 aa, synthetic construct
  REGION 1..122: note = humanized 8F9A4P3 Heavy chain variable region sequence
QVQLVQSGAEVKKPGASVKVSCKVSGNTFTEYTMHWVRQAPGKGLEWMGGFNPNNGVTNYNQKFKGRVTMTEDTSTDTAYMELSSLRSEDTAVYYCARRYYHSLYVFYFDYWGQGTLVTVSS

SEQ ID NO: 1005 - protein, 122 aa, synthetic construct; identical to SEQ ID NO: 1004
  REGION 1..122: note = humanized 8F9A4P3 Heavy chain variable region sequence (codon optimized)

SEQ ID NO: 1006 - protein, 122 aa, synthetic construct
  REGION 1..122: note = Modified humanized 8F9A4P3 Heavy chain variable region sequence
QVQLVQSGAEVKKPGASVKVSCKVSGNTFTEYTMHWVRQAPGKGLEWIGGFNPNNGVTNYNQKFKGKVTLTVDTSSSTAYMELSSLRSEDTAVYYCARRYYHSLYVFYFDYWGQGTLVTVSS

SEQ ID NO: 1007 - protein, 122 aa, synthetic construct; identical to SEQ ID NO: 1006
  REGION 1..122: note = Modified humanized 8F9A4P3 Heavy chain variable region sequence (codon optimized)

SEQ ID NO: 1008 - protein, 108 aa, synthetic construct; identical to SEQ ID NO: 185
  REGION 1..108: note = 8F9A4P3 Light chain variable region sequence mouse

SEQ ID NO: 1009 - protein, 88 aa, synthetic construct
  REGION 1..88: note = IGKV5-2*01 V-REGION sequence human (closest match hu antibody sequence)
ETTLTQSPAFMSATPGDKVNISCKASQDIDDDMNWYQQKPGEAAIFIIQEATTLVPGIPPRFSGSGYGTDFTLTINNIESEDAAYYFC

SEQ ID NO: 1010 - protein, 12 aa, synthetic construct
  REGION 1..12: note = IGKJ4*02 J-REGION sequence human (closest match hu antibody sequence)
LTFGGGTKVEIK

SEQ ID NO: 1011 - protein, 108 aa, synthetic construct
  REGION 1..108: note = humanized 8F9A4P3 Light chain variable region sequence
ETTLTQSPAFMSATPGDKVNISCITSTDIDDDMNWYQQKPGEAAIFIIQEGNTLRPGIPPRFSGSGYGTDFTLTINNIESEDAAYYFCLQSDNLPLTFGGGTKVEIKR

SEQ ID NO: 1012 - protein, 108 aa, synthetic construct; identical to SEQ ID NO: 1011
  REGION 1..108: note = humanized 8F9A4P3 Light chain variable region sequence (codon optimized)

SEQ ID NO: 1013 - protein, 108 aa, synthetic construct
  REGION 1..108: note = Modified humanized 8F9A4P3 Light chain variable region sequence
ETTVTQSPAFMSATPGDKVTISCITSTDIDDDMNWYQQKPGEAAILLISEGNTLRPGIPPRFSSSGYGTDFTLTINNIESEDAAYYFCLQSDNLPLTFGGGTKVEIKR

SEQ ID NO: 1014 - protein, 108 aa, synthetic construct; identical to SEQ ID NO: 1013
  REGION 1..108: note = Modified humanized 8F9A4P3 Light chain variable region sequence (codon optimized)

SEQ ID NO: 1015 - protein, 245 aa, synthetic construct
  REGION 1..245: note = Modified humanized 8F9A4P3 sequence (codon optimized) - humanized heavy and light chains joined via a flexible linker
QVQLVQSGAEVKKPGASVKVSCKVSGNTFTEYTMHWVRQAPGKGLEWIGGFNPNNGVTNYNQKFKGKVTLTVDTSSSTAYMELSSLRSEDTAVYYCARRYYHSLYVFYFDYWGQGTLVTVSSGGGGSGGGGSGGGGSETTVTQSPAFMSATPGDKVTISCITSTDIDDDMNWYQQKPGEAAILLISEGNTLRPGIPPRFSSSGYGTDFTLTINNIESEDAAYYFCLQSDNLPLTFGGGTKVEIKR

SEQ ID NO: 1016 - protein, 119 aa, synthetic construct; identical to SEQ ID NO: 172
  REGION 1..119: note = 8F9A5A1 Heavy chain variable region sequence

SEQ ID NO: 1017 - protein, 98 aa, synthetic construct
  REGION 1..98: note = IGHV7-81*01 V-REGION sequence
QVQLVQSGHEVKQPGASVKVSCKASGYSFTTYGMNWVPQAPGQGLEWMGWFNTYTGNPTYAQGFTGRFVFSMDTSASTAYLQISSLKAEDMAMYYCAR

SEQ ID NO: 1018 - protein, 15 aa, synthetic construct
  REGION 1..15: note = IGHJ4*03 J-REGION sequence
YFDYWGQGTLVTVSS

SEQ ID NO: 1019 - protein, 120 aa, synthetic construct
  REGION 1..120: note = humanized 8F9A5A1 Heavy chain variable region sequence
QVQLVQSGHEVKQPGASVKVSCKASGYTFTNYGMNWVPQAPGQGLEWMGWINTYTGEPTYVDDFKGRFVFSMDTSASTAYLQISSLKAEDMAMYYCARLRGIRPGPLAYWGQGTLVTVSS

SEQ ID NO: 1020 - protein, 120 aa, synthetic construct; identical to SEQ ID NO: 1019
  REGION 1..120: note = humanized 8F9A5A1 Heavy chain variable region sequence (codon optimized)

SEQ ID NO: 1021 - protein, 120 aa, synthetic construct
  REGION 1..120: note = Modified humanized 8F9A5A1 Heavy chain variable region sequence
QIQLVQSGPEVKQPGASVKVSCKASGYTFTNYGMNWVKQAPGQGLEWMGWINTYTGEPTYVDDFKGRFAFSMDTSASTAYLQISSLKAEDTATYYCARLRGIRPGPLAYWGQGTLVTVSS

SEQ ID NO: 1022 - protein, 120 aa, synthetic construct; identical to SEQ ID NO: 1021
  REGION 1..120: note = Modified humanized 8F9A5A1 Heavy chain variable region sequence (codon optimized)

SEQ ID NO: 1023 - protein, 107 aa, synthetic construct; identical to SEQ ID NO: 191
  REGION 1..107: note = 8F9A5A1 Light chain variable region sequence

SEQ ID NO: 1024 - protein, 93 aa, synthetic construct
  REGION 1..93: note = IGKV3D-15*02 V-REGION sequence
EIVMMQSPATLSVSPGERATLSCRASQSVSSNLAWYQQKPGQAPRLLIYGASTRATGIPARFSGSGSGTEFTLTISSLQSEDFAVYYCQQYNN

SEQ ID NO: 1025 - protein, 12 aa, synthetic construct
  REGION 1..12: note = IGKJ4*02 J-REGION sequence
LTFGGGTKVEIK

SEQ ID NO: 1026 - protein, 107 aa, synthetic construct
  REGION 1..107: note = humanized 8F9A5A1 Light chain variable region sequence
EIVMMQSPATLSVSPGERATLSCSASSSVSYMNWYQQKPGQAPRLLIYGISNLASGIPARFSGSGSGTEFTLTISSLQSEDFAVYYCQQRSSYPPTFGGGTKVEIKR

SEQ ID NO: 1027 - protein, 107 aa, synthetic construct; identical to SEQ ID NO: 1026
  REGION 1..107: note = humanized 8F9A5A1 Light chain variable region sequence (codon optimized)

SEQ ID NO: 1028 - protein, 107 aa, synthetic construct
  REGION 1..107: note = Modified humanized 8F9A5A1 Light chain variable region sequence
EIVLTQSPATLSVSPGERATLSCSASSSVSYMNWYQQKPGQAPRLWIYGISNLASGIPARFSGSGSGTSFSLTISSLQSEDFAVYYCQQRSSYPPTFGGGTKVEIKR

SEQ ID NO: 1029 - protein, 107 aa, synthetic construct; identical to SEQ ID NO: 1028
  REGION 1..107: note = Modified humanized 8F9A5A1 Light chain variable region sequence (codon optimized)

SEQ ID NO: 1030 - protein, 242 aa, synthetic construct
  REGION 1..242: note = Modified humanized 8F9A5A1 scFV sequence (codon optimized)
QIQLVQSGPEVKQPGASVKVSCKASGYTFTNYGMNWVKQAPGQGLEWMGWINTYTGEPTYVDDFKGRFAFSMDTSASTAYLQISSLKAEDTATYYCARLRGIRPGPLAYWGQGTLVTVSSGGGGSGGGGSGGGGSEIVLTQSPATLSVSPGERATLSCSASSSVSYMNWYQQKPGQAPRLWIYGISNLASGIPARFSGSGSGTSFSLTISSLQSEDFAVYYCQQRSSYPPTFGGGTKVEIKR

SEQ ID NO: 1031 - protein, 121 aa, synthetic construct; identical to SEQ ID NO: 175
  REGION 1..121: note = 8H5H5G4 Heavy chain variable region sequence

SEQ ID NO: 1032 - protein, 98 aa, synthetic construct; identical to SEQ ID NO: 1002
  REGION 1..98: note = IGHV1-24*01 V-REGION sequence

SEQ ID NO: 1033 - protein, 15 aa, synthetic construct
  REGION 1..15: note = IGHJ4*03 J-REGION sequence
YFDYWGQGTLVTVSS

SEQ ID NO: 1034 - protein, 122 aa, synthetic construct
  REGION 1..122: note = Humanized 8H5H5G4 Heavy chain variable region sequence
QVQLVQSGAEVKKPGASVKVSCKVSGNTFTEYTMHWVRQAPGKGLEWMGGFNPNNGVTNYNQKFKGRVTMTEDTSTDTAYMELSSLRSEDTAVYYCARRYYHSTYVFYFDSWGQGTLVTVSS

SEQ ID NO: 1035 - protein, 122 aa, synthetic construct; identical to SEQ ID NO: 1034
  REGION 1..122: note = Humanized 8H5H5G4 Heavy chain variable region sequence (codon optimized)

SEQ ID NO: 1036 - protein, 122 aa, synthetic construct
  REGION 1..122: note = Modified Humanized 8H5H5G4 Heavy chain variable region sequence
QVQLVQSGAEVKKPGASVKVSCKVSGNTFTEYTMHWVRQAPGKGLEWIGGFNPNNGVTNYNQKFKGKVTLTVDTSSSTAYMELSSLRSEDTAVYYCARRYYHSTYVFYFDSWGQGTLVTVSS

SEQ ID NO: 1037 - protein, 122 aa, synthetic construct; identical to SEQ ID NO: 1036
  REGION 1..122: note = Modified Humanized 8H5H5G4 Heavy chain variable region sequence (codon optimized)

SEQ ID NO: 1038 - protein, 108 aa, synthetic construct; identical to SEQ ID NO: 186
  REGION 1..108: note = 8H5H5G4 Light chain variable region sequence

SEQ ID NO: 1039 - protein, 95 aa, synthetic construct
  REGION 1..95: note = IGKV1-27*01 V-REGION sequence
DIQMTQSPSSLSASVGDRVTITCRASQGISNYLAWYQQKPGKVPKLLIYAASTLQSGVPSRFSGSGSGTDFTLTISSLQPEDVATYYCQKYNSAP

SEQ ID NO: 1040 - protein, 12 aa, synthetic construct
  REGION 1..12: note = IGKJ4*02 J-REGION sequence
LTFGGGTKVEIK

SEQ ID NO: 1041 - protein, 108 aa, synthetic construct
  REGION 1..108: note = humanized 8H5H5G4 Light chain variable region sequence
DIQMTQSPSSLSASVGDRVTITCSASQGISNYLNWYQQKPGKVPKLLIYYTSSLHSGVPSRFSGSGSGTDFTLTISSLQPEDVATYYCQQYSKLPYTFGGGTKVEIKR

SEQ ID NO: 1042 - protein, 108 aa, synthetic construct; identical to SEQ ID NO: 1041
  REGION 1..108: note = humanized 8H5H5G4 Light chain variable region sequence (codon optimized)

SEQ ID NO: 1043 - protein, 108 aa, synthetic construct
  REGION 1..108: note = Modified humanized 8H5H5G4 Light chain variable region sequence
DIQMTQSPSSLSASVGDRVTITCSASQGISNYLNWYQQKPGKVPKLLIYYTSSLHSGVPSRFSGSGSGTDYTLTISSLQPEDVATYYCQQYSKLPYTFGGGTKVEIKR

SEQ ID NO: 1044 - protein, 108 aa, synthetic construct; identical to SEQ ID NO: 1043
  REGION 1..108: note = Modified humanized 8H5H5G4 Light chain variable region sequence (codon optimized)

SEQ ID NO: 1045 - protein, 245 aa, synthetic construct
  REGION 1..245: note = Modified humanized 8H5H5G4 scFV sequence (codon optimized)
QVQLVQSGAEVKKPGASVKVSCKVSGNTFTEYTMHWVRQAPGKGLEWIGGFNPNNGVTNYNQKFKGKVTLTVDTSSSTAYMELSSLRSEDTAVYYCARRYYHSTYVFYFDSWGQGTLVTVSSGGGGSGGGGSGGGGSDIQMTQSPSSLSASVGDRVTITCSASQGISNYLNWYQQKPGKVPKLLIYYTSSLHSGVPSRFSGSGSGTDYTLTISSLQPEDVATYYCQQYSKLPYTFGGGTKVEIKR

SEQ ID NO: 1046 - protein, 330 aa, synthetic construct
  REGION 1..330: note = Human IgG1 heavy chain constant region sequence: (for making full antibody - pair with either kappa or lambda constant region; 2 plasmids, express together)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1047 - protein, 326 aa, synthetic construct
  REGION 1..326: note = Human IgG2 heavy chain constant region sequence: (for making full antibody - pair with either kappa or lambda constant region; 2 plasmids, express together)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 1048 - protein, 107 aa, synthetic construct [sequence omitted]

SEQ ID NO: 1049 - protein, 106 aa, synthetic construct
  REGION 1..106: note = Human Lambda light chain constant region sequence
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 1050 - protein, 232 aa, synthetic construct
  REGION 1..232: note = Human IgG1 Fc region sequence: (to be fused to scFv for homo-dimerizes)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1051 - protein, 228 aa, synthetic construct
  REGION 1..228: note = Human IgG2 Fc region sequence
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 1052-1100 - skipped (49 entries)

SEQ ID NO: 1101 - DNA, 366 bp, synthetic construct
  misc_feature 1..366: note = Humanized 8F9A4A3 H-1.46 heavy chain variable domain sequence
  source 1..366: mol_type = other DNA
caagtgcagctggtgcagagcggcgccgaggtgaagaaacctggcgccagcgtgaaagtgtcctgcaaggccagcggcaatacattcaccgagtacacaatgcactgggtcagacaggcccccggccagggcctggaatggatcggcggatttaaccccaacaacggcgtgacaaactacaaccagaagttcaagggcaaggtgaccatcacaagagacaccagcagcagcaccgtgtacatggaactgtcttctctgcggagcgaggataccgccgtgtactattgtgccagacggtactaccacagcctgtacgtgttctacttcgactactggggacagggcaccctggttaccgtgtcctct

SEQ ID NO: 1102 - protein, 122 aa, synthetic construct
  REGION 1..122: note = Humanized 8F9A4A3 H-1.46 heavy chain variable domain sequence
QVQLVQSGAEVKKPGASVKVSCKASGNTFTEYTMHWVRQAPGQGLEWIGGFNPNNGVTNYNQKFKGKVTITRDTSSSTVYMELSSLRSEDTAVYYCARRYYHSLYVFYFDYWGQGTLVTVSS

SEQ ID NO: 1103 - DNA, 321 bp, synthetic construct
  misc_feature 1..321: note = Humanized 8F9A4A3 L-1.6 light chain variable domain sequence
  source 1..321: mol_type = other DNA
gatatccagatgacacagagccctagctccctgagcgccagcgtgggcgaccgggtcaccattacatgcagcgcttctcagggcatctccaactacctgaactggtaccagcagaaacccggcaaggcccctaagctgctgatcttctacaccagctctctgcacagcggcgtgccatctagattcagcggatctggcagcggcaccgactacaccctgaccatcagctccctccagcctgaggacttcgccacctactactgtcagcaatacagcaagctgccttatacctttggcggcggaacaaaggtggaaatcaag

SEQ ID NO: 1104 - protein, 107 aa, synthetic construct
  REGION 1..107: note = Humanized 8F9A4A3 L-1.6 light chain variable domain sequence
DIQMTQSPSSLSASVGDRVTITCSASQGISNYLNWYQQKPGKAPKLLIFYTSSLHSGVPSRFSGSGSGTDYTLTISSLQPEDFATYYCQQYSKLPYTFGGGTKVEIK

SEQ ID NO: 1105 - DNA, 366 bp, synthetic construct
  misc_feature 1..366: note = Humanized 8F9A4A3 H-4.4 heavy chain variable domain sequence
  source 1..366: mol_type = other DNA
caagtgcagctgcaggagagcggacctggcctggttaagcctggaggcaccctgtctctgacatgtgctgtgtctggcaatacctttaccgagtacaccatgcactgggtgcggcagcctccaggcaagggcctggaatggatcggcggcttcaaccccaacaacggcgtgacaaattacaaccagaaattcaagggaaaagtgaccatcaccgtggataagtccaagaacaccttcagcctcaagctgagcagcgtgacagccgccgacaccgccgtgtactactgcgccagaagatactatcacagcctgtacgtgttctacttcgactactggggccagggcacactggtcaccgtgtccagc

SEQ ID NO: 1106 - protein, 122 aa, synthetic construct
  REGION 1..122: note = Humanized 8F9A4A3 H-4.4 heavy chain variable domain sequence
QVQLQESGPGLVKPGGTLSLTCAVSGNTFTEYTMHWVRQPPGKGLEWIGGFNPNNGVTNYNQKFKGKVTITVDKSKNTFSLKLSSVTAADTAVYYCARRYYHSLYVFYFDYWGQGTLVTVSS

SEQ ID NO: 1107 - DNA, 321 bp, synthetic construct
  misc_feature 1..321: note = Humanized 8F9A4A3 L-4.1 light chain variable domain sequence
  source 1..321: mol_type = other DNA
gatatcgtgatgacccagagcccagacagcctggcagtgagtctgggtgagcgtgctacaatcaactgcagcgccagccagggcatctccaactacctgaattggtatcagcagaaacctggccaggctcctaagctgctgatcttctacaccagcagcctgcacagcggcgtgccagatagattcagcggcagcggatctggcaccgactacacactgaccatttcttctctccaggccgaggacgtggccgtctactactgtcagcaatacagcaagctgccttacacctttggcggaggcacaaaggtggaaatcaag

SEQ ID NO: 1108 - protein, 107 aa, synthetic construct
  REGION 1..107: note = Humanized 8F9A4A3 L-4.1 light chain variable domain sequence
DIVMTQSPDSLAVSLGERATINCSASQGISNYLNWYQQKPGQAPKLLIFYTSSLHSGVPDRFSGSGSGTDYTLTISSLQAEDVAVYYCQQYSKLPYTFGGGTKVEIK

SEQ ID NO: 1109 - protein, 107 aa, synthetic construct
  REGION 1..107: note = 8F9A4A3L
DIQMTQTTSSLSASLGDRVTLSCSASQGISNYLNWYQQKPDGTVELLIFYTSSLHSGVPSRFSGSGSGTDYSLTISNLEPEDIATYYCQQYSKLPYTFGGGTKLEIK

SEQ ID NO: 1110 - protein, 187 aa, synthetic construct
  REGION 1..187: note = NME4
MGGLFWRSALRGLRCGPRAPGPSLLVRHGSGGPSWTRERTLVAVKPDGVQRRLVGDVIQRFERRGFTLVGMKMLQAPESVLAEHYQDLRRKPFYPALIRYMSSGPVVAMVWEGYNVVRASRAMIGHTDSAEAAPGTIRGDFSVHISRNVIHASDSVEGAQREIQLWFQSSELVSWADGGQHSSIHPA

SEQ ID NO: 1111 - protein, 254 aa, synthetic construct
  REGION 1..254: note = NME5
MEISMPPPQIYVEKTLAIIKPDIVDKEEEIQDIILRSGFTIVQRRKLRLSPEQCSNFYVEKYGKMFFPNLTAYMSSGPLVAMILARHKAISYWLELLGPNNSLVAKETHPDSLRAIYGTDDLRNALHGSNDFAAAEREIRFMFPEVIVEPIPIGQAAKDYLNLHIMPTLLEGLTELCKQKPADPLFWYMCCRREHWTLRSILLVCMSGIRMSLPHCADYCSFVEGFEIWLADWLLKNNPNKPKLCHHPIVEEPY

SEQ ID NO: 1112 - protein, 588 aa, synthetic construct
  REGION 1..588: note = NME8
MASKKREVQLQTVINNQSLWDEMLQNKGLTVIDVYQAWCGPCRAMQPLFRKLKNELNEDEILHFAVAEADNIVTLQPFRDKCEPVFLFSVNGKIIEKIQGANAPLVNKKVINLIDEERKIAAGEMARPQYPEIPLVDSDSEVSEESPCESVQELYSIAIIKPDAVISKKVLEIKRKITKAGFIIEAEHKTVLTEEQVVNFYSRIADQCDFEEFVSFMTSGLSYILVVSQGSKHNPPSEETEPQTDTEPNERSEDQPEVEAQVTPGMMKNKQDSLQEYLERQHLAQLCDIEEDAANVAKFMDAFFPDFKKMKSMKLEKTLALLRPNLFHERKDDVLRIIKDEDFKILEQRQVVLSEKEAQALCKEYENEDYFNKLIENMTSGPSLALVLLRDNGLQYWKQLLGPRTVEEAIEYFPESLCAQFAMDSLPVNQLYGSDSLETAEREIQHFFPLQSTLGLIKPHATSEQREQILKIVKEAGFDLTQVKKMFLTPEQIEKIYPKVTGKDFYKDLLEMLSVGPSMVMILTKWNAVAEWRRLMGPTDPEEAKLLSPDSIRAQFGISKLKNIVHGASNAYEAKEVVNRLFEDPEEN

SEQ ID NO: 1113 - DNA, 78 bp, synthetic construct
  misc_feature 1..78: note = Mouse 8F9A4A3 heavy chain variable domain framework 1
  source 1..78: mol_type = other DNA
gaggtccagctgcaacagtctggacctgaactggtgaagcctggggcttcagtgaagatatcctgcaagacttctgga

SEQ ID NO: 1114 - protein, 26 aa, synthetic construct
  REGION 1..26: note = Mouse 8F9A4A3 heavy chain variable domain framework 1
EVQLQQSGPELVKPGASVKISCKTSG

SEQ ID NO: 1115 - DNA, 27 bp, synthetic construct
  misc_feature 1..27: note = Heavy chain variable region CDR1
  source 1..27: mol_type = other DNA
aacacattcactgaatacaccatgcac

SEQ ID NO: 1116 - DNA, 42 bp, synthetic construct
  misc_feature 1..42: note = Mouse 8F9A4A3 heavy chain variable domain framework 2
  source 1..42: mol_type = other DNA
tgggtgaagcagagccatggaaagagccttgagtggattgga

SEQ ID NO: 1117 - protein, 14 aa, synthetic construct
  REGION 1..14: note = Mouse 8F9A4A3 heavy chain variable domain framework 2
WVKQSHGKSLEWIG

SEQ ID NO: 1118 - DNA, 51 bp, synthetic construct
  misc_feature 1..51: note = Mouse 8F9A4A3 Heavy chain variable region CDR2
  source 1..51: mol_type = other DNA
ggttttaatcctaacaatggtgttactaactacaaccagaagttcaagggc

SEQ ID NO: 1119 - DNA, 96 bp, synthetic construct
  misc_feature 1..96: note = Mouse 4A3 heavy chain variable domain framework 3
  source 1..96: mol_type = other DNA
aaggccacattgactgtagacaagtcctccagcacagcctacatggagctccgcagcctgacatctgaggattctgcagtctattactgtgcaaga

SEQ ID NO: 1120 - protein, 32 aa, synthetic construct
  REGION 1..32: note = Mouse 4A3 heavy chain variable domain framework 3
KATLTVDKSSSTAYMELRSLTSEDSAVYYCAR

SEQ ID NO: 1121 - DNA, 39 bp, synthetic construct
  misc_feature 1..39: note = Mouse 8F9A4A3 Heavy chain variable region CDR3
  source 1..39: mol_type = other DNA
cggtactaccatagtctctacgtgttttactttgactac

SEQ ID NO: 1122 - DNA, 69 bp, synthetic construct
  misc_feature 1..69: note = Mouse 8F9A4A3 light chain variable domain framework 1
  source 1..69: mol_type = other DNA
gatatccagatgacacagactacatcctccctgtctgcctctctgggagacagagtcaccctcagttgc

SEQ ID NO: 1123 - protein, 23 aa, synthetic construct
  REGION 1..23: note = Mouse 8F9A4A3 light chain variable domain framework 1
DIQMTQTTSSLSASLGDRVTLSC

SEQ ID NO: 1124 - DNA, 33 bp, synthetic construct
  misc_feature 1..33: note = Mouse 8F9A4A3 Light chain variable region CDR 1
  source 1..33: mol_type = other DNA
agtgcaagtcagggcattagcaattatttaaac

SEQ ID NO: 1125 - DNA, 45 bp, synthetic construct
  misc_feature 1..45: note = Mouse 4A3 light chain variable domain framework 2
  source 1..45: mol_type = other DNA
tggtatcagcagaaaccagatggaactgttgaactcctgatcttt

SEQ ID NO: 1126 - protein, 15 aa, synthetic construct
  REGION 1..15: note = Mouse 4A3 light chain variable domain framework 2
WYQQKPDGTVELLIF

SEQ ID NO: 1127 - DNA, 21 bp, synthetic construct
  misc_feature 1..21: note = Mouse 8F9A4A3 Light chain variable region CDR2
  source 1..21: mol_type = other DNA
tacacatcaagtttacactca

SEQ ID NO: 1128 - DNA, 96 bp, synthetic construct
  misc_feature 1..96: note = Mouse 8F9A4A3 light chain variable domain framework 3
  source 1..96: mol_type = other DNA
ggagtcccatcaaggttcagtggcagtgggtctgggacagattattctctcaccatcagcaacctggaacctgaagatattgccacttactattgt

SEQ ID NO: 1129 - protein, 32 aa, synthetic construct
  REGION 1..32: note = Mouse 8F9A4A3 light chain variable domain framework 3
GVPSRFSGSGSGTDYSLTISNLEPEDIATYYC

SEQ ID NO: 1130 - DNA, 27 bp, synthetic construct
  misc_feature 1..27: note = Mouse 8F9A4A3 Light chain variable region CDR3
  source 1..27: mol_type = other DNA
cagcagtatagtaagcttccttacacg

SEQ ID NO: 1131 - DNA, 366 bp, synthetic construct
  misc_feature 1..366: note = Humanized 8F9A4A3 H-ori heavy chain variable domain sequence
  source 1..366: mol_type = other DNA
caggttcagctggttcagtctggtgcagaagtgaagaaacctggcgcctctgtgaaggtgtcctgcaaggtgtccggaaataccttcaccgagtacaccatgcactgggtccgacaggcccctggcaaaggacttgaatggatgggcggcttcaaccccaacaacggcgtgaccaactacaaccagaaattcaagggccgcgtgaccatgaccgaggacacaagcacagacaccgcctacatggaactgagcagcctgagaagcgaggacaccgccgtgtactactgcgccagaaggtactaccacagcctgtacgtgttctacttcgactactggggccagggcaccctggtcacagtttcttct

SEQ ID NO: 1132 - DNA, 366 bp, synthetic construct
  misc_feature 1..366: note = Humanized 8F9A4A3 H-3.15 heavy chain variable domain sequence
  source 1..366: mol_type = other DNA
gaggtgcagctggtggaaagcggcggcggcctggttaagcctggcggatctctgagactgagctgtgccgcttctggcaataccttcaccgagtacaccatgcactgggtgcggcaggcccctggaaaaggcctggaatggatcggcggatttaaccccaacaacggcgtgacaaattacaaccagaaattcaagggcaagttcaccatcacaagagataagagcaagaacaccctgtacctgcaaatgaacagcctgaagtccgaggacaccgccgtgtactactgcgccagacggtactaccacagcctctatgtgttctacttcgactactggggccagggcacactggtcaccgtgtccagc

SEQ ID NO: 1133 - protein, 122 aa, synthetic construct
  REGION 1..122: note = Humanized 8F9A4A3 H-3.15 heavy chain variable domain sequence
EVQLVESGGGLVKPGGSLRLSCAASGNTFTEYTMHWVRQAPGKGLEWIGGFNPNNGVTNYNQKFKGKFTITRDKSKNTLYLQMNSLKSEDTAVYYCARRYYHSLYVFYFDYWGQGTLVTVSS

SEQ ID NO: 1134 - DNA, 321 bp, synthetic construct
  misc_feature 1..321: note = Humanized 8F9A4A3 L-3.15 light chain variable domain sequence
  source 1..321: mol_type = other DNA
gagatcgtgatgacccagagcccagctacacttagtgtgagtccaggtgaacgggctaccctgtcctgcagcgccagccagggcatcagcaactacctgaactggtaccagcagaaacctggccaggcccctagactgctgatcttctacaccagcagcctgcacagcggcatccccgccagattcagcggcagcggctctggaacagactacaccctgacaatctctagcctgcagtctgaagattttgccgtctactactgtcagcaatacagcaagctgccttataccttcggcggcggaaccaaggtggaaattaag

SEQ ID NO: 1135 - protein, 107 aa, synthetic construct
  REGION 1..107: note = Humanized 8F9A4A3 L-3.15 light chain variable domain sequence
EIVMTQSPATLSVSPGERATLSCSASQGISNYLNWYQQKPGQAPRLLIFYTSSLHSGIPARFSGSGSGTDYTLTISSLQSEDFAVYYCQQYSKLPYTFGGGTKVEIK

SEQ ID NO: 1136 - DNA, 321 bp, synthetic construct
  misc_feature 1..321: note = Mouse 8F9A4A3 Light chain variable region sequence
  source 1..321: mol_type = other DNA
gatatccagatgacacagactacatcctccctgtctgcctctctgggagacagagtcaccctcagttgcagtgcaagtcagggcattagcaattatttaaactggtatcagcagaaaccagatggaactgttgaactcctgatcttttacacatcaagtttacactcaggagtcccatcaaggttcagtggcagtgggtctgggacagattattctctcaccatcagcaacctggaacctgaagatattgccacttactattgtcagcagtatagtaagcttccttacacgttcggaggggggaccaagctggaaataaaa

SEQ ID NO: 1137 - protein, 152 aa, Homo sapiens
MANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 1138 - DNA, 366 bp, synthetic construct
  misc_feature 1..366: note = Monoclonal antibody 8F9A4A3 "4A3"
  source 1..366: mol_type = other DNA
gaggtccagctgcaacagtctggacctgaactggtgaagcctggggcttcagtgaagatatcctgcaagacttctggaaacacattcactgaatacaccatgcactgggtgaagcagagccatggaaagagccttgagtggattggaggttttaatcctaacaatggtgttactaactacaaccagaagttcaagggcaaggccacattgactgtagacaagtcctccagcacagcctacatggagctccgcagcctgacatctgaggattctgcagtctattactgtgcaagacggtactaccatagtctctacgtgttttactttgactactggggccaaggcaccactctcacagtctcctca

SEQ ID NO: 1139 - protein, 122 aa, synthetic construct
  REGION 1..122: note = Monoclonal antibody 8F9A4A3 "4A3"
EVQLQQSGPELVKPGASVKISCKTSGNTFTEYTMHWVKQSHGKSLEWIGGFNPNNGVTNYNQKFKGKATLTVDKSSSTAYMELRSLTSEDSAVYYCARRYYHSLYVFYFDYWGQGTTLTVSS

SEQ ID NO: 1140 - protein, 131 aa, Homo sapiens; identical to SEQ ID NO: 199

SEQ ID NO: 1141 - protein, 376 aa, Homo sapiens; identical to SEQ ID NO: 82

SEQ ID NO: 1142 - protein, 30 aa, synthetic construct
  REGION 1..30: note = B3 region of NME7
AIFGKTKIQNAVHCTDLPEDGLLEVQYFFC

SEQ ID NO: 1143 - protein, 139 aa, Homo sapiens
NCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 1144 - protein, 11 aa, synthetic construct
  REGION 1..11: note = Light Chain CDR1 region
ITSTDIDDDMN

SEQ ID NO: 1145 - protein, 7 aa, synthetic construct
  REGION 1..7: note = Light Chain CDR2 region
EGNTLRP

SEQ ID NO: 1146 - protein, 9 aa, synthetic construct
  REGION 1..9: note = Light Chain CDR3 region
LQSDNLPLT

SEQ ID NO: 1147 - protein, 23 aa, synthetic construct
  REGION 1..23: note = Mouse 5F3A5D4-2 light chain variable domain framework 1
DIQMTQTTSSLSASLGDRVTISC

SEQ ID NO: 1148 - DNA, 45 bp, synthetic construct
  misc_feature 1..45: note = Mouse 5F3A5D4-2 light chain variable domain framework 2
  source 1..45: mol_type = other DNA
tggtttcagcagaaaccagatggaactattaagctcctgatctat

SEQ ID NO: 1149 - protein, 15 aa, synthetic construct
  REGION 1..15: note = Mouse 5F3A5D4-2 light chain variable domain framework 2
WFQQKPDGTIKLLIY

SEQ ID NO: 1150 - DNA, 21 bp, synthetic construct
  misc_feature 1..21: note = Mouse 5F3A5D4-2 light chain variable domain Complementarity -determining region 2
  source 1..21: mol_type = other DNA
tacacatcaagtttacattca

SEQ ID NO: 1151 - DNA, 96 bp, synthetic construct
  misc_feature 1..96: note = Mouse 5F3A5D4-2 light chain variable domain framework 3
  source 1..96: mol_type = other DNA
ggagtcccatcaaggttcagtggcagtgggtctgggacagattattctctcaccatcagtaatgtggaacctgaagatattgccacttactattgt

SEQ ID NO: 1152 - protein, 32 aa, synthetic construct
  REGION 1..32: note = Mouse 5F3A5D4-2 light chain variable domain framework 3
GVPSRFSGSGSGTDYSLTISNVEPEDIATYYC

SEQ ID NO: 1153 - DNA, 30 bp, synthetic construct
  misc_feature 1..30: note = Mouse 5F3A5D4-2 light chain variable domain framework 4
  source 1..30: mol_type = other DNA
ttcggaggggggaccaagctggagataaaa

SEQ ID NO: 1154 - protein, 10 aa, synthetic construct
  REGION 1..10: note = Mouse 5F3A5D4-2 light chain variable domain framework 4
FGGGTKLEIK

SEQ ID NO: 1155 - DNA, 366 bp, synthetic construct
  misc_feature 1..366: note = Mouse 5F3A5D4-V2 heavy chain variable domain sequence
  source 1..366: mol_type = other DNA
gaggtccagctgcaacagtctggacctgatctggtgaagcctgggacttcagtgaagatatcctgtaagacttctggaaacacattcactgaatacaccatgcactgggtgaagcagagccatggaaagagccttgagtggattggaggttttaatcctaacaatggtgttactaactacaaccagaagttcaagggcaaggccacattgactgtagacaagtcctccagcacagcctacatggagctccgcagcctgacatctgaggattctgcagtctattactgtgcaagacgttactaccatagtacctacgtgttctactttgactcctggggccaaggcaccactctcacagtctcctca

SEQ ID NO: 1156 - DNA, 69 bp, synthetic construct; identical to SEQ ID NO: 1122
  misc_feature 1..69: note = Mouse 5F3A5D4-3 light chain variable domain framework 1
  source 1..69: mol_type = other DNA

SEQ ID NO: 1157 - DNA, 30 bp, synthetic construct
  misc_feature 1..30: note = Mouse 5F3A5D4-3 light chain variable domain framework 4
  source 1..30: mol_type = other DNA
ttcggaggggggaccaagctggaaataaaa

SEQ ID NO: 1158 - DNA, 366 bp, synthetic construct
  misc_feature 1..366: note = Humanized 5F3A5D4-V2 H-1.27 heavy chain variable domain sequence
  source 1..366: mol_type = other DNA
caagtgcagctggtccagagcggcgccgaggtgaaaaagcctggcgccagcgtgaaggtgtcctgcaaggtgtctggcaataccttcaccgagtacaccatgcactgggtgcggcaggcccctggaaaaggcctggaatggatcggcggatttaaccccaacaacggcgtgaccaactacaaccagaagttcaagggcaaggttacactgaccgtggacaccagctcttctaccgcctacatggaactgagcagcctgagaagcgaggatacagccgtgtactattgtgccagaagatactaccacagcacctacgtgttctacttcgacagctggggccagggcacactggtgacagtgtccagc

SEQ ID NO: 1159 - DNA, 366 bp, synthetic construct
  misc_feature 1..366: note = Humanized 5F3A5D4-V2 H-1.46 heavy chain variable domain sequence
  source 1..366: mol_type = other DNA
caagtgcagctggtgcagagcggagccgaggtgaaaaagcccggcgcttctgtgaaggtgtcttgtaaagccagcggcaacaccttcaccgagtacaccatgcactgggtgcggcaggcccctggccagggcctggaatggatcggcggctttaatcctaacaacggcgtgacaaactacaaccagaagttcaagggcaaggttacaatcaccagagataccagcagctctaccgtgtacatggaactgagcagcctgagaagcgaggacaccgccgtgtattactgcgccagacggtactaccacagcacctacgtgttctacttcgacagctggggccagggaacactggtcacagtgtcctcc

SEQ ID NO: 1160 - protein, 122 aa, synthetic construct
  REGION 1..122: note = Humanized 5F3A5D4-V2 H-1.46 heavy chain variable domain sequence
QVQLVQSGAEVKKPGASVKVSCKASGNTFTEYTMHWVRQAPGQGLEWIGGFNPNNGVTNYNQKFKGKVTITRDTSSSTVYMELSSLRSEDTAVYYCARRYYHSTYVFYFDSWGQGTLVTVSS

SEQ ID NO: 1161 - DNA, 366 bp, synthetic construct
  misc_feature 1..366: note = Humanized 5F3A5D4-V2 H-3.15 heavy chain variable domain sequence
  source 1..366: mol_type = other DNA
gaggtgcagctggtggaaagcggaggaggcctggttaagcctggaggcagcctgagactgagctgtgccgcttctggcaataccttcaccgagtacaccatgcactgggtgcggcaggcccctggcaaaggcctggaatggatcggcggcttcaaccccaacaacggcgtgacaaattacaaccagaaattcaagggcaagtttacaatcaccagagataagtctaagaacacactctatctgcaaatgaacagcctgaagtccgaggacaccgccgtgtactactgcgccagacggtactaccacagcacatacgtgttctacttcgacagctggggccagggcaccctggtcaccgtgtccagc

SEQ ID NO: 1162 - protein, 122 aa, synthetic construct
  REGION 1..122: note = Humanized 5F3A5D4-V2 H-3.15 heavy chain variable domain sequence
EVQLVESGGGLVKPGGSLRLSCAASGNTFTEYTMHWVRQAPGKGLEWIGGFNPNNGVTNYNQKFKGKFTITRDKSKNTLYLQMNSLKSEDTAVYYCARRYYHSTYVFYFDSWGQGTLVTVSS

SEQ ID NO: 1163 - DNA, 366 bp, synthetic construct
  misc_feature 1..366: note = Humanized 5F3A5D4-V2 H-4.4 heavy chain variable domain sequence
  source 1..366: mol_type = other DNA
caagtgcagctgcaggagagcggacctggcctggtcaagcctggcggcaccctgagcctcacctgtgctgtttctggcaataccttcaccgagtacaccatgcactgggtgcggcagcctccaggcaaaggcctggaatggatcggcggatttaaccccaacaacggcgtgacaaattacaaccagaaattcaagggcaaggtgaccatcacagtggataagtccaagaacaccttcagcctgaagctgtctagcgtgacagccgccgacaccgccgtgtactactgcgccagaagatactatcacagcacctacgtgttctacttcgacagctggggacagggcacactggtgacagtgtccagc

SEQ ID NO: 1164 - protein, 122 aa, synthetic construct
  REGION 1..122: note = Humanized 5F3A5D4-V2 H-4.4 heavy chain variable domain sequence
QVQLQESGPGLVKPGGTLSLTCAVSGNTFTEYTMHWVRQPPGKGLEWIGGFNPNNGVTNYNQKFKGKVTITVDKSKNTFSLKLSSVTAADTAVYYCARRYYHSTYVFYFDSWGQGTLVTVSS

SEQ ID NO: 1165 - DNA, 321 bp, synthetic construct
  misc_feature 1..321: note = Humanized 5F3A5D4-2 L-1.6 light chain variable domain sequence
  source 1..321: mol_type = other DNA
gatatccagatgacacagagccctagctccctgagcgccagcgtgggcgaccgggtcaccattacatgcagcgcttctcagggcatctccaactacctgaactggtttcagcagaaacccggcaaggcccctaagctgctgatctattacaccagctctctgcacagcggcgtgccatctagattcagcggatctggcagcggcaccgactacaccctgaccatcagctccctccagcctgaggacttcgccacctactactgtcagcaatacagcaagctgccttatacctttggcggcggaacaaaggtggaaatcaag

SEQ ID NO: 1166 - protein, 107 aa, synthetic construct
  REGION 1..107: note = Humanized 5F3A5D4-2 L-1.6 light chain variable domain sequence
DIQMTQSPSSLSASVGDRVTITCSASQGISNYLNWFQQKPGKAPKLLIYYTSSLHSGVPSRFSGSGSGTDYTLTISSLQPEDFATYYCQQYSKLPYTFGGGTKVEIK

SEQ ID NO: 1167 - DNA, 321 bp, synthetic construct
  misc_feature 1..321: note = Humanized 5F3A5D4-2 L-3.15 light chain variable domain sequence
  source 1..321: mol_type = other DNA
gagatcgtgatgacccagagcccagctacacttagtgtgagtccaggtgaacgggctaccctgtcctgcagcgccagccagggcatcagcaactacctgaactggtttcagcagaaacctggccaggcccctagactgctgatctattacaccagcagcctgcacagcggcatccccgccagattcagcggcagcggctctggaacagactacaccctgacaatctctagcctgcagtctgaagattttgccgtctactactgtcagcaatacagcaagctgccttataccttcggcggcggaaccaaggtggaaattaag

SEQ ID NO: 1168 - protein, 107 aa, synthetic construct
  REGION 1..107: note = Humanized 5F3A5D4-2 L-3.15 light chain variable domain sequence
EIVMTQSPATLSVSPGERATLSCSASQGISNYLNWFQQKPGQAPRLLIYYTSSLHSGIPARFSGSGSGTDYTLTISSLQSEDFAVYYCQQYSKLPYTFGGGTKVEIK

SEQ ID NO: 1169 - DNA, 321 bp, synthetic construct
  misc_feature 1..321: note = Humanized 5F3A5D4-2 L-4.1 light chain variable domain sequence
  source 1..321: mol_type = other DNA
gatatcgtgatgacccagagcccagacagcctggcagtgagtctgggtgagcgtgctacaatcaactgcagcgccagccagggcatctccaactacctgaattggtttcagcagaaacctggccaggctcctaagctgctgatctattacaccagcagcctgcacagcggcgtgccagatagattcagcggcagcggatctggcaccgactacacactgaccatttcttctctccaggccgaggacgtggccgtctactactgtcagcaatacagcaagctgccttacacctttggcggaggcacaaaggtggaaatcaag

SEQ ID NO: 1170 - protein, 107 aa, synthetic construct
  REGION 1..107: note = Humanized 5F3A5D4-2 L-4.1 light chain variable domain sequence
DIVMTQSPDSLAVSLGERATINCSASQGISNYLNWFQQKPGQAPKLLIYYTSSLHSGVPDRFSGSGSGTDYTLTISSLQAEDVAVYYCQQYSKLPYTFGGGTKVEIK

SEQ ID NO: 1171 - DNA, 69 bp, synthetic construct
  misc_feature 1..69: note = Mouse 5F3A5D4-2 light chain variable domain framework 1
  source 1..69: mol_type = other DNA
gatatccagatgacacagactacatcctccctgtctgcctctctgggagacagagtcaccatcagttgc

SEQ ID NO: 1172 - protein, 122 aa, synthetic construct
  REGION 1..122: note = Mouse 5F3A5D4-V2 heavy chain variable domain sequence
EVQLQQSGPDLVKPGTSVKISCKTSGNTFTEYTMHWVKQSHGKSLEWIGGFNPNNGVTNYNQKFKGKATLTVDKSSSTAYMELRSLTSEDSAVYYCARRYYHSTYVFYFDSWGQGTTLTVSS

SEQ ID NO: 1173 - DNA, 78 bp, synthetic construct
  misc_feature 1..78: note = Mouse 5F3A5D4-V2 heavy chain variable domain framework 1
  source 1..78: mol_type = other DNA
gaggtccagctgcaacagtctggacctgatctggtgaagcctgggacttcagtgaagatatcctgtaagacttctgga

SEQ ID NO: 1174 - protein, 26 aa, synthetic construct
  REGION 1..26: note = Mouse 5F3A5D4-V2 heavy chain variable domain framework 1
EVQLQQSGPDLVKPGTSVKISCKTSG

SEQ ID NO: 1175 - protein, 32 aa, synthetic construct; identical to SEQ ID NO: 1120
  REGION 1..32: note = Mouse 5F3A5D4-V2 heavy chain variable domain framework 3

SEQ ID NO: 1176 - DNA, 39 bp, synthetic construct
  misc_feature 1..39: note = Mouse 5F3A5D4-V2 heavy chain variable domain Complementarity -determining region 3
  source 1..39: mol_type = other DNA
cgttactaccatagtacctacgtgttctactttgactcc

SEQ ID NO: 1177 - DNA, 33 bp, synthetic construct
  misc_feature 1..33: note = Mouse 5F3A5D4-V2 heavy chain variable domain framework 4
  source 1..33: mol_type = other DNA
tggggccaaggcaccactctcacagtctcctca

SEQ ID NO: 1178 - protein, 11 aa, synthetic construct
  REGION 1..11: note = Mouse 5F3A5D4-V2 heavy chain variable domain framework 4
WGQGTTLTVSS

SEQ ID NO: 1179 - DNA, 321 bp, synthetic construct
  misc_feature 1..321: note = Mouse 5F3A5D4-2 light chain variable domain sequence
  source 1..321: mol_type = other DNA
gatatccagatgacacagactacatcctccctgtctgcctctctgggagacagagtcaccatcagttgcagtgcaagtcagggcattagcaattatttaaactggtttcagcagaaaccagatggaactattaagctcctgatctattacacatcaagtttacattcaggagtcccatcaaggttcagtggcagtgggtctgggacagattattctctcaccatcagtaatgtggaacctgaagatattgccacttactattgtcagcagtatagtaagcttccttacacgttcggaggggggaccaagctggagataaaa

SEQ ID NO: 1180 - protein, 107 aa, synthetic construct
  REGION 1..107: note = Mouse 5F3A5D4-2 light chain variable domain sequence
DIQMTQTTSSLSASLGDRVTISCSASQGISNYLNWFQQKPDGTIKLLIYYTSSLHSGVPSRFSGSGSGTDYSLTISNVEPEDIATYYCQQYSKLPYTFGGGTKLEIK

SEQ ID NO: 1181 - protein, 122 aa, synthetic construct; identical to SEQ ID NO: 1036
  REGION 1..122: note = Humanized 5F3A5D4-V2 H-1.27 heavy chain variable domain sequence

SEQ ID NO: 1182 - DNA, 321 bp, synthetic construct; identical to SEQ ID NO: 1103
  misc_feature 1..321: note = Humanized 5F3A5D4-3 L-1.6 light chain variable domain sequence
  source 1..321: mol_type = other DNA

SEQ ID NO: 1183 - protein, 107 aa, synthetic construct; identical to SEQ ID NO: 1104
  REGION 1..107: note = Humanized 5F3A5D4-3 L-1.6 light chain variable domain sequence

SEQ ID NO: 1184 - DNA, 321 bp, synthetic construct; identical to SEQ ID NO: 1107
  misc_feature 1..321: note = Humanized 5F3A5D4-3 L-4.1 light chain variable domain sequence
  source 1..321: mol_type = other DNA

SEQ ID NO: 1185 - protein, 107 aa, synthetic construct; identical to SEQ ID NO: 1108
  REGION 1..107: note = Humanized 5F3A5D4-3 L-4.1 light chain variable domain sequence

SEQ ID NO: 1186 - protein, 107 aa, synthetic construct; identical to SEQ ID NO: 1109
  REGION 1..107: note = Mouse 5F3A5D4-3 light chain variable domain sequence

SEQ ID NO: 1187 - DNA, 27 bp, synthetic construct
  misc_feature 1..27: note = Mouse 5F3A5D4-V2 heavy chain variable domain Complementarity -determining region 1
  source 1..27: mol_type = other DNA
aacacattcactgaatacaccatgcac

SEQ ID NO: 1188 - DNA, 42 bp, synthetic construct; identical to SEQ ID NO: 1116
  misc_feature 1..42: note = Mouse 5F3A5D4-V2 heavy chain variable domain framework 2
  source 1..42: mol_type = other DNA

SEQ ID NO: 1189 - protein, 14 aa, synthetic construct
  REGION 1..14: note = Mouse 5F3A5D4-V2 heavy chain variable domain framework 2
WVKQSHGKSLEWIG

SEQ ID NO: 1190 - DNA, 51 bp, synthetic construct; identical to SEQ ID NO: 1118
  misc_feature 1..51: note = Mouse 5F3A5D4-V2 heavy chain variable domain Complementarity -determining region 2
  source 1..51: mol_type = other DNA

SEQ ID NO: 1191 - DNA, 96 bp, synthetic construct; identical to SEQ ID NO: 1119
  misc_feature 1..96: note = Mouse 5F3A5D4-V2 heavy chain variable domain framework 3
  source 1..96: mol_type = other DNA

SEQ ID NO: 1192 - protein, 23 aa, synthetic construct
  REGION 1..23: note = Mouse 5F3A5D4-3 light chain variable domain framework 1
DIQMTQTTSSLSASLGDRVTLSC

SEQ ID NO: 1193 - DNA, 33 bp, synthetic construct; identical to SEQ ID NO: 1124
  misc_feature 1..33: note = Mouse 5F3A5D4-2 light chain variable domain Complementarity -determining region 1
  source 1..33: mol_type = other DNA

SEQ ID NO: 1194 - DNA, 33 bp, synthetic construct; identical to SEQ ID NO: 1124
  misc_feature 1..33: note = Mouse 5F3A5D4-3 light chain variable domain Complementarity -determining region 1
  source 1..33: mol_type = other DNA

SEQ ID NO: 1195 - DNA, 45 bp, synthetic construct; identical to SEQ ID NO: 1125
  misc_feature 1..45: note = Mouse 5F3A5D4-3 light chain variable domain framework 2
  source 1..45: mol_type = other DNA

SEQ ID NO: 1196 - protein, 15 aa, synthetic construct
  REGION 1..15: note = Mouse 5F3A5D4-3 light chain variable domain framework 2
WYQQKPDGTVELLIF

SEQ ID NO: 1197 - DNA, 21 bp, synthetic construct
  misc_feature 1..21: note = Mouse 5F3A5D4-3 light chain variable domain Complementarity -determining region 2
  source 1..21: mol_type = other DNA
tacacatcaagtttacactca

SEQ ID NO: 1198 - DNA, 96 bp, synthetic construct; identical to SEQ ID NO: 1128
  misc_feature 1..96: note = Mouse 5F3A5D4-3 light chain variable domain framework 3
  source 1..96: mol_type = other DNA

SEQ ID NO: 1199 - protein, 32 aa, synthetic construct; identical to SEQ ID NO: 1129
  REGION 1..32: note = Mouse 5F3A5D4-3 light chain variable domain framework 3

SEQ ID NO: 1200 - DNA, 27 bp, synthetic construct
  misc_feature 1..27: note = Mouse 5F3A5D4-2 light chain variable domain Complementarity -determining region 3
  source 1..27: mol_type = other DNA
cagcagtatagtaagcttccttacacg

SEQ ID NO: 1201 - DNA, 27 bp, synthetic construct
  misc_feature 1..27: note = Mouse 5F3A5D4-3 light chain variable domain Complementarity -determining region 3
  source 1..27: mol_type = other DNA
cagcagtatagtaagcttccttacacg

SEQ ID NO: 1202 - DNA, 321 bp, synthetic construct; identical to SEQ ID NO: 1134
  misc_feature 1..321: note = Humanized 5F3A5D4-3 L-3.15 light chain variable domain sequence
  source 1..321: mol_type = other DNA

SEQ ID NO: 1203 - protein, 107 aa, synthetic construct; identical to SEQ ID NO: 1135
  REGION 1..107: note = Humanized 5F3A5D4-3 L-3.15 light chain variable domain sequence

SEQ ID NO: 1204 - DNA, 321 bp, synthetic construct; identical to SEQ ID NO: 1136
  misc_feature 1..321: note = Mouse 5F3A5D4-3 light chain variable domain sequence
  source 1..321: mol_type = other DNA

SEQ ID NO: 1205 - protein, 121 aa, synthetic construct; identical to SEQ ID NO: 173
  REGION 1..121: note = 8F9A4A3H

SEQ ID NO: 1206 - protein, 122 aa, synthetic construct; identical to SEQ ID NO: 1004
  REGION 1..122: note = Humanized 8F9A4A3 H-ori heavy chain variable domain sequence

SEQ ID NO: 1207 - protein, 10 aa, synthetic construct
  REGION 1..10: note = Mouse 5F3A5D4-3 light chain variable domain framework 4 (FR4) sequence
FGGGTKLEIK

SEQ ID NO: 1208 - protein, 11 aa, synthetic construct
  REGION 1..11: note = Mouse 5F3A5D4-2 light chain variable domain Complementarity -determining region 1 (CDR1) sequence
SASQGISNYLN

SEQ ID NO: 1209 - protein, 11 aa, synthetic construct
  REGION 1..11: note = Mouse 5F3A5D4-3 light chain variable domain Complementarity -determining region 1 (CDR1) sequence
SASQGISNYLN

SEQ ID NO: 1210 - protein, 7 aa, synthetic construct
  REGION 1..7: note = Mouse 5F3A5D4-2 light chain variable domain Complementarity -determining region 2 (CDR2) sequence
YTSSLHS

SEQ ID NO: 1211 - protein, 7 aa, synthetic construct
  REGION 1..7: note = Mouse 5F3A5D4-3 light chain variable domain Complementarity -determining region 2 (CDR2) sequence
YTSSLHS

SEQ ID NO: 1212 - protein, 9 aa, synthetic construct
  REGION 1..9: note = Mouse 5F3A5D4-2 light chain variable domain Complementarity -determining region 3 (CDR3) sequence
QQYSKLPYT

SEQ ID NO: 1213 - protein, 9 aa, synthetic construct
  REGION 1..9: note = Mouse 5F3A5D4-3 light chain variable domain Complementarity -determining region 3 (CDR3) sequence
QQYSKLPYT